SEQUENCE LISTING (3 sequences)

SEQ ID NO: 1 - DNA, 21 bp, Artificial Sequence
  Description of Artificial Sequence Synthetic primer
ggaacccctagtgatggagtt

SEQ ID NO: 2 - DNA, 16 bp, Artificial Sequence
  Description of Artificial Sequence Synthetic primer
cggcctcagtgagcga

SEQ ID NO: 3 - DNA, 21 bp, Artificial Sequence
  Description of Artificial Sequence Synthetic probe
cactccctctctgcgcgctcg

(19) United States
(12) Patent Application Publication
(10) Pub. No.: US 2024/0084268 A1
(43) Pub. Date: Mar. 14, 2024

(54) METHOD FOR PURIFYING RECOMBINANT VIRAL PARTICLES

(71) Applicant: ASKLEPIOS BIOPHARMACEUTICAL, INC., Research Triangle Park, NC (US)

(72) Inventors: Tamara Zekovic, Research Triangle Park, NC (US); Connor Smith, Research Triangle Park, NC (US); Paul Greback-Clarke, Research Triangle Park, NC (US); Eric Vorst, Research Triangle Park, NC (US); Eva Graham, Research Triangle Park, NC (US); Jacob Smith, Research Triangle Park, NC (US); Irnela Bajrovic, Research Triangle Park, NC (US); Jordan Hobbs, Research Triangle Park, NC (US); Robert Tikkanen, Research Triangle Park, NC (US); Josh Grieger, Research Triangle Park, NC (US)

(73) Assignee: ASKLEPIOS BIOPHARMACEUTICAL, INC., Research Triangle Park, NC (US)

(21) Appl. No.: 18/272,973

(22) PCT Filed: Jan. 21, 2022
(86) PCT No.: PCT/US22/13279

§ 371 Date: Jul. 18, 2023

Related U.S. Application Data
(60) Provisional application No. 63/139,997, filed on Jan. 21, 2021.
(60) Provisional application No. 63/222,087, filed on Jul. 15, 2021.
(60) Provisional application No. 63/282,001, filed on Nov. 22, 2021.

(51) Int. Cl. C12N 7/00; B01D 15/24; B01D 15/36; B01D 15/38; B01D 15/42

(52) U.S. Cl. CPC C12N 7/00; B01D 15/245; B01D 15/363; B01D 15/3804; B01D 15/426; C12N 2750/14123; C12N 2750/14151

(57) ABSTRACT

Provided herein are purification, production and manufacturing methods for recombinant viral vector particles such as recombinant adeno-associated viral (rAAV) vector particles substantially free of empty viral particles; a population of recombinant adeno-associated vims (rAAV) particles purified using the method described herein, and a pharmaceutical composition comprising the purified rAAV.

### CROSS-REFERENCE TO RELATED APPLICATIONS

This application is a 35 U.S.C. § 371 National Phase Entry Application of International Application No. PCT/US2022/013279 filed Jan. 21, 2022, which designated the U.S., which claims benefit under 35 U.S.C. § 119(e) of the U.S. Provisional Application No. 63/139,997, filed Jan. 21, 2021, U.S. Provisional Application No. 63/222,087, filed Jul. 15, 2021, and U.S. Provisional Application No. 63/282,001, filed Nov. 22, 2021, the contents of each of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Mar. 10, 2022, is named 046192-098780WOPT_SL.txt and is 1,056 bytes in size.

### FIELD OF THE INVENTION

The disclosure is directed generally to purification, production and manufacturing methods for recombinant viral vector particles such as recombinant adeno-associated viral (rAAV) vector particles.

### BACKGROUND

One challenge facing isolating and purification of recombinant viral vectors is ensuring an efficient separation of the functional viral particles from contaminating, closely related molecular species, such as inactive vector forms including empty and partially filled viral capsids, helper virus, and cell membrane vesicles. Thus, there is a need in the art for methods, systems and compositions for isolating and/or purifying recombinant virus particles from contaminating impurities. This disclosure addresses in part some of these needs.

### SUMMARY

In one aspect, provided herein is a method for purifying or isolating recombinantly expressed virus particles, e.g., recombinant adeno-associated virus (rAAV), optionally comprising a transgene, from a preparation comprising recombinant vector particles, empty capsids and host cell impurities, thereby providing a product substantially free of empty viral particles. Generally, the method comprises contacting a preparation, e.g., harvest media comprising recombinant virus particles with an affinity chromatography media under conditions that allow binding of virus particles to the affinity chromatography media. The bound viral particles are eluted from the affinity chromatography media using an elution buffer and recovering an eluate, comprising the eluted viral particles. In some embodiments, the affinity elution buffer comprises a weak acid or, salt thereof. In some embodiments, the affinity elution buffer is substantially free of weak acids or salts thereof. The eluate from the affinity chromatography is also referred to as affinity eluate herein.

In some embodiments of any one of the aspects, the affinity eluate comprises glycine. In some embodiments, the affinity elution buffer comprises glycine optionally, in combination with acetic acid or, a salt thereof; or, succinic acid or, a salt thereof; or, citric acid or, a salt thereof; or, propionic acid, or a salt thereof. In one aspect of the embodiment, the affinity elution buffer comprises glycine and citric acid, or a salt thereof, e.g., citrate.

In some embodiments of any one of the aspects, the affinity eluate comprises histidine. In some embodiments, the affinity elution buffer comprises histidine.

In some embodiments of any one of the aspects, the affinity eluate comprises glycine and histidine. For example, the affinity eluate comprises glycine and histidine.

In some embodiments of any one of the aspects, the eluate from the affinity chromatography comprises a predetermined amount of an anionic compound. For example, the method comprises adjusting the affinity eluate such that the adjusted eluate comprises a predetermined amount of an anionic compound. An “anionic compound” refers to a compound comprising a negatively charged moiety. The predetermined amount of the anionic compound can be at least about 0.5 mM or higher. It is noted that the anionic compound can be any suitable anionic compound. For example, the anionic compound can be an acid or a salt thereof. In some embodiments of any one of the aspects, the anionic compound is a weak acid. For example, the anionic compound is citric acid or a salt thereof, acetic acid or a salt thereof, or succinic acid or a salt thereof.

In some embodiments of any one of the aspects described herein, the method comprises adding an anionic compound to the affinity eluate. For example, the method comprises adding an acid or a salt thereof to the affinity eluate. In some embodiments of any one of the aspects, the method comprises adding a predetermined amount of a weak acid or salt thereof, e.g., citric acid or a salt thereof, acetic acid or a salt thereof, or succinic acid or a salt thereof to the affinity eluate. For example, the method comprises adding a predetermined amount of citric acid or a salt thereof, acetic acid or a salt thereof, or succinic acid or a salt thereof to the affinity eluate. In some embodiments, the method comprises adding a predetermined amount of citric acid or a salt thereof, e.g., citrate to the affinity eluate.

The affinity eluate can be diluted prior to contact with the anion exchange chromatography media. For example, the affinity eluate can be diluted by a factor of 2× or more, e.g., 3-6× or 12-15×. In some embodiments of any one of the aspects described herein, the affinity eluate can be diluted by adding a dilution buffer to the affinity eluate comprising a predetermined amount of an anionic compound. For example, the dilution buffer comprises a predetermined amount of an acid or a salt thereof. In some embodiments of any one of the aspects, the dilution buffer comprises a predetermined amount of a weak acid or a salt thereof. For example, the dilution buffer comprises a predetermined amount of citric acid or a salt thereof, acetic acid or a salt thereof, or succinic acid or a salt thereof. In some embodiments, the dilution buffer comprises a predetermined amount of citric acid or a salt thereof, e.g., citrate. The predetermined amount of the anionic compound in the dilution buffer can be at least about 0.5 mM.

The affinity eluate is contacted with an anion exchange chromatography media under conditions that allow binding of viral particles to the anion exchange chromatography media. Inventors have discovered inter alia that presence of an ionic compound, such as an anionic compound, e.g. an acid or salt thereof, such as a weak acid or a salt thereof in the buffer used for equilibrating and/or conditioning the chromatography prior to contacting with the affinity eluate surprising and unexpectedly leads to preferential binding of genome-containing viral particles, e.g., Adeno associated virus (AAV) particles having packaged genomic sequences (i.e., full viral particles) over genome-deficient AAV particles (i.e., empty). This preferential binding of full viral particle, e.g full AAV particle and thereby the preferential elimination of empty viral particles e.g, empty AAV particles is referred herein, as partitioning effect or, partition effect. Accordingly, in some embodiments of any one of the aspects, the anion exchange chromatography media is equilibrated with a buffer, e.g., an equilibration buffer for anion exchange chromatography or AEX equilibration buffer comprising a predetermined amount of an ionic compound, such as an anionic compound and/or a cationic compound. For example, the AEX equilibration buffer comprises a predetermined amount of an acid or a salt thereof, e.g., a weak acid or a salt thereof. In some embodiments of any one of the aspects, the AEX equilibration buffer comprises a predetermined amount of a citric acid or a salt thereof, acetic acid or a salt thereof, or succinic acid or a salt thereof. In some embodiments, the AEX equilibration buffer comprises a predetermined amount of citric acid or a salt thereof, e.g., citrate. In some aspects provided herein, the addition of an ionic compound, such as an anionic compound, e.g. an acid or salt thereof, such as a weak acid or a salt thereof in the dilution buffer used for diluting the Affinity eluate results in preferential elimination of empty viral particles e.g, empty AAV particles from binding the anion exchange chromatography. In some embodiments, the addition of an ionic compound, such as an anionic compound, e.g. an acid or salt thereof, such as a weak acid or a salt thereof in the buffer used for equilibrating and/or conditioning the chromatography prior to contacting with the affinity eluate; and/or, the addition of an ionic compound, such as an anionic compound, e.g. an acid or salt thereof, such as a weak acid or a salt thereof in the dilution buffer used for diluting the Affinity eluate results in preferential elimination of empty viral particles e.g, empty AAV particles from binding the anion exchange chromatography, ie. results in the partitioning effect.

The bound viral particles can be eluted from the anion exchange chromatography media using an appropriate elution buffer. Generally, at least about 70%, or, at least about 75% or, at least about 80%, or more of the viral particles in the anion exchange eluate are full rAAV viral particle. In some preferred embodiments, at least about 85%, Or at least about 86%, or, at least about 88%, or at least about 90%, at least about 92%, at least about 95% or more of the viral particles in the anion exchange eluate are full rAAV viral particle. For example, less than 30%, less than 20%, less than 19.5%, less than 19%, less than 18.5%, less than 18%, less than 17.5%, less than 17%, less than 16.5%, less than 16%, less than 15.5%, less than 15%, less than 14.5%, less than 14%, less than 13.5%, less than 13%, less than 12.5%, less than 12%, less than 11.5%, less than 11%, less than 10.5%, less than 10%, less than 9.5%, less than 9%, less than 8.5%, less than 8%, less than 7.5%, less than 7%, less than 6.5%, less than 6%, less than 5.5%, less than 5%, less than 4.5%, less than 4%, less than 3.5%, less than 3%, less than 2.5%, less than 2%, less than 1.5%, less than 1%, less than 0.75%, less than 0.5%, less than 0.25%, less than 0.2%, less than 0.15%, less than 0.1%, or, less than 0.05% of the AAV viral particles in the eluate from the anion exchange are empty viral particles. In some embodiments, the anion exchange eluate comprises less than 0.04%, or, preferably less than 0.02% empty AAV viral particle. In some embodiments, the eluate from anion exchange is substantially free of empty AAV viral particles. In some embodiments, from about 10% to about 15% of the AAV viral particles in the eluate from the anion exchange are empty viral particles. In some embodiments, the method described herein produces AAV particles where the empty particle is reduced by more than 86 fold, or, more than 90 fold, or, more than 95 fold or, preferably more than 99 fold. In some embodiments, 30% or less, or 25% or less, or 20% or less, or 15% or less, or 10% or less, or 5% or less, or even less of the AAV purified with the method disclosed herein are partially filled AAV particle. In one aspect of the embodiment, the eluate of the anion exchange column comprises less than 11%, less than 10%, less than 9%, less than 8%, less than 6% or, less than 5% of partially filled AAV particle.

In some embodiments of any one of the aspects described herein, a ratio of total rAAV viral particles (e.g., full, partially full and empty AAV viral particles) to empty rAAV viral particles in the anion exchange eluate is at least about 1.25× higher than the ratio of total rAAV viral particles to empty particles in the affinity eluate. For example, the ratio of total rAAV viral particles to empty rAAV viral particles in the anion exchange eluate is at least about 1.5×, at least about 1.6×, at least about 1.7×, at least about 1.8×, at least about 1.9×, at least about 2×, at least about 2.1×, at least about 2.2×, at least about 2.3×, at least about 2.4×, at least about 2.5×, at least about 2.6×, at least about 2.7×, at least about 2.8×, at least about 2.9×, at least about 3×, at least about 3.1×, at least about 3.2×, at least about 3.3×, at least about 3.4×, at least about 3.5×, at least about 3.6×, at least about 3.7×, at least about 3.8×, at least about 3.9×, at least about 4×, at least about 4.1×, at least about 4.2×, at least about 4.3×, at least about 4.4×, at least about 4.5×, at least about 4.6×, at least about 4.7×, at least about 4.8×, at least about 4.9×, at least about 5×, at least about 5.1×, at least about 5.2×, at least about 5.3×, at least about 5.4×, at least about 5.5×, at least about 5.6×, at least about 5.7×, at least about 5.8×, at least about 5.9×, at least about 6×, at least about 6.1×, at least about 6.2×, at least about 6.3×, at least about 6.4×, at least about 6.5×, at least about 6.6×, at least about 6.7×, at least about 6.8×, at least about 6.9×, at least about 7×, at least about 7.1×, at least about 7.2×, at least about 7.3×, at least about 7.4×, at least about 7.5×, at least about 7.6×, at least about 7.7×, at least about 7.8×, at least about 7.9×, at least about 8×, at least about 8.1×, at least about 8.2×, at least about 8.3×, at least about 8.4×, at least about 8.5×, at least about 8.6×, at least about 8.7×, at least about 8.8×, at least about 8.9×, at least about 9×, at least about 9.1×, at least about 9.2×, at least about 9.3×, at least about 9.4×, at least about 9.5×, at least about 9.6×, at least about 9.7×, at least about 9.8×, at least about 9.9×, or at least about 10× higher relative to the ratio of total rAAV viral particles to empty rAAV particles in the affinity eluate.

In some embodiments, the ratio of total rAAV viral particles to empty rAAV viral particles in the anion exchange eluate is at least about 2×, at least about 2.5×, at least about 3×, at least about 3.5×, at least about 4×, at least about 4.5×, at least about 5×, at least about 5.5×, at least about 6×, at least about 6.5×, at least about 7×, at least about 7.5×, at least about 8×, at least about 8.5×, at least about 9×, at least at least about 9.5×, or at least about 10× higher relative to the ratio of total rAAV viral particles to empty rAAV particles in the affinity eluate. For example, the ratio of total rAAV viral particles to empty rAAV particles in the purified population is at least about 2.5× higher relative to the ratio of total rAAV viral particles to empty rAAV particles in the affinity eluate. In another non-limiting example, the ratio of total rAAV viral particles to empty rAAV viral particles in the purified population is at least about 3× higher relative to the ratio of total rAAV viral particles to empty rAAV viral particles in the affinity eluate. Yet in another non-limiting example, the ratio of total rAAV viral particles to empty rAAV viral particles in the purified population is at least about 3.5× higher relative to the ratio of total rAAV viral particles to empty rAAV viral particles in the affinity eluate. Still in another non-limiting example, the ratio of total rAAV viral particles to empty rAAV viral particles in the purified population is at least about 4× higher relative to the ratio of total rAAV viral particles to empty rAAV viral particles in the affinity eluate. Yet still in another non-limiting example, the ratio of total rAAV viral particles to empty rAAV viral particles in the purified population is at least about 5× higher relative to the ratio of total rAAV viral particles to empty rAAV viral particles in the affinity eluate.

In some embodiments of any one of the aspects described herein, a ratio of full and partially full rAAV particles to empty rAAV viral particles in the anion exchange eluate is at least about 1.25× higher than the ratio of full and partially full rAAV particles to empty particles in the affinity eluate. For example, the ratio of full and partially full rAAV particles to empty rAAV viral particles in the anion exchange eluate is at least about 1.5×, at least about 1.6×, at least about 1.7×, at least about 1.8×, at least about 1.9×, at least about 2×, at least about 2.1×, at least about 2.2×, at least about 2.3×, at least about 2.4×, at least about 2.5×, at least about 2.6×, at least about 2.7×, at least about 2.8×, at least about 2.9×, at least about 3×, at least about 3.1×, at least about 3.2×, at least about 3.3×, at least about 3.4×, at least about 3.5×, at least about 3.6×, at least about 3.7×, at least about 3.8×, at least about 3.9×, at least about 4×, at least about 4.1×, at least about 4.2×, at least about 4.3×, at least about 4.4×, at least about 4.5×, at least about 4.6×, at least about 4.7×, at least about 4.8×, at least about 4.9×, at least about 5×, at least about 5.1×, at least about 5.2×, at least about 5.3×, at least about 5.4×, at least about 5.5×, at least about 5.6×, at least about 5.7×, at least about 5.8×, at least about 5.9×, at least about 6×, at least about 6.1×, at least about 6.2×, at least about 6.3×, at least about 6.4×, at least about 6.5×, at least about 6.6×, at least about 6.7×, at least about 6.8×, at least about 6.9×, at least about 7×, at least about 7.1×, at least about 7.2×, at least about 7.3×, at least about 7.4×, at least about 7.5×, at least about 7.6×, at least about 7.7×, at least about 7.8×, at least about 7.9×, at least about 8×, at least about 8.1×, at least about 8.2×, at least about 8.3×, at least about 8.4×, at least about 8.5×, at least about 8.6×, at least about 8.7×, at least about 8.8×, at least about 8.9×, at least about 9×, at least about 9.1×, at least about 9.2×, at least about 9.3×, at least about 9.4×, at least about 9.5×, at least about 9.6×, at least about 9.7×, at least about 9.8×, at least about 9.9×, or at least about 10× higher relative to the ratio of full and partially full rAAV particles to empty rAAV particles in the affinity eluate.

In some embodiments, the ratio of full and partially full rAAV particles to empty rAAV viral particles in the anion exchange eluate is at least about 2×, at least about 2.5×, at least about 3×, at least about 3.5×, at least about 4×, at least about 4.5×, at least about 5×, at least about 5.5×, at least about 6×, at least about 6.5×, at least about 7×, at least about 7.5×, at least about 8×, at least about 8.5×, at least about 9×, at least at least about 9.5×, or at least about 10× higher relative to the ratio of full and partially full rAAV particles to empty rAAV particles in the affinity eluate. For example, the ratio of full and partially full rAAV particles to empty rAAV particles in the purified population is at least about 2.5× higher relative to the ratio of full and partially full rAAV particles to empty rAAV particles in the affinity eluate. In another non-limiting example, the ratio of full and partially full rAAV particles to empty rAAV viral particles in the purified population is at least about 3× higher relative to the ratio of full and partially full rAAV particles to empty rAAV viral particles in the affinity eluate. Yet in another non-limiting example, the ratio of full and partially full rAAV particles to empty rAAV viral particles in the purified population is at least about 3.5× higher relative to the ratio of full and partially full rAAV particles to empty rAAV viral particles in the affinity eluate. Still in another non-limiting example, the ratio of full and partially full rAAV particles to empty rAAV viral particles in the purified population is at least about 4× higher relative to the ratio of full and partially full rAAV particles to empty rAAV viral particles in the affinity eluate. Yet still in another non-limiting example, the ratio of full and partially full rAAV particles to empty rAAV viral particles in the purified population is at least about 5× higher relative to the ratio of full and partially full rAAV particles to empty rAAV viral particles in the affinity eluate.

In some embodiments, a UV₂₆₀ to UV₂₈₀ ratio of the anion exchange eluate is at least 1.25 or more. For example, the UV₂₆₀ to UV₂₈₀ ratio of the anion exchange eluate is at least 1.25, at least 1.26, at least 1.27, at least 1.28, at least 1.29, at least 1.3, at least 1.31, at least 1.32, at least 1.33, at least 1.34, or at least 1.35 or more.

In some embodiments, a UV₂₆₀ to UV₂₈₀ ratio of the affinity eluate is 1.15 less. For example, the UV₂₆₀ to UV₂₈₀ ratio of the affinity eluate is 1.10 or less, 1.05 or less, 1 or less, 0.95 or less, 0.9 or less, 0.85 or less, or 0.8 or less.

In some embodiments, the UV₂₆₀ to UV₂₈₀ ratio of the affinity eluate is 1.15 or less and the UV₂₆₀ to UV₂₈₀ ratio of the anion exchange eluate obtained from said affinity eluate is at least 1.25 or more. For example, the UV₂₆₀ to UV₂₈₀ ratio of the affinity eluate is 1.10 or less and the UV₂₆₀ to UV₂₈₀ ratio of the anion exchange eluate obtained from said affinity eluate is at least 1.25 or more. In some embodiments, the UV₂₆₀ to UV₂₈₀ ratio of the affinity eluate is 1.05 or less and the UV₂₆₀ to UV₂₈₀ ratio of the anion exchange eluate obtained from said affinity eluate is at least 1.30 or more.

In some embodiments, a ratio of UV₂₆₀ to UV₂₈₀ in the anion exchange eluate is at least about 1.15× higher than a ratio of UV₂₆₀ to UV₂₈₀ ratio in the adjusted affinity eluate. For example, the ratio of UV₂₆₀ to UV₂₈₀ in the anion exchange eluate is at least about 1.2×, at least about 1.25×, at least about 1.3×, at least about 1.35×, at least about 1.4×, at least about 1.45×, at least about 1.5×, at least about 1.55×, at least about 1.6×, at least about 1.65×, at least at least about 1.7×, or at least about 1.75×, at least about 1.8×, at least about 1.859×, at least about 1.9×, at least about 1.95×, or at least about 2× or higher than the ratio of UV₂₆₀ to UV₂₈₀ ratio in the adjusted affinity eluate. In some embodiments, the ratio of UV₂₆₀ to UV₂₈₀ in the anion exchange eluate is at least about 1.15× to at least about at least about 2× or higher, or at least about 1.15× to about at least about 1.95× or higher, or at least about 1.15× to about at least about 1.9× or higher, or at least about 1.15× to about at least about 1.85× or higher, or at least about 1.15× to about at least about 1.8× or higher, or at least about 1.15× to about at least about 1.75× or higher, or at least about 1.15× to about at least about 1.7× or higher, or at least about 1.15× to about at least about 1.65× or higher, or at least about 1.15× to about at least about 1.6× or higher, or at least about 1.15× to about at least about 1.55× or higher, or at least about 1.15× to about at least about 1.5× or higher, or at least about 1.15× to about at least about 1.45× or higher, or at least about 1.15× to about at least about 1.4× or higher, or at least about 1.15× to about at least about 1.35× or higher, or at least about 1.15× to about at least about 1.3× or higher, or at least about 1.15× to about at least about 1.25× or higher, or at least about 1.15× to about at least about 1.2× or higher, or at least about 1.15× to about at least about 1.15× or higher than the ratio of UV₂₆₀ to UV₂₈₀ ratio in the adjusted affinity eluate.

The method of purifying or, isolating AAV particles as described herein allows binding of less empty particle to the anion exchange column and, the flow through of anion exchange column comprises more empty particles. In some embodiments, less than 10%, less than 8%, less than 6%, less than 5%, less than 4%, less than 3%, less than 2%, less than 1%, less than 0.5%, less than 0.2%, less than 0.1%, less than 0.05%, or, less than 0.02% or, even less empty AAV particles bind to anion exchange column. In preferred embodiments, empty AAV particles do not bind to Anion exchange column. In some aspects of the embodiment, anion exchange column flow-through comprises at least 5%, at least 6%, at least 8%, at least 10%, at least 15%, or, at least 20%, or, more of the empty AAV particle.

In some embodiments, the presence of amino acids or, the presence of amino acid in combination with a weak acid or, a salt thereof, in the affinity elution buffer is used to purify the recombinant AAV particle. In some aspect of the embodiments, the affinity elution buffer comprises glycine optionally, in combination with acetic acid or, a salt thereof; or, succinic acid or, a salt thereof; or, citric acid or, a salt thereof; or, propionic acid, or a salt thereof. In one aspect of the embodiment, the affinity elution buffer comprises glycine and citric acid, or a salt thereof, e.g citrate. In another aspect of the embodiment, the affinity elution buffer comprises glycine with other amino acids e.g. with histidine. In some embodiments, the affinity eluate as disclosed herein comprises from about 10% to about 50% empty AAV particles in the affinity eluate. For example, the affinity eluate as disclosed herein comprises less than 50%, less than 45%, less than 40%, less than 35%, less than 30%, less than 25%, less than 20%, less than 15%, less than 10%, less than 8%, or less than 5% empty AAV particles in the affinity eluate.

The methods disclosed herein can comprise producing the harvest media for contacting with the affinity chromatography media by a method comprising upstream processing such as, for example, harvest of a cell culture and/or clarification of the harvested cell culture. Accordingly, in any one of the aspect, the method comprises a step of clarification of a cell culture media. For example, clarification of the harvested cell culture by depth filtration.

In some embodiments of any one of the aspects, the method comprises a step of lysing a host cell in the harvested cell culture prior to clarification. Methods and compositions for lysing host cells are well known in the art. For example, a surfactant, e.g., a non-ionic surfactant, can be added to the harvested cell culture for lysing a host cell present in the harvested cell culture. In some embodiments of all aspects, the methods do not comprise a step of lysing a host cell in the harvested cell culture prior to clarification. In one aspect of the embodiment, the recombinant viral particle (rAAV) is isolated or purified from the supernatant of host cell culture.

The harvested cell culture may comprise impurities, e.g., host cell DNA (hcDNA). Therefore, the method can comprise a step of removing or reducing amount of impurities from the harvested cell culture, e.g., prior to the clarification step. Methods and compositions for reducing the amount of host cell DNA in harvest media are well known in the art. For example, a cationic amine or nuclease can be added to the harvest media.

In some embodiments, the methods disclosed herein further encompass processing the viral particles eluted from the anion exchange chromatography by downstream processing steps such as, for example, tangential flow filtration and/or sterile filtration, or any combinations thereof. It is noted that the upstream and/or downstream processing can be used alone or in various combinations.

The method described herein is easily adaptable for different viruses and serotypes. Thus, in some embodiments of any one of the aspects, the recombinant virus particles are recombinant adeno associated virus (rAAV) particles. For example, the rAAV particles can be AAV-1, AAV-2, AAV-2i8, AAV-3, AAV-4, AAV-5, AAV-6, AAV-7, AAV-8, AAV-9, AAV-10, AAVrh10, AAV-11, AAV-12, AAV-13, AAV-14, AAV-15, AAV-16 or a chimera, derivative, modification, or pseudotype thereof. In some embodiments, the rAAV is a rational polyploid (also referred as haploid) AAV. In some embodiments, the method described herein is used to purify/isolate rAAV, wherein the rAAV comprises at least one capsid protein (e.g., VP1, VP2, or, VP3) from the AAV serotypes listed in Table 1.

TABLE 1
AAV Serotypes and exemplary published corresponding capsid sequence

| Serotype and where capsid | Serotype and where capsid |
|---|---|
| sequence is published | sequence is published |
| AAV3.3b (See SEQ ID NO: 72 in US20030138772) | AAV3-3 (See SEQ ID NO: 200 US20150315612) |
| AAV3-3 (See SEQ ID NO: 217 US20150315612) | AAV3a ((See SEQ ID NO: 5 in U.S. Pat. No. 6,156,303) |
| AAV3a (See SEQ ID NO: 9 in U.S. Pat. No. 6,156,303) | AAV3b (See SEQ ID NO: 6 in U.S. Pat. No. 6,156,303) |
| AAV3b (See SEQ ID NO: 10 in U.S. Pat. No. 6,156,303) | AAV3b (See SEQ ID NO: 1 in U.S. Pat. No. 6,156,303) |
| AAV4 (See SEQ ID NO: 17 US20140348794) | AAV4 ((See SEQ ID NO: 5 in US20140348794) |
| AAV4 (See SEQ ID NO: 3 in US20140348794) | AAV4 (See SEQ ID NO: 14 in US20140348794) |
| AAV4 (See SEQ ID NO: 15 in US20140348794) | AAV4 (See SEQ ID NO: 19 in US20140348794) |
| AAV4 (See SEQ ID NO: 12 in US20140348794) | AAV4 (See SEQ ID NO: 13 in US20140348794) |
| AAV4 (See SEQ ID NO: 7 in US20140348794) | AAV4 (See SEQ ID NO: 8 in US20140348794) |
| AAV4 (See SEQ ID NO: 9 in US20140348794) | AAV4 (See SEQ ID NO: 2 in US20140348794) |
| AAV4 (See SEQ ID NO: 10 in US20140348794) | AAV4 (See SEQ ID NO: 11 in US20140348794) |
| AAV4 (See SEQ ID NO: 18 in US20140348794) | AAV4 (See SEQ ID NO: 63 in |
| | US20030138772) and US20160017295 SEQ |
| ID NO: (See SEQ ID NO: 4 in US20140348794) | AAV4 (See SEQ ID NO: 16 in US20140348794) |
| AAV4 (See SEQ ID NO: 20 in US20140348794) | AAV4 (See SEQ ID NO: 6 in US20140348794) |
| AAV4 (See SEQ ID NO: 1 in US20140348794) | AAV42.2 (See SEQ ID NO: 9 in US20030138772) |
| AAV42.2 (See SEQ ID NO: 102 in US20030138772) | AAV42.3b (See SEQ ID NO: 36 in US20030138772) |
| AAV42.3B (See SEQ ID NO: 107 in US20030138772) | AAV42.4 (See SEQ ID NO: 33 in US20030138772) |
| AAV42.4 (See SEQ ID NO: 88 in US20030138772) | AAV42.8 (See SEQ ID NO: 27 in US20030138772) |
| AAV42.8 (See SEQ ID NO: 85 in US20030138772) | AAV43.1 (See SEQ ID NO: 39 in US20030138772) |
| AAV43.1 (See SEQ ID NO: 92 in US20030138772) | AAV43.12 (See SEQ ID NO: 41 in US20030138772) |
| AAV43.12 (See SEQ ID NO: 93 in US20030138772) | AAV8 (See SEQ ID NO: 15 in US20150159173) |
| AAV8 (See SEQ ID NO: 7 in US20150376240) | AAV8 (See SEQ ID NO: 4 in US20030138772; |
| | US20150315612 SEQ |
| ID NO: 182 | AAV8 (See SEQ ID NO: 95 in |
| | US20030138772), US20140359799 SEQ |
| AAV8 (See SEQ ID NO: 31 in US20150159173) | AAV8 (See, e.g., SEQ ID NO: 8 in |
| | US20160017295, or SEQ ID NO: 7 in |
| | U.S. Pat. No. 7,198,951, or SEQ ID NO: 223 in |
| | US20150315612) |
| AAV8 (See SEQ ID NO: 8 in US20150376240) | AAV8 (See SEQ ID NO: 214 in US20150315612) |
| AAV-8b (See SEQ ID NO: 5 in US20150376240) | AAV-8b (See SEQ ID NO: 3 in US20150376240) |
| AAV-8h (See SEQ ID NO: 6 in US20150376240) | AAV-8h (See SEQ ID NO: 4 in US20150376240) |
| AAV9 (See SEQ ID NO: 5 in US20030138772) | AAV9 (See SEQ ID NO: 1 in U.S. Pat. No. 7,198,951) |
| AAV9 (See SEQ ID NO: 9 in US20160017295) | AAV9 (See SEQ ID NO: 100 in |
| | US20030138772), U.S. Pat. No. 7,198,951 SEQ ID NO: 2 |
| | AAV9 (See SEQ ID NO: 3 in U.S. Pat. No. 7,198,951) |
| AAV9 (AAVhu.14) (See SEQ ID NO: 3 in | AAV9 (AAVhu.14) (See SEQ ID NO: 123 in |
| US20150315612) | US20150315612) |
| AAVA3.1 (See SEQ ID NO: 120 in | AAVA3.3 (See SEQ ID NO: 57 in |
| US20030138772) | US20030138772) |
| AAVA3.3 (See SEQ ID NO: 66 in | AAVA3.4 (See SEQ ID NO: 54 in |
| US20030138772) | US20030138772) |
| AAVA3.4 (See SEQ ID NO: 68 in | AAVA3.5 (See SEQ ID NO: 55 in |
| US20030138772) | US20030138772) |
| AAVA3.5 (See SEQ ID NO: 69 in | AAVA3.7 (See SEQ ID NO: 56 in |
| US20030138772) | US20030138772) |
| AAVA3.7 (See SEQ ID NO: 67 in | AAV29. (See SEQ ID NO: 11 in (AAVbb. I) |
| US20030138772) | 161 US20030138772) |
| AAVC2 (See SEQ ID NO: 61 in US20030138772) | AAVCh.5 (See SEQ ID NO: 46 in |
| | US20150159173); US20150315612 SEQ |
| ID NO: 234 | AAVcy.2 (AAV13.3) (See SEQ ID NO: 15 in |
| | US20030138772) |
| AAV24.1 (See SEQ ID NO: 101 in | AAVcy.3 (AAV24.1) (See SEQ ID NO: 16 in |
| US20030138772) | US20030138772) |
| AAV27.3 (See SEQ ID NO: 104 in | AAVcy.4 (AAV27.3) (See SEQ ID NO: 17 in |
| US20030138772) | US20030138772) |
| AAVcy.5 (See SEQ ID NO: 227 in | AAV7.2 (See SEQ ID NO: 103 in |
| US20150315612) | US20030138772) |
| AAVcy.5 (AAV7.2) (See SEQ ID NO: 18 in | AAV16.3 (See SEQ ID NO: 105 in |
| US20030138772) | US20030138772) |
| AAVcy.6 (AAV16.3) (See SEQ ID NO: 10 in | AAVcy.5 (See SEQ ID NO: 8 in |
| US20030138772) | US20150159173) |
| AAVcy.5 (See SEQ ID NO: 24 in | AAVCy.5RI (See SEQ ID NO: in |
| US20150159173) | US20150159173 |
| AAVCy.5R2 (See SEQ ID NO: in | AAVCy.5R3 (See SEQ ID NO: in |
| US20150159173) | US20150159173 |
| AAVCy.5R4 (See SEQ ID NO: in | AAVDJ (See SEQ ID NO: 3 in |
| US20150159173) | US20140359799) and SEQ ID NO: 2 in |
| | U.S. Pat. No. 7,588,772) |
| | AAVDJ (See SEQ ID NO: 2 in |
| | US20140359799; and SEQ ID NO: 1 in |
| | U.S. Pat. No. 7,588,772) |
| | AAVDJ-8 (See SEQ ID NO: in U.S. Pat. No. 7,588,772; |
| | Grimm et al 2008 |
| AAVDJ-8 (See SEQ ID NO: in U.S. Pat. No. 7,588,772; | AAVF5 (See SEQ ID NO: 110 in |
| Grimm et al 2008 | US20030138772) |
| AAVH2 (See SEQ ID NO: 26 in US20030138772) | AAVH6 (See SEQ ID NO: 25 in |
| | US20030138772) |
| AAVhEI.I (See SEQ ID NO: 44 in U.S. Pat. No. 9,233,131) | AAVhErI.14 (See SEQ ID NO: 46 in |
| | U.S. Pat. No. 9,233,131) |
| AAVhErI.16 (See SEQ ID NO: 48 in U.S. Pat. No. 9,233,131) | AAVhErI.18 (See SEQ ID NO: 49 in |
| | U.S. Pat. No. 9,233,131) |
| AAVhErI.23 (AAVhEr2.29) (See SEQ ID NO: 53 | AAVhErI.35 (See SEQ ID NO: 50 in |
| in U.S. Pat. No. 9,233,131) | U.S. Pat. No. 9,233,131) |
| AAVhErI.36 (See SEQ ID NO: 52 in U.S. Pat. No. 9,233,131) | AAVhErI.5 (See SEQ ID NO: 45 in |
| | U.S. Pat. No. 9,233,131) |
| AAVhErI.7 (See SEQ ID NO: 51 in U.S. Pat. No. 9,233,131) | AAVhErI.8 (See SEQ ID NO: 47 in |
| | U.S. Pat. No. 9,233,131) |
| AAVhEr2.16 (See SEQ ID NO: 55 in U.S. Pat. No. 9,233,131) | AAVhEr2.30 (See SEQ ID NO: 56 in |
| | U.S. Pat. No. 9,233,131) |
| AAVhEr2.31 (See SEQ ID NO: 58 in U.S. Pat. No. 9,233,131) | AAVhEr2.36 (See SEQ ID NO: 57 in |
| | U.S. Pat. No. 9,233,131) |
| AAVhEr2.4 (See SEQ ID NO: 54 in U.S. Pat. No. 9,233,131) | AAVhEr3.1 (See SEQ ID NO: 59 in |
| | U.S. Pat. No. 9,233,131) |
| AAVhu.I (See SEQ ID NO: 46 in US20150315612) | AAVhu.I (See SEQ ID NO: 144 in |
| | US20150315612) |
| AAVhu.IO (AAV16.8) (See SEQ ID NO: 56 in | AAVhu.IO (AAV16.8) (See SEQ ID NO: 156 |
| US20150315612) | in US20150315612) |
| AAVhu.I I(AAV16.12) (See SEQ ID NO: 57 in | AAVhu.I I(AAV16.12) (See SEQ ID NO: 153 |
| US20150315612) | in US20150315612) |
| AAVhu.12 (See SEQ ID NO: 59 in | AAVhu.12 (See SEQ ID NO: 154 in |
| US20150315612) | US20150315612) |
| AAVhu.13 (See SEQ ID NO: 16 in | |
| US2015015917 and ID NO: 71 in US20150315612) | |
| AAVhu.13 (See SEQ ID NO: 32 in | |
| US20150159173 and ID NO: 129 US20150315612) | |
| AAVhu.136.1 (See SEQ ID NO: 165 in | AAVhu.140.1 (See SEQ ID NO: 166 in |
| US20150315612) | US20150315612) |
| AAVhu.140.2 (See SEQ ID NO: 167 in | AAVhu.145.6 (See SEQ ID NO: 178 in |
| US20150315612) | US20150315612) |
| AAVhu.15 (See SEQ ID NO: 147 in | AAVhu.15 (AAV33.4) (See SEQ ID NO: 50 in |
| US20150315612) | US20150315612) |
| AAVhu.156.1 (See SEQ ID NO: 179 in | AAVhu.16 (See SEQ ID NO: 148 in |
| US20150315612) | US20150315612) |
| AAVhu.I6 (AAV33.8) (See SEQ ID NO: 51 in | AAVhu.17 (See SEQ ID NO: 83 in |
| US20150315612) | US20150315612) |
| AAVhu.I7 (AAV33.12) (See SEQ ID NO: 4 in | AAVhu.172.1 (See SEQ ID NO: 171 in |
| US20150315612) | US20150315612) |
| AAVhu.172.2 (See SEQ ID NO: 172 in | AAVhu.173.4 (See SEQ ID NO: 173 in |
| US20150315612) | US20150315612) |
| AAVhu.173.8 (See SEQ ID NO: 175 in | AAVhu.18 (See SEQ ID NO: 52 in |
| US20150315612) | US20150315612) |
| AAVhu.18 (See SEQ ID NO: 149 in | AAVhu.19 (See SEQ ID NO: 62 in |
| US20150315612) | US20150315612) |
| AAVhu.19 (See SEQ ID NO: 133 in | AAVhu.2 (See SEQ ID NO: 48 in |
| US20150315612) | US20150315612) |
| AAVhu.2 (See SEQ ID NO: 143 in | AAVhu.20 (See SEQ ID NO: 63 in |
| US20150315612) | US20150315612) |
| AAVhu.20 (See SEQ ID NO: 134 in | AAVhu.21 (See SEQ ID NO: 65 in |
| US20150315612) | US20150315612) |
| AAVhu.21 (See SEQ ID NO: 135 in | AAVhu.22 (See SEQ ID NO: 67 in |
| US20150315612) | US20150315612) |
| AAVhu.22 239 (See SEQ ID NO: 138 in | AAVhu.23 (See SEQ ID NO: 60 in |
| US20150315612) | US20150315612) |
| AAVhu.23.2 (See SEQ ID NO: 137 in | AAVhu.24 (See SEQ ID NO: 66 in |
| US20150315612) | US20150315612) |
| AAVhu.24 (See SEQ ID NO: 136 in | AAVhu.25 (See SEQ ID NO: 49 in |
| US20150315612) | US20150315612) |
| AAVhu.25 (See SEQ ID NO: 146 in | AAVhu.26 (See SEQ ID NO: 17 in |
| US20150315612) | US20150159173 and SEQ ID NO: 61 in |
| | US20150315612) |
| | AAVhu.26 (See SEQ ID NO: 33 in |
| | US20150159173), US20150315612 SEQ |
| | AAVhu.27 (See SEQ ID NO: 64 in |
| | US20150315612) |
| AAVhu.27 (See SEQ ID NO: 140 in | AAVhu.28 (See SEQ ID NO: 68 in |
| US20150315612) | US20150315612) |
| AAVhu.28 (See SEQ ID NO: 130 in | AAVhu.29 (See SEQ ID NO: 69 in |
| US20150315612) | US20150315612) |
| AAVhu.29 (See SEQ ID NO: 42 in | |
| US20150159173 and SEQ ID NO: 132 in | |
| US20150315612) | |
| AAVhu.29 (See SEQ ID NO: 225 in | AAVhu.29R (See SEQ ID NO: in |
| US20150315612) | US20150159173 |
| AAVhu.3 (See SEQ ID NO: 44 in | AAVhu.3 (See SEQ ID NO: 145 in |
| US20150315612) | US20150315612) |
| AAVhu.30 (See SEQ ID NO: 70 in | AAVhu.30 (See SEQ ID NO: 131 in |
| US20150315612) | US20150315612) |
| AAVhu.31 (See SEQ ID NO: 1 in | AAVhu.31 (See SEQ ID NO: 121 in |
| US20150315612) | US20150315612) |
| AAVhu.32 (See SEQ ID NO: 2 in | AAVhu.32 (See SEQ ID NO: 122 in |
| US20150315612) | US20150315612) |
| AAVhu.33 (See SEQ ID NO: 75 in | AAVhu.33 (See SEQ ID NO: 124 in |
| US20150315612) | US20150315612) |
| AAVhu.34 (See SEQ ID NO: 72 in | AAVhu.34 (See SEQ ID NO: 125 in |
| US20150315612) | US20150315612) |
| AAVhu.35 (See SEQ ID NO: 73 in | AAVhu.35 (See SEQ ID NO: 164 in |
| US20150315612) | US20150315612) |
| AAVhu.36 (See SEQ ID NO: 74 in | AAVhu.36 (See SEQ ID NO: 126 in |
| US20150315612) | US20150315612) |
| AAVhu.37 (See SEQ ID NO: 34 in | |
| US20150159173 and SEQ ID NO: 88 in | |
| US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in | AAVhu.39 (See SEQ ID NO: 102 in |
| US20150315612) | US20150315612) |
| AAVhu.39 (AAVLG-9) (See SEQ ID NO: 24 in | AAVhu.4 (See SEQ ID NO: 47 in |
| US20150315612) | US20150315612) |
| AAVhu.4 (See SEQ ID NO: 141 in | AAVhu.40 (See SEQ ID NO: 87 in |
| US20150315612) | US20150315612) |
| AAVhu.40 (AAV114.3) (See SEQ ID NO: 11 in | AAVhu.41 (See SEQ ID NO: 91 in |
| US20150315612) | US20150315612) |
| AAVhu.41 (AAV127.2) (See SEQ ID NO: 6 in | AAVhu.42 (See SEQ ID NO: 85 in |
| US20150315612) | US20150315612) |
| AAVhu.42 (AAV127.5) (See SEQ ID NO: 8 in | AAVhu.43 (See SEQ ID NO: 160 in |
| US20150315612) | US20150315612) |
| AAVhu.43 (See SEQ ID NO: 236 in | AAVhu.43 (AAV128.1) (See SEQ ID NO: 80 |
| US20150315612) | in US20150315612) |
| AAVhu.44 (See SEQ ID NO: 45 in | |
| US20150159173 and SEQ ID NO: 158 in | |
| US20150315612) | |
| AAVhu.44 (AAV128.3) (See SEQ ID NO: 81 in | AAVhu.44RI (See SEQ ID NO: in |
| US20150315612) | US20150159173 |
| AAVhu.44R2 (See SEQ ID NO: in | AAVhu.44R3 (See SEQ ID NO: in |
| US20150159173 | US20150159173 |
| AAVhu.45 (See SEQ ID NO: 76 in | AAVhu.45 (See SEQ ID NO: 127 in |
| US20150315612) | US20150315612) |
| AAVhu.46 (See SEQ ID NO: 82 in | AAVhu.46 (See SEQ ID NO: 159 in |
| US20150315612) | US20150315612) |
| AAVhu.46 (See SEQ ID NO: 224 in | AAVhu.47 (See SEQ ID NO: 77 in |
| US20150315612) | US20150315612) |
| AAVhu.47 (See SEQ ID NO: 128 in | AAVhu.48 (See SEQ ID NO: 38 in |
| US20150315612) | US20150159173) |
| AAVhu.48 (See SEQ ID NO: 157 in | AAVhu.48 (AAV130.4) (See SEQ ID NO: 78 |
| US20150315612) | in US20150315612) |
| AAVhu.48RI (See SEQ ID NO: in | AAVhu.48R2 (See SEQ ID NO: in |
| US20150159173 | US20150159173 |
| AAVhu.48R3 (See SEQ ID NO: in | AAVhu.49 (See SEQ ID NO: 209 in |
| US20150159173 | US20150315612) |
| AAVhu.49 (See SEQ ID NO: 189 in | AAVhu.5 (See SEQ ID NO: 45 in |
| US20150315612) | US20150315612) |
| AAVhu.5 (See SEQ ID NO: 142 in | AAVhu.51 (See SEQ ID NO: 208 in |
| US20150315612) | US20150315612) |
| AAVhu.51 (See SEQ ID NO: 190 in | AAVhu.52 (See SEQ ID NO: 210 in |
| US20150315612) | US20150315612) |
| AAVhu.52 (See SEQ ID NO: 191 in | AAVhu.53 (See SEQ ID NO: 19 in |
| US20150315612) | US20150159173) |
| AAVhu.53 (See SEQ ID NO: 35 in | AAVhu.53 (AAV145.1) (See SEQ ID NO: 176 |
| US20150159173) | in US20150315612) |
| AAVhu.54 (See SEQ ID NO: 188 in | AAVhu.54 (AAV145.5) (See SEQ ID NO: 177 |
| US20150315612) | in US20150315612) |
| AAVhu.55 (See SEQ ID NO: 187 in | AAVhu.56 (See SEQ ID NO: 205 in |
| US20150315612) | US20150315612) |
| AAVhu.56 (AAV145.6) (See SEQ ID NO: 168 in | AAVhu.56 (AAV145.6) (See SEQ ID NO: 192 |
| US20150315612) | in US20150315612) |
| AAVhu.57 (See SEQ ID NO: 206 in | AAVhu.57 (See SEQ ID NO: 169 in |
| US20150315612) | US20150315612) |
| AAVhu.57 (See SEQ ID NO: 193 in | AAVhu.58 (See SEQ ID NO: 207 in |
| US20150315612) | US20150315612) |
| AAVhu.58 (See SEQ ID NO: 194 in | AAVhu.6 (AAV3.1) (See SEQ ID NO: 5 in |
| US20150315612) | US20150315612) |
| AAVhu.6 (AAV3.1) (See SEQ ID NO: 84 in | AAVhu.60 (See SEQ ID NO: 184 in |
| US20150315612) | US20150315612) |
| AAVhu.60 (AAV161.10) (See SEQ ID NO: 170 in | AAVhu.61 (See SEQ ID NO: 185 in |
| US20150315612) | US20150315612) |
| AAVhu.61 (AAV161.6) (See SEQ ID NO: 174 in | AAVhu.63 (See SEQ ID NO: 204 in |
| US20150315612) | US20150315612) |
| AAVhu.63 (See SEQ ID NO: 195 in | AAVhu.64 (See SEQ ID NO: 212 in |
| US20150315612) | US20150315612) |
| AAVhu.64 (See SEQ ID NO: 196 in | AAVhu.66 (See SEQ ID NO: 197 in |
| US20150315612) | US20150315612) |
| AAVhu.67 (See SEQ ID NO: 215 in | AAVhu.67 (See SEQ ID NO: 198 in |
| US20150315612) | US20150315612) |
| AAVhu.7 (See SEQ ID NO: 226 in | AAVhu.7 (See SEQ ID NO: 150 in |
| US20150315612) | US20150315612) |
| AAVhu.7 (AAV7.3) (See SEQ ID NO: 55 in | AAVhu.71 (See SEQ ID NO: 79 in |
| US20150315612) | US20150315612) |
| AAVhu.8 (See SEQ ID NO: 53 in | AAVhu.8 (See SEQ ID NO: 12 in |
| US20150315612) | US20150315612) |
| AAVhu.8 (See SEQ ID NO: 151 in | AAVhu.9 (AAV3.1) (See SEQ ID NO: 58 in |
| US20150315612) | US20150315612) |
| AAVhu.9 (AAV3.1) (See SEQ ID NO: 155 in | AAV-LK01 (See SEQ ID NO: 2 in |
| US20150315612) | US20150376607) |
| AAV-LK01 (See SEQ ID NO: 29 in | AAV-LK02 (See SEQ ID NO: 3 in |
| US20150376607) | US20150376607) |
| AAV-LK02 (See SEQ ID NO: 30 in | AAV-LK03 (See SEQ ID NO: 4 in |
| US20150376607) | US20150376607) |
| AAV-LK03 (See SEQ ID NO: 12 in | |
| WO2015121501 and SEQ ID NO: 31 in | |
| US20150376607) | |
| AAV-LK04 (See SEQ ID NO: 5 in | AAV-LK04 (See SEQ ID NO: 32 in |
| US20150376607) | US20150376607) |
| AAV-LK05 (See SEQ ID NO: 6 in | AAV-LK05 (See SEQ ID NO: 33 in |
| US20150376607) | US20150376607) |
| AAV-LK06 (See SEQ ID NO: 7 in | AAV-LK06 (See SEQ ID NO: 34 in |
| US20150376607) | US20150376607) |
| AAV-LK07 (See SEQ ID NO: 8 in | AAV-LK07 (See SEQ ID NO: 35 in |
| US20150376607) | US20150376607) |
| AAV-LK08 (See SEQ ID NO: 9 in | AAV-LK08 (See SEQ ID NO: 36 in |
| US20150376607) | US20150376607) |
| AAV-LK09 (See SEQ ID NO: 10 in | AAV-LK09 (See SEQ ID NO: 37 in |
| US20150376607) | US20150376607) |
| AAV-LK10 (See SEQ ID NO: 11 in | AAV-LK10 (See SEQ ID NO: 38 in |
| US20150376607) | US20150376607) |
| AAV-LK11 (See SEQ ID NO: 12 in | AAV-LK11 (See SEQ ID NO: 39 in |
| US20150376607) | US20150376607) |
| AAV-LK12 (See SEQ ID NO: 13 in | AAV-LK12 (See SEQ ID NO: 40 in |
| US20150376607) | US20150376607) |
| AAV-LK13 (See SEQ ID NO: 14 in | AAV-LK13 (See SEQ ID NO: 41 in |
| US20150376607) | US20150376607) |
| AAV-LK14 (See SEQ ID NO: 15 in | AAV-LK14 (See SEQ ID NO: 42 in |
| US20150376607) | US20150376607) |
| AAV-LK15 (See SEQ ID NO: 16 in | AAV-LK15 (See SEQ ID NO: 43 in |
| US20150376607) | US20150376607) |
| AAV-LK16 (See SEQ ID NO: 17 in | AAV-LK16 (See SEQ ID NO: 44 in |
| US20150376607) | US20150376607) |
| AAV-LK17 (See SEQ ID NO: 18 in | AAV-LK17 (See SEQ ID NO: 45 in |
| US20150376607) | US20150376607) |
| AAV-LK18 (See SEQ ID NO: 19 in | AAV-LK18 (See SEQ ID NO: 46 in |
| US20150376607) | US20150376607) |
| AAV-LK19 (See SEQ ID NO: 20 in | AAV-LK19 (See SEQ ID NO: 47 in |
| US20150376607) | US20150376607) |
| AAV-PAEC (See SEQ ID NO: 1 in | AAV-PAEC (See SEQ ID NO: 48 in |
| US20150376607) | US20150376607) |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in | AAV-PAEC2 (See SEQ ID NO: 56 in |
| US20150376607) | US20150376607) |
| AAV-PAEC4 (See SEQ ID NO: 22 in | AAV-PAEC4 (See SEQ ID NO: 55 in |
| US20150376607) | US20150376607) |
| AAV-PAEC6 (See SEQ ID NO: 23 in | AAV-PAEC6 (See SEQ ID NO: 52 in |
| US20150376607) | US20150376607) |
| AAV-PAEC7 (See SEQ ID NO: 24 in | AAV-PAEC7 (See SEQ ID NO: 53 in |
| US20150376607) | US20150376607) |
| AAV-PAEC8 (See SEQ ID NO: 25 in | AAV-PAEC8 (See SEQ ID NO: 50 in |
| US20150376607) | US20150376607) |
| AAVpi.I (See SEQ ID NO: 28 in US20150315612) | AAVpi.I (See SEQ ID NO: 93 in |
| | US20150315612; AAVpi.2 408, see SEQ ID |
| | NO: 30 in US20150315612) |
| AAVpi.2 (See SEQ ID NO: 95 in | AAVpi.3 (See SEQ ID NO: 29 in |
| US20150315612) | US20150315612) |
| AAVpi.3 (See SEQ ID NO: 94 in | AAVrh.10 (See SEQ ID NO: 9 in |
| US20150315612) | US20150159173) |
| AAVrh.10 (See SEQ ID NO: 25 in | AAV44.2 (See SEQ ID NO: 59 in |
| US20150159173) | US20030138772) |
| AAVrh.10 (AAV44.2) (See SEQ ID NO: 81 in | AAV42.1B (See SEQ ID NO: 90 in |
| US20030138772) | US20030138772) |
| AAVrh.I2 (AAV42.1b) (See SEQ ID NO: 30 in | AAVrh.13 (See SEQ ID NO: 10 in |
| US20030138772) | US20150159173) |
| AAVrh.13 (See SEQ ID NO: 26 in | AAVrh.13 (See SEQ ID NO: 228 in |
| US20150159173) | US20150315612) |
| AAVrh.I3R (See SEQ ID NO: in US20150159173 | AAV42.3A (See SEQ ID NO: 87 in |
| | US20030138772) |
| AAVrh.I4 (AAV42.3a) (See SEQ ID NO: 32 in | AAV42.5A (See SEQ ID NO: 89 in |
| US20030138772) | US20030138772) |
| AAVrh.I7 (AAV42.5a) (See SEQ ID NO: 34 in | AAV42.5B (See SEQ ID NO: 91 in |
| US20030138772) | US20030138772) |
| AAVrh.I8 (AAV42.5b) (See SEQ ID NO: 29 in | AAV42.6B (See SEQ ID NO: 112 in |
| US20030138772) | US20030138772) |
| AAVrh.I9 (AAV42.6b) (See SEQ ID NO: 38 in | AAVrh.2 (See SEQ ID NO: 39 in |
| US20030138772) | US20150159173) |
| AAVrh.2 (See SEQ ID NO: 231 in | AAVrh.20 (See SEQ ID NO: 1 in |
| US20150315612) | US20150159173) |
| AAV42.10 (See SEQ ID NO: 106 in | AAVrh.21 (AAV42.10) (See SEQ ID NO: 35 |
| US20030138772) | in US20030138772) |
| AAV42.11 (See SEQ ID NO: 108 in | AAVrh.22 (AAV42.11) (See SEQ ID NO: 37 |
| US20030138772) | in US20030138772) |
| AAV42.12 (See SEQ ID NO: 113 in | AAVrh.23 (AAV42.12) (See SEQ ID NO: 58 |
| US20030138772) | in US20030138772) |
| AAV42.13 (See SEQ ID NO: 86 in | AAVrh.24 (AAV42.13) (See SEQ ID NO: 31 |
| US20030138772) | in US20030138772) |
| AAV42.15 (See SEQ ID NO: 84 in | AAVrh.25 (AAV42.15) (See SEQ ID NO: 28 |
| US20030138772) | in US20030138772) |
| AAVrh.2R (See SEQ ID NO: in US20150159173 | AAVrh.31 (AAV223.1) (See SEQ ID NO: 48 |
| | in US20030138772) |
| AAVC1 (See SEQ ID NO: 60 in US20030138772) | AAVrh.32 (AAVC1) (See SEQ ID NO: 19 in |
| | 446 US20030138772) |
| AAVrh.32/33 (See SEQ ID NO: 2 in | AAVrh.51 (AAV2-5) (See SEQ ID NO: 104 in |
| US20150159173) | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in |
| US20150315612) | US20150315612) |
| AAVrh.54 (See SEQ ID NO: 49 in | |
| US20150159173 and SEQ ID NO: 116 in | |
| US20150315612) | |
| AAVrh.55 (See SEQ ID NO: 37 in | AAVrh.55 (AAV4-19) (See SEQ ID NO: 117 |
| US20150315612) | in US20150315612) |
| AAVrh.56 (See SEQ ID NO: 54 in | AAVrh.56 (See SEQ ID NO: 152 in |
| US20150315612) | US20150315612) |
| AAVrh.57 (See SEQ ID NO: in 497 | AAVrh.57 (See SEQ ID NO: 105 in |
| US20150315612 SEQ ID NO: 26 | US20150315612) |
| AAVrh.58 (See SEQ ID NO: 27 in | AAVrh.58 (See SEQ ID NO: 48 in |
| US20150315612) | US20150159173 and SEQ ID NO: 106 in |
| | US20150315612) |
| | AAVrh.58 (See SEQ ID NO: 232 in |
| | US20150315612) |
| AAVrh.59 (See SEQ ID NO: 42 in | AAVrh.59 (See SEQ ID NO: 110 in |
| US20150315612) | US20150315612) |
| AAVrh.60 (See SEQ ID NO: 31 in | AAVrh.60 (See SEQ ID NO: 120 in |
| US20150315612) | US20150315612) |
| AAVrh.61 (See SEQ ID NO: 107 in | AAVrh.61 (AAV2-3) (See SEQ ID NO: 21 in |
| US20150315612) | US20150315612) |
| AAVrh.62 (AAV2-15) (See SEQ ID NO: 33 in | AAVrh.62 (AAV2-15) (See SEQ ID NO: 114 |
| US20150315612) | in US20150315612) |
| AAVrh.64 (See SEQ ID NO: 15 in | AAVrh.64 (See SEQ ID NO: 43 in |
| US20150315612) | US20150159173 and SEQ ID NO: 99 in |
| | US20150315612) |
| | AAVrh.64 (See SEQ ID NO: 233 in |
| | US20150315612) |
| AAVRh.64RI (See SEQ ID NO: in | AAVRh.64R2 (See SEQ ID NO: in |
| US20150159173 | US20150159173 |
| AAVrh.65 (See SEQ ID NO: 35 in | AAVrh.65 (See SEQ ID NO: 112 in |
| US20150315612) | US20150315612) |
| AAVrh.67 (See SEQ ID NO: 36 in | AAVrh.67 (See SEQ ID NO: 230 in |
| US20150315612) | US20150315612) |
| AAVrh.67 (See SEQ ID NO: 47 in | |
| US20150159173 and SEQ ID NO: 47 in | |
| US20150315612) | |
| AAVrh.68 (See SEQ ID NO: 16 in | AAVrh.68 (See SEQ ID NO: 100 in |
| US20150315612) | US20150315612) |
| AAVrh.69 (See SEQ ID NO: 39 in | AAVrh.69 (See SEQ ID NO: 119 in |
| US20150315612) | US20150315612) |
| AAVrh.70 (See SEQ ID NO: 20 in | AAVrh.70 (See SEQ ID NO: 98 in |
| US20150315612) | US20150315612) |
| AAVrh.71 (See SEQ ID NO: 162 in | AAVrh.72 (See SEQ ID NO: 9 in |
| US20150315612) | US20150315612) |
| AAVrh.73 (See SEQ ID NO: 5 in | AAVrh.74 (See SEQ ID NO: 6 in |
| US20150159173) | US20150159173) |
| AAVrh.8 (See SEQ ID NO: 41 in | AAVrh.8 (See SEQ ID NO: 235 in |
| US20150159173) | US20150315612) |
| AAVrh.8R (See SEQ ID NO: 9 in | AAVrh.8R A586R mutant (See SEQ ID NO: 10 |
| US20150159173, WO2015168666) | in WO2015168666) |
| AAVrh.8R R533A mutant (See SEQ ID NO: 11 in | BAAV (bovine AAV) (See SEQ ID NO: 8 in |
| WO2015168666) | U.S. Pat. No. 9,193,769) |
| BAAV (bovine AAV) (See SEQ ID NO: 10 in | BAAV (bovine AAV) (See SEQ ID NO: 4 in |
| U.S. Pat. No. 9,193,769) | U.S. Pat. No. 9,193,769) |
| BAAV (bovine AAV) (See SEQ ID NO: 2 in | BAAV (bovine AAV) (See SEQ ID NO: 6 in |
| U.S. Pat. No. 9,193,769) | U.S. Pat. No. 9,193,769) |
| BAAV (bovine AAV) (See SEQ ID NO: 1 in | BAAV (bovine AAV) (See SEQ ID NO: 5 in |
| U.S. Pat. No. 9,193,769) | U.S. Pat. No. 9,193,769) |
| BAAV (bovine AAV) (See SEQ ID NO: 3 in | BAAV (bovine AAV) (See SEQ ID NO: 11 in |
| U.S. Pat. No. 9,193,769) | U.S. Pat. No. 9,193,769) |
| BAAV (bovine AAV) (See SEQ ID NO: 5 in | BAAV (bovine AAV) (See SEQ ID NO: 6 in |
| U.S. Pat. No. 7,427,396) | U.S. Pat. No. 7,427,396) |
| BAAV (bovine AAV) (See SEQ ID NO: 7 in | BAAV (bovine AAV) (See SEQ ID NO: 9 in |
| U.S. Pat. No. 9,193,769) | U.S. Pat. No. 9,193,769) |
| BNP61 AAV (See SEQ ID NO: 1 in | BNP61 AAV (See SEQ ID NO: 2 in |
| US20150238550) | US20150238550) |
| BNP62 AAV (See SEQ ID NO: 3 in | BNP63 AAV (See SEQ ID NO: 4 in |
| US20150238550) | US20150238550) |
| caprine AAV (See SEQ ID NO: 3 in U.S. Pat. No. 7,427,396) | caprine AAV (See SEQ ID NO: 4 in |
| | U.S. Pat. No. 7,427,396) |
| true type AAV (ttAAV) (See SEQ ID NO: 2 in | AAAV (Avian AAV) (See SEQ ID NO: 12 in |
| WO2015121501) | U.S. Pat. No. 9,238,800) |
| AAAV (Avian AAV) (See SEQ ID NO: 2 in | AAAV (Avian AAV) (See SEQ ID NO: 6 in |
| U.S. Pat. No. 9,238,800) | U.S. Pat. No. 9,238,800) |
| AAAV (Avian AAV) (See SEQ ID NO: 4 in | AAAV (Avian AAV) (See SEQ ID NO: 8 in |
| U.S. Pat. No. 9,238,800) | U.S. Pat. No. 9,238,800) |
| AAAV (Avian AAV) (See SEQ ID NO: 14 in | AAAV (Avian AAV) (See SEQ ID NO: 10 in |
| U.S. Pat. No. 9,238,800) | U.S. Pat. No. 9,238,800) |
| AAAV (Avian AAV) (See SEQ ID NO: 15 in | AAAV (Avian AAV) (See SEQ ID NO: 5 in |
| U.S. Pat. No. 9,238,800) | U.S. Pat. No. 9,238,800) |
| AAAV (Avian AAV) (See SEQ ID NO: 9 in | AAAV (Avian AAV) (See SEQ ID NO: 3 in |
| U.S. Pat. No. 9,238,800) | U.S. Pat. No. 9,238,800) |
| AAAV (Avian AAV) (See SEQ ID NO: 7 in | AAAV (Avian AAV) (See SEQ ID NO: 11 in |
| U.S. Pat. No. 9,238,800) | U.S. Pat. No. 9,238,800) |
| AAAV (Avian AAV) (See SEQ ID NO: in | AAAV (Avian AAV) (See SEQ ID NO: 1 in |
| U.S. Pat. No. 9,238,800) | U.S. Pat. No. 9,238,800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in |
| US20160017295) | US20160017295) |
| AAV SM 10-2 (See SEQ ID NO: 10 in | AAV SM 10-2 (See SEQ ID NO: 22 in |
| US20160017295) | US20160017295) |
| AAV SM 10-8 (See SEQ ID NO: 39 in | AAV SM 10-8 (See SEQ ID NO: 31 in |
| US20160017295) | US20160017295) |
| AAV CBr-7.1 (See SEQ ID NO: 4 in | AAV CBr-7.1 (See SEQ ID NO: 54 in |
| WO2016065001) | WO2016065001) |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in | AAV CBr-7.2 (See SEQ ID NO: 55 in |
| WO2016065001) | WO2016065001) |
| AAV CBr-7.3 (See SEQ ID NO: 6 in | AAV CBr-7.3 (See SEQ ID NO: 56 in |
| WO2016065001) | WO2016065001) |
| AAV CBr-7.4 (See SEQ ID NO: 7 in | AAV CBr-7.4 (See SEQ ID NO: 57 in |
| WO2016065001) | WO2016065001) |
| AAV CBr-7.5 (See SEQ ID NO: 8 in | AAV CHt-6.6 (See SEQ ID NO: 35 in |
| WO2016065001) | WO2016065001) |
| AAV CHt-6.6 (See SEQ ID NO: 85 in | AAV CHt-6.7 (See SEQ ID NO: 36 in |
| WO2016065001) | WO2016065001) |
| AAV CHt-6.7 (See SEQ ID NO: 86 in | AAV CHt-6.8 (See SEQ ID NO: 37 in |
| WO2016065001) | WO2016065001) |
| AAV CHt-6.8 (See SEQ ID NO: 87 in | AAV CHt-PI (See SEQ ID NO: 29 in |
| WO2016065001) | WO2016065001) |
| AAV CHt-PI (See SEQ ID NO: 79 in | AAV CHt-P2 (See SEQ ID NO: 1 in |
| WO2016065001) | WO2016065001) |
| AAV CHt-P2 (See SEQ ID NO: 51 in | AAV CHt-P5 (See SEQ ID NO: 2 in |
| WO2016065001) | WO2016065001) |
| AAV CHt-P5 (See SEQ ID NO: 52 in | AAV CHt-P6 (See SEQ ID NO: 30 in |
| WO2016065001) | WO2016065001) |
| AAV CHt-P6 (See SEQ ID NO: 80 in | AAV CHt-P8 (See SEQ ID NO: 31 in |
| WO2016065001) | WO2016065001) |
| AAV CHt-P8 (See SEQ ID NO: 81 in | AAV CHt-P9 (See SEQ ID NO: 3 in |
| WO2016065001) | WO2016065001) |
| AAV CHt-P9 (See SEQ ID NO: 53 in | AAV CKd-1 (See SEQ ID NO: 57 in |
| WO2016065001) | U.S. Pat. No. 8,734,809) |
| AAV CKd-1 (See SEQ ID NO: 131 in | AAV CKd-10 (See SEQ ID NO: 58 in |
| U.S. Pat. No. 8,734,809) | U.S. Pat. No. 8,734,809) |
| AAV CKd-10 (See SEQ ID NO: 132 in | AAV CKd-2 (See SEQ ID NO: 59 in |
| U.S. Pat. No. 8,734,809) | U.S. Pat. No. 8,734,809) |
| AAV CKd-2 (See SEQ ID NO: 133 in | AAV CKd-3 (See SEQ ID NO: 60 in |
| U.S. Pat. No. 8,734,809) | U.S. Pat. No. 8,734,809) |
| AAV CKd-3 (See SEQ ID NO: 134 in | AAV CKd-4 (See SEQ ID NO: 61 in |
| U.S. Pat. No. 8,734,809) | U.S. Pat. No. 8,734,809) |
| AAV CKd-4 (See SEQ ID NO: 135 in | AAV CKd-6 (See SEQ ID NO: 62 in |
| U.S. Pat. No. 8,734,809) | U.S. Pat. No. 8,734,809) |
| AAV CKd-6 (See SEQ ID NO: 136 in | AAV CKd-7 (See SEQ ID NO: 63 in |
| U.S. Pat. No. 8,734,809) | U.S. Pat. No. 8,734,809) |
| AAV CKd-7 (See SEQ ID NO: 137 in | AAV CKd-8 (See SEQ ID NO: 64 in |
| U.S. Pat. No. 8,734,809) | U.S. Pat. No. 8,734,809) |
| AAV CKd-8 (See SEQ ID NO: 138 in | AAV CKd-B 1 (See SEQ ID NO: 73 in |
| U.S. Pat. No. 8,734,809) | U.S. Pat. No. 8,734,809) |
| AAV CKd-B 1 (See SEQ ID NO: 147 in | AAV CKd-B2 (See SEQ ID NO: 74 in |
| U.S. Pat. No. 8,734,809) | U.S. Pat. No. 8,734,809) |
| AAV CKd-B2 (See SEQ ID NO: 148 in | AAV CKd-B3 (See SEQ ID NO: 75 in |
| U.S. Pat. No. 8,734,809) | U.S. Pat. No. 8,734,809) |
| AAV CKd-B3 (See SEQ ID NO: in U.S. Pat. No. 8,734,809 | AAV CKd-B3 (See SEQ ID NO: 149 in |
| | U.S. Pat. No. 8,734,809) |
| AAV CLv-1 (See SEQ ID NO: 65 in U.S. Pat. No. 8,734,809) | AAV CLv-1 (See SEQ ID NO: 139 in |
| | U.S. Pat. No. 8,734,809) |
| AAV CLvl-1 (See SEQ ID NO: 171 in | AAV Civ 1-10 (See SEQ ID NO: 178 in |
| U.S. Pat. No. 8,734,809) | U.S. Pat. No. 8,734,809) |
| AAV CLvl-2 (See SEQ ID NO: 172 in | AAV CLv-12 (See SEQ ID NO: 66 in |
| U.S. Pat. No. 8,734,809) | U.S. Pat. No. 8,734,809) |
| AAV CLv-12 (See SEQ ID NO: 140 in | AAV CLvl-3 (See SEQ ID NO: 173 in |
| U.S. Pat. No. 8,734,809) | U.S. Pat. No. 8,734,809) |
| AAV CLv-13 (See SEQ ID NO: 67 in | AAV CLv-13 (See SEQ ID NO: 141 in |
| U.S. Pat. No. 8,734,809) | U.S. Pat. No. 8,734,809) |
| AAV CLvl-4 (See SEQ ID NO: 174 in | AAV Civ 1-7 (See SEQ ID NO: 175 in |
| U.S. Pat. No. 8,734,809) | U.S. Pat. No. 8,734,809) |
| AAV Civ 1-8 (See SEQ ID NO: 176 in | AAV Civ 1-9 (See SEQ ID NO: 177 in |
| U.S. Pat. No. 8,734,809) | U.S. Pat. No. 8,734,809) |
| AAV CLv-2 (See SEQ ID NO: 68 in U.S. Pat. No. 8,734,809) | AAV CLv-2 (See SEQ ID NO: 142 in |
| | U.S. Pat. No. 8,734,809) |
| AAV CLv-3 (See SEQ ID NO: 69 in U.S. Pat. No. 8,734,809) | AAV CLv-3 (See SEQ ID NO: 143 in |
| | U.S. Pat. No. 8,734,809) |
| AAV CLv-4 (See SEQ ID NO: 70 in U.S. Pat. No. 8,734,809) | AAV CLv-4 (See SEQ ID NO: 144 in |
| | U.S. Pat. No. 8,734,809) |
| AAV CLv-6 (See SEQ ID NO: 71 in U.S. Pat. No. 8,734,809) | AAV CLv-6 (See SEQ ID NO: 145 in |
| | U.S. Pat. No. 8,734,809) |
| AAV CLv-8 (See SEQ ID NO: 72 in U.S. Pat. No. 8,734,809) | AAV CLv-8 (See SEQ ID NO: 146 in |
| | U.S. Pat. No. 8,734,809) |
| AAV CLv-DI (See SEQ ID NO: 22 in | AAV CLv-DI (See SEQ ID NO: 96 in |
| U.S. Pat. No. 8,734,809) | U.S. Pat. No. 8,734,809) |
| AAV CLv-D2 (See SEQ ID NO: 23 in | AAV CLv-D2 (See SEQ ID NO: 97 in |
| U.S. Pat. No. 8,734,809) | U.S. Pat. No. 8,734,809) |
| AAV CLv-D3 (See SEQ ID NO: 24 in | AAV CLv-D3 (See SEQ ID NO: 98 in |
| U.S. Pat. No. 8,734,809) | U.S. Pat. No. 8,734,809) |
| AAV CLv-D4 (See SEQ ID NO: 25 in | AAV CLv-D4 (See SEQ ID NO: 99 in |
| U.S. Pat. No. 8,734,809) | U.S. Pat. No. 8,734,809) |
| AAV CLv-D5 (See SEQ ID NO: 26 in | AAV CLv-D5 (See SEQ ID NO: 100 in |
| U.S. Pat. No. 8,734,809) | U.S. Pat. No. 8,734,809) |
| AAV CLv-D6 (See SEQ ID NO: 27 in | AAV CLv-D6 (See SEQ ID NO: 101 in |
| U.S. Pat. No. 8,734,809) | U.S. Pat. No. 8,734,809) |
| AAV CLv-D7 (See SEQ ID NO: 28 in | AAV CLv-D7 (See SEQ ID NO: 102 in |
| U.S. Pat. No. 8,734,809) | U.S. Pat. No. 8,734,809) |
| AAV CLv-D8 (See SEQ ID NO: 29 in | AAV CLv-D8 (See SEQ ID NO: 103 in |
| U.S. Pat. No. 8,734,809) | US8734809); AAV CLv-KI 762, see SEQ ID |
| | NO: 18 in WO2016065001) |
| AAV CLv-KI (See SEQ ID NO: 68 in | AAV CLv-K3 (See SEQ ID NO: 19 in |
| WO2016065001) | WO2016065001) |
| AAV CLv-K3 (See SEQ ID NO: 69 in | AAV CLv-K6 (See SEQ ID NO: 20 in |
| WO2016065001) | WO2016065001) |
| AAV CLv-K6 (See SEQ ID NO: 70 in | AAV CLv-L4 (See SEQ ID NO: 15 in |
| WO2016065001) | WO2016065001) |
| AAV CLv-L4 (See SEQ ID NO: 65 in | AAV CLv-L5 (See SEQ ID NO: 16 in |
| WO2016065001) | WO2016065001) |
| AAV CLv-L5 (See SEQ ID NO: 66 in | AAV CLv-L6 (See SEQ ID NO: 17 in |
| WO2016065001) | WO2016065001) |
| AAV CLv-L6 (See SEQ ID NO: 67 in | AAV CLv-MI (See SEQ ID NO: 21 in |
| WO2016065001) | WO2016065001) |
| AAV CLv-MI (See SEQ ID NO: 71 in | AAV CLv-MII (See SEQ ID NO: 22 in |
| WO2016065001) | WO2016065001) |
| AAV CLv-MI 1 (See SEQ ID NO: 72 in | AAV CLv-M2 (See SEQ ID NO: 23 in |
| WO2016065001) | WO2016065001) |
| AAV CLv-M2 (See SEQ ID NO: 73 in | AAV CLv-M5 (See SEQ ID NO: 24 in |
| WO2016065001) | WO2016065001) |
| AAV CLv-M5 (See SEQ ID NO: 74 in | AAV CLv-M6 (See SEQ ID NO: 25 in |
| WO2016065001) | WO2016065001) |
| AAV CLv-M6 (See SEQ ID NO: 75 in | AAV CLv-M7 (See SEQ ID NO: 26 in |
| WO2016065001) | WO2016065001) |
| AAV CLv-M7 (See SEQ ID NO: 76 in | AAV CLv-M8 (See SEQ ID NO: 27 in |
| WO2016065001) | WO2016065001) |
| AAV CLv-M8 (See SEQ ID NO: 77 in | AAV CLv-M9 (See SEQ ID NO: 28 in |
| WO2016065001) | WO2016065001) |
| AAV CLv-M9 (See SEQ ID NO: 78 in | AAV CLv-RI (See SEQ ID NO: 30 in |
| WO2016065001) | U.S. Pat. No. 8,734,809) |
| AAV CLv-RI (See SEQ ID NO: 104 in | AAV CLv-R2 (See SEQ ID NO: 31 in |
| U.S. Pat. No. 8,734,809) | U.S. Pat. No. 8,734,809) |
| AAV CLv-R2 (See SEQ ID NO: 105 in | AAV CLv-R3 (See SEQ ID NO: 32 in |
| U.S. Pat. No. 8,734,809) | U.S. Pat. No. 8,734,809) |
| AAV CLv-R3 (See SEQ ID NO: 106 in | AAV CLv-R4 (See SEQ ID NO: 33 in |
| U.S. Pat. No. 8,734,809) | U.S. Pat. No. 8,734,809) |
| AAV CLv-R4 (See SEQ ID NO: 107 in | AAV CLv-R5 (See SEQ ID NO: 34 in |
| U.S. Pat. No. 8,734,809) | U.S. Pat. No. 8,734,809) |
| AAV CLv-R5 (See SEQ ID NO: 108 in | AAV CLv-R6 (See SEQ ID NO: 35 in |
| U.S. Pat. No. 8,734,809) | U.S. Pat. No. 8,734,809) |
| AAV CLv-R6 (See SEQ ID NO: 109 in | AAV CLv-R7 (See SEQ ID NO: 110 in |
| U.S. Pat. No. 8,734,809); AAV CLv-R7 802 (see SEQ ID NO: | U.S. Pat. No. 8,734,809) |
| 36 in U.S. Pat. No. 8,734,809) | |
| AAV CLv-R8 (See SEQ ID NO: 37 in | AAV CLv-R8 (See SEQ ID NO: 111 in |
| U.S. Pat. No. 8,734,809) | U.S. Pat. No. 8,734,809) |
| AAV CLv-R9 (See SEQ ID NO: 38 in | AAV CLv-R9 (See SEQ ID NO: 112 in |
| U.S. Pat. No. 8,734,809) | U.S. Pat. No. 8,734,809) |
| AAV CSp-1 (See SEQ ID NO: 45 in U.S. Pat. No. 8,734,809) | AAV CSp-1 (See SEQ ID NO: 119 in |
| | U.S. Pat. No. 8,734,809) |
| AAV CSp-10 (See SEQ ID NO: 46 in U.S. Pat. No. 8,734,809) | AAV CSp-10 (See SEQ ID NO: 120 in |
| | U.S. Pat. No. 8,734,809) |
| AAV CSp-11 (See SEQ ID NO: 47 in U.S. Pat. No. 8,734,809) | AAV CSp-11 (See SEQ ID NO: 121 in |
| | U.S. Pat. No. 8,734,809) |
| AAV CSp-2 (See SEQ ID NO: 48 in U.S. Pat. No. 8,734,809) | AAV CSp-2 (See SEQ ID NO: 122 in |
| | U.S. Pat. No. 8,734,809) |
| AAV CSp-3 (See SEQ ID NO: 49 in U.S. Pat. No. 8,734,809) | AAV CSp-3 (See SEQ ID NO: 123 in |
| | U.S. Pat. No. 8,734,809) |
| AAV CSp-4 (See SEQ ID NO: 50 in U.S. Pat. No. 8,734,809) | AAV CSp-4 (See SEQ ID NO: 124 in |
| | U.S. Pat. No. 8,734,809) |
| AAV CSp-6 (See SEQ ID NO: 51 in U.S. Pat. No. 8,734,809) | AAV CSp-6 (See SEQ ID NO: 125 in |
| | U.S. Pat. No. 8,734,809) |
| AAV CSp-7 (See SEQ ID NO: 52 in U.S. Pat. No. 8,734,809) | AAV CSp-7 (See SEQ ID NO: 126 in |
| | U.S. Pat. No. 8,734,809) |
| AAV CSp-8 (See SEQ ID NO: 53 in U.S. Pat. No. 8,734,809) | AAV CSp-8 (See SEQ ID NO: 127 in |
| | U.S. Pat. No. 8,734,809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in | AAV CSp-8.2 (See SEQ ID NO: 89 in |
| WO2016065001) | WO2016065001) |
| AAV CSp-8.4 (See SEQ ID NO: 40 in | AAV CSp-8.4 (See SEQ ID NO: 90 in |
| WO2016065001) | WO2016065001) |
| AAV CSp-8.5 (See SEQ ID NO: 41 in | AAV CSp-8.5 (See SEQ ID NO: 91 in |
| WO2016065001) | WO2016065001) |
| AAV CSp-8.6 (See SEQ ID NO: 42 in | AAV CSp-8.6 (See SEQ ID NO: 92 in |
| WO2016065001) | WO2016065001) |
| AAV CSp-8.7 (See SEQ ID NO: 43 in | AAV CSp-8.7 (See SEQ ID NO: 93 in |
| WO2016065001) | WO2016065001) |
| AAV CSp-8.8 (See SEQ ID NO: 44 in | AAV CSp-8.8 (See SEQ ID NO: 94 in |
| WO2016065001) | WO2016065001) |
| AAV CSp-8.9 (See SEQ ID NO: 45 in | AAV CSp-8.9 (See SEQ ID NO: 95 in |
| WO2016065001) | WO2016065001) |
| AAV CSp-9 842 (See SEQ ID NO: 54 in | AAV CSp-9 (See SEQ ID NO: 128 in |
| U.S. Pat. No. 8,734,809) | U.S. Pat. No. 8,734,809) |
| AAV.hu.48R3 (See SEQ ID NO: 183 in | AAV.VR-355 (See SEQ ID NO: 181 in |
| U.S. Pat. No. 8,734,809) | U.S. Pat. No. 8,734,809) |
| AAV3B (See SEQ ID NO: 48 in WO2016065001) | AAV3B (See SEQ ID NO: 98 in WO2016065001) |
| AAV4 (See SEQ ID NO: 49 in WO2016065001) | AAV4 (See SEQ ID NO: 99 in WO2016065001) |
| AAV5 (See SEQ ID NO: 50 in WO2016065001) | AAV5 (See SEQ ID NO: 100 in WO2016065001) |
| AAVF1/HSC1 (See SEQ ID NO: 20 in | AAVF1/HSC1 (See SEQ ID NO: 2 in |
| WO2016049230) | WO2016049230) |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in |
| WO2016049230) | WO2016049230) |
| AAVF3/HSC3 (See SEQ ID NO: 22 in | AAVF3/HSC3 (See SEQ ID NO: 5 in |
| WO2016049230) | WO2016049230) |
| AAVF4/HSC4 (See SEQ ID NO: 23 in | AAVF4/HSC4 (See SEQ ID NO: 6 in |
| WO2016049230) | WO2016049230) |
| AAVF5/HSC5 (See SEQ ID NO: 25 in | AAVF5/HSC5 (See SEQ ID NO: 11 in |
| WO2016049230) | WO2016049230) |
| AAVF6/HSC6 (See SEQ ID NO: 24 in | AAVF6/HSC6 (See SEQ ID NO: 7 in |
| WO2016049230) | WO2016049230) |
| AAVF7/HSC7 (See SEQ ID NO: 27 in | AAVF7/HSC7 (See SEQ ID NO: 8 in |
| WO2016049230) | WO2016049230) |
| AAVF8/HSC8 (See SEQ ID NO: 28 in | AAVF8/HSC8 (See SEQ ID NO: 9 in |
| WO2016049230) | WO2016049230) |
| AAVF9/HSC9 (See SEQ ID NO: 10 in | AAVF9/HSC9 882 (see SEQ ID NO: 29 in |
| WO2016049230) | WO2016049230) |

In some embodiments of any one of the aspects, the viral particle comprises a heterologous polynucleotide, e.g., a transgene or a part thereof.

In some aspects provided herein is a population of purified recombinant adeno-associated virus (rAAV) that optionally lacks prokaryotic sequences, wherein the purified virus has a particle to infectivity ratio less than 2×10⁴ vg/TCID50, wherein the population of purified rAAV comprises less than about 10% empty viral capsids. In some embodiments, the purified virus is obtained by a method comprising transfecting a suspension mammalian cell line wherein cells are transfected in suspension. In some embodiments, the population of purified rAAV comprises less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.75%, less than about 0.5%, less than about 0.25%, less than about 0.2%, less than about 0.15%, less than about 0.1%, less than about 0.05%, less than about 0.03%, less than about 0.02%, or, less than about 0.01% empty viral capsids. In several embodiments, the population of purified rAAV is substantially devoid of empty viral capsids. In some embodiments, the purified virus has a particle to infectivity ratio less than 1.5×10⁴ vg/TCID50, less than 1×10⁴ vg/TCID50, less than 9×10³ vg/TCID50, less than 8×10³ vg/TCID50, less than 6×10³ vg/TCID50, less than 5×10³ vg/TCID50, less than 4×10³ vg/TCID50, less than 3×10³ vg/TCID50, less than 2×10³ vg/TCID50, less than 9×10² vg/TCID50, less than 8×10² vg/TCID50, less than 7×10² vg/TCID50, less than 6×10² vg/TCID50, less than 5×10² vg/TCID50, less than 4×10² vg/TCID50, less than 3×10² vg/TCID50, less than 2×10² vg/TCID50, or, less than 1×10² vg/TCID50, or, less than 0.5×10² vg/TCID50 or, even less. In some embodiments, provided herein is a population of purified recombinant adeno-associated virus (rAAV) that does not lack prokaryotic sequences.

In some aspects provided herein is a population of purified recombinant adeno-associated virus (rAAV) that optionally lacks prokaryotic sequence, wherein the purified virus has a particle to infectivity ratio less than 2×10⁴ vg/TCID50. In some embodiments, the purified virus is obtained by a method comprising transfecting a suspension mammalian cell line wherein cells are transfected in suspension. In some embodiments, the purified virus has a particle to infectivity ratio less than 1.5×10⁴ vg/TCID50, less than 1×10⁴ vg/TCID50, less than 9×10³ vg/TCID50, less than 8×10³ vg/TCID50, less than 6×10³ vg/TCID50, less than 5×10³ vg/TCID50, less than 4×10³ vg/TCID50, less than 3×10³ vg/TCID50, less than 2×10³ vg/TCID50, less than 9×10² vg/TCID50, less than 8×10² vg/TCID50, less than 7×10² vg/TCID50, less than 6×10² vg/TCID50, less than 5×10² vg/TCID50, less than 4×10² vg/TCID50, less than 3×10² vg/TCID50, less than 2×10² vg/TCID50, or, less than 1×10² vg/TCID50, or, less than 0.5×10² vg/TCID50 or, even less.

Several of the aspects described herein provide a population of purified recombinant adeno-associated virus (rAAV), wherein, the population of purified rAAV comprises less than about 50% empty viral capsids, wherein the population of rAAV optionally is purified by a process described herein. For example, the population of purified rAAV comprises about 45% or lower, about 40% or lower, about 35% or lower, about 30% or lower, about 25% or lower, about 20% or lower, about 15% or lower, or 10% or lower empty viral capsids. In some embodiments, the population of purified rAAV comprises less than about 10%, less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.75%, less than about 0.5%, less than about 0.25%, less than about 0.2%, less than about 0.15%, less than about 0.1%, less than about 0.05%, less than about 0.03%, less than about 0.02%, or, less than about 0.01% empty viral capsids. In several embodiments, the population of purified rAAV is substantially devoid of empty viral capsids.

In some embodiments, the population of purified rAAV comprises: (i) about 35% or lower empty viral capsids; and/or (ii) the purified rAAV has a particle to infectivity ratio less than 2×10⁴ vg/TCID50; and/or (iii) a ratio of UV₂₆₀ to UV₂₈₀ in the anion exchange eluate is at least about 1.15× to at least about 1.5× higher than a ratio of UV₂₆₀ to UV₂₈₀ ratio in the adjusted affinity eluate.

Further aspects described herein provide a pharmaceutical composition, wherein the pharmaceutical composition comprises the population of purified recombinant adeno-associated virus (rAAV) described herein. The pharmaceutical composition comprising the rAAV, comprises a buffer of pH about 6.5 to about 8.0. In some embodiments, the pH is about 6.5 to about 7.5. For example, the pH is from about 6.5, about 6.6, about 6.7, about 6.8, about 6.9, about 7.0, about 7.1, about 7.2, about 7.3, about 7.4 or about 7.5. In some preferred embodiments, the pH is less than about 7.5. For example, the pH is less than about 7.4, less than about 7.3, less than about 7.2, less than about 7.1, less than about 7.0, less than about 6.9, less than about 6.8, less than about 6.7, or less than about 6.6. In some embodiments, the pharmaceutical composition comprises one or, more excipients, comprising one or, more multivalent ions and/or, salts thereof. In some embodiments, the multivalent ions can be selected or, optionally selected from the group consisting of citrate, sulfate, magnesium and phosphate. In some embodiments, the pharmaceutical composition comprises one or, more excipients, comprising one or, more ions selected or, optionally selected from the group consisting of, sodium, potassium, chloride, ammonium, carbonate, nitrate, chlorate, chlorite, and calcium. In some embodiments, the pharmaceutical composition comprising the rAAV, further comprises a non-ionic surfactant. In some embodiments, the non-ionic surfactant is selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof. In some embodiments, non-ionic surfactant is selected from the group consisting of TWEEN 60 nonionic detergent, PPG-PEG-PPG Pluronic 10R5, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P338 IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, PF-68, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof. In some embodiments, the pharmaceutical composition further comprises polyol, or, sugar, or similar.

### BRIEF DESCRIPTION OF THE FIGURES

The patent or application file contains at least one drawing executed in color. Copies of this patent or patent application publication with color drawings will be provided by the Office upon request and payment of the necessary fee.

FIGS. 1A-1D show analysis of empty and full capsids using CryoTEM (FIGS. 1A and 1B), analytical ultracentrifugation (AUC, FIG. 1C) and SEC-HPLC UV/260/280 ratio (FIG. 1D).

FIG. 2 shows partitioning of empty (E) and full (F) capsids via anion exchange chromatography with different anion exchange modalities.

FIGS. 3A and 3B show separation of empty and full capsids via anion exchange chromatography from different batches. In FIGS. 3A and 3B, dashed line ( . . . ) is UV 260 nm, indicative of DNA, and solid line (-) is UV 280 nm, indicative of protein.

FIGS. 4A-4D show that increasing the amount of citric acid in the dilution buffer and anion exchange equilibration buffer for purification from affinity eluate comprising glycine and citrate shifted the empty particle (E) to the unbound fraction. FIG. 4A, control with no citric acid addition in equilibration buffer and dilution buffer; FIG. 4B, 0 mM citric acid in equilibration buffer and 1.5 mM citric acid in dilution buffer; FIG. 4C, 1.5 mM citric acid in equilibration buffer and 1.5 mM citric acid in dilution buffer; and FIG. 4D, 3 mM citric acid in equilibration buffer and 1.5 mM citric acid in dilution buffer. FIG. 4D show only full particles (F) was in the bound fraction. In FIGS. 4A-4D, dashed line ( . . . ) is UV 260 nm, indicative of DNA, and solid line (-) is UV 280 nm, indicative of protein.

FIGS. 5A-5D show that increasing the amount of citric acid in the dilution buffer for purification from affinity eluate comprising glycine and histidine but lacking citrate shifted the empty particle (E) to the unbound fraction. FIG. 5A, control; FIG. 5B, addition of 3 mM citric acid to the dilution buffer; FIG. 5C, addition of 5 mM citric acid to the dilution buffer; and FIG. 5D, addition of 7 mM citric acid to the dilution buffer. In FIGS. 5A-5D, dashed line ( . . . ) is UV 260 nm, indicative of DNA, and solid line (-) is UV 280 nm, indicative of protein.

FIG. 6 shows SEC HPLC analysis of separation of empty and full capsids via anion exchange chromatography from affinity eluate comprising glycine and histidine but lacking citrate shifted with increasing amounts of citric acid in the dilution buffer.

FIG. 7 is an exemplary anion exchange partitioning chromatograph for purification from affinity eluate comprising glycine and histidine but lacking citrate.

FIG. 8 is a contour plot showing the effect of histidine and glycylglycine concentrations in the affinity elution buffer on vector genome titer for affinity purification.

FIG. 9 shows separation of empty and full particles using different anion exchange buffers.

FIGS. 10A and 10B are different anion exchange chromatograph modalities showing reproducibility of anion exchange purification scale up. In FIGS. 10A and 10B, dashed line ( . . . ) is UV 260 nm, indicative of DNA, and solid line (-) is UV 280 nm, indicative of protein.

FIG. 11 shows effect of gradient length (CV) and elution Q (CV/min) on yield (viral particles, VP, and viral genomes, Vg).

FIG. 12 is an exemplary anion exchange partitioning chromatograph for purification from affinity eluate comprising glycine and histidine but lacking citrate.

FIG. 13 is a graph showing SEC-HPLC elution profile of capsids using different modalities.

FIG. 14 shows UV_{260/280} ratio correlates with MALS % full.

FIGS. 15A-15C show that in absence of a weak acid in the dilution buffer, there is complete binding and complete elution of both full (F) and empty (E) capsids regardless of the % of full capsids in the starting material. FIG. 15A, starting material (S/M) UV_{260/280} ratio 0.96; FIG. 15B, starting material UV_{260/280} ratio 1.05; and FIG. 15C, starting material UV_{260/280} ratio 1.19. In FIGS. 15A-15C, dashed line ( . . . ) is UV 260 nm, indicative of DNA, and solid line (-) is UV 280 nm, indicative of protein.

FIGS. 16A and 16B show inclusion of a weak acid, e.g., citric acid in the dilution buffer used to dilute the affinity eluate for AEX chromatography leads to partitioning of empty capsids in two different starting materials tested.

FIGS. 17A-17C show that modulation of citric acid in the anion exchange dilution buffer results in reduction of empty shoulder elution. FIG. 17A, 8 mM citric acid in the anion exchange dilution buffer and 3 mM citric acid in the equilibration buffer, showing inflection point of empty shoulder at ˜42 mAU and UV_{260/280} ratio of 1.31; FIG. 17B, 9 M citric acid in the anion exchange dilution buffer and 3 mM citric acid in the equilibration buffer, showing no empty shoulder inflection point and UV_{260/280} ratio 1.30; and FIG. 17C, 10 mM citric acid in the anion exchange dilution buffer and 3 mM citric acid in the equilibration buffer, showing no empty shoulder inflection point and UV_{260/280} ratio of 1.30. In FIGS. 17A-17C, dashed line ( . . . ) is UV 260 nm, indicative of DNA, and solid line (-) is UV 280 nm, indicative of protein.

FIGS. 18A-18C show scale comparability of empty/full separation on 1, 4, and 8 mL monolith scale according to exemplary embodiments of the invention. FIG. 18A, 1 mL monolith with 8 mM citric acid in the anion exchange dilution buffer and 3 mM citric acid in the equilibration buffer; FIG. 18B, 4 mL monolith with 9 mM citric acid in the anion exchange dilution buffer and 3 mM citric acid in the equilibration buffer; and FIG. 18C, 8 mL monolith with 10 mM citric acid in the anion exchange dilution buffer and 3 mM citric acid in the equilibration buffer. In FIGS. 18A-18C, dashed line ( . . . ) is UV 260 nm, indicative of DNA, and solid line (-) is UV 280 nm, indicative of protein.

FIGS. 19A-19C show effect of viral particle packaging of starting material on empty/full separation via AEX purification according to exemplary embodiments of the invention. FIG. 19A, starting material UV_{260/280} ratio 0.87; FIG. 19B, starting material UV_{260/280} ratio 0.96; and FIG. 19C, starting material UV_{260/280} ratio 1.04. In FIGS. 19A-19C, dashed line ( . . . ) is UV 260 nm, indicative of DNA, and solid line (-) is UV 280 nm, indicative of protein.

FIGS. 20A-20G show that the amount of the weak acid, e.g., citric acid, in the AEX dilution buffer or AEX equilibration buffer affects the peak shape/purity/recover of AEX purification. FIG. 20A, no citric acid in the dilution buffer or the equilibration buffer; FIG. 20B, 3 mM citric acid in the dilution buffer and no citric acid in the equilibration buffer; FIG. 20C, 8 mM citric acid in the dilution buffer and no citric acid in the equilibration buffer; FIG. 20D, 5 mM citric acid in the dilution buffer and 1 mM citric acid in the equilibration buffer; FIG. 20E, 6 mM citric acid in the dilution buffer and 1 mM citric acid in the equilibration buffer; FIG. 20F, 8 mM citric acid in the dilution buffer and 1 mM citric acid in the equilibration buffer; and FIG. 20G, 8 mM citric acid in the dilution buffer and 3 mM citric acid in the equilibration buffer. In FIGS. 20A-20G, dashed line ( . . . ) is UV 260 nm, indicative of DNA, and solid line (-) is UV 280 nm, indicative of protein.

FIGS. 21A-21D show that the amount of the weak acid, e.g., citric acid, in the AEX equilibration buffer also affects the peak shape/purity/recover of AEX purification. FIG. 21A, 6 mM citric acid in the dilution buffer and 1 mM citric acid in the equilibration buffer; FIG. 21B, 6 mM citric acid in the dilution buffer and 3 mM citric acid in the equilibration buffer; FIG. 21C, 6 mM citric acid in the dilution buffer and 4 mM citric acid in the equilibration buffer; and FIG. 21D, 8 mM citric acid in the dilution buffer and 3 mM citric acid in the equilibration buffer. In FIGS. 21A-21D, dashed line ( . . . ) is UV 260 nm, indicative of DNA, and solid line (-) is UV 280 nm, indicative of protein.

FIGS. 22A-22F show that increasing the histidine concentration in the anion exchange dilution buffer for AEX purification (QA Monolith) from affinity eluate minimized the inversion or infection point of UV_{260/280} ratio indicating increased enrichment of full capsids in the elution phase. FIG. 22A, addition of to the 70 mM histidine anion exchange dilution buffer; FIG. 22B, addition of 90 mM histidine to the anion exchange dilution buffer; FIG. 22C, addition of 100 mM histidine to the anion exchange dilution buffer; FIG. 22D, addition of 120 mM histidine to the anion exchange dilution buffer; FIG. 22E, addition of 150 mM histidine to the anion exchange dilution buffer; and FIG. 22F, addition of 170 mM histidine to the anion exchange dilution buffer. In FIGS. 22A-22F, dashed line ( . . . ) is UV 260 nm, indicative of DNA, and solid line (-) is UV 280 nm, indicative of protein.

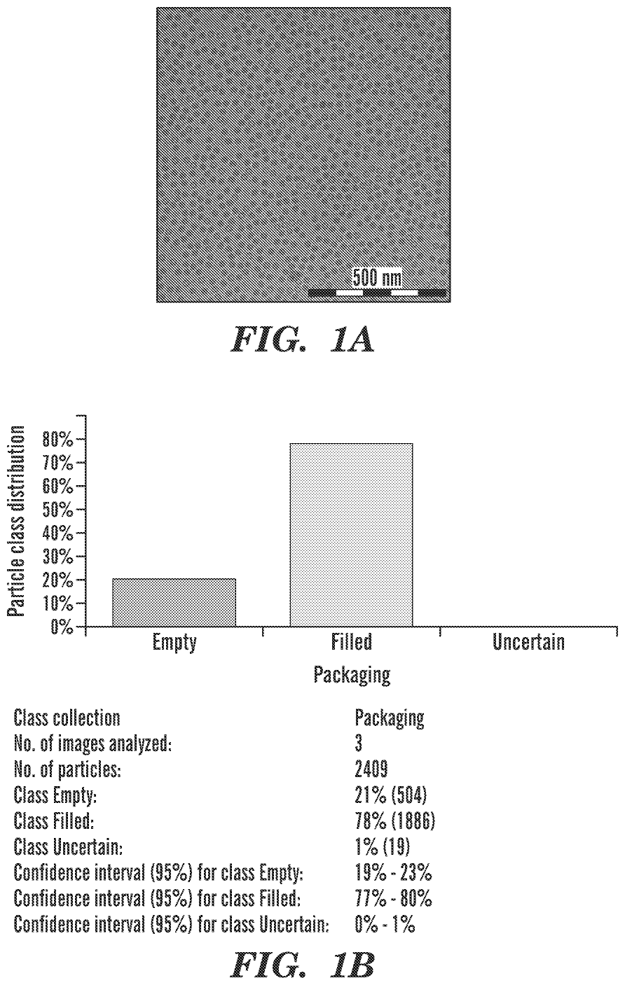

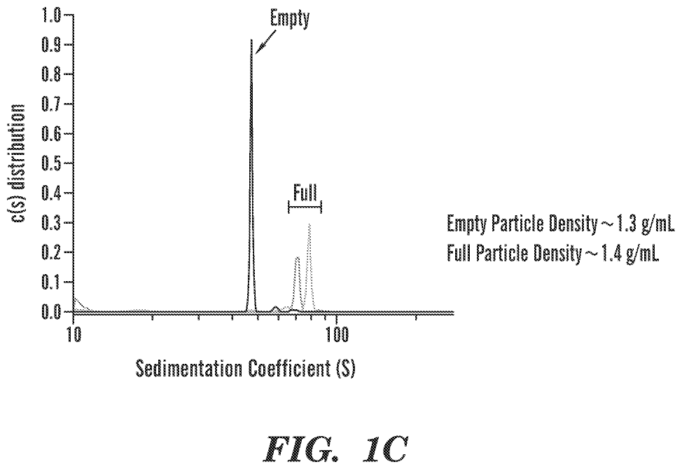

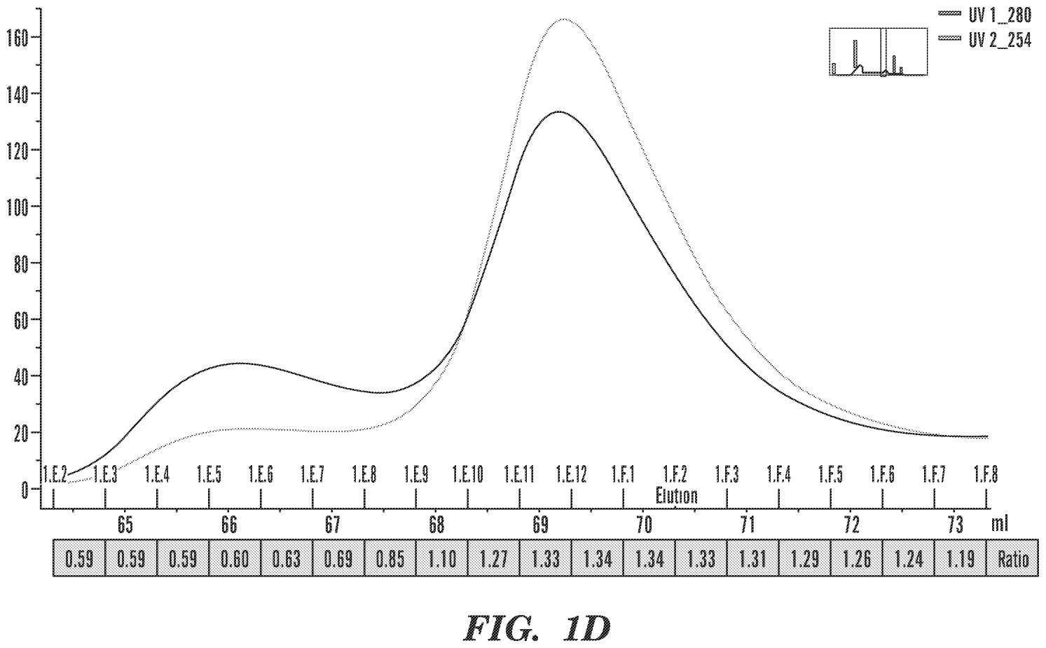

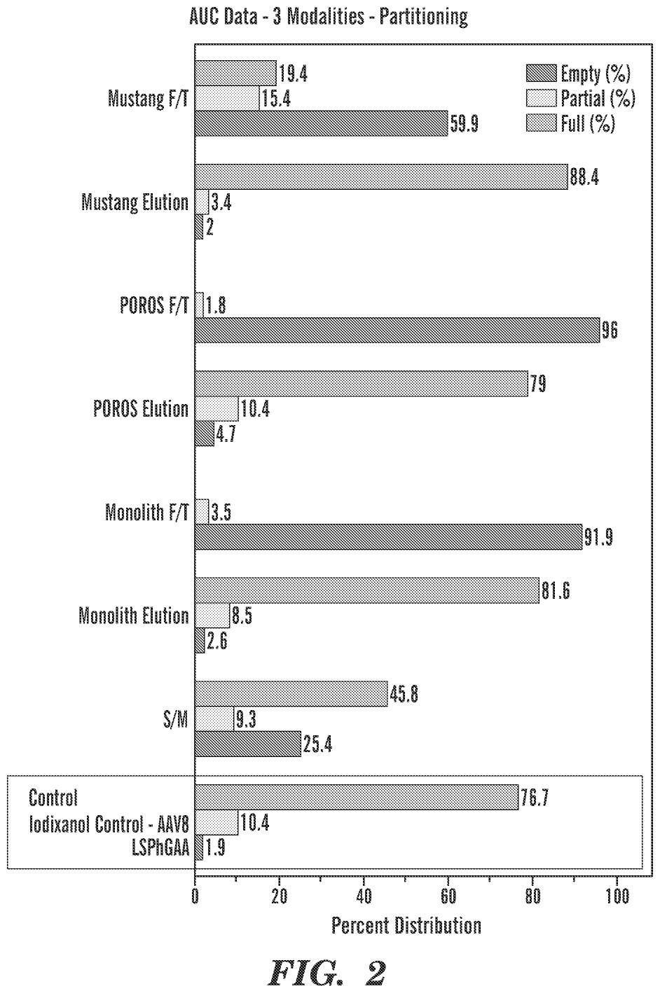

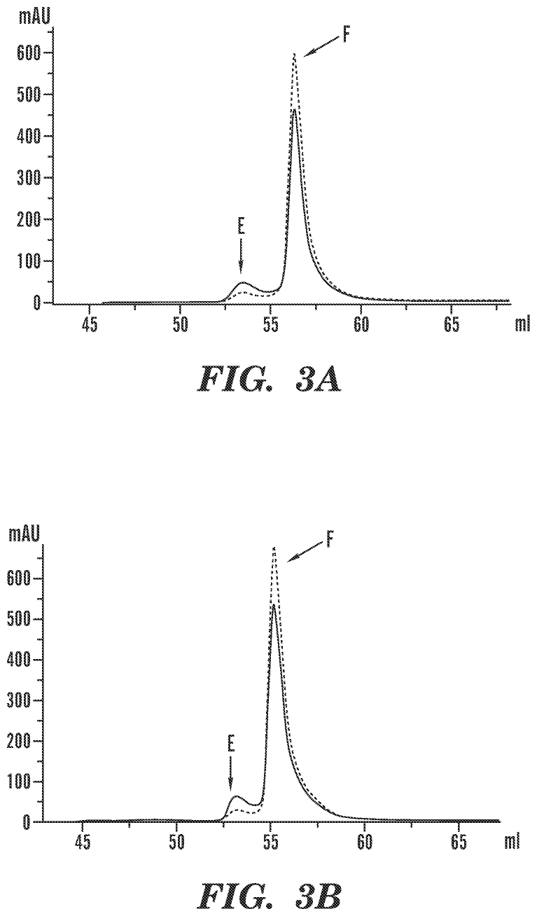

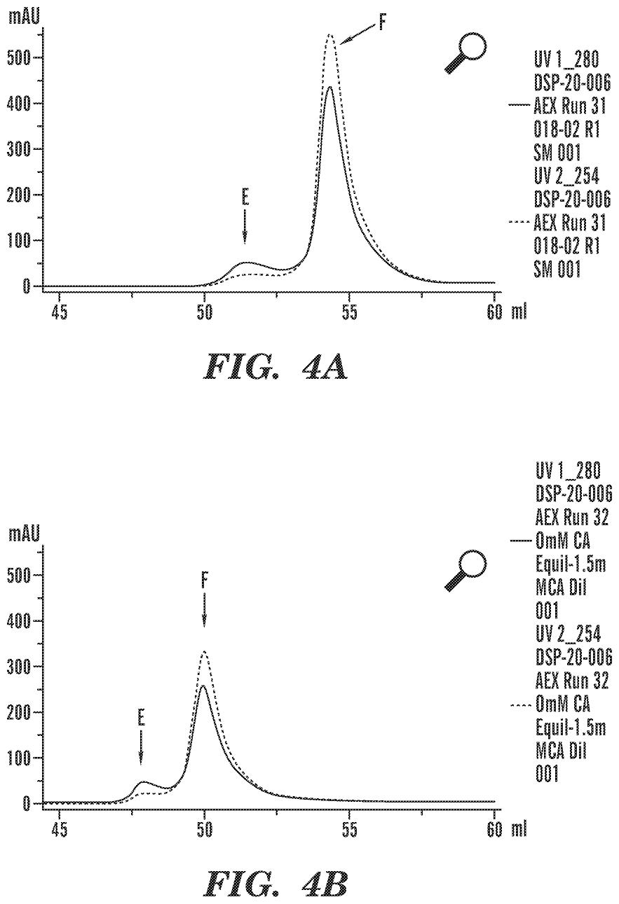

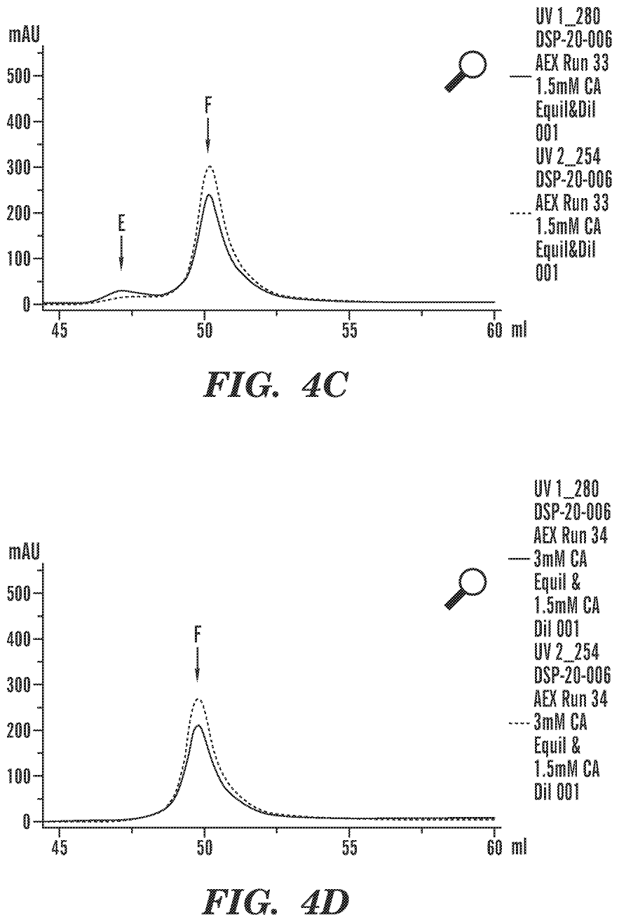

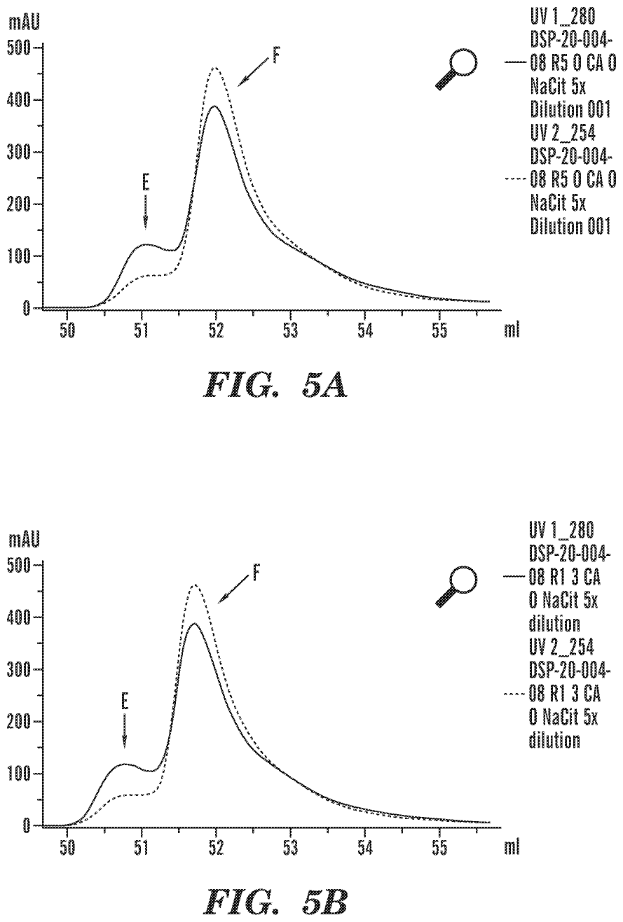

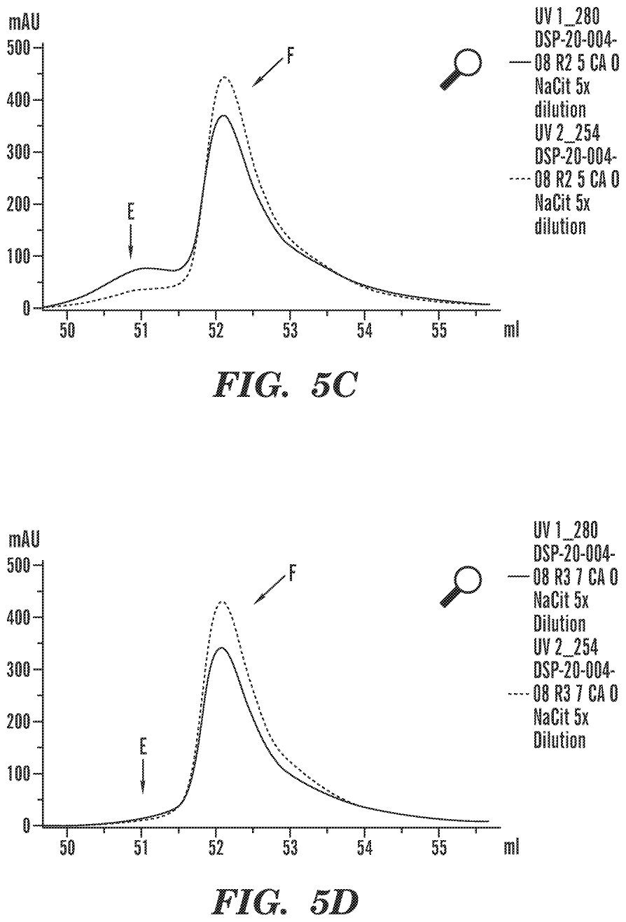

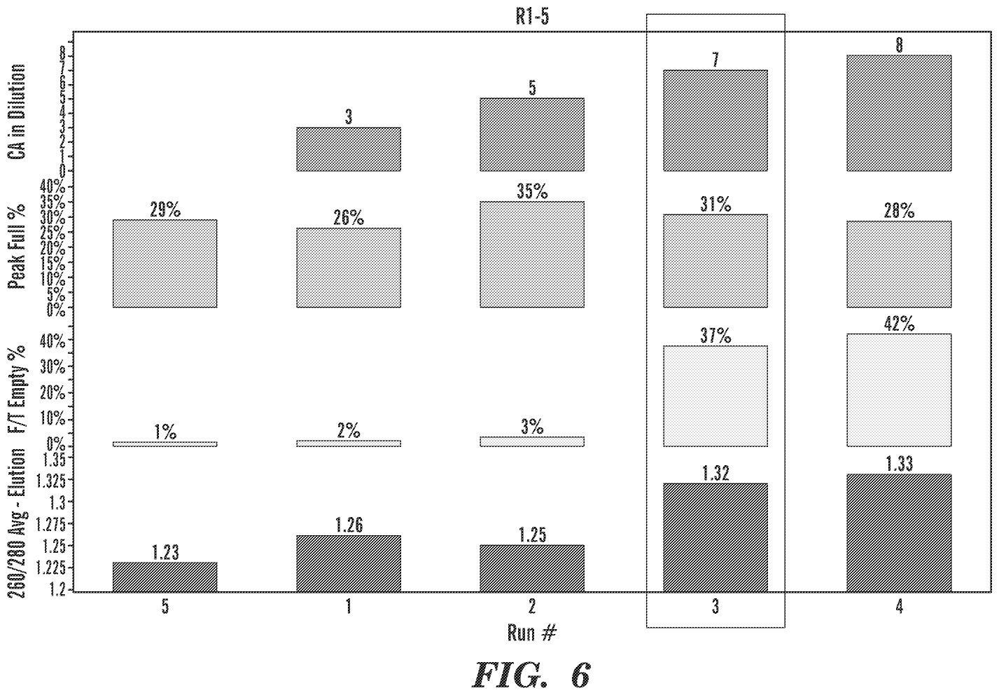

### DETAILED DESCRIPTION OF THE INVENTION

While methods for purifying viral particles are known in the art, methods that are fast, efficient, scalable and economical while maintaining the viral particles in a viable conformation are not well known. The methods disclosed herein allow for fast, efficient, scalable and economical purification of viral particles, e.g., rAAV while maintaining the viral particles in a viable confirmation. The methods disclosed herein provide several significant advantages. The methods are easily adaptable for different viral particles and serotypes, e.g., the methods are easily amenable to purification of different serotypes of rAAV. For example, the methods disclosed herein can be used to purify/isolate rAAV particles of serotype selected from the group consisting of AAV-1, AAV-2, AAV-2i8, AAV-3, AAV-4, AAV-5, AAV-6, AAV-7, AAV-8, AAV-9, AAV rh10, AAV-10, AAV-11, AAV-12, AAV-13, AAV-14, AAV-15, AAV-16 or a chimera, derivative, modification, or pseudotype thereof, or a rational polyploid (also referred as haploid) AAV. The rational polyploid or haploid AAV comprise VP1, VP2 and VP3 as described in PCT/US18/22725 and U.S. Pat. No. 10,550,405, which are incorporated by reference herein.

The methods disclosed herein are scalable and can be applied to the efficient and scalable production of recombinant virus particles, e.g., rAAV. In other words, the methods described herein can be used with volumes of few ml to volumes of thousands of liters. As such, the methods described can be used for the industrial scale production of therapeutic viral particle compositions, e.g., rAAV compositions. The increased yield of filled recombinant virus particles can provide significant cost savings for the industrial scale production of therapeutic viral particle compositions. Further, as discussed herein, the methods can be easily adapted for different viruses or serotypes. The selective elimination of empty virus particle as described herein results in transduction of the recombinant AAV particle in the target tissue type of a subject that is comparable to, or may be better than, recombinant AAV particle purified using density gradient/ultracentrifugation methods. In addition, a recombinant AAV formulation having fewer empty AAV particles can effectively reduce the immune response against the AAV particle (or, capsid) elicited by the subject receiving the recombinant AAV gene therapy thus making it a desired product for AAV gene therapy. The methods described herein can also reduce operator errors and/or increase operator safety.

Embodiments of the various aspects disclosed herein are based on inventors' discovery inter alia that separation of filled recombinant virus particles from empty or partially filled recombinant virus particles in a feed composition can be increased in an anion exchange chromatography media by equilibrating the anion exchange chromatography media with a buffer comprising an anionic compound, e.g., a weak acid or a salt thereof, such as citric acid or a salt thereof prior to contacting the anion exchange media with the feed composition comprising the virus particles, and wherein the feed composition further comprises a predetermined amount of an anionic compound, e.g., a weak acid or a salt thereof.

As used herein, the term “weak acid” refers to an acid that only partially dissociates in aqueous solution. Generally, a “weak acid is a compound that has an acid dissociation constant of less than about 10⁻⁴. Exemplary weak acids include, but are not limited to, acetic acid, citric acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid, and 3-methylbutanoic acid. In some embodiments, the weak acid or salt thereof can be citric acid, acetic acid, succinic acid or salts thereof.

The recombinant AAV particles can be produced by any means known to the person skilled in the art.

In some embodiments, a population of purified recombinant adeno-associated virus (rAAV), purified by the process described herein, comprises less than about 10% empty viral capsids. The process of purifying or, isolating the rAAV comprises: (a) purifying/isolating a plurality of recombinantly expressed virus particles from a harvesting media via affinity chromatography to produce an eluate (affinity chromatography eluate) comprising the plurality of recombinantly expressed virus particles, wherein an elution buffer for affinity chromatography (affinity elution buffer) comprises a predetermined amount of glycine, optionally, the affinity elution buffer is substantially free of weak acids or salts thereof; and optionally, the affinity elution buffer comprises an amino acid that is not glycine; (b) adjusting the affinity chromatography eluate for subsequent purification through anion exchange chromatography, wherein the adjusted eluate comprises a predetermined amount of an anionic compound; (c) purifying/isolating the plurality of recombinantly expressed virus particles from the adjusted eluate of affinity chromatography by anion exchange chromatography to produce a solution comprising a plurality of purified/isolated recombinantly expressed virus particles, wherein an equilibration buffer for anion exchange chromatography comprises a predetermined amount of a weak acid or a salt thereof, optionally the weak acid is citric acid, acetic acid or succinic acid. In some embodiments, the population of purified rAAV, purified by the process described herein, comprises less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.75%, less than about 0.5%, less than about 0.25%, less than about 0.2%, less than about 0.15%, less than about 0.1%, less than about 0.05%, less than about 0.03%, less than about 0.02%, or, less than about 0.01% empty viral capsids. In some embodiments, the population of purified rAAV, purified by the process described herein, is substantially devoid of empty viral capsids.

### Anion Exchange

In one aspect provided herein is a method for separating filled recombinant virus particles from empty or partially filled recombinant virus particles in a feed composition by anion exchange chromatography. Generally, the method comprises equilibrating an exchange chromatography media with an anion exchange equilibration media (AEX equilibration media or, buffer), comprising a predetermined amount of an anionic compound, contacting a feed composition comprising full recombinant virus particles and empty and partially filled recombinant virus particles with the equilibrated anion exchange media under conditions that allow binding of virus particles to the anion exchange chromatography media, and recovering an eluate comprising full recombinant virus particles, wherein the feed composition, and/or adjusted or, diluted feed composition comprises a predetermined amount of an anionic compound.

Generally, less than 20% of the empty or partially filled recombinant virus particles in the feed composition bind to the anion exchange chromatography media. For example, less than 19.5%, less than 19%, less than 18.5%, less than 18%, less than 17.5%, less than 17%, less than 16.5%, less than 16%, less than 15.5%, less than 15%, less than 14.5%, less than 14%, less than 13.5%, less than 13%, less than 12.5%, less than 12%, less than 11.5%, less than 11%, less than 10.5%, less than 10%, less than 9.5%, less than 9%, less than 8.5%, less than 8%, less than 7.5%, less than 7%, less than 6.5%, less than 6%, less than 5.5%, less than 5%, less than 4.5%, less than 4%, less than 3.5%, less than 3%, less than 2.5%, less than 2%, less than 1.5%, less than 1%, less than 0.75%, less than 0.5%, less than 0.25%, less than 0.2%, less than 0.15%, less than 0.1%, less than 0.05% of the empty recombinant virus particles in the feed composition bind to the anion exchange chromatography media. In some embodiments, substantially none of the empty recombinant virus particles in the feed composition bind to the anion exchange chromatography media.

In some embodiments, less than about 50%, or, less than about 45%, or, less than about 40%, or, less than about 35%, or, less than about 30% or, less than about 25%, or, less than about 21%, or, less than about, 20% or even less empty virus particles in the feed composition bind to the anion exchange chromatography media.

As discussed herein, the recovered eluate from the anion exchange is enriched in full recombinant virus particles compared to feed composition. For example, isolated or purified recombinant virus particles, e.g., rAAV particles comprise less than about 40%, or, less than about 35%, or less than about 30% or preferably less than about 25% of empty virus particles. In some embodiments, the isolated or purified recombinant virus particles, e.g., rAAV particles comprise about 20% or less of the of empty virus particles. In some embodiments, the isolated or purified recombinant virus particles, e.g., rAAV particles comprise about 19% or less of the of empty virus particles. In some embodiments, the isolated or purified recombinant virus particles, e.g., rAAV particles comprise about 18% or less of the of empty virus particles. In some embodiments, the isolated or purified recombinant virus particles, e.g., rAAV particles comprise about 17% or less of the of empty virus particles. In some embodiments, the isolated or purified recombinant virus particles, e.g., rAAV particles comprise about 16% or less of the of empty virus particles. In some embodiments, the isolated or purified recombinant virus particles, e.g., rAAV particles comprise about 15% or less of the of empty virus particles. In some embodiments, the isolated or purified recombinant virus particles, e.g., rAAV particles comprise about 14% or less of the of empty virus particles. In some embodiments, the isolated or purified recombinant virus particles, e.g., rAAV particles comprise about 13% or less of the of empty virus particles. In some embodiments, the isolated or purified recombinant virus particles, e.g., rAAV particles comprise about 12% or less of the of empty virus particles. In some embodiments, the isolated or purified recombinant virus particles, e.g., rAAV particles comprise about 11% or less of the of empty virus particles. In some embodiments, the isolated or purified recombinant virus particles, e.g., rAAV particles comprise about 10% or less of the of empty virus particles. In some embodiments, the isolated or purified recombinant virus particles, e.g., rAAV particles comprise about 9% or less of the of empty virus particles. In some embodiments, the isolated or purified recombinant virus particles, e.g., rAAV particles comprise about 8% or less of the of empty virus particles. In some embodiments, the isolated or purified recombinant virus particles, e.g., rAAV particles comprise about 7.5% or less of the of empty virus particles. In some embodiments, the isolated or purified recombinant virus particles, e.g., rAAV particles comprise about 7% or less of the of empty virus particles. In some embodiments, the isolated or purified recombinant virus particles, e.g., rAAV particles comprise about 6.5% or less of the of empty virus particles. In some embodiments, the isolated or purified recombinant virus particles, e.g., rAAV particles comprise about 6% or less of the of empty virus particles. In some embodiments, the isolated or purified recombinant virus particles, e.g., rAAV particles comprise about 5.5% or less of the of empty virus particles. In some embodiments, the isolated or purified recombinant virus particles, e.g., rAAV particles comprise about 5% or less of the of empty virus particles. In some embodiments, the isolated or purified recombinant virus particles, e.g., rAAV particles comprise about 4.5% or less of the of empty virus particles. In some embodiments, the isolated or purified recombinant virus particles, e.g., rAAV particles comprise about 4% or less of the of empty virus particles. In some embodiments, the isolated or purified recombinant virus particles, e.g., rAAV particles comprise about 3.5% or less of the of empty virus particles. In some embodiments, the isolated or purified recombinant virus particles, e.g., rAAV particles comprise about 3% or less of the of empty virus particles. In some embodiments, the isolated or purified recombinant virus particles, e.g., rAAV particles comprise about 2.5% or less of the of empty virus particles. In some embodiments, the isolated or purified recombinant virus particles, e.g., rAAV particles comprise about 2% or less of the of empty virus particles. In some embodiments, the isolated or purified recombinant virus particles, e.g., rAAV particles comprise about 1.5% or less of the of empty virus particles. In some embodiments, the isolated or purified recombinant virus particles, e.g., rAAV particles comprise about 1% or less of the of empty virus particles. In some embodiments, the isolated or purified recombinant virus particles, e.g., rAAV particles comprise about 0.75% or less of the of empty virus particles. In some embodiments, the isolated or purified recombinant virus particles, e.g., rAAV particles comprise about 0.5% or less of the of empty virus particles. In some embodiments, the isolated or purified recombinant virus particles, e.g., rAAV particles comprise about 0.25% or less of the of empty virus particles. In some embodiments, the isolated or purified recombinant virus particles, e.g., rAAV particles comprise about 0.2% or less of the of empty virus particles. In some embodiments, the isolated or purified recombinant virus particles, e.g., rAAV particles comprise about 0.15% or less of the of empty virus particles. In some embodiments, the isolated or purified recombinant virus particles, e.g., rAAV particles comprise about 0.1% or less of the of empty virus particles. In some embodiments, the isolated or purified recombinant virus particles, e.g., rAAV particles comprise about 0.05% or less of the of empty virus particles. In some embodiments, the isolated or purified recombinant virus particles, e.g., rAAV particles are substantially free of empty virus particles.

In some embodiments, isolated or purified recombinant virus particles, e.g., rAAV particles comprise less than about 70%, or, less than about 65%, or, less than about 60%, or less than about 55% of partially full virus particles. In some embodiments, isolated or purified recombinant virus particles, e.g., rAAV particles comprise less than about 50% of partially full virus particles. In some embodiments, isolated or purified recombinant virus particles, e.g., rAAV particles comprise less than about 45% of partially full virus particles. In some embodiments, isolated or purified recombinant virus particles, e.g., rAAV particles comprise less than about 40% of partially full virus particles. In some embodiments, isolated or purified recombinant virus particles, e.g., rAAV particles comprise less than about 35% of partially full virus particles. In some embodiments, isolated or purified recombinant virus particles, e.g., rAAV particles comprise less than about 30% of partially full virus particles. In some embodiments, isolated or purified recombinant virus particles, e.g., rAAV particles comprise less than about 25% of partially full virus particles. In some embodiments, the isolated or purified recombinant virus particles, e.g., rAAV particles comprise about 20% or less of the of partially full virus particles. In some embodiments, the isolated or purified recombinant virus particles, e.g., rAAV particles comprise about 19% or less of the of partially full virus particles. In some embodiments, the isolated or purified recombinant virus particles, e.g., rAAV particles comprise about 18% or less of the of partially full virus particles. In some embodiments, the isolated or purified recombinant virus particles, e.g., rAAV particles comprise about 17% or less of the of partially full virus particles. In some embodiments, the isolated or purified recombinant virus particles, e.g., rAAV particles comprise about 16% or less of the of partially full virus particles. In some embodiments, the isolated or purified recombinant virus particles, e.g., rAAV particles comprise about 15% or less of the of partially full virus particles. In some embodiments, the isolated or purified recombinant virus particles, e.g., rAAV particles comprise about 14% or less of the of partially full virus particles. In some embodiments, the isolated or purified recombinant virus particles, e.g., rAAV particles comprise about 13% or less of the of partially full virus particles. In some embodiments, the isolated or purified recombinant virus particles, e.g., rAAV particles comprise about 12% or less of the of partially full virus particles. In some embodiments, the isolated or purified recombinant virus particles, e.g., rAAV particles comprise about 11% or less of the of partially full virus particles. In some embodiments, the isolated or purified recombinant virus particles, e.g., rAAV particles comprise about 10% or less of the of partially full virus particles. In some embodiments, the isolated or purified recombinant virus particles, e.g., rAAV particles comprise about 9% or less of the of partially full virus particles. In some embodiments, the isolated or purified recombinant virus particles, e.g., rAAV particles comprise about 8% or less of the of partially full virus particles. In some embodiments, the isolated or purified recombinant virus particles, e.g., rAAV particles comprise about 7% or less of the of partially full virus particles. In some embodiments, the isolated or purified recombinant virus particles, e.g., rAAV particles comprise about 6% or less of the of partially full virus particles. In some embodiments, the isolated or purified recombinant virus particles, e.g., rAAV particles comprise about 5% or less of the of partially full virus particles. In some embodiments, the isolated or purified recombinant virus particles, e.g., rAAV particles comprise about 4% or less of the of partially full virus particles. In some embodiments, the isolated or purified recombinant virus particles, e.g., rAAV particles comprise about 3% or less of the of partially full virus particles. In some embodiments, the isolated or purified recombinant virus particles, e.g., rAAV particles comprise about 9% or less of the of partially full virus particles. In some embodiments, the isolated or purified recombinant virus particles, e.g., rAAV particles comprise about 2.5% or less of the of partially full virus particles. In some embodiments, the isolated or purified recombinant virus particles, e.g., rAAV particles comprise about 2% or less of the of partially full virus particles. In some embodiments, the isolated or purified recombinant virus particles, e.g., rAAV particles comprise about 1.5% or less of the of partially full virus particles. In some embodiments, the isolated or purified recombinant virus particles, e.g., rAAV particles comprise about 1% or less of the of partially full virus particles. In some embodiments, the isolated or purified recombinant virus particles, e.g., rAAV particles comprise about 0.75% or less of the of partially full virus particles. In some embodiments, the isolated or purified recombinant virus particles, e.g., rAAV particles comprise about 0.5% or less of the of partially full virus particles. In some embodiments, the isolated or purified recombinant virus particles, e.g., rAAV particles comprise about 0.25% or less of the of partially full virus particles. In some embodiments, the isolated or purified recombinant virus particles, e.g., rAAV particles comprise about 0.2% or less of the of partially full virus particles. In some embodiments, the isolated or purified recombinant virus particles, e.g., rAAV particles comprise about 0.15% or less of the of partially full virus particles. In some embodiments, the isolated or purified recombinant virus particles, e.g., rAAV particles comprise about 0.1% or less of the of partially full virus particles. In some embodiments, the isolated or purified recombinant virus particles, e.g., rAAV particles comprise about 0.05% or less of the of partially full virus particles. In some embodiments, the isolated or purified recombinant virus particles, e.g., rAAV particles are substantially free of partially full virus particles.

In some embodiments, at least about 45% of the isolated or purified viral particles, e.g., rAAV particles are intact or full particles. In some embodiments, at least about 50% of the isolated or purified viral particles, e.g., rAAV particles are intact or full particles. In some embodiments, at least about 55% of the isolated or purified viral particles, e.g., rAAV particles are intact or full particles. In some embodiments, at least about 60% of the isolated or purified viral particles, e.g., rAAV particles are intact or full particles. In some embodiments, at least about 65% of the isolated or purified viral particles, e.g., rAAV particles are intact or full particles. In some embodiments, at least about 70% of the isolated or purified viral particles, e.g., rAAV particles are intact or full particles. In some embodiments, at least about 75% of the isolated or purified viral particles, e.g., rAAV particles are intact or full particles. In some embodiments, at least about 80% of the isolated or purified viral particles, e.g., rAAV particles are intact or full particles. In some embodiments, at least about 85% of the isolated or purified viral particles, e.g., rAAV particles are intact or full particles. In some embodiments, at least about 90% of the isolated or purified viral particles, e.g., rAAV particles are intact or full particles. In some embodiments, at least about 91% of the isolated or purified viral particles, e.g., rAAV particles are intact or full particles. In some embodiments, at least about 92% of the isolated or purified viral particles, e.g., rAAV particles are intact or full particles. In some embodiments, at least about 93% of the isolated or purified viral particles, e.g., rAAV particles are intact or full particles. In some embodiments, at least about 94% of the isolated or purified viral particles, e.g., rAAV particles are intact or full particles. In some embodiments, at least about 95% of the isolated or purified viral particles, e.g., rAAV particles are intact or full particles. In some embodiments, at least about 96% of the isolated or purified viral particles, e.g., rAAV particles are intact or full particles. In some embodiments, at least about 97% of the isolated or purified viral particles, e.g., rAAV particles are intact or full particles. In some embodiments, at least about 98% of the isolated or purified viral particles, e.g., rAAV particles are intact or full particles. In some embodiments, at least about 99% of the isolated or purified viral particles, e.g., rAAV particles are intact or full particles. In some embodiments, substantially all of the isolated or purified viral particles, e.g., rAAV particles are intact or full particles.

In some embodiments, the feed composition for the anion exchange chromatography is an eluate from an affinity chromatography step.

As disclosed herein, the anion exchange chromatography media is washed or equilibrated with a buffer prior to contact with the feed composition. The buffer for washing or equilibrating the anion exchange media (also referred to as AEX equilibration buffer) comprises a predetermined amount of an anionic compound. Generally, the AEX equilibration buffer comprises a predetermined amount of an acid or a salt thereof. For example, the AEX equilibration buffer comprises a predetermined amount of a weak acid. In some embodiments of any one of the aspects, the AEX equilibration buffer comprises a predetermined amount of a citric acid or a salt thereof, acetic acid or a salt thereof, or succinic acid or a salt thereof. For example, the AEX equilibration buffer comprises a predetermined amount citric acid or a salt thereof, acetic acid or a salt thereof, succinic acid or a salt thereof, propionic acid or a salt thereof, or trimesic acid or a salt thereof. In some embodiments, the AEX equilibration buffer comprises a predetermined amount citric acid or a salt thereof, i.e., citrate.

The amount of the anionic compound anionic compound such as an acid or a salt thereof, e.g., a weak acid or salt thereof, such as citric acid or citrate in the AEX equilibration buffer can be optimized for the viral particles to be separated. For example, the AEX equilibration buffer can comprise the anionic compound such as an acid or a salt thereof, e.g., a weak acid or salt thereof, such as citric acid or citrate at a concentration of at least 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM or higher. For example, the AEX equilibration buffer comprises the anionic compound such as an acid or a salt thereof, e.g., a weak acid or salt thereof, such as citric acid or citrate at a concentration of at least about 3 mM, 5 mM, 7 mM or higher.

In some embodiments of any of the aspects, the AEX equilibration buffer comprises the anionic compound such as an acid or a salt thereof, e.g., a weak acid or salt thereof, such as citric acid or citrate at a concentration of from about 0.5 mM to about 15 mM, from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM.

In some embodiments of any one of the aspects, the AEX equilibration buffer comprises the anionic compound such as an acid or a salt thereof, e.g., a weak acid or salt thereof, such as citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM. In some embodiments, the AEX equilibration buffer comprises upto 4 mM of citric acid.

The AEX equilibration buffer can comprise additional components. For example, the AEX equilibration buffer can comprise a predetermined amount of a buffering agent. Exemplary buffering agents include, but are not limited to, acetate, histidine, phosphate, citrate, propionate, tricine, borate and tris(hydroxymethyl)aminomethane (tris). In some embodiments of any one of the aspects, the AEX equilibration buffer comprises a predetermined amount of bis-tris propane (BTP), tris, borate and/or tricine.

The amount of the buffering agent, e.g., BTP, tris, borate and/or tricine in the AEX equilibration buffer can be optimized for the viral particles to be separated. For example, the AEX equilibration buffer can comprise the buffering agent, e.g., BTP, tris, borate and/or tricine at a concentration of at least about 25 mM, 50 mM, 75 mM, 100 mM, 125 mM, 150 mM or higher. For example, the AEX equilibration buffer comprises the buffering agent, e.g., BTP, tris, borate and/or tricine at a concentration of from about 5 mM to about 125 mM, from about 10 mM to about 120 mM, from about 15 mM to about 115 mM, from about 20 mM to about 110 mM, or from about 25 mM to about 10 mM. In some embodiments of any one of the aspects, the AEX equilibration buffer can comprise the buffering agent, e.g., BTP, tris, borate and/or tricine at a concentration of about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, or about 110 mM. In some embodiments of any one of the aspects, the AEX equilibration buffer comprises BTP, tris, borate and/or tricine at a concentration of 0-100 mM. For example, the AEX equilibration buffer comprises upto 100 mM of BTP.

In some embodiments of any one of the aspects, the AEX equilibration buffer comprises a predetermined amount of an amino acid, e.g., a natural or non-natural amino acid. For example, the AEX equilibration buffer comprises a predetermined amount of histidine or arginine. In some embodiments, the equilibration buffer comprises histidine

The amount of the amino acid, e.g., histidine or arginine in the AEX equilibration buffer can be optimized for the viral particles to be separated. For example, the AEX equilibration buffer can comprise the amino acid, e.g., histidine or arginine at a concentration of at least about 25 mM, 50 mM, 75 mM, 100 mM, 125 mM, 150 mM, 175 mM, 200 mM or higher. In some embodiments, the AEX equilibration buffer comprises the amino acid, e.g., histidine or arginine at a concentration of from about 25 mM to about 200 mM, from about 35 mM to about 175 mM or from about 50 mM to about 150 mM. For example, the AEX equilibration buffer can comprise the amino acid, e.g., histidine or arginine at a concentration of about 50 mM, about 75 mM, about 80 mM, about 85 mM, about 90 mM, about 95 mM, about 100 mM, about 105 mM, about 110 mM, about 115 mM, about 120 mM, about 125 mM, about 150 mM or about 175 mM. In some embodiments, the AEX equilibration buffer comprises upto 100 mM of histidine

The AEX equilibration buffer can comprise a predetermined amount of glycerol. Thus, in some embodiments of any one of the aspects, the AEX equilibration buffer comprises glycerol at a concentration of at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10% (w/v, w/w or v/v) or higher. For example, wherein the AEX equilibration buffer comprises glycerol at a concentration of from about 0.5% to about 9.5%, from about 1% to about 9%, from about 2% to about 8.5%, from about 2.5% to about 8% from about 3% to about 7.5%, from about 3.5% to about 7%, from about 4% to about 6.5% or from about 4.5% to about 5.5%. In some embodiments of any one of the aspects, the AEX equilibration buffer comprises glycerol at a concentration of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%. In some embodiments, the AEX equilibration buffer glycerol at a concentration of 0-5%.

The AEX equilibration buffer can also comprise a surfactant. For example, the AEX equilibration buffer comprises a non-ionic surfactant selected from the group consisting of polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic dials; polyalkoxylated alkylphenols; fatty acid alkoxylates; sorbitan alkoxylates; sorbitol esters; C₈ to C22 alkyl or alkenyl polyglycosides; polyalkoxy styrylaryl ethers; alkylamine oxides; block copolymer ethers; polyalkoxylated fatty glyceride; polyalkylene glycol ethers; linear aliphatic or aromatic polyesters; organo silicones; polyaryl phenols; sorbitol ester alkoxylates; and mono- and diesters of ethylene glycol and mixtures thereof; ethoxylated tristyrylphenol; ethoxylated fatty alcohol; ethoxylated lauryl alcohol; ethoxylated castor oil; and ethoxylated nonylphenol; alkoxylated alcohols, amines or acids. In some embodiments of any one of the aspects, the AEX equilibration buffer comprises a non-ionic surfactant selected from the group comprising of polyoxyethylene fatty alcohol ethers, polyoxyethylene alkylphenyl ethers, polyoxyethylene-polyoxypropylene block copolymers, alkylglucosides, alkylphenol ethoxylates, preferably polysorbates, polyoxyethylene alkyl phenyl ethers, and any combinations thereof. Exemplary non-ionic surfactants include, but are not limited to, polysorbates such as polysorbate 20 (TWEEN 20), polysorbate 28, polysorbate 40, polysorbate 60, polysorbate 65, polysorbate 80, polysorbate 81, and polysorbate 85; poloxamers such as poloxamer 188, poloxamer 407; polyethylene polypropylene glycol; or polyethylene glycol (PEG).

In some embodiments of any one of the aspects, the AEX equilibration buffer comprises a surfactant that is a non-ionic fluorosurfactant. For example, the equilibration buffer comprises a non-ionic fluorosurfactant selected from the group consisting of polyethylene glycol polymers, polypropylene glycol polymers, and copolymers thereof. In some embodiments, the equilibration buffer comprises a non-ionic fluorosurfactant selected from the group consisting of PF68, the ZONYL® series of fluoropolymers including Zonyl® FSA, FSP, FSE, UR, FSJ, FSO, FSO-IOO, FS-300, FSN, FSN-100 and TBS available from Sigma-Aldrich, 3M™ Novec FC-4434 from 3M (St. Paul, MN.), Maflon® Lineplus PDM series from Maflon and Masurf® FS-1400, FS-1900 and FS-2000 from Mason Chemical Company (Arlington Heights, IL).

The AEX equilibration buffer can comprise a non-ionic surfactant in an amount of at least about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, about 0.9%, 0.95%, 1% (w/v, w/w, v/v) or higher. For example, the AEX equilibration buffer can comprise a non-ionic surfactant at a concentration of about 0.05% to about 0.95%, from about 0.1% to about 0.9%, from about 0.15% to about 0.85%, from about 0.2% to about 0.8%, from about 0.25% to about 0.75%, from about 0.3% to about 7%, from about 0.35% to about 0.65% from about 0.4% to about 0.6% or from about 0.45% to about 0.55%. In some embodiments of any one of the aspects, the AEX equilibration buffer comprises a non-ionic surfactant at a concentration of about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9% or about 0.95%. In some embodiments, the AEX equilibration buffer comprises a non-ionic surfactant at a concentration of 0-0.5%.

The AEX equilibration buffer can also comprise a cation, e.g., a monovalent or divalent cation. Exemplary monovalent ions for the AEX equilibration buffer include, but are not limited to, sodium (Na⁺), potassium (K⁺), ammonium (NH₄⁺) and alkylamino, e.g., tetramethylammonium. Exemplary divalent cations for the AEX equilibration buffer include, but are not limited to, magnesium (Mg²⁺), calcium (Ca²⁺), copper (Cu²⁺), cobalt (Co²⁺), manganese (Mn²⁺), nickel (Ni²⁺) and zinc (Zn²⁺). The cation can be added in the form of salt. In some embodiments, the AEX equilibration buffer comprises a divalent cation, e.g., Mg²⁺.

In some embodiments, the AEX equilibration buffer comprises Mg²⁺, e.g., MgCl₂ at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher. In some embodiments, the AEX buffer comprises Mg²⁺, e.g., MgCl₂ at a concentration of 0-22 mM.

In some embodiments, the AEX equilibration buffer comprises Na⁺, e.g., NaCl at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher.

In some embodiments, the AEX equilibration buffer comprises K⁺, e.g., KCl or potassium acetate at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher.

In some embodiments, the AEX equilibration buffer comprises ammonium, e.g., ammonium acetate at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher.

In some embodiments, the AEX equilibration buffer comprises tetramethylammonium, e.g., tetramethylammonium chloride at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher.

The AEX equilibration buffer can comprise a polymer. For example, the AEX equilibration buffer can comprise a non-ionic polymer such as hydroxypropylmethyl cellulose, polyvinylpyrrolidone, Plasdone, polyvinyl alcohol, poloxamer (also known by the trade names SYNPERONICS™, PLURONIC™, and KOLLIPHOR™), polysorbate, polyvinylpyrrolidone/vinyl acetate co-polymer (PVP-VA) or polyethyleneglycols (PEGs).

In some embodiments, the AEX equilibration buffer comprises a poloxomer. For example, the AEX equilibration buffer comprises a poloxomer selected from the group consisting of Poloxamer 188 (P188), Pluronic® F127, Pluronic® F38, Pluronic® F68, Pluronic® F87, Pluronic® F108, Pluronic® 10R5, Pluronic® 17R2, Pluronic® 17R4, Pluronic® 25R2, Pluronic® 25R4, Pluronic® 31R1, Pluronic® F108 Cast Solid Surfacta, Pluronic® F108 NF, Pluronic® F108 Pastille, Pluronic® F108NF Prill Poloxamer 338, Pluronic® F127 NF, Pluronic® F127 NF 500 BHT Prill, Pluronic® F127 NF Prill Poloxamer 407, Pluronic® F38 Pastille, Pluronic® F68 LF Pastille, Pluronic® F68 NF, Pluronic® F68 NF Prill, Pluronic® F68 Pastille, Pluronic® F77, Pluronic® F77 Micropastille, Pluronic® F87 NF, Pluronic® F87 NF Prill Poloxamer 237, Pluronic® F 88, Pluronic® F 88 Pastille, Pluronic® F 98, Pluronic® FT L 61, Pluronic® L10, Pluronic® L101, Pluronic® L121, Pluronic® L31, Pluronic® L35, Pluronic® L43, Pluronic® L61, Pluronic® L62, Pluronic® L62 LF, Pluronic® L62D, Pluronic® L64, Pluronic® L81, Pluronic® L92, Pluronic® L44 NF INH surfactant Poloxamer 124, Pluronic® N3, Pluronic® P103, Pluronic® P104, Pluronic® P105, Pluronic® P123 Surfactant, Pluronic® P65, Pluronic® P84, Pluronic® P85, and the like. In some embodiments, the AEX equilibration buffer comprises the poloxomer P188.

The amount of the polymer, e.g., poloxomer in the AEX equilibration buffer can be varied. For example, the amount of the polymer, e.g., poloxomer in the AEX equilibration buffer can be at least about 0.1%, 0.15%, 0.2%, 0.25%, 0.3%, 0.35%, 0.4%, 0.45%, 0.5% (w/w, w/v or v/v) or more. In some embodiments, the AEX equilibration buffer comprises the polymer, e.g., poloxomer at a concentration of from about 0.1% to about 0.5%, from about 0.15% to about 0.45%, from about 0.2% to about 0.4%, or from about 0.25% to about 0.35%. 0.1%. For example, the AEX equilibration buffer comprises the polymer, e.g., poloxomer at a concentration of about 0.1%, about 0.15%, about 0.2%, about 0.25%, about 0.3%, about 0.35%, about 0.4%, about 0.45%, or about 0.5%. In some embodiments, the AEX equilibration buffer comprises the polymer, e.g., poloxomer, such as P188 at a concentration of 0-0.5%.

In some embodiments, the AEX equilibration buffer can comprise a predetermined amount of polyethylene glycol (PEG), e.g., PEG-2K, PEG-5K, PEG-6K, PEG-10K, PEG-12K, PEG-15K, PEG-20K, PEG-40K and the like. For example, the AEX equilibration buffer comprises PEG at a concentration of at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10% (w/v, w/w or v/v) or higher. In some embodiments, the AEX equilibration buffer comprises PEG at a concentration of from about 0.5% to about 9.5%, from about 1% to about 9%, from about 2% to about 8.5%, from about 2.5% to about 8% from about 3% to about 7.5%, from about 3.5% to about 7%, from about 4% to about 6.5% or from about 4.5% to about 5.5%. In some embodiments of any one of the aspects, the AEX equilibration buffer comprises PEG at a concentration of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%. In some embodiments, the AEX equilibration buffer comprises 0-10% PEG. In some embodiments, PEG is PEG-6K.

In some embodiments of any one of the aspects, the anion exchange equilibration buffer, also referred to as anion exchange conditioning buffer herein, comprises: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂). For example, the anion exchange equilibration buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂).

In some embodiments, the anion exchange equilibration buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof).

In some embodiments, the anion exchange equilibration buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments of any one of the aspects, the anion exchange equilibration buffer comprises: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), a weak acid or salt thereof (e.g., citric acid or a salt thereof, acetic acid or a salt thereof, or succinic acid or a salt thereof), of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For example, the anion exchange equilibration buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 0.5-5 mM citric acid, about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). In some embodiments, the anion exchange equilibration buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 0.5-5 mM citric acid, about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 3-7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments, the anion exchange equilibration buffer comprises: about 75 mM, equilibration about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 0.5 mM, about 1 mM, about 1.5 mM, about 2 mM, about 2.5 mM, 3 mM, about 3.5 mM, about 4 mM, about 4.5 mM or about 5 mM of citric acid or a salt thereof; about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof).

In some embodiments, the anion exchange equilibration buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 0.5 mM, about 1 mM, about 1.5 mM, about 2 mM, about 2.5 mM, 3 mM, about 3.5 mM, about 4 mM, about 4.5 mM or about 5 mM of citric acid or a salt thereof; about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments of any one of the aspects, the anion exchange equilibration buffer comprises: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a weak acid or salt thereof (e.g., citric acid or a salt thereof, acetic acid or a salt thereof, or succinic acid or a salt thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For example, the anion exchange equilibration buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 0.5-5 mM of a weak acid or salt thereof (e.g., citric acid or a salt thereof, acetic acid or a salt thereof, or succinic acid or a salt thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). In some embodiments, the anion exchange equilibration buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 3-10 mM of a weak acid or salt thereof (e.g., citric acid or a salt thereof, acetic acid or a salt thereof, or succinic acid or a salt thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 3-7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments, the anion exchange equilibration buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 0.5 mM, about 1 mM, about 1.5 mM, about 2 mM, about 2.5 mM, 3 mM, about 3.5 mM, about 4 mM, about 4.5 mM or about 5 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof).

In some embodiments, the anion exchange equilibration buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 0.5 mM, about 1 mM, about 1.5 mM, about 2 mM, about 2.5 mM, 3 mM, about 3.5 mM, about 4 mM, about 4.5 mM or about 5 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments, the anion exchange equilibration buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 2-4 mM a weak acid or salt thereof (e.g., citric acid or a salt thereof, acetic acid or a salt thereof, or succinic acid or a salt thereof), about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), about 0.5% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For Example, the anion exchange equilibration buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 2 mM, about 2.5 mM, about 3 mM, about 3.5 mM or about 4 mM of a weak acid or salt thereof (e.g., citric acid or a salt thereof, acetic acid or a salt thereof, or succinic acid or a salt thereof); about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), about 0.5%, and about 5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments of any one of the aspects, the anion exchange equilibration buffer comprises: a buffering agent (e.g., a buffering agent selected from the group consisting of acetate, histidine, phosphate, citrate, propionate, tricine, borate, tris(hydroxymethF68 of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof)yl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a weak acid or salt thereof (e.g., citric acid or a salt thereof, acetic acid or a salt thereof, or succinic acid or a salt thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and optionally the buffer is substantially free of glycine. For example, the anion exchange equilibration buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 0.5-5 mM of a weak acid or salt thereof (e.g., citric acid or a salt thereof, acetic acid or a salt thereof, or succinic acid or a salt thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and optionally the buffer is substantially free of glycine. In some embodiments, the anion exchange equilibration buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 3-10 mM citric acid, about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and the buffer has a high pH (e.g., a pH of about 8.5-9.5, such as a pH of about 8.5, about 9 or about 9.5), and optionally the buffer is substantially free of glycine.

In some embodiments, the anion exchange equilibration buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 0.5 mM, about 1 mM, about 1.5 mM, about 2 mM, about 2.5 mM, 3 mM, about 3.5 mM, about 4 mM, about 4.5 mM or about 5 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); and about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and optionally the buffer is substantially free of glycine.

In some embodiments, the anion exchange equilibration buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 0.5 mM, about 1 mM, about 1.5 mM, about 2 mM, about 2.5 mM, 3 mM, about 3.5 mM, about 4 mM, about 4.5 mM or about 5 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); and about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and the buffer has a high pH (e.g., a pH of about 8.5-9.5, such as a pH of about 8.5, about 9 or about 9.5), and optionally the buffer is substantially free of glycine.

In some embodiments, the anion exchange equilibration buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 2-4 mM of a weak acid or salt thereof (e.g., citric acid or a salt thereof, acetic acid or a salt thereof, or succinic acid or a salt thereof), about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and about 0.4% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and optionally the buffer is substantially free of glycine. For Example, the anion exchange equilibration buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 2 mM, about 2.5 mM, about 3 mM, about 3.5 mM or about 4 mM of a weak acid or salt thereof (e.g., citric acid or a salt thereof, acetic acid or a salt thereof, or succinic acid or a salt thereof); about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and about 0.4% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and the buffer has a high pH (e.g., a pH of about 8.5-9.5, such as a pH of about 8.5, about 9 or about 9.5), and optionally the buffer is substantially free of glycine.

In some embodiments, the anion exchange equilibration buffer comprises: about 100 mM BTP, about 100 mM histidine, about 2-4 mM citric acid or a salt thereof, about 1 mM MgCl₂, and about 0.4% PF68. For Example, the anion exchange equilibration buffer comprises: about 100 mM BTP; about 100 mM histidine; about 2 mM, about 2.5 mM, about 3 mM, about 3.5 mM or about 4 mM citric acid or a salt thereof; about 1 mM MgCl₂, and about 0.4% PF68, and the buffer has a high pH (e.g., a pH of about 8.5-9.5, such as a pH of about 8.5, about 9 or about 9.5), and optionally the buffer is substantially free of glycine.

It is noted that the anion exchange chromatography media can be equilibrated by washing the media with the AEX equilibration buffer. For example, the anion exchange chromatography media can be washed with at least 1 column volume (CV) of the AEX equilibration media. In some embodiments, the anion exchange chromatography media is equilibrated with at least about 1 CV, 1.5 CV, 2 CV, 2.5 CV, 3 CV, 3.5 CV, 4 CV, 4.5 CV, 5 CV, 5.5 CV, 6 CV, 6.5 CV, 7 CV, 7.5 CV, 8 CV, 8.5 CV, 9 CV, 9.5 CV, 10 CV, 10.5 CV, 11 CV, 11.5 CV, 12 CV, 12.5 CV, 13 CV, 13.5 CV, 14 CV, 14.5 CV, 15 CV, 15.5 CV, 16 CV, 16.5 CV, 17 CV, 17.5 CV, 18 CV, 18.5 CV, 19 CV, 19.5 CV, 20 CV or more of the AEX equilibration buffer.

The anion exchange chromatography media can be equilibrated with the AEX equilibration buffer at any desired temperature. Generally, the anion exchange chromatography media can be equilibrated with the AEX equilibration buffer at room temperature.

### AEX Elution Buffer

Generally, the AEX elution buffer comprises a buffering agent. Exemplary buffering agents include, but are not limited to, acetate, histidine, phosphate, citrate, propionate, tricine, borate and tris(hydroxymethyl)aminomethane (tris). In some embodiments of any one of the aspects, the AEX elution buffer comprises a predetermined amount of bis-tris propane (BTP).

The amount of the buffering agent, e.g., BTP in the AEX elution buffer can be optimized for the viral particles to be separated. For example, the AEX elution buffer can comprise the buffering agent, e.g., BTP, tris, borate and/or tricine at a concentration of at least about 25 mM, 50 mM, 75 mM, 100 mM, 125 mM, 150 mM or higher. For example, the AEX elution buffer comprises the buffering agent, e.g., BTP at a concentration of from about 50 mM to about 150 mM, from about 75 mM to about 125 mM, from about 85 mM to about 115 mM, or from about 90 mM to about 110 mM. In some embodiments of any one of the aspects, the AEX elution buffer can comprise the buffering agent, e.g., BTP at a concentration of about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM or about 125 mM.

The AEX elution buffer can also comprise a surfactant. For example, the AEX elution buffer comprises a non-ionic surfactant. Exemplary non-ionic surfactants include, but are not limited to, polysorbates such as polysorbate 20 (TWEEN 20), polysorbate 28, polysorbate 40, polysorbate 60, polysorbate 65, polysorbate 80, polysorbate 81, and polysorbate 85; poloxamers such as poloxamer 188, poloxamer 407; polyethylene polypropylene glycol; or polyethylene glycol (PEG). In some embodiments of any one of the aspects, the surfactant is a non-ionic fluorosurfactant. Exemplary non-ionic fluorosurfactants include fluorosurfactants containing polyethylene glycol polymers, polypropylene glycol polymers, and copolymers thereof. Specific exemplary non-ionic fluorosurfactants include, but are not limited to, PF68.

The AEX elution buffer can comprise the non-ionic surfactant in an amount of at least about 0.001%, 0.0025%, 0.005%, 0.0075%, 0.01%, 0.0125%, 0.015%, 0.0175%, 0.02%, 0.025%, 0.03%, 0.0325%, 0.035%, 0.0375%, 0.04%, 0.045%, 0.05% (w/v, w/w, v/v) or higher. For example, the AEX elution buffer can comprise a non-ionic surfactant at a concentration of about 0.001% to about 0.02%, from about 0.0025% to about 0.0175%, from about 0.005% to about 0.015%, or from about 0.0075% to about 0.0125%. In some embodiments of any one of the aspects, the AEX elution buffer comprises a non-ionic surfactant at a concentration of about 0.001%, about 0.0025%, about 0.005%, about 0.0075%, about 0.01%, about 0.0125%, about 0.015%, about 0.0175%, about 0.02%, about 0.025%, about 0.03%, about 0.0325%, about 0.035%, about 0.0375%, about 0.04%, about 0.045% or about 0.05%. In some embodiments, the AEX elution buffer comprises a non-ionic surfactant at a concentration of 0-0.05%.

The AEX equilibration buffer can also comprise a cation, e.g., a monovalent or divalent cation. Exemplary monovalent ions for the AEX elution buffer include, but are not limited to, sodium (Na⁺), potassium (K⁺), ammonium (NH₄⁺) and alkylamino, e.g., tetramethylammonium. Exemplary divalent cations for the AEX elution buffer include, but are not limited to, magnesium (Mg²⁺), calcium (Ca²⁺), copper (Cu²⁺), cobalt (Co²⁺), manganese (Mn²⁺) nickel (Ni²⁺) and zinc (Zn²⁺). The cation can be added in the form of salt. In some embodiments, the AEX elution buffer comprises a divalent cation, e.g., Mg²⁺.

In some embodiments, the AEX elution buffer comprises Mg²⁺, e.g., MgCl₂ at a concentration of at least about 0.5 mM, 1 mM, 1.5 mM, 2 mM, 2.5 mM, 3 mM, 3.5 mM, 4 mM, 4.5 mM, 5 mM or higher. In some embodiments, the AEX elution buffer comprises Mg²⁺, e.g., MgCl₂ at a concentration of 0-2 mM.

In some embodiments, the AEX elution buffer comprises Na⁺, e.g., NaCl at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher.

In some embodiments, the AEX elution buffer comprises K⁺, e.g., potassium acetate at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher.

In some embodiments, the AEX elution buffer comprises ammonium, e.g., ammonium acetate at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher.

In some embodiments, the AEX elution buffer comprises tetramethylammonium, e.g., tetramethylammonium chloride at a concentration of at least about 1 mM, 5 mM, 10 mM, 25 mM, 50 mM, 75 mM, 100 mM, 125 mM, 150 mM, 175 mM, 200 mM, 225 mM, 250 mM, 275 mM, 300 mM, 325 mM, 350 mM or higher.

In some embodiments, the AEX elution buffer comprises: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), sodium acetate and PF68. For example, the AEX elution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 250-350 mM sodium acetate and about 0.005-0.015% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof).

In some embodiments, the AEX elution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 250 mM, about 255 mM, about 260 mM, about 265 mM, about 270 mM, about 275 mM, about 280 mM, about 285 mM, about 290 mM, about 300 mM, about 305 mM, about 310 mM, about 315 mM, about 320 mM, about 325 mM, about 330 mM, about 335 mM, about 340 mM, about 345 mM or about 350 mM sodium acetate; and about 0.005%, about 0.0075%, about 0.01%, about 0.0125% or about 0.015% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof).

In some embodiments, the AEX elution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 250 mM, about 255 mM, about 260 mM, about 265 mM, about 270 mM, about 275 mM, about 280 mM, about 285 mM, about 290 mM, about 300 mM, about 305 mM, about 310 mM, about 315 mM, about 320 mM, about 325 mM, about 330 mM, about 335 mM, about 340 mM, about 345 mM or about 350 mM sodium acetate; and about 0.005%, about 0.0075%, about 0.01%, about 0.0125% or about 0.015% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and the buffer has a high pH (e.g., a pH of about 8.5-9.5, such as a pH of about 8.5, about 9 or about 9.5).

The methods described herein can be used with any anion exchange chromatography media known and available to a practitioner. Exemplary anion exchange chromatography media include without limitation, MACRO PREP Q (BioRad, Hercules, Calif.); UNOSPHERE Q (BioRad, Hercules, Calif.); POROS 50HQ (Applied Biosystems, Foster City, Calif.); POROS 50D (Applied Biosystems, Foster City, Calif.); POROS 50PI (Applied Biosystems, Foster City, Calif.); POROS XO (Applied Biosystems, Foster City, Calif.); SOURCE 30Q (GE Healthcare, N.J.); SOURCE 15Q (GE Healthcare, N.J.); DEAE SEPHAROSE (GE Healthcare, Piscataway, N.J.); Q SEPHAROSE (GE Healthcare Biosciences, Piscataway, N.J.), Capto Q and Capto Adhere (GE Healthcare, N.J.); EMPHAZE (3M Corp./Pierce); and multimodal chromatography e.g., Prima S(BIA Separations-Sartorius; Slovenia-Germany).

In some embodiments of any one of the aspects, the anion exchange chromatography media is a monolith anion exchange chromatography media. The term “monolith chromatography column” or “monolith chromatography media” is a term of art and refers to chromatography columns that contain three dimensional macroporous structures, i.e., a monolith, as the stationary phase. Non-limiting monolith chromatography columns include CIMmultus™ Disposable pre-packed chromatographic monolithic columns, CIMac™ Analytical Columns, CIM® line monolithic columns, UNO® Monolith Columns, and Chromolith® Monolithic HPLC Columns. Non limiting monolith chromatography resins include CIMmultus™ QA-1 Advanced Composite Column (Quaternary amine), CIMmultus™ DEAE-1 Advanced Composite Column (Diethylamino), QA Disk (Quaternary amine), CIM® DEAE, EDA Disk (Ethylene diamino), UNO® Monolith Anion Exchange Columns.

In some embodiments of any one of the aspects, the feed composition for the anion exchange chromatography comprises a predetermined amount of an ionic compound, e.g., a cationic compound or an anionic compound, e.g., an acid or salt thereof. In some embodiments, the feed composition for the anion exchange chromatography comprises a predetermined amount of an anionic compound, e.g., an acid or a salt thereof. For example, the feed composition for the anion exchange chromatography comprises a predetermined amount of a weak acid. As, a further example, the feed composition is diluted and/or, adjusted with a buffer e.g., anion exchange dilution buffer that comprises a predetermined amount of weak acid, or, salt thereof. In some embodiments of any one of the aspects, the feed composition or, adjusted, or, diluted feed composition comprises a predetermined amount of a citric acid or a salt thereof, acetic acid or a salt thereof, or succinic acid or a salt thereof. For example, the feed composition comprises a predetermined amount citric acid or a salt thereof, i.e., citrate.

The amount of the anionic compound anionic compound such as an acid or a salt thereof, e.g., citric acid or citrate in the feed composition for the anion exchange chromatography can be optimized for the viral particles to be separated. For example, the feed composition can comprise the anionic compound such as an acid or a salt thereof, e.g., citric acid or citrate at a concentration of at least 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM or higher. For example, the feed composition comprises the anionic compound such as an acid or a salt thereof, e.g., citric acid or citrate at a concentration of at least about 3 mM, 5 mM, 7 mM or higher.

In some embodiments of any of the aspects, the feed composition comprises the anionic compound such as an acid or a salt thereof, e.g., citric acid or citrate at a concentration of from about 0.5 mM to about 15 mM, from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM.

In some embodiments of any one of the aspects, the feed composition comprises the anionic compound such as an acid or a salt thereof, e.g., citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM.

In some embodiments, the feed composition for the anion exchange chromatography comprises a predetermined amount of a cationic compound, e.g., a monovalent or divalent cation. Exemplary monovalent ions for the feed composition include, but are not limited to, sodium (Na t), potassium (K⁺), alkylamino and ammonium. Exemplary divalent cations for the dilution buffer include, but are not limited to, magnesium (Mg²⁺), calcium (Ca²⁺), copper (Cu²⁺), cobalt (Co²⁺), manganese (Mn²⁺), nickel (Ni²⁺) and zinc (Zn²⁺). The cation can be added in the form of salt. In some embodiments, the feed composition comprises a divalent cation, e.g., Mg²⁺.

The amount of the cationic compound in the feed composition for the anion exchange chromatography can be optimized for the viral particles to be separated. For example, the feed composition can comprise the cationic compound at a concentration of at least 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM or higher. For example, the feed composition comprises the cationic compound at a concentration of at least about 3 mM, 5 mM, 7 mM or higher. In some embodiments of any of the aspects, the feed composition comprises the cationic compound at a concentration of from about 0.5 mM to about 15 mM, from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM. In some embodiments of any one of the aspects, the feed composition comprises the cationic compound at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM.

### Affinity Chromatography

As described herein, the feed composition for the anion exchange chromatography can be an eluate from an affinity chromatography step (also referred to as affinity eluate). For example, eluate from affinity chromatography of a harvest media, e.g., a cell culture media.

The term “affinity chromatography” or “affinity purification” as used herein designates any method that uses specific binding interactions between molecules. A particular ligand is chemically immobilized or “coupled” to a solid support so that when a complex mixture is passed over the column, those molecules having specific binding affinity to the ligand become bound. After other sample components are washed away, the bound molecule is stripped from the support, resulting in its purification from the original sample. Affinity chromatography encompasses immunoaffinity chromatography. The term “immunoaffinity chromatography” as used herein designates any method that uses immobilized antibodies, or fragments thereof, in affinity chromatography.

Exemplary affinity chromatography media include, but are not limited to, Affi-Gel (Biorad); Affinica Agarose/Polymeric Supports (Schleicher and Schuell); AvidGel (BioProbe); Bio-Gel (BioRad); Fractogel (EM Separations); HEMA-AFC (Alltech); Reacti-Gel (Pierce); Sephacryl(Pharmacia); Sepharose (Pharmacia); Superose (Pharmacia); Trisacryl (IBF); TSK Gel Toyopearl (TosoHaas); Ultragel (IBF); AvidGel CPG (BioProbe); HiPAC (ChromatoChem); Protein-Pak Affinity Packing (Waters); Ultraffinity-EP (Bodman); CAPTO AAVB (GE Healthcare, N.J.), AAVB Sepharose; and oligonucleotide conjugated (e.g., aptamer conjugated) affinity media. Other affinity chromatography media include affinity monolith chromatography supports, and POROS® affinity chromatography supports.

It is noted that the affinity elute can be used as the feed composition for the anion exchange purification. Thus, in some embodiments of any one of the aspects, the eluate from affinity chromatography comprises a predetermined amount of an anionic compound. For example, an anionic compound can be added to the eluate from affinity chromatography prior to anion exchange chromatography. Generally, an acid or a salt thereof is added to the eluate from affinity chromatography. Accordingly, in some embodiments of any one of the aspects, the affinity eluate comprises a predetermined amount of a weak acid. For example, affinity eluate or, adjusted/diluted affinity eluate (e.g., adjusted/diluted feed composition) comprises a predetermined amount of a citric acid or a salt thereof, acetic acid or a salt thereof, or succinic acid or a salt thereof. For example, the affinity eluate comprises a predetermined amount citric acid or a salt thereof, i.e., citrate.

The amount of the anionic compound anionic compound such as weak acid or a salt thereof, e.g., citric acid or citrate in the affinity eluate can be optimized for the viral particles to be separated. For example, the affinity eluate can comprise the anionic compound such as an acid or a salt thereof, e.g., citric acid or citrate at a concentration of at least 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM or higher. For example, the affinity eluate comprises the anionic compound such as a weak acid or a salt thereof, e.g., citric acid or citrate at a concentration of at least about 3 mM, 5 mM, 7 mM or higher.

In some embodiments of any of the aspects, the affinity eluate comprises the anionic compound such as a weak acid or a salt thereof, e.g., citric acid or citrate at a concentration of from about 0.5 mM to about 15 mM, from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM.

In some embodiments of any one of the aspects, the affinity eluate comprises the anionic compound such as a weak acid or a salt thereof, e.g., citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM.

In some embodiments of any one of the aspects, the affinity eluate can be diluted prior to anion exchange chromatography. For example, a buffer (e.g., a dilution buffer) can be added to the affinity eluate. It is noted that the affinity eluate can be diluted by a factor of 1×-40×. For example, the affinity eluate can be diluted by a factor of at least 1×, 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 10.5×, 11×, 11.5×, 12×, 12.5×, 13×, 13.5×, 14×, 14.5×, 15×, 15.5×, 16×, 16.5×, 17×, 17.5×, 18×, 18.5×, 19×, 19.5×, 20× or more. Generally, the affinity eluate can be diluted 2-6×, 7-10×, 11-15× or 6-20×. For example, the affinity eluate can be diluted by a factor of about 1.5× to about 5×, about 5× to about 10×, about 10× to about 15×, or about 15× to about 20×. In some embodiments, the affinity eluate can be diluted by a factor of about 4.5× to about 5.5× with a dilution buffer, e.g., anion exchange dilution buffer. As used herein, the term “anion exchange dilution buffer” refers to the buffer used to dilute the affinity eluate for use as the feed composition for the anion exchange chromatography.

In some embodiments, the affinity eluate can be diluted with both an affinity elution buffer and an anion exchange dilution buffer. For example, the affinity eluate can be diluted by a factor of at about 1× to about 6× with an affinity elution buffer, e.g., the affinity buffer used for the affinity eluate, and then further diluted by a factor of about 1× to about 10× with an anion exchange buffer. In some embodiments, the affinity eluate can be diluted by a factor of about 1×, about 1.5×, about 2×, about 2.5×, about 3×, about 3.5×, about 4×, about 4.5×, about 5×, about 5.5× or about 6× with the affinity elution buffer, and the diluted affinity eluate can be further diluted with the anion exchange dilution buffer. In some embodiments, the affinity eluate can be first diluted with the affinity elution buffer and the diluted affinity eluate can be further diluted with the anion exchange dilution buffer by a factor of about 1×, about 1.5×, about 2×, about 2.5×, about 3×, about 3.5×, about 4×, about 4.5×, about 5×, about 5.5×, about 6×, about 6.5×, about 7×, about 7.5×, about 8×, about 8.5×, about 9×, about 9.5× or about 10×. For example, the affinity eluate can be first diluted with the affinity elution buffer by a factor of about 1×, about 1.5×, about 2×, about 2.5×, about 3×, about 3.5×, about 4×, about 4.5×, about 5×, about 5.5× or about 6×, and the diluted affinity eluate can be further diluted with the anion exchange dilution buffer by a factor of about 1×, about 1.5×, about 2×, about 2.5×, about 3×, about 3.5×, about 4×, about 4.5×, about 5×, about 5.5×, about 6×, about 6.5×, about 7×, about 7.5×, about 8×, about 8.5×, about 9×, about 9.5× or about 10×.

In some embodiments of any one of the aspects, the dilution buffer for diluting the affinity eluate comprises a predetermined amount of an anionic compound. For example, the dilution buffer comprises a predetermined amount of an acid or a salt thereof, e.g., a weak acid or a salt thereof. In some embodiments of any one of the aspects, the dilution buffer comprises a predetermined amount of a weak acid. For example, the dilution buffer comprises a predetermined amount of citric acid or a salt thereof, acetic acid or a salt thereof, or succinic acid or a salt thereof. In some embodiments of any one of the aspects, the dilution buffer comprises a predetermined amount citric acid or a salt thereof, i.e., citrate.

The amount of the anionic compound such as an acid or a salt thereof, e.g., citric acid or citrate in the dilution buffer can be optimized for the viral particles to be separated. For example, the dilution buffer can comprise the anionic compound such as an acid or a salt thereof, e.g., citric acid or citrate at a concentration of at least 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM or higher.

In some embodiments of any of the aspects, the dilution buffer comprises the anionic compound such as a weak acid or a salt thereof, e.g., citric acid or citrate at a concentration of from about 0.5 mM to about 15 mM, from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM.

In some embodiments of any one of the aspects, the dilution buffer comprises the anionic compound such as a weak acid or a salt thereof, e.g., citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM. For example, the dilution buffer comprises the anionic compound such as a weak acid or a salt thereof, e.g., citric acid or citrate at a concentration of about 1.5 mM.

In some embodiments, the amount of the anionic compound, e.g., a weak acid or a salt thereof in the dilution buffer can be at least 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM or higher. For example, the amount of the anionic compound, e.g., a weak acid or a salt thereof in the dilution buffer can be from about 10 mM to about 100 mM, from about 20 mM to about 90 mM, from about 30 mM to about 80 mM, from about 40 mM to about 70 mM or from 50 mM to about 60 mM.

The dilution buffer can comprise additional components. For example, the dilution buffer can comprise a predetermined amount of a buffering agent. Exemplary buffering agents include, but are not limited to, acetate, histidine, phosphate, citrate, and propionate. In some embodiments of any one of the aspects, the dilution buffer comprises a predetermined amount of bis-tris propane (BTP).

The amount of the buffering agent, e.g., BTP in the dilution buffer can be optimized for the viral particles to be separated. For example, the dilution buffer can comprise the buffering agent, e.g., BTP at a concentration of at least about 25 mM, 50 mM, 75 mM, 100 mM, 125 mM, 150 mM or higher. For example, the dilution buffer comprises the buffering agent, e.g., BTP at a concentration of from about 25 mM to about 175 mM, from about 50 mM to about 150 mM, from about 75 mM to about 125 mM, from about 80 mM to about 120 mM, from about 85 mM to about 115 mM, from about 90 mM to about 110 mM or from about 95 mM to about 105 mM. In some embodiments of any one of the aspects, the dilution buffer can comprise the buffering agent, e.g., BTP at a concentration of about 50 mM, about 75 mM, about 80 mM, about 85 mM, about 90 mM, about 95 mM, about 100 mM, about 105 mM, about 110 mM, about 115 mM, about 120 mM, about 125 mM, about 150 mM or about 175 mM. For example, dilution buffer can comprise the buffering agent, e.g., BTP at a concentration of about 100 mM.

In some embodiments of any one of the aspects, the dilution buffer comprises a predetermined amount of an amino acid, e.g., a natural or non-natural amino acid. For example, the dilution buffer comprises a predetermined amount of histidine, arginine, lysine or asparagine.

The amount of the amino acid, e.g., histidine, arginine, lysine or asparagine in the dilution buffer can be optimized for the viral particles to be separated. For example, the dilution buffer can comprise the amino acid, e.g., histidine, arginine, lysine or asparagine at a concentration of at least about 25 mM, 50 mM, 75 mM, 100 mM, 125 mM, 150 mM or higher. In some embodiments, the dilution buffer comprises the amino acid, e.g., histidine, arginine, lysine or asparagine at a concentration of from about 25 mM to about 175 mM, from about 50 mM to about 150 mM, from about 75 mM to about 125 mM, from about 80 mM to about 120 mM, from about 85 mM to about 115 mM, from about 90 mM to about 110 mM or from about 95 mM to about 105 mM. For example, the dilution buffer can comprise the amino acid, e.g., histidine, arginine, lysine or asparagine at a concentration of about 50 mM, about 75 mM, about 80 mM, about 85 mM, about 90 mM, about 95 mM, about 100 mM, about 105 mM, about 110 mM, about 115 mM, about 120 mM, about 125 mM, about 150 mM or about 175 mM. In some embodiments of any one of the aspects, the dilution buffer comprises the amino acid, e.g., histidine, arginine, lysine or asparagine at a concentration of about 100 mM.

The dilution buffer can comprise a predetermined amount of glycerol. Thus, in some embodiments of any one of the aspects, the dilution buffer comprises glycerol at a concentration of at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% (w/v, w/w or v/v) or higher. For example, wherein the dilution buffer comprises glycerol at a concentration of from about 0.5% to about 9.5%, from about 1% to about 9%, from about 2% to about 8.5%, from about 2.5% to about 8% from about 3% to about 7.5%, from about 3.5% to about 7%, from about 4% to about 6.5% or from about 4.5% to about 5.5%. In some embodiments of any one of the aspects, the dilution buffer comprises glycerol at a concentration of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9% or about 9.5%. For example, the dilution buffer comprises glycerol at a concentration of about 5%.

The dilution buffer can also comprise a surfactant. Exemplary non-ionic surfactants include, but are not limited to, polysorbates such as polysorbate 20 (TWEEN 20), polysorbate 28, polysorbate 40, polysorbate 60, polysorbate 65, polysorbate 80, polysorbate 81, and polysorbate 85; poloxamers such as poloxamer 188, poloxamer 407; polyethylene polypropylene glycol; or polyethylene glycol (PEG).

In some embodiments of any one of the aspects, the surfactant is a non-ionic fluorosurfactant. Exemplary non-ionic fluorosurfactants include fluorosurfactants containing polyethylene glycol polymers, polypropylene glycol polymers, and copolymers thereof. Specific exemplary non-ionic fluorosurfactants include, but are not limited to, PF68.

The dilution buffer can comprise a non-ionic surfactant in an amount of at least about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, about 0.9%, 0.95% (w/v, w/w, v/v) or higher. For example, the dilution buffer can comprise a non-ionic surfactant at a concentration of about 0.05% to about 0.95%, from about 0.1% to about 0.9%, from about 0.15% to about 0.85%, from about 0.2% to about 0.8%, from about 0.25% to about 0.75%, from about 0.3% to about 7%, from about 0.35% to about 0.65% from about 0.4% to about 0.6% or from about 0.45% to about 0.55%. In some embodiments of any one of the aspects, the dilution buffer comprises a non-ionic surfactant at a concentration of about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9% or about 0.95%. For example, the dilution buffer comprises a non-ionic surfactant at a concentration of about 0.5%.

The dilution buffer can also comprise a cation, e.g., a monovalent or divalent cation. Exemplary monovalent ions for the dilution buffer include, but are not limited to, sodium (Na⁺), potassium (K⁺), alkylamino and ammonium. Exemplary divalent cations for the dilution buffer include, but are not limited to, magnesium (Mg²⁺), calcium (Ca²⁺), copper (Cu²⁺), cobalt (Co²⁺), manganese (Mn²⁺), nickel (Ni²⁺) and zinc (Zn²⁺). The cation can be added in the form of salt. In some embodiments, the dilution buffer comprises a divalent cation, e.g., Mg²⁺.

It is noted that the dilution buffer can comprise the cation, e.g., a divalent cation such as Mg²⁺ at a concentration of at least about 0.1 mM, 0.25 mM, 0.5 mM, 0.75 mM, 1 mM, 1.25 mM, 1.5 mM, 1.75 mM, 2 mM or higher. In some embodiments, the dilution buffer comprises a cation, e.g., a divalent cation such as Mg²⁺ at a concentration of from about 0.1 mM to about 2 mM, from about 0.25 mM to about 1.75 mM, from about 0.5 mM to about 1.5 mM, or from about 0.75 mM to about 1.25 mM. For example, the dilution buffer comprises a cation, e.g., a divalent cation such as Mg²⁺ at a concentration of about 0.1 mM, about 0.25 mM, about 0.5 mM, about 0.75 mM, about 1 mM, about 1.25 mM, about 1.5 mM, about 1.75 mM or about 2 mM. in some embodiments, the dilution buffer comprises a cation, e.g., a divalent cation such as Mg²⁺ at a concentration of about 1 mM.

In some embodiments of any one of the aspects, the dilution buffer has a high pH. For example, the dilution buffer has a pH greater than or equal to about 7.5, about 8, about 8.5, about 9, about 9.5 or about 10. In some embodiments, the dilution buffer has a pH greater than or equal to about 8.5. For example, the dilution buffer has a pH of about 9.

In some embodiments of any one of the aspects, the anion exchange dilution buffer comprises: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of a bis-tris propane (BTP), tris, borate, tricine, and any combinations thereof, or a buffering agent selected from the group consisting of BTP, tris and any combinations thereof), an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For example, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 50-250 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 3-7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). In some embodiments, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 120-170 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For example, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 120-170 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH, e.g., a pH of about 8.5-9.5.

In some embodiments, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 120 mM, about 125 mM, about 130 mM, about 135 mM, about 140 mM, about 145 mM or about 150 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 0.3%, about 0.35%, about 0.4%, about 0.45%, about 0.5%, about 0.55%, about 0.6%, about 0.65% or about 0.7% of; about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof).

In some embodiments, the anion exchange dilution buffer comprises: about 75 mM, about 80 mM, about 85 mM, about 90 mM of a non-ionic surfactant (e.g., a non-ionic surfactant selected from the group consisting of fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 120 mM, about 125 mM, about 130 mM, about 135 mM, about 140 mM, about 145 mM or about 150 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or anon-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments of any one of the aspects, the anion exchange dilution buffer comprises: of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), a weak acid or salt thereof (e.g., citric acid or a salt thereof, acetic acid or a salt thereof, or succinic acid or a salt thereof), of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For example, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 3-12 mM of a weak acid or salt thereof (e.g., citric acid or a salt thereof, acetic acid or a salt thereof, or succinic acid or a salt thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). In some embodiments, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 3-12 mM of a weak acid or salt thereof (e.g., citric acid or a salt thereof, acetic acid or a salt thereof, or succinic acid or a salt thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 3-7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 3 mM, about 3.5 mM, about 4 mM, about 4.5 mM, about 5 mM, about 5.5 mM, about 6 mM, about 6.5 mM, about 7 mM, about 7.5 mM, about 7.5 mM, about 8 mM, about 8.5, about 9 mM, about 9.5 mM, about 10 mM, about 10.5 mM, about 11 mM, about 11.5 mM or about 12 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or anon-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof).

In some embodiments, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 3 mM, about 3.5 mM, about 4 mM, about 4.5 mM, about 5 mM, about 5.5 mM, about 6 mM, about 6.5 mM, about 7 mM, about 7.5 mM, about 7.5 mM, about 8 mM, about 8.5, about 9 mM, about 9.5 mM, about 10 mM, about 10.5 mM, about 11 mM, about 11.5 mM or about 12 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or anon-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments of any one of the aspects, the anion exchange dilution buffer comprises: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a weak acid or salt thereof (e.g., citric acid or a salt thereof, acetic acid or a salt thereof, or succinic acid or a salt thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For example, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 3-12 mM of a weak acid or salt thereof (e.g., citric acid or a salt thereof, acetic acid or a salt thereof, or succinic acid or a salt thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). In some embodiments, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 3-10 mM of a weak acid or salt thereof (e.g., citric acid or a salt thereof, acetic acid or a salt thereof, or succinic acid or a salt thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or anon-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 3-7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 3 mM, about 3.5 mM, about 4 mM, about 4.5 mM, about 5 mM, about 5.5 mM, about 6 mM, about 6.5 mM, about 7 mM, about 7.5 mM, about 7.5 mM, about 8 mM, about 8.5, about 9 mM, about 9.5 mM, about 10 mM, about 10.5 mM, about 11 mM, about 11.5 mM or about 12 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof).

In some embodiments, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 3 mM, about 3.5 mM, about 4 mM, about 4.5 mM, about 5 mM, about 5.5 mM, about 6 mM, about 6.5 mM, about 7 mM, about 7.5 mM, about 7.5 mM, about 8 mM, about 8.5, about 9 mM, about 9.5 mM, about 10 mM, about 10.5 mM, about 11 mM, about 11.5 mM or about 12 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments, the anion exchange dilution buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 6-8 mM of a weak acid or salt thereof (e.g., citric acid or a salt thereof, acetic acid or a salt thereof, or succinic acid or a salt thereof), about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), about 0.5% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 5% Glycerol. For Example, the anion exchange dilution buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 6 mM, about 6.5 mM, about 7 mM, about 7.5 mM or about 8 mM of a weak acid or salt thereof (e.g., citric acid or a salt thereof, acetic acid or a salt thereof, or succinic acid or a salt thereof); about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), about 0.5% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments of any one of the aspects, the anion exchange dilution buffer comprises: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a weak acid or salt thereof (e.g., citric acid or a salt thereof, acetic acid or a salt thereof, or succinic acid or a salt thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). In some embodiments of any one of the aspects, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 1-10 mM of a weak acid or salt thereof (e.g., citric acid or a salt thereof, acetic acid or a salt thereof, or succinic acid or a salt thereof), about 0.005-0.015% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For example, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 1-10 mM of a weak acid or salt thereof (e.g., citric acid or a salt thereof, acetic acid or a salt thereof, or succinic acid or a salt thereof), about 0.005-0.015% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and about 2.5-7.5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments of any one of the aspects described herein, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 1 mM, about 1.5 mM, about 2 mM, about 2.5 mM, 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM or about 10 mM of a weak acid or salt thereof (e.g., citric acid or a salt thereof, acetic acid or a salt thereof, or succinic acid or a salt thereof); about 0.005%, about 0.0075%, about 0.01%, about 0.0125% or about 0.015% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof).

In some embodiments, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 1 mM, about 1.5 mM, about 2 mM, about 2.5 mM, 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM or about 10 mM of a weak acid or salt thereof (e.g., citric acid or a salt thereof, acetic acid or a salt thereof, or succinic acid or a salt thereof); about 0.005%, about 0.0075%, about 0.01%, about 0.0125% or about 0.015% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 2.5%, about 3%, about 3.5%, about 4%, about 4.5%, about 5%, about 5.5%, about 6%, about 6.5% or about 7% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments, the anion exchange dilution buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 1-10 mM of a weak acid or salt thereof (e.g., citric acid or a salt thereof, acetic acid or a salt thereof, or succinic acid or a salt thereof), about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), about 0.01% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof). For Example, the anion exchange dilution buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 1 mM, about 1.5 mM, about 2 mM, about 2.5 mM, 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM or about 10 mM of a weak acid or salt thereof (e.g., citric acid or a salt thereof, acetic acid or a salt thereof, or succinic acid or a salt thereof); about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), about 0.5% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 5% of a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof), and the buffer has a high pH (e.g., a pH of about 8.5-9.5, such as a pH of about 8.5, about 9 or about 9.5).

In some embodiments of any one of the aspects, the anion exchange dilution buffer comprises: of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a weak acid or salt thereof (e.g., citric acid or a salt thereof, acetic acid or a salt thereof, or succinic acid or a salt thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and optionally the buffer is substantially free of glycine. For example, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 3-12 mM citric acid, about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and optionally the buffer is substantially free of glycine. In some embodiments, the anion exchange dilution buffer comprises: about 75-125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 75-125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 3-10 mM of a weak acid or salt thereof (e.g., citric acid or a salt thereof, acetic acid or a salt thereof, or succinic acid or a salt thereof), about 0.3-0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and about 0.5-1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and the buffer has a high pH (e.g., a pH of about 8.5-9.5, such as a pH of about 8.5, about 9 or about 9.5), and optionally the buffer is substantially free of glycine.

In some embodiments, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 3 mM, about 3.5 mM, about 4 mM, about 4.5 mM, about 5 mM, about 5.5 mM, about 6 mM, about 6.5 mM, about 7 mM, about 7.5 mM, about 7.5 mM, about 8 mM, about 8.5, about 9 mM, about 9.5 mM, about 10 mM, about 10.5 mM, about 11 mM, about 11.5 mM or about 12 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); and about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and optionally the buffer is substantially free of glycine.

In some embodiments, the anion exchange dilution buffer comprises: about 75 mM, about 80 mM, about 85 mM, about 90 mM, about 95 mM, about 100 mM, about 105 mM, about 110 mM, about 115 mM, about 120 mM or about 125 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 75 mM, about 80 mM, about 85 mM, about 90 mM, about 95 mM, about 100 mM, about 105 mM, about 110 mM, about 115 mM, about 120 mM or about 125 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 3 mM, about 3.5 mM, about 4 mM, about 4.5 mM, about 5 mM, about 5.5 mM, about 6 mM, about 6.5 mM, about 7 mM, about 7.5 mM, about 7.5 mM, about 8 mM, about 8.5, about 9 mM, about 9.5 mM, about 10 mM, about 10.5 mM, about 11 mM, about 11.5 mM or about 12 mM of a weak acid or salt thereof (e.g., citric acid or a salt thereof, acetic acid or a salt thereof, or succinic acid or a salt thereof); about 0.3%, about 0.35%, about 0.4%, about 0.45%, about 0.5%, about 0.55%, about 0.6%, about 0.65% or about 0.7% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPGPluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, BrijS10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof); and about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and the buffer has a high pH (e.g., a pH of about 8.5-9.5, such as a pH of about 8.5, about 9 or about 9.5), and optionally the buffer is substantially free of glycine.

In some embodiments, the anion exchange dilution buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 6-8 mM of a weak acid or salt thereof (e.g., citric acid or a salt thereof, acetic acid or a salt thereof, or succinic acid or a salt thereof), about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), and about 0.5% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and optionally the buffer is substantially free of glycine. For Example, the anion exchange dilution buffer comprises: about 100 mM of a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof); about 100 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 6 mM, about 6.5 mM, about 7 mM, about 7.5 mM or about 8 mM of a weak acid or salt thereof (e.g., citric acid or a salt thereof, acetic acid or a salt thereof, or succinic acid or a salt thereof); about 1 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); and about 0.5% of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and the buffer has a high pH (e.g., a pH of about 8.5-9.5, such as a pH of about 8.5, about 9 or about 9.5), and optionally the buffer is substantially free of glycine.

In some embodiments, the anion exchange dilution buffer comprises: about 100 mM BTP, about 100 mM histidine, about 6-8 mM of citric acid or a salt thereof, about 1 mM MgCl₂, and about 0.5% PF68, and optionally the buffer is substantially free of glycine. For Example, the anion exchange dilution buffer comprises: about 100 mM BTP; about 100 mM histidine; about 6 mM, about 6.5 mM, about 7 mM, about 7.5 mM or about 8 mM of citric acid or a salt thereof; about 1 mM MgCl₂; and about 0.5% PF68, and the buffer has a high pH (e.g., a pH of about 8.5-9.5, such as a pH of about 8.5, about 9 or about 9.5), and optionally the buffer is substantially free of glycine.

In some embodiments of any one of the aspects, the dilution buffer comprises: BTP, histidine, glycerol, PF68, MgCl₂ and has a high pH. For example, the dilution buffer comprises: about 75-125 mM BTP, about 75-125 mM histidine, about 2.5-7.5% glycerol, about 0.3-0.7% PF68, about 0.5-1.5 mM MgCl₂ and has a pH of about 8.5-9.5.

In some embodiments, the dilution buffer comprises: about 75 mM, about 80 mM, about 85 mM, about 90 mM, about 95 mM, about 100 mM, about 105 mM, about 110 mM, about 115 mM, about 120 mM or about 125 mM of BTP; about 75 mM, about 80 mM, about 85 mM, about 90 mM, about 95 mM, about 100 mM, about 105 mM, about 110 mM, about 115 mM, about 120 mM or about 125 mM of histidine; about 0.3%, about 0.35%, about 0.4%, about 0.45%, about 0.5%, about 0.55%, about 0.6%, about 0.65% or about 0.7% of PF68; about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of MgCl₂; and about 2.5%, about 3%, about 3.5%, about 4%, about 4.5%, about 5%, about 5.5%, about 6%, about 6.5% or about 7% of glycerol.

In some embodiments, the dilution buffer comprises: about 75 mM, about 80 mM, about 85 mM, about 90 mM, about 95 mM, about 100 mM, about 105 mM, about 110 mM, about 115 mM, about 120 mM or about 125 mM of BTP; about 75 mM, about 80 mM, about 85 mM, about 90 mM, about 95 mM, about 100 mM, about 105 mM, about 110 mM, about 115 mM, about 120 mM or about 125 mM of histidine; about 0.3%, about 0.35%, about 0.4%, about 0.45%, about 0.5%, about 0.55%, about 0.6%, about 0.65% or about 0.7% of PF68; about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of MgCl₂; and about 2.5%, about 3%, about 3.5%, about 4%, about 4.5%, about 5%, about 5.5%, about 6%, about 6.5% or about 7% of glycerol, and the buffer has a high pH, e.g., a pH of about 8.5-9.5, such as a pH of about 8.5, about 9 or about 9.5.

In some embodiments of any one of the aspects, the dilution buffer comprises: BTP, histidine, PF68, MgCl₂ and has a high pH. For example, the dilution buffer comprises: about 75-125 mM BTP, about 75-125 mM histidine, about 0.3-0.7% PF68, about 0.5-1.5 mM MgCl₂ and has a pH of about 8.5-9.5.

In some embodiments, the dilution buffer comprises: about 75 mM, about 80 mM, about 85 mM, about 90 mM, about 95 mM, about 100 mM, about 105 mM, about 110 mM, about 115 mM, about 120 mM or about 125 mM of BTP; about 75 mM, about 80 mM, about 85 mM, about 90 mM, about 95 mM, about 100 mM, about 105 mM, about 110 mM, about 115 mM, about 120 mM or about 125 mM of histidine; and about 0.3%, about 0.35%, about 0.4%, about 0.45%, about 0.5%, about 0.55%, about 0.6%, about 0.65% or about 0.7% of PF68; about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of MgCl₂.

In some embodiments, the dilution buffer comprises: about 75 mM, about 80 mM, about 85 mM, about 90 mM, about 95 mM, about 100 mM, about 105 mM, about 110 mM, about 115 mM, about 120 mM or about 125 mM of BTP; about 75 mM, about 80 mM, about 85 mM, about 90 mM, about 95 mM, about 100 mM, about 105 mM, about 110 mM, about 115 mM, about 120 mM or about 125 mM of histidine; about 0.3%, about 0.35%, about 0.4%, about 0.45%, about 0.5%, about 0.55%, about 0.6%, about 0.65% or about 0.7% of PF68; and about 0.5 mM, about 0.6 mM, about 0.7 mM, about 0.8 mM, about 0.9 mM, about 1 mM, about 1.1 mM, about 1.2 mM, about 1.3 mM, about 1.4 mM or about 1.5 mM of MgCl₂, and the buffer has a high pH, e.g., a pH of about 8.5-9.5, such as a pH of about 8.5, about 9 or about 9.5.

In some embodiments, the anion exchange dilution buffer comprises citric acid or citrate at a concentration of from about 0.5 mM to about 15 mM and less than 10% of the empty AAV particles bind to anion exchange column. For example, the anion exchange dilution buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the dilution buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM), and less than 8%, less than 6%, less than 5%, less than 4%, less than 3%, less than 2%, less than 1%, less than 0.5%, less than 0.2%, less than 0.1%, less than 0.05%, or, less than 0.02% or, even less (e.g., substantially none) of the empty AAV particles bind to anion exchange column.

In some embodiments, the anion exchange dilution buffer comprises citric acid or citrate at a concentration of from about 0.5 mM to about 15 mM and less than 30% of the AAV viral particles in the eluate from the anion exchange are empty viral particles. For example, the anion exchange dilution buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the dilution buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM), and less than 20%, less than 19.5%, less than 19%, less than 18.5%, less than 18%, less than 17.5%, less than 17%, less than 16.5%, less than 16%, less than 15.5%, less than 15%, less than 14.5%, less than 14%, less than 13.5%, less than 13%, less than 12.5%, less than 12%, less than 11.5%, less than 11%, less than 10.5%, less than 10%, less than 9.5%, less than 9%, less than 8.5%, less than 8%, less than 7.5%, less than 7%, less than 6.5%, less than 6%, less than 5.5%, less than 5%, less than 4.5%, less than 4%, less than 3.5%, less than 3%, less than 2.5%, less than 2%, less than 1.5%, less than 1%, less than 0.75%, less than 0.5%, less than 0.25%, less than 0.2%, less than 0.15%, less than 0.1%, less than 0.05%, less than 0.04%, or less than 0.02% (e.g., substantially none) of the AAV viral particles in the eluate from the anion exchange are empty viral particles.

In some embodiments, the anion exchange dilution buffer comprises citric acid or citrate at a concentration of from about 0.5 mM to about 15 mM and the amount of empty viral particles is reduced by 86 fold or more in the eluate from the anion exchange compared to the amount of empty viral particles in the affinity eluate. For example, the anion exchange dilution buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the dilution buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM), and the amount of empty viral particles is reduced by 90 fold or more, 95 fold or more, or 99 fold or more in the eluate from the anion exchange compared to the amount of empty viral particles in the affinity eluate.

In some embodiments, the anion exchange dilution buffer comprises citric acid or citrate at a concentration of from about 0.5 mM to about 15 mM and a ratio of total rAAV viral particles (e.g., full, partially full and empty AAV viral particles) to empty rAAV viral particles in the anion exchange eluate is at least about 1.25× higher than the ratio of total rAAV viral particles to empty particles in the affinity eluate. For example, the anion exchange dilution buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the dilution buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM), and the ratio of total rAAV viral particles to empty rAAV viral particles in the anion exchange eluate is at least about 1.5×, at least about 1.6×, at least about 1.7×, at least about 1.8×, at least about 1.9×, at least about 2×, at least about 2.1×, at least about 2.2×, at least about 2.3×, at least about 2.4×, at least about 2.5×, at least about 2.6×, at least about 2.7×, at least about 2.8×, at least about 2.9×, at least about 3×, at least about 3.1×, at least about 3.2×, at least about 3.3×, at least about 3.4×, at least about 3.5×, at least about 3.6×, at least about 3.7×, at least about 3.8×, at least about 3.9×, at least about 4×, at least about 4.1×, at least about 4.2×, at least about 4.3×, at least about 4.4×, at least about 4.5×, at least about 4.6×, at least about 4.7×, at least about 4.8×, at least about 4.9×, at least about 5×, at least about 5.1×, at least about 5.2×, at least about 5.3×, at least about 5.4×, at least about 5.5×, at least about 5.6×, at least about 5.7×, at least about 5.8×, at least about 5.9×, at least about 6×, at least about 6.1×, at least about 6.2×, at least about 6.3×, at least about 6.4×, at least about 6.5×, at least about 6.6×, at least about 6.7×, at least about 6.8×, at least about 6.9×, at least about 7×, at least about 7.1×, at least about 7.2×, at least about 7.3×, at least about 7.4×, at least about 7.5×, at least about 7.6×, at least about 7.7×, at least about 7.8×, at least about 7.9×, at least about 8×, at least about 8.1×, at least about 8.2×, at least about 8.3×, at least about 8.4×, at least about 8.5×, at least about 8.6×, at least about 8.7×, at least about 8.8×, at least about 8.9×, at least about 9×, at least about 9.1×, at least about 9.2×, at least about 9.3×, at least about 9.4×, at least about 9.5×, at least about 9.6×, at least about 9.7×, at least about 9.8×, at least about 9.9×, or at least about 10× higher relative to the ratio of total rAAV viral particles to empty rAAV particles in the affinity eluate.

In some embodiments, the anion exchange dilution buffer comprises citric acid or citrate at a concentration of from about 0.5 mM to about 15 mM and a ratio of full and partially full rAAV particles to empty rAAV viral particles in the anion exchange eluate is at least about 1.25× higher than the ratio of total rAAV viral particles to empty particles in the affinity eluate. For example, the anion exchange dilution buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the dilution buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM), and the ratio of full and partially full rAAV particles to empty rAAV viral particles in the anion exchange eluate is at least about 1.5×, at least about 1.6×, at least about 1.7×, at least about 1.8×, at least about 1.9×, at least about 2×, at least about 2.1×, at least about 2.2×, at least about 2.3×, at least about 2.4×, at least about 2.5×, at least about 2.6×, at least about 2.7×, at least about 2.8×, at least about 2.9×, at least about 3×, at least about 3.1×, at least about 3.2×, at least about 3.3×, at least about 3.4×, at least about 3.5×, at least about 3.6×, at least about 3.7×, at least about 3.8×, at least about 3.9×, at least about 4×, at least about 4.1×, at least about 4.2×, at least about 4.3×, at least about 4.4×, at least about 4.5×, at least about 4.6×, at least about 4.7×, at least about 4.8×, at least about 4.9×, at least about 5×, at least about 5.1×, at least about 5.2×, at least about 5.3×, at least about 5.4×, at least about 5.5×, at least about 5.6×, at least about 5.7×, at least about 5.8×, at least about 5.9×, at least about 6×, at least about 6.1×, at least about 6.2×, at least about 6.3×, at least about 6.4×, at least about 6.5×, at least about 6.6×, at least about 6.7×, at least about 6.8×, at least about 6.9×, at least about 7×, at least about 7.1×, at least about 7.2×, at least about 7.3×, at least about 7.4×, at least about 7.5×, at least about 7.6×, at least about 7.7×, at least about 7.8×, at least about 7.9×, at least about 8×, at least about 8.1×, at least about 8.2×, at least about 8.3×, at least about 8.4×, at least about 8.5×, at least about 8.6×, at least about 8.7×, at least about 8.8×, at least about 8.9×, at least about 9×, at least about 9.1×, at least about 9.2×, at least about 9.3×, at least about 9.4×, at least about 9.5×, at least about 9.6×, at least about 9.7×, at least about 9.8×, at least about 9.9×, or at least about 10× higher relative to the ratio of full and partially full rAAV particles to empty rAAV particles in the affinity eluate.

In some embodiments, the anion exchange dilution buffer comprises citric acid or citrate at a concentration of from about 0.5 mM to about 15 mM and the UV₂₆₀ to UV₂₈₀ ratio of the anion exchange eluate is at least 1.25 or more. For example, the anion exchange dilution buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the dilution buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM), and the UV₂₆₀ to UV₂₈₀ ratio of the anion exchange eluate is at least 1.25, at least 1.26, at least 1.27, at least 1.28, at least 1.29, at least 1.3, at least 1.31, at least 1.32, at least 1.33, at least 1.34, or at least 1.35 or more.

In some embodiments, the anion exchange dilution buffer comprises citric acid or citrate at a concentration of from about 0.5 mM to about 15 mM and the ratio of UV₂₆₀ to UV₂₈₀ in the anion exchange eluate is at least about 1.15× or higher than a ratio of UV₂₆₀ to UV₂₈₀ ratio in the adjusted affinity eluate. For example, the anion exchange dilution buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the dilution buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM), and the ratio of UV₂₆₀ to UV₂₈₀ in the anion exchange eluate is at least about 1.2×, at least about 1.25×, at least about 1.3×, at least about 1.35×, at least about 1.4×, at least about 1.45×, at least about 1.5×, at least about 1.55×, at least about 1.6×, at least about 1.65×, at least at least about 1.7×, or at least about 1.75×, at least about 1.8×, at least about 1.859×, at least about 1.9×, at least about 1.95×, or at least about 2× or higher than the ratio of UV₂₆₀ to UV₂₈₀ ratio in the adjusted affinity eluate.

In some embodiments, the anion exchange equilibration buffer comprises citric acid or citrate at a concentration of from about 0.5 mM to about 15 mM and less than 10% of the empty AAV particles bind to anion exchange column. For example, the anion exchange equilibration buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the equilibration buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM, or the equilibration buffer comprises citric acid or citrate at a concentration of upto about 4 mM), and less than 8%, less than 6%, less than 5%, less than 4%, less than 3%, less than 2%, less than 1%, less than 0.5%, less than 0.2%, less than 0.1%, less than 0.05%, or, less than 0.02% or, even less (e.g., substantially none) of the empty AAV particles bind to anion exchange column.

In some embodiments, the anion exchange equilibration buffer comprises citric acid or citrate at a concentration of from about 0.5 mM to about 15 mM and less than 30% of the AAV viral particles in the eluate from the anion exchange are empty viral particles. For example, the anion exchange equilibration buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the equilibration buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM, or the equilibration buffer comprises citric acid or citrate at a concentration of upto about 4 mM), and less than 20%, less than 19.5%, less than 19%, less than 18.5%, less than 18%, less than 17.5%, less than 17%, less than 16.5%, less than 16%, less than 15.5%, less than 15%, less than 14.5%, less than 14%, less than 13.5%, less than 13%, less than 12.5%, less than 12%, less than 11.5%, less than 11%, less than 10.5%, less than 10%, less than 9.5%, less than 9%, less than 8.5%, less than 8%, less than 7.5%, less than 7%, less than 6.5%, less than 6%, less than 5.5%, less than 5%, less than 4.5%, less than 4%, less than 3.5%, less than 3%, less than 2.5%, less than 2%, less than 1.5%, less than 1%, less than 0.75%, less than 0.5%, less than 0.25%, less than 0.2%, less than 0.15%, less than 0.1%, less than 0.05%, less than 0.04%, or less than 0.02% (e.g., substantially none) of the AAV viral particles in the eluate from the anion exchange are empty viral particles.

In some embodiments, the anion exchange equilibration buffer comprises citric acid or citrate at a concentration of from about 0.5 mM to about 15 mM and the amount of empty viral particles is reduced by 86 fold or more in the eluate from the anion exchange compared to the amount of empty viral particles in the affinity eluate. For example, the anion exchange equilibration buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the equilibration buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM, or the equilibration buffer comprises citric acid or citrate at a concentration of upto about 4 mM), and the amount of empty viral particles is reduced by 90 fold or more, 95 fold or more, or 99 fold or more in the eluate from the anion exchange compared to the amount of empty viral particles in the affinity eluate.

In some embodiments, the anion exchange equilibration buffer comprises citric acid or citrate at a concentration of from about 0.5 mM to about 15 mM and a ratio of total rAAV viral particles (e.g., full, partially full and empty AAV viral particles) to empty rAAV viral particles in the anion exchange eluate is at least about 1.25× higher than the ratio of total rAAV viral particles to empty particles in the affinity eluate. For example, the anion exchange equilibration buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the equilibration buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM, or the equilibration buffer comprises citric acid or citrate at a concentration of upto about 4 mM), and the ratio of total rAAV viral particles to empty rAAV viral particles in the anion exchange eluate is at least about 1.5×, at least about 1.6×, at least about 1.7×, at least about 1.8×, at least about 1.9×, at least about 2×, at least about 2.1×, at least about 2.2×, at least about 2.3×, at least about 2.4×, at least about 2.5×, at least about 2.6×, at least about 2.7×, at least about 2.8×, at least about 2.9×, at least about 3×, at least about 3.1×, at least about 3.2×, at least about 3.3×, at least about 3.4×, at least about 3.5×, at least about 3.6×, at least about 3.7×, at least about 3.8×, at least about 3.9×, at least about 4×, at least about 4.1×, at least about 4.2×, at least about 4.3×, at least about 4.4×, at least about 4.5×, at least about 4.6×, at least about 4.7×, at least about 4.8×, at least about 4.9×, at least about 5×, at least about 5.1×, at least about 5.2×, at least about 5.3×, at least about 5.4×, at least about 5.5×, at least about 5.6×, at least about 5.7×, at least about 5.8×, at least about 5.9×, at least about 6×, at least about 6.1×, at least about 6.2×, at least about 6.3×, at least about 6.4×, at least about 6.5×, at least about 6.6×, at least about 6.7×, at least about 6.8×, at least about 6.9×, at least about 7×, at least about 7.1×, at least about 7.2×, at least about 7.3×, at least about 7.4×, at least about 7.5×, at least about 7.6×, at least about 7.7×, at least about 7.8×, at least about 7.9×, at least about 8×, at least about 8.1×, at least about 8.2×, at least about 8.3×, at least about 8.4×, at least about 8.5×, at least about 8.6×, at least about 8.7×, at least about 8.8×, at least about 8.9×, at least about 9×, at least about 9.1×, at least about 9.2×, at least about 9.3×, at least about 9.4×, at least about 9.5×, at least about 9.6×, at least about 9.7×, at least about 9.8×, at least about 9.9×, or at least about 10× higher relative to the ratio of total rAAV viral particles to empty rAAV particles in the affinity eluate.

In some embodiments, the anion exchange equilibration buffer comprises citric acid or citrate at a concentration of from about 0.5 mM to about 15 mM and a ratio of full and partially full rAAV particles to empty rAAV viral particles in the anion exchange eluate is at least about 1.25× higher than the ratio of total rAAV viral particles to empty particles in the affinity eluate. For example, the anion exchange equilibration buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the equilibration buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM, or the equilibration buffer comprises citric acid or citrate at a concentration of upto about 4 mM), and the ratio of full and partially full rAAV particles to empty rAAV viral particles in the anion exchange eluate is at least about 1.5×, at least about 1.6×, at least about 1.7×, at least about 1.8×, at least about 1.9×, at least about 2×, at least about 2.1×, at least about 2.2×, at least about 2.3×, at least about 2.4×, at least about 2.5×, at least about 2.6×, at least about 2.7×, at least about 2.8×, at least about 2.9×, at least about 3×, at least about 3.1×, at least about 3.2×, at least about 3.3×, at least about 3.4×, at least about 3.5×, at least about 3.6×, at least about 3.7×, at least about 3.8×, at least about 3.9×, at least about 4×, at least about 4.1×, at least about 4.2×, at least about 4.3×, at least about 4.4×, at least about 4.5×, at least about 4.6×, at least about 4.7×, at least about 4.8×, at least about 4.9×, at least about 5×, at least about 5.1×, at least about 5.2×, at least about 5.3×, at least about 5.4×, at least about 5.5×, at least about 5.6×, at least about 5.7×, at least about 5.8×, at least about 5.9×, at least about 6×, at least about 6.1×, at least about 6.2×, at least about 6.3×, at least about 6.4×, at least about 6.5×, at least about 6.6×, at least about 6.7×, at least about 6.8×, at least about 6.9×, at least about 7×, at least about 7.1×, at least about 7.2×, at least about 7.3×, at least about 7.4×, at least about 7.5×, at least about 7.6×, at least about 7.7×, at least about 7.8×, at least about 7.9×, at least about 8×, at least about 8.1×, at least about 8.2×, at least about 8.3×, at least about 8.4×, at least about 8.5×, at least about 8.6×, at least about 8.7×, at least about 8.8×, at least about 8.9×, at least about 9×, at least about 9.1×, at least about 9.2×, at least about 9.3×, at least about 9.4×, at least about 9.5×, at least about 9.6×, at least about 9.7×, at least about 9.8×, at least about 9.9×, or at least about 10× higher relative to the ratio of full and partially full rAAV particles to empty rAAV particles in the affinity eluate.

In some embodiments, the anion exchange equilibration buffer comprises citric acid or citrate at a concentration of from about 0.5 mM to about 15 mM and the UV₂₆₀ to UV₂₈₀ ratio of the anion exchange eluate is at least 1.25 or more. For example, the anion exchange equilibration buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the equilibration buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM, or the equilibration buffer comprises citric acid or citrate at a concentration of upto about 4 mM), and the UV₂₆₀ to UV₂₈₀ ratio of the anion exchange eluate is at least 1.25, at least 1.26, at least 1.27, at least 1.28, at least 1.29, at least 1.3, at least 1.31, at least 1.32, at least 1.33, at least 1.34, or at least 1.35 or more.

In some embodiments, the anion exchange equilibration buffer comprises citric acid or citrate at a concentration of from about 0.5 mM to about 15 mM and the ratio of UV₂₆₀ to UV₂₈₀ in the anion exchange eluate is at least about 1.15× or higher than a ratio of UV₂₆₀ to UV₂₈₀ ratio in the adjusted affinity eluate. For example, the anion exchange equilibration buffer comprises citric acid or citrate at a concentration of from about 1 mM to about 10 mM, from about 1.5 mM to about 7.5 mM, about 2 mM to about 7 mM, about 1.5 mM to about 9 mM, about 2 mM to about 8 mM or from about 2.5 mM to about 7.5 mM (e.g., the equilibration buffer comprises citric acid or citrate at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM, or the equilibration buffer comprises citric acid or citrate at a concentration of upto about 4 mM), and the ratio of UV₂₆₀ to UV₂₈₀ in the anion exchange eluate is at least about 1.2×, at least about 1.25×, at least about 1.3×, at least about 1.35×, at least about 1.4×, at least about 1.45×, at least about 1.5×, at least about 1.55×, at least about 1.6×, at least about 1.65×, at least at least about 1.7×, or at least about 1.75×, at least about 1.8×, at least about 1.859×, at least about 1.9×, at least about 1.95×, or at least about 2× or higher than the ratio of UV₂₆₀ to UV₂₈₀ ratio in the adjusted affinity eluate.

In some embodiments of any one of the aspects described herein, the affinity eluate comprises a weak acid or salt thereof (e.g., citric acid or a salt thereof, acetic acid or a salt thereof, or succinic acid or a salt thereof), and the affinity elute is diluted with an anion exchange dilution buffer comprising: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a weak acid or salt thereof (e.g., citric acid or a salt thereof, acetic acid or a salt thereof, or succinic acid or a salt thereof), a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂) and a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), optionally the equilibration buffer is substantially free of glycine.

In some embodiments of any one of the aspects described herein, the affinity eluate is substantially free of a weak acid or salt thereof (e.g., citric acid or a salt thereof, acetic acid or a salt thereof, or succinic acid or a salt thereof), and the affinity elute is diluted with an anion exchange dilution buffer comprising: a buffering agent (e.g., a buffering agent selected from the group consisting of acetate, histidine, phosphate, citrate, propionate, tricine, borate, tris(hydroxymethyl)aminomethane (tris), and any combinations thereof; or a buffering agent selected from the group consisting of BTP, tris, borate, tricine, and any combinations thereof, or a buffering agent selected from the group consisting of BTP, tris and any combinations thereof), an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), a weak acid or salt thereof (e.g., citric acid or a salt thereof, acetic acid or a salt thereof, or succinic acid or a salt thereof), a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or a non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68 (PF68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, PF68, Ecosurf SA-15, Brij S20, Tergitol NP-10, IGEPAL CA 720, Tween 80 and any combinations thereof, or a non-ionic surfactant selected from the group consisting of Pluronic 10R5 and PF68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof) and a viscosity modifier (e.g., a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of glycerol, sorbitol, mannitol, dulcitol, sucrose, lactose, maltose, trehalose and any combinations thereof, or polyol selected from the group consisting of glycerol, sucrose, mannitol, sorbitol and any combinations thereof, or a polyol selected from the group consisting of propylene glycol, polyethylene glycol, dextran and any combinations thereof).

Generally, the dilution buffer has a conductivity in a range from about 0.5 mS/cm to about 3 mS/cm. For example, the dilution buffer a conductivity in a range from about 1 mS/cm to about 2.5 mS/cm, from about 1.25 mS/cm to about 2.25 mS/cm, or from about 1.5 mS/cm to about 1.75 mS/cm. In some embodiments, the dilution buffer has a conductivity of about 1.67 mS/cm.

The osmolarity of the dilution buffer is less than 900 mOsm. For example, the osmolarity of the dilution buffer is from about 200 mOsm to about 900 mOsm.

As discussed herein, the feed composition for the anion exchange chromatography can be an eluate from affinity chromatography. Thus, in another aspect provided herein is a method for obtaining a composition comprising recombinantly expressed virus particles. Generally, the method comprises contacting a harvest media comprising recombinantly expressed virus particles with an affinity chromatography media under conditions that allow binding of virus particles to the affinity chromatography media, and recovering an eluate (affinity eluate) comprising recombinant virus particles. The recovered eluate can be used as the feed composition for anion exchange chromatography.

In various embodiments, the elution buffer (affinity elution buffer) for recovering the recombinant virus particles from the affinity chromatography comprises glycine. For example, the affinity elution buffer comprises glycine at a concentration of at least about 20 mM, 25 mM, 30 mM, 35 mM, 40 mM, 45 mM, 50 mM, 55 mM, 60 mM, 65 mM, 70 mM, 75 mM, 80 mM, 90 mM, 95 mM, 100 mM or more. In some embodiments of any one of the aspects, the affinity elution buffer comprises glycine at a concentration of from about 25 mM to about 100 mM, from about 30 mM to about 95 mM, from about 35 mM to about 90 mM, from about 40 mM to about 80 mM, or from about 45 mM to about 75 mM.

In some embodiments of any one of the aspects, the affinity elution buffer comprises glycine at a concentration of about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 90 mM, about 95 mM or about 100 mM. For example, the affinity elution buffer comprises glycine at a concentration of about 50 mM. In another non-limiting example, the affinity elution buffer comprises glycine at a concentration of about 75 mM.

The affinity elution buffer can also comprise a cation, e.g., a monovalent or divalent cation. Exemplary monovalent ions for the elution buffer include, but are not limited to, sodium (Na⁺), lithium (Li⁺), potassium (K⁺), rubidium (Rb⁺), cesium (Cs⁺), francium (Fr⁺), alkylamino and ammonium. Exemplary divalent cations for the elution buffer include, but are not limited to, magnesium (Mg²⁺), calcium (Ca²⁺), copper (Cu²⁺), cobalt (Co²⁺), manganese (Mn²⁺), nickel (Ni²⁺) and zinc (Zn²⁺). The cation can be added in the form of salt. In some embodiments, the elution buffer comprises a divalent cation, e.g., Mg²⁺.

It is noted that the affinity elution buffer can comprise the cation, e.g., a divalent cation such as Mg²⁺ at a concentration of at least about 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 50 mM, 55 mM or more. For example, the affinity elution buffer can comprise a cation, e.g., a divalent cation such as Mg²⁺ at a concentration of from about 5 mM to about 15 mM, from about 6 mM to about 14 mM, from about 7 mM to about 13 mM, from about 8 mM to about 12 mM or from about 9 mM to about 11 mM. In some embodiments, the affinity elution buffer comprises a cation, e.g., a divalent cation such as Mg²⁺ at a concentration of about 5 mM, about 6 mM, about 7 mM, about 8 mM, about 9 mM, about 10 mM, about 11 mM, about 12 mM, about 13 mM, about 14 mM, or about 15 mM. For example, the affinity elution buffer comprises a cation, e.g., a divalent cation such as Mg²⁺ at a concentration of about 10 mM.

In some embodiments of any one of the aspects, the affinity elution buffer comprises a magnesium salt, e.g., MgCl₂.

The affinity elution buffer can also comprise a polymer. For example, the affinity elution buffer can comprise a non-ionic polymer. Exemplary non-ionic polymers include poloxamer (also known by the trade names SYNPERONICS™, PLURONIC™, and KOLLIPHOR™), and polyethyleneglycols (PEGs).

In some embodiments, the affinity elution buffer comprises a poloxomer. Exemplary poloxamers include, but are not limited to, Poloxamer 188 (P188), Pluronic® F127, Pluronic® F38, Pluronic® F68, Pluronic® F87, Pluronic® F108, Pluronic® 10R5, Pluronic® 17R2, Pluronic® 17R4, Pluronic® 25R2, Pluronic® 25R4, Pluronic® 31R1, Pluronic® F108 Cast Solid Surfacta, Pluronic® F108 NF, Pluronic® F108 Pastille, Pluronic® F108NF Prill Poloxamer 338, Pluronic® F127 NF, Pluronic® F127 NF 500 BHT Prill, Pluronic® F127 NF Prill Poloxamer 407, Pluronic® F38 Pastille, Pluronic® F68 LF Pastille, Pluronic® F68 NF, Pluronic® F68 NF Prill, Pluronic® F68 Pastille, Pluronic® F77, Pluronic® F77 Micropastille, Pluronic® F87 NF, Pluronic® F87 NF Prill Poloxamer 237, Pluronic® F 88, Pluronic® F 88 Pastille, Pluronic® F 98, Pluronic® FT L 61, Pluronic® L10, Pluronic® L101, Pluronic® L121, Pluronic® L31, Pluronic® L35, Pluronic® L43, Pluronic® L61, Pluronic® L62, Pluronic® L62 LF, Pluronic® L62D, Pluronic® L64, Pluronic® L81, Pluronic® L92, Pluronic® L44 NF INH surfactant Poloxamer 124, Pluronic® N3, Pluronic® P103, Pluronic® P104, Pluronic® P105, Pluronic® P123 Surfactant, Pluronic® P65, Pluronic® P84, Pluronic® P85, and the like

The amount of the polymer, e.g., poloxomer in the affinity elution buffer can be varied. For example, the amount of the polymer, e.g., poloxomer in the affinity elution buffer can be at least about 0.1%, 0.15%, 0.2%, 0.25%, 0.3%, 0.35%, 0.4%, 0.45%, 0.5% (w/w, w/v or v/v) or more. In some embodiments, the affinity elution buffer comprises the polymer, e.g., poloxomer at a concentration of from about 0.1% to about 0.5%, from about 0.15% to about 0.45%, from about 0.2% to about 0.4%, or from about 0.25% to about 0.35%. 0.1%. For example, the affinity elution buffer comprises the polymer, e.g., poloxomer at a concentration of about 0.1%, about 0.15%, about 0.2%, about 0.25%, about 0.3%, about 0.35%, about 0.4%, about 0.45%, or about 0.5%.

Generally, the affinity elution buffer has a low pH. For example, the affinity elution buffer has a pH lower than or equal to about 6.5, 6.0, 5.5, 5.0, 4.5, 4.0, 3.5, 3.0, 2.5, 2.2, 2.0, 1.5 or lower. In some embodiments, the affinity elution buffer has a pH lower than or equal to about 4.5. For example, the affinity elution buffer has a pH of about 2.0 to about 3.0. For example, the affinity elution buffer has a pH of about 2.0, 2.1, 2.2, 2.3, 2.4, 2.5, 2.6, 2.7, 2.8, 2.9 or 3.0. In some embodiments, the affinity elution buffer has a pH of from about 2.0 to about 3.0. In some embodiments, the affinity elution buffer has a pH of about 2.2.

The affinity elution buffer can also comprise histidine. When the affinity elution buffer comprises histidine, the histidine can be at a concentration of at least about 1 mM, 5 mM, 10 mM, 15 mM, 20 mM, 25 mM, 30 mM, 35 mM, 40 mM, 45 mM, 50 mM or more. For example, the affinity elution buffer can comprise histidine at a concentration from about 1 mM to about 50 mM, from about 5 mM to about 45 mM, from about 10 mM to about 40 mM, from about 15 mM to about 35 mM or from about 20 mM to about 30 mM. In some embodiments, the affinity elution buffer comprises histidine at a concentration of about 1 mM, about 5 mM, about 10 mM, about 15 mM, about 20 mM, about 25 mM, about 30 mM, about 35 mM, about 40 mM, about 45 mM or about 50 mM. For example, the affinity elution buffer comprises histidine at a concentration of about 25 mM.

In some embodiments, the affinity elution buffer comprises: about 25-75 mM glycine, about 50-100 mM histidine, about 2-25 mM MgCl₂, about 0.1-0.5% P188 and has a pH of about 2.5-3.5. For example, the affinity elution buffer comprises: about 75 mM glycine, about 75 mM histidine, about 10 mM MgCl₂, about 0.3% P188 and has a pH of about 3.0.

In some embodiments, the affinity elution buffer is substantially free of histidine.

In some embodiments, the affinity elution buffer comprises citric acid or a salt thereof, e.g., citrate. The affinity elution buffer can comprise citric acid or a salt thereof at a concentration of at least about 50 mM, 55 mM, 60 mM, 65 mM, 70 mM, 75 mM, 80 mM, 85 mM, 90 mM, 95 mM, 100 mM or more. For example, the affinity elution buffer comprises citric acid or a salt thereof at a concentration of from about 50 mM to about 100 mM, from about 55 mM to about 95 mM, from about 60 mM to about 90 mM, from about 65 mM to about 85 mM or from about 70 mM to about 75 mM. In some embodiments, the affinity elution buffer comprises citric acid or a salt thereof at a concentration of about 50 mM, about 55 mM, about 60 mM, about 65 mM, about 70 mM, about 75 mM, about 80 mM, about 85 mM, about 90 mM, about 95 mM, or about 100 mM. For example, the affinity elution buffer comprises citric acid or a salt thereof at a concentration of about 75 mM.

In some embodiments, the affinity elution buffer comprises: about 50-100 mM glycine, about 10-50 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 2-25 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), about 0.1-0.5% P188. For example, the affinity elution buffer comprises: about 75 mM glycine, about 75 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine), about 10 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂), about 0.3% P188 and has a low pH, e.g., a pH of about 2-5 such as a pH of about 2, bout 2.5, about 3, about 3.5 or about 4.

In some embodiments, the affinity elution buffer comprises: about 50 mM, about 55 mM, about 60 mM, about 65 mM, about 70 mM, about 75 mM, about 80 mM, about 85 mM, about 90 mM, about 95 mM or about 100 mM glycine; about 10 mM, about 15 mM, about 20 mM, about 25 mM, about 30 mM, about 35 mM, about 40 mM, about 45 mM or about 50 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 2.5 mM, about 3 mM, about 3.5 mM, about 4 mM, about 4.5 mM, about 5 mM, about 7.5 mM, about 10 mM, about 12.5 mM, about 15 mM, about 17.5 mM, about 20 mM, about 22.5 mM or about 25 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); about 0.1%, about 0.15%, about 0.2%, about 0.25%, about 0.3%, about 0.35%, about 0.4%, about 0.45% or about 0.5% P188.

In some embodiments, the affinity elution buffer comprises: about 50 mM, about 55 mM, about 60 mM, about 65 mM, about 70 mM, about 75 mM, about 80 mM, about 85 mM, about 90 mM, about 95 mM or about 100 mM glycine; about 10 mM, about 15 mM, about 20 mM, about 25 mM, about 30 mM, about 35 mM, about 40 mM, about 45 mM or about 50 mM of an amino acid (e.g., an amino acid selected from the group consisting of aspartate, glutamate, histidine, arginine, lysine, cysteine and tyrosine; or an amino acid selected from the group consisting of aspartate, glutamate, and histidine; or an amino acid selected from the group consisting of histidine and lysine; or an amino acid selected from the group consisting of cysteine and tyrosine); about 2.5 mM, about 3 mM, about 3.5 mM, about 4 mM, about 4.5 mM, about 5 mM, about 7.5 mM, about 10 mM, about 12.5 mM, about 15 mM, about 17.5 mM, about 20 mM, about 22.5 mM or about 25 mM of a salt (e.g., a salt selected from the group consisting of sodium salt, potassium salt, ammonium salt, magnesium salt, calcium salt, copper salt, cobalt salt, manganese salt, nickel salt and zinc salt; or a salt selected from the group consisting of potassium salt, ammonium salt, magnesium salt, calcium salt, manganese salt and zinc salt; or a salt selected from the group consisting of potassium salt, magnesium salt and calcium salt; or a salt selected from the group consisting of potassium salt and magnesium salt, such as MgCl₂); about 0.1%, about 0.15%, about 0.2%, about 0.25%, about 0.3%, about 0.35%, about 0.4%, about 0.45% or about 0.5% P188, and the buffer has a low pH, e.g., a pH of about 2-5 such as a pH of about 2, bout 2.5, about 3, about 3.5 or about 4.

In some embodiments, the affinity elution buffer comprises: about 75 mM glycine, about 25 mM histidine and about 10 mM MgCl₂. For example, the affinity elution buffer comprises: about 75 mM glycine, about 25 mM histidine and about 10 mM MgCl₂, and the buffer has a low pH, e.g., a pH of about 2-5 such as a pH of about 2, bout 2.5, about 3, about 3.5 or about 4.

In some embodiments, the affinity elution buffer comprises: about 50 mM glycine, about 75 mM citrate, about 10 mM MgCl₂, about 0.3% P188 and has a pH of about 3.0. Generally, the affinity elution buffer has a conductivity in a range from about 5 mS/cm to about 8 mS/cm. For example, the affinity elution buffer has a conductivity in a range from about 5.5 mS/cm to about 7 mS/cm, from about 5.75 mS/cm to about 6.75 mS/cm, or from about 6.15 mS/cm to about 6.25 mS/cm. In some embodiments, the affinity elution buffer has a conductivity of about 6.18 mS/cm.

It is noted that conductivity can be measured using standard methods and devices in the art.

The affinity elution buffer can have an osmolarity in a range from about 100 mOms to about 225 mOms. For example, the affinity elution buffer can have an osmolarity in a range from about 125 mOms to about 200 mOms, from about 150 mOms to about 175 mOms, or from about 155 mOms to about 165 mOms. In some embodiments, the affinity elution buffer has an osmolarity of about 161 mOms.

Osmolality, a term well understood in the art, is defined as number of solute molecules per kg water. Osmolality can be measured using standard techniques in the art, such as freezing point depression using, for example, an osmometer.

In some embodiments, the affinity elution buffer is substantially free of citric acid or a salt thereof.

### Column Sizes

The methods described herein are scalable. Thus, chromatographic steps described herein can used with various column sizes. Accordingly, a column size for the affinity and/or anion exchange chromatography can range from ml to thousands of liters. For example, the column can be a 0.5 ml column, a 1.5 ml column, a 10 ml column, a 20 ml column, a 30 ml column, a 50 ml column, a 100 ml column, a 200 ml column, a 300 ml column, a 400 ml column, a 500 ml column, a 600 ml column, a 700 ml column, an 800 ml column, a 900 ml column, a 1000 ml (1 L) column a 2000 ml (2 L) a 10 L column, a 20 L column, a 30 L column, a 40 L column, a 50 L column, a 60 L column, a 70 L column, an 80 L column a 90 L column, a 100 L column or a column with a capacity greater than 100 L as well as any other column with a capacity between the volumes listed above.

### Wash Steps

The methods described herein can comprise one or more wash steps. For example, the recombinant virus particles bound to affinity chromatography media can be washed, e.g., with a buffer prior to eluting or recovering the recombinant particles from the affinity chromatography media. Similarly, the recombinant virus particles bound to anion exchange chromatography media can be washed, e.g., with a buffer prior to eluting or recovering the recombinant particles from the anion exchange chromatography media. It is noted that the chromatography media can be washed with at least about 1 CV, 1.5 CV, 2 CV, 2.5 CV, 3 CV, 3.5 CV, 4 CV, 4.5 CV, 5 CV, 5.5 CV, 6 CV, 6.5 CV, 7 CV, 7.5 CV, 8 CV, 8.5 CV, 9 CV, 9.5 CV, 10 CV, 10.5 CV, 11 CV, 11.5 CV, 12 CV, 12.5 CV, 13 CV, 13.5 CV, 14 CV, 14.5 CV, 15 CV, 15.5 CV, 16 CV, 16.5 CV, 17 CV, 17.5 CV, 18 CV, 18.5 CV, 19 CV, 19.5 CV, 20 CV or more of the appropriate media, e.g., a wash buffer. In some embodiments, the anion exchange elution gradient length can range from about 2 CV to about 100 CV, or from about 5 CV to about 80 CV, or from about 5 CV to about 60 CV, or from about 5 CV to about 50 CV, or from about 5 CV to about 40 CV, or from about 5 CV to about 30 CV. In some embodiments, the elution flow (CV/min) can range from about 0.05 CV/min to about 2 CV/min, or from about 0.1 CV/min to about 1.5 CV/min, or from about 0.1 CV/min to about 1 CV/min.

It is noted that the media, e.g., a wash buffer for washing the recombinant virus particles bound to affinity chromatography media can be optimized for the viral particles to be separated and/or the elution buffer to employed. For example, when the affinity elution buffer comprises histidine, a wash buffer comprising histidine can be used for the wash step. Thus, in some embodiments, the wash buffer for the affinity chromatography comprises histidine at a concentration of at least about 10 mM, 15 mM, 20 mM, 25 mM, 30 mM, 35 mM, 40 mM, 45 mM, 50 mM or higher. In some embodiments, the wash buffer for the affinity chromatography comprises histidine at a concentration of from about 10 mM to about 50 mM, from about 15 mM to about 40 mM, or from about 20 mM to about 30 mM.

In various embodiments, the affinity elution buffer comprises citric acid or a salt thereof. In such embodiments, a wash buffer comprising citric acid or a salt thereof, e.g., citrate can be used for the wash step. Thus, in some embodiments, the wash buffer for the affinity chromatography comprises citric acid or a salt thereof at a concentration of at least about 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM 95 mM, 100 mM or higher. In some embodiments, the wash buffer for the affinity chromatography comprises citric acid or a salt thereof at a concentration of from about 10 mM to about 100 mM, from about 15 mM to about 90 mM, or from about 20 mM to about 75 mM.

Generally, a wash buffer for the affinity chromatography has a high pH. For example, the wash buffer for the affinity chromatography has a pH greater than or equal to about 7.5, about 8, about 8.5, about 9, about 9.5 or about 10. In some embodiments, the wash buffer for the affinity chromatography has a pH greater than or equal to about 8.5. For example, the wash buffer for the affinity chromatography has a pH of about 9.

### Additional Parameters for Chromatography

Generally, the chromatography steps described herein are carried out ambient temperature. The residence time is from about 1 minute to about 8 minutes. If a wash step is included, the chromatography media can be washed with from about 5 CV to about 15 CV of the appropriate wash buffer. If needed, the chromatography media can be equilibrated with from about 5 CV to 15 CV of an equilibration buffer. Generally, bound AAV particles can be eluted from the chromatography media using from about 5 CV to 100 CV of the elution media.

In one aspect, provided herein is method for purifying or isolating recombinantly expressed virus particles, e.g., recombinant adeno-associated virus (rAAV), optionally comprising a transgene, from a preparation comprising recombinant vector particles, empty capsids and host cell impurities, to provide a product substantially free of empty viral particles. Generally, the method comprises contacting a preparation, e.g., harvest media comprising recombinant virus particles with an affinity chromatography media under conditions that allow binding of virus particles to the affinity chromatography media. The bound viral particles are eluted from the affinity chromatography media using an elution buffer and recovering an eluate, comprising the eluted viral particles. In some embodiments, the affinity elution buffer comprises a weak acid or, salt thereof. In some embodiments, the affinity elution buffer is substantially free of weak acids or salts thereof. The eluate from the affinity chromatography is also referred to as affinity eluate herein. The affinity eluate is contacted with an anion exchange chromatography media under conditions that allow binding of viral particles to the anion exchange chromatography media. Inventors have discovered inter alia that presence of an ionic compound, such as an anionic compound, e.g. an acid or salt thereof, such as a weak acid or a salt thereof in the buffer used for equilibrating and/or conditioning the chromatography prior to contacting with the affinity eluate surprising and unexpectedly leads to preferential binding of genome-containing viral particles, e.g., Adeno associated virus (AAV) particles having packaged genomic sequences (i.e., full viral particles) over genome-deficient particles (i.e., empty), thereby generating recombinant virus particles e.g., recombinant AAV virus particles that have less empty particles. In some embodiments, a population of purified recombinant adeno-associated virus (rAAV), purified by the method described herein, comprises less than about 10% empty viral capsids. In some embodiments, the population of purified rAAV comprises less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.75%, less than about 0.5%, less than about 0.25%, less than about 0.2%, less than about 0.15%, less than about 0.1%, less than about 0.05%, less than about 0.03%, less than about 0.02%, or, less than about 0.01% empty viral capsids. In some embodiments, the population of purified rAAV is substantially devoid or, substantially free of empty viral capsids.

In some aspects, provided herein is a population of purified recombinant adeno-associated virus (rAAV) that optionally lacks prokaryotic sequences, wherein the purified virus has a particle to infectivity ratio less than 2×10⁴ vg/TCID50, wherein the population of purified rAAV comprises less than about 10% empty viral capsids, and wherein the purified virus optionally is obtained by transfecting a suspension mammalian cell line wherein cells are transfected in suspension. In some embodiments, the purified recombinant adeno-associated virus has a particle to infectivity ratio less than 1.5×10⁴ vg/TCID50, less than 1×10⁴ vg/TCID50, less than 9×10³ vg/TCID50, less than 8×10³ vg/TCID50, less than 6×10³ vg/TCID50, less than 5×10³ vg/TCID50, less than 4×10³ vg/TCID50, less than 3×10³ vg/TCID50, less than 2×10³ vg/TCID50, less than 9×10² vg/TCID50, less than 8×10² vg/TCID50, less than 7×10² vg/TCID50, less than 6×10² vg/TCID50, less than 5×10² vg/TCID50, less than 4×10² vg/TCID50, less than 3×10² vg/TCID50, less than 2×10² vg/TCID50, or, less than 1×10² vg/TCID50, or, less than 0.5×10² vg/TCID50 or, even less. In certain embodiments, the population of purified rAAV comprises less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.75%, less than about 0.5%, less than about 0.25%, less than about 0.2%, less than about 0.15%, less than about 0.1%, less than about 0.05%, less than about 0.03%, less than about 0.02%, or, less than about 0.01% empty viral capsids. In some preferred embodiments, the population of purified rAAV is substantially devoid of empty viral capsids. In some embodiments, the population of purified rAAV that lacks prokaryotic sequence, is manufactured using close ended linear duplexed DNA (celDNA or, clDNA) as a template. In some embodiments, the purified recombinant adeno-associated virus (rAAV) that lacks prokaryotic sequence is produced by transfecting a mammalian cell line in suspension with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements. In certain embodiments, the mammalian cell line is a human embryonic cell line, wherein, the human embryonic cell line is suspension adapted serum free cell line that is derived from a human embryonic kidney cell line. In some embodiments, the population of purified recombinant adeno-associated virus (rAAV) does not lack prokaryotic sequences.

In some embodiments, the purified rAAV is obtained by transfecting non-adherent human embryonic cell line in suspension. In certain embodiments, the purified virus is obtained by transfecting cells in suspension of a human embryonic cell line, wherein, the human embryonic cell line is suspension adapted serum free cell line that is derived from a human embryonic kidney cell line. In some embodiments, the cells optionally are transfected in suspension. In some embodiments, the purified rAAV lacks bacterial sequences.

In one aspect, provided herein is a population of purified recombinant adeno-associated virus (rAAV) that optionally lacks prokaryotic sequences, wherein the purified virus has a particle to infectivity ratio less than 2×10⁴ vg/TCID50. In some embodiments, the purified recombinant adeno-associated virus has a particle to infectivity ratio less than 1.5×10⁴ vg/TCID50, less than 1×10⁴ vg/TCID50, less than 9×10³ vg/TCID50, less than 8×10³ vg/TCID50, less than 6×10³ vg/TCID50, less than 5×10³ vg/TCID50, less than 4×10³ vg/TCID50, less than 3×10³ vg/TCID50, less than 2×10³ vg/TCID50, less than 9×10² vg/TCID50, less than 8×10² vg/TCID50, less than 7×10² vg/TCID50, less than 6×10² vg/TCID50, less than 5×10² vg/TCID50, less than 4×10² vg/TCID50, less than 3×10² vg/TCID50, less than 2×10² vg/TCID50, or, less than 1×10² vg/TCID50, or less than 0.5×10² vg/TCID50 or, even less. In some embodiments, the purified recombinant adeno-associated virus (rAAV) that lacks prokaryotic sequence is produced by transfecting a mammalian cell line in suspension with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements. In certain embodiments, the mammalian cell line is a human embryonic cell line, wherein, the human embryonic cell line is suspension adapted serum free cell line that is derived from a human embryonic kidney cell line. In some embodiments, the purified recombinant AAV lacks bacterial sequences.

In some embodiments of any one of the aspects described herein, the population of purified recombinant adeno-associated virus (rAAV) has infectious particle titer of about 1×10⁵ TCID50/ml (Median Tissue Culture Infectious Dose) to about 1×10¹¹ TCID50. In certain embodiments, the infectious particle titer is at least 3×10⁹ TCID50/ml. In several embodiments, the infectious particle titer is at least 2×10⁵ TCID50/ml, 5×10⁵ TCID50/ml, at least 7.5×10⁵ TCID50/ml, at least 8×10⁵ TCID50/ml, at least 8.5×10⁵ TCID50/ml, at least 9×10⁵ TCID50/ml, at least 9.5×10⁵ TCID50/ml, at least 1×10⁶ TCID50/ml, at least 2×10⁶ TCID50/ml, at least 5×10⁶ TCID50/ml, at least 7.5×10⁶ TCID50/ml, at least 8×10⁶ TCID50/ml, at least 8.5×10⁶ TCID50/ml, at least 9×10⁶ TCID50/ml, at least 9.5×10⁶ TCID50/ml, at least 1×10⁷ TCID50/ml, at least 2×10⁷ TCID50/ml, at least 5×10⁷ TCID50/ml, at least 7.5×10⁷ TCID50/ml, at least 8×10⁷ TCID50/ml, at least 9×10⁷ TCID50/ml, at least 1×10⁸ TCID50/ml, at least 2.5×10⁸ TCID50/ml, at least 5×10⁸ TCID50/ml, at least 7.5×10⁸ TCID50/ml, at least 8×10⁸ TCID50/ml, at least 8.5×10⁸ TCID50/ml, at least 9×10⁸ TCID50/ml, at least 9.5×10⁸ TCID50/ml, at least 0.5×10⁹ TCID50/ml, at least 1×10⁹ TCID50/ml, at least 1.5×10⁹ TCID50/ml, at least 2×10⁹ TCID50/ml, at least 2.5×10⁹ TCID50/ml, at least 3×10⁹ TCID50/ml, at least 3.5×10⁹ TCID50/ml, at least 4×10⁹ TCID50/ml, at least 4.5×10⁹ TCID50/ml, at least 5×10⁹ TCID50/ml, at least 5.5×10⁹ TCID50/ml, at least 6×10⁹ TCID50/ml, at least 6.5×10⁹ TCID50/ml, at least 7×10⁹ TCID50/ml, at least 7.5×10⁹ TCID50/ml, at least 8×10⁹ TCID50/ml, at least 8.5×10⁹ TCID50/ml, at least 9×10⁹ TCID50/ml, at least 9.5×10⁹ TCID50/ml, at least 1×10¹⁰ TCID50/ml, at least 2×10¹⁰ TCID50/ml, at least 5×10¹⁰ TCID50/ml, at least 7.5×10¹⁰ TCID50/ml, at least 8×10¹⁰ TCID50/ml, at least 8.5×10¹⁰ TCID50/ml, at least 9×10¹⁰ TCID50/ml, at least 9.5×10¹⁰ TCID50/ml, or, at least about 10¹¹ TCID50/ml. In some embodiments, the infectious titer TCID50/ml is preferably normalized to vg/ml.

In some embodiments of one or more aspect described herein, the method of producing recombinant AAV comprises a transient transfection method. In some embodiments, the method of producing recombinant AAV comprises a stable transfection method. In some embodiments, the transfection is performed in suspension.

In certain embodiments, the mammalian cell line is a suspension cell or cell line i.e. non-adherent cell or cell line and the cells are transfected in suspension. In certain embodiments, the cell line is derived from a human embryonic kidney 293 cell line (HEK 293). In certain embodiments, the human embryonic kidney cells lack an SV40 antigen or other transformation antigens. In certain embodiments, the mammalian cell line is a suspension adapted serum free cell line. In certain embodiments, the cell lines are derived from primary blood cells e.g. lymphocytes, monocytes, macrophages, granulocytes, dendritic cells, erythrocytes. In certain embodiments, the cell lines are derived from cell biopsies and include, for example, lymph node cells, bone marrow cells, cord blood cells. In certain embodiments, the cell lines are derived from circulating tumor cells. In certain embodiments, the cell lines are derived from blood cell lines, for example, Jurkat and Molt4 T cell lines, U937 and THP pro-monocytes cell lines, B cell hybridomas. In certain embodiments, the cell lines are derived from stem cells. In certain embodiments, the cell line used for production of recombinant AAV is a stable cell line. In some embodiments, the mammalian cell line is an adherent cell line.

In some embodiments, the purified recombinant AAV is obtained by culturing the human embryonic cell line in suspension and transfecting the same in suspension.

Cell culture work involved in rAAV production including expansion, seeding and transfection of adherent cells is cumbersome and resource intensive. Therefore, using cells suspended in aqueous liquid medium (“suspension cells”) for rAAV vector production is desirable due to its scalability and cost effectiveness. Accordingly, in certain embodiments of any one of the aspects described herein, the host cell line can be suspension-adapted. For example, host cells can be transfected with the nucleic acid vector(s) in suspension. In certain embodiments, the host cell is a mammalian cell, i.e., the host cell line is a mammalian cell line. For example, the host cell, i.e., the host cell line, is human cell, such as a human embryonic cell line. In certain embodiments of any one of the aspects described herein, the host cell line is a human embryonic kidney cell line.

Methods of making AAV vectors are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO 1999014354, WO1999/015685, WO1999/047691, WO2000/055342, WO2000/075353 and WO2001/023597; Methods In Molecular Biology, ed. Richard, Humana Press, N J (1995); O'Reilly et al, Baculovirus Expression Vectors, A Laboratory Manual, Oxford Univ. Press (1994); Samulski et al., J Fir. 63:3822-8 (1989); Kajigaya et al, Proc. Nat'l. Acad. Sci. USA 88: 4646-50 (1991); Ruffing et al., J. Vir. 66:6922-30 (1992); Kimbauer et al, Vir., 219:37-44 (1996); Zhao et al, Vir. 272: 382-93 (2000); the contents of each of which are herein incorporated by reference. Viral replication cells commonly used for production of recombinant AAV viral particles include but are not limited to HEK293 cells, COS cells, HeLa cells, KB cells, and other mammalian cell lines.

Components for AAV production (e.g., adenovirus E1a, E1b, E2a, and/or E4ORF6 gene products, rep or a fragment(s) thereof, cap, the expression cassette, as well as any other desired helper functions), may be delivered to the packaging host cell separately, or in combination, in the form of any genetic element which transfer the sequences carried thereon. One example of making AAV is by Triple transfection method wherein, the host cell is transfected with three separate nucleic acids; and wherein one nucleic acid encodes replication sufficient helper proteins, e.g, adenoviral helper proteins that helps in efficient replication and packaging of the vector but lacks essential Adenoviral structural and replication genes to generate an Adenovirus, one nucleic acid encodes AAV rep and cap proteins and the other nucleic acid encodes AAV ITR flanking transgene or, heterologous transgene. As used herein, a genetic element (vector) includes, e.g., naked DNA, a plasmid, phage, transposon, cosmid, episome, a protein in a non-viral delivery vehicle (e.g., a lipid-based carrier), virus, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. See, e.g., K. Fisher et al, J Virol., 70:520-532 (1993) and U.S. Pat. No. 5,478,745.

In one embodiment, a stable or transient host cell will contain the required component(s) under the control of an inducible or regulatable promoter. However, the required component(s) may be under the control of a constitutive promoter or a synthetic promoter.

It is to be understood that a viral expression system will further be modified to include any necessary elements required to complement a given viral vector during its production using methods described herein. For example, in certain embodiments, the nucleic acid cassette is flanked by terminal repeat sequences. In one embodiment, for the production of rAAV vectors, the AAV expression system will further comprise at least one of a recombinant AAV plasmid, a plasmid expressing Rep, a plasmid expressing Cap, and an adenovirus helper plasmid. Complementary elements for a given viral vector are well known the art and a skilled practitioner would be capable of modifying the viral expression system described herein accordingly.

In some embodiments, the recombinant AAV is produced from plasmid DNA. In some embodiments, the recombinant AAV is produced form close ended linear duplexed DNA. Closed linear DNA molecules typically comprise covalently closed ends also described as hairpin loops, where base-pairing between complementary DNA strands is not present. The hairpin loops join the ends of complementary DNA strands. Structures of this type typically form at the telomeric ends of chromosomes in order to protect against loss or damage of chromosomal DNA by sequestering the terminal nucleotides in a closed structure. In examples of closed linear DNA molecules described herein, hairpin loops flank complementary base-paired DNA strands, forming a closed linear (cl) DNA shaped structure. Closed linear DNA molecules include barbell shaped DNA.

One or more of the nucleic acids a)-c) i.e.: a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements, may be present on close ended linear duplex nucleic acids. Close ended linear duplex nucleic acids can be generated by a variety of known methods, including in vitro cell-free synthesis and in vivo methods. One method of generating the covalently closed ended linear duplex nucleic acids is by incorporation of protelomerase binding sites in a precursor molecule such that the protelomerase binding sites flank the nucleic acid of interest and exposure of the molecule to protelomerase to thereby cleave and ligate the DNA at the site. The nucleic acid of interest can comprise one or more of a), b, and c), i.e. a), b) and c); any combination of a), b) and c); or only a), only b), or only c). Examples of making close ended linear duplexed DNA are well known in the art e.g., as described in Nucleic Acids Res. 2015 Oct. 15; 43(18): e120; Antisense & nucleic acid drug development 11:149-153 (2001); U.S. Pat. Nos. 9,109,250, 9,499,847, U.S. Ser. No. 10/501,782, U.S. Ser. No. 10/286,399; and/or, US publication nos. US 20190185924, US20190203282; the content of all of which are incorporated herein by reference in entirety.

Alternate methods of generating covalently closed end linear DNA (e.g., close ended linear duplexed DNA) that lack prokaryotic sequence or, bacterial sequences, are known in the art e.g., by formation of mini-circle DNA from plasmids (e.g. as described in U.S. Pat. Nos. 8,828,726, and 7,897,380, the contents of each of which are incorporated by reference in their entirety). For example, one method of cell-free synthesis combines the use of two enzymes—Phi29 DNA polymerase and a protelomerase, and generates high fidelity, covalently closed, linear DNA constructs. The constructs contain no antibiotic resistance markers, and therefore eliminate the packaging of these sequences. The process can amplify AAV genome DNA in a 2-week process at commercial scale and maintain the ITR sequences required for virus production.

In certain embodiments, the in vivo cell system is used to produce a non-viral DNA vector construct for delivery of a predetermined nucleic acid sequence into a target cell for sustained expression. The non-viral DNA vector comprises, two DD-ITRs each comprising: an inverted terminal repeat having an A, A′, B, B′, C, C′ and D region; a D′ region; and wherein the D and D′ region are complementary palindromic sequences of about 5-20 nt in length, are positioned adjacent the A and A′ region; the predetermined nucleic acid sequence (e.g. a heterologous gene for expression); wherein the two DD-ITRs flank the nucleic acid in the context of covalently closed non-viral DNA and wherein the closed linear vector comprises a ½ protelomerase binding site on each end as for example as described in International publication no. WO 2019246544, which is incorporated herein by reference in its entirety.

### Harvest and Clarification

The methods disclosed herein can comprise a step of producing the harvest media for contacting with the affinity chromatography media by a method comprising upstream processing such as, for example, harvest of a cell culture or cell culture supernatant and/or clarification of the harvested cell culture or cell culture supernatant. Accordingly, in another aspect provided herein is a method for preparing a cell culture or cell culture supernatant for affinity chromatography. Generally, the method comprises harvesting a cell culture or cell culture supernatant and/or clarifying a cell culture or cell culture supernatant to produce a harvest media comprising recombinantly expressed virus particles. The method described herein produces recombinant AAV particles substantially free of empty AAV particle irrespective of the cell density of the cells transfected. Generally, cell density of the transfected cells is from about 1E6 cells/ml to about 80E6 cells/ml. The similar effect of substantially eliminating empty particles from recombinant AAV particles is applicable when the recombinant AAV is produced using triple transfection method or, stable producer and/or packaging cell line, e.g. Pro 10 cells. In some embodiments, the recombinant AAV is produced, with the method described herein, from close ended linear duplexed nucleic acid, e.g., close ended linear duplexed DNA. In some embodiments, the recombinant AAV is produced using other forms of nucleic acid, e.g, plasmid DNA.

In some embodiments, the method for preparing a cell culture or cell culture supernatant for affinity chromatography comprises lysing a host cell in the cell culture or cell culture supernatant. Methods for lysing host cells in a cell culture or cell culture supernatant. For example, a non-ionic surfactant can be added to the cell culture or cell culture supernatant.

The non-ionic surfactant is added to the cell culture or cell culture supernatant to a final concentration of at least about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1% (w/v, w/w or v/v) or higher. For example, the non-ionic surfactant is added to the cell culture or cell culture supernatant to a final concentration of from about 0.05% to about 1%, from about 0.1% to about 0.95%, from about 0.15% to about 0.9%, from about 0.2% to about 0.85%, from about 0.25% to about 0.8%, from about 0.3% to about 0.75%, from about 0.35% to about 0.65% from about 0.4% to about 0.6% or from 0.45% to about 0.55%. In some embodiments, the non-ionic surfactant is added to the cell culture or cell culture supernatant to a final concentration of about 0.05%, 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1%. For example, the non-ionic surfactant can be added to the cell culture or cell culture supernatant to a final concentration of about 0.5%.

Generally, the non-ionic surfactant is allowed to mix with the cell culture or cell culture supernatant for a sufficient period of time to lyse host cells present in the cell culture or cell culture supernatant. For example, the non-ionic surfactant is mixed with the cell culture or cell culture supernatant for a period of from about 15 minutes to about 2 hours. In some embodiments, the non-ionic surfactant is mixed with the cell culture or cell culture supernatant for a period of from about 30 minutes to about 60 minutes.

The mixing can be at ambient temperature or an elevated temperature. For example, the mixing with the non-ionic surfactant can be at a temperature from about 15° C. to about 37° C. In some embodiments, the mixing with the non-ionic surfactant can be at a temperature of about 18° C., 19° C., 20° C., 21° C., 22° C., 23° C., 24° C., 25° C., 26° C., 27° C., 28° C., 28° C., 30° C., 31° C., 32° C., 33° C., 34° C., 35° C., 36° C., or 37° C.

It is noted that, any desired non-ionic surfactant can be used for lysing the host cells. Exemplary non-ionic surfactants and classes of non-ionic surfactants for lysing host cells can include polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic dials; polyalkoxylated alkylphenols; fatty acid alkoxylates; sorbitan alkoxylates; sorbitol esters; C₈ to C₂₂ alkyl or alkenyl polyglycosides; polyalkoxy styrylaryl ethers; alkylamine oxides; block copolymer ethers; polyalkoxylated fatty glyceride; polyalkylene glycol ethers; linear aliphatic or aromatic polyesters; organo silicones; polyaryl phenols; sorbitol ester alkoxylates; and mono- and diesters of ethylene glycol and mixtures thereof; ethoxylated tristyrylphenol; ethoxylated fatty alcohol; ethoxylated lauryl alcohol; ethoxylated castor oil; and ethoxylated nonylphenol; alkoxylated alcohols, amines or acids. In some embodiments of any one of the aspects, the the non-ionic surfactant for lysing the host cells is selected from the group consisting of polyoxyethylene fatty alcohol ethers, polyoxyethylene alkylphenyl ethers, polyoxyethylene-polyoxypropylene block copolymers, alkylglucosides, alkylphenol ethoxylates, preferably polysorbates, polyoxyethylene alkyl phenyl ethers, and any combinations thereof.

Specific exemplary non-ionic surfactants for lysing host cells include, but are not limited to, ECOSURF EH-9, polysorbates (such as polysorbate 20 (TWEEN 20), polysorbate 28, polysorbate 40, polysorbate 60, polysorbate 65, polysorbate 80, polysorbate 81, and polysorbate 85), ECOSURF EH-14, TWEEN 60 nonionic detergent, PPG-PEG-PPG Pluronic 10R5, Polyoxyethylene (18) tridecyl ether, Polyoxyethylene (12) tridecyl ether, MERPOL SH surfactant, MERPOL OJ surfactant, MERPOL HCS surfactant, IGEPAL CO-720, IGEPAL CO-630, IGEPAL CA-720, Brij S20, BrijS10, Brij 010, Brij C10, BRIJ 020, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, and TERGITOLNP-13 and any combinations thereof.

Preferably, the non-ionic surfactant for lysing host cells is not Triton X-100.

In some embodiments, a zwitterionic surfactant can be added to the cell culture or cell culture supernatant for lysing the host cell. Exemplary zwitterionic surfactants include, but are not limited to, sulfonates, such as CHAPS (3-[(3-Cholamidopropyl)dimethylammonio]-1-propanesulfonate), CHAPSO (3-{(3-cholamidopropyl)dimethylammonio}-2-hydroxy-1-propane-sulfonate), 3-(decyl dimethyl ammonio)propanesulfonate, 3-(dodecyldimethylammonio) propanesulfonate, 3-(N,N-dimethylmyristylammonio)propanesulfonate, 3-(N,N-dimethyl octadecylammonio)propanesulfonate, 3-(N,N-dimethyloctylammonio) propanesulfonate, and 3-(N,N dimethylpalmitylammonio)propanesulfonate; sultaines, such as cocamidopropyl hydroxysultaine; betaines, e.g., cocamidopropyl betaine; and phosphates, such as lecithin.

In some embodiments, the surfactant, e.g., the zwitterionic surfactant can be an amine oxide surfactant. For example, an amine oxide surfactant can be added to the cell culture or cell culture supernatant for lysing the host cell. An amine oxide surfactant that can be used in methods described herein can be a trialkyl amine N-oxide, e.g., an amine oxide of formula R¹R²R³NO, wherein R¹ is a substituted or unsubstituted alkyl or alkenyl containing from about 8 to about 30 carbon atoms; and R² and R³ are independently substituted or unsubstituted alkyl or alkenyl groups containing from about 1 to about 18 carbon atoms. Non limiting examples of trialkyl amine N-oxide and trialkyl amine N-oxide surfactants of use are described in WO1998055581, which is incorporated herein by reference in its entirety.

The cell culture or cell culture supernatant may comprise impurities, e.g., host cell DNA (hcDNA). Therefore, the method for preparing a cell culture or cell culture supernatant for affinity chromatography can comprise a post-lysis step of removing or reducing amount of impurities, e.g., hcDNA from the cell culture or cell culture supernatant. Methods and compositions for reducing the amount of host cell DNA cell cultures or cell culture supernatants are well known in the art. For example, a cationic amine or nuclease can be added to the cell culture or cell culture supernatant.

In some embodiments, the post-lysis step comprises adding a selective precipitation agent to reduce or remove impurities such as hcDNA from the cell culture or cell culture supernatant. As used herein, a “selective precipitation agent” refers to any agent, compound or such which, when added to a preparation comprising a population of recombinant virus particles and contaminating nucleic acid molecules, will affect the selective precipitation of at least a substantial amount of contaminating nucleic acid molecules away from the recombinant virus particles. Exemplary agents for adding to the cell culture or cell culture supernatant in the post-lysis step include, but are not limited to cetyl trimethylammonium bromide, cetylpyridinium chloride, benzethonium chloride, tetradecyltrimethyl-ammonium chloride, polyethylene imine and combinations thereof.

In some embodiments, a nuclease, e.g., an endonuclease is added to the cell culture or cell culture supernatant for reducing or removing impurities such as hcDNA. Exemplary endonucleases include endonucleases derived from both Prokaryotes and Eukaryotes. In some embodiments, the nuclease is BENZONASE® or a salt active nuclease (SAN).

Generally, the nuclease is added to the cell culture or cell culture supernatant to a final concentration of at least about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1% (w/v, w/w or v/v) or higher. For example, the nuclease is added to the cell culture or cell culture supernatant to a final concentration of from about 0.05% to about 1%, from about 0.1% to about 0.95%, from about 0.15% to about 0.9%, from about 0.2% to about 0.85%, from about 0.25% to about 0.8%, from about 0.3% to about 0.75%, from about 0.35% to about 0.65% from about 0.4% to about 0.6%, from 0.45% to about 0.55% from about 0.05% to about 0.4%, or from about 0.2% to about 0.4%. In some embodiments, the nuclease is added to the cell culture or cell culture supernatant to a final concentration of about 0.05%, 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1%. For example, the nuclease can be added to the cell culture or cell culture supernatant to a final concentration of about 0.2%. In some embodiments, the nuclease can be added to the cell culture or cell culture supernatant to a final concentration of about 0.05% to about 0.4%.

Generally, the agent or nuclease is allowed to mix with the cell culture or cell culture supernatant for a period of about 15, 20, 30, 35, 40, 45, 50, 55 minutes or longer. In some embodiments, the agent or nuclease is allowed to mix with the cell culture or cell culture supernatant for a period of from about 10 minutes to about 4 hours. For example, the agent or nuclease is mixed with the cell culture or cell culture supernatant for a period of from about 15 minutes to about 3 hours. In some embodiments, the agent or nuclease is mixed with the cell culture or cell culture supernatant for a period of from about 30 minutes to about 120 minutes. For example, the agent or nuclease is mixed with the cell culture or cell culture supernatant for a period of about 30 minutes.

In some embodiment, method for preparing a cell culture or cell culture supernatant for affinity chromatography comprises a step of clarifying the cell culture or cell culture supernatant. For example, the method comprises a step of clarifying the cell culture or cell culture supernatant by depth filtration to produce the clarified composition (e.g., harvest media) for affinity chromatography. Exemplary depth filters for use in the methods described herein include, but are not limited to, CUNO® Zeta PLUS® Delipid filters, CUNO® Emphaze AEX filters, CUNO® 30/60ZA filters, CUNO® 90ZBO8A filters, CUNO® DELIO8A Delipid filters, and CUNO® DELIPO8A Delipid plus filters (3M, St. Paul, Minn.), Clarisolve® grade 60HX, 40MS, 20MS, Millistak+® HC grade COHC, DOHC, A1HC, B1HC, XOHC, FOHC, Millistak+® HC Pro grade DOSP, COSP, and XOSP Millipore filters (EMD Millipore, Billerica, Mass.), and Sartopore® bi-layer filter cartridges.

In some embodiments, the method further comprises a step of concentrating the clarified cell culture or cell culture supernatant. For example, the method comprises a step of concentrating the clarified cell culture or cell culture supernatant by tangential flow filtration.

### Further Downstream Processing

The eluate from the anion exchange chromatography comprising isolated recombinant virus particles can be further processed using methods known in the art. Exemplary downstream processing includes, for example, tangential flow filtration, affinity chromatography, size exclusion chromatography, cation exchange chromatography, anion exchange chromatography, hydroxylapatite chromatography, and hydrophobic interaction chromatography. In some embodiments, downstream processing comprises a step of tangential flow filtration. In some embodiments, downstream processing comprises a step of sterile filtration.

### Recombinant Virus Particles

As used herein, the term “recombinant,” as applied to a virus particle means that the virus particle is the product of one or more procedures that result in a virus particle construct that is distinct from a naturally occurring virus particle. Methods for producing recombinant virus particles are well known in the art and available to one of skill in the art.

A “filled particle” or “full particle” (also interchangeably referred to as “full AAV particle”) refers to viral particle that comprises an intact viral particle vector comprising a heterologous polynucleotide (such as a transgene, i.e. a polynucleotide other than a wild-type virus genome). A “filled” or, “full” particle can also be interchangeably used as “packaged particle” or, “packaged virus” or “packaged AAV” or, “recombinantly expressed AAV”. An “empty particle” is also interchangeably referred to as “empty AAV particle” that refers to a viral particle that comprises at least one viral protein but lacks all of the genome e.g virus genome or, recombinant genome. A “partially full particle” is also interchangeably referred to as “partially full AAV particle” or “partially filled AAV particle” that refers to a viral particle that comprises at least one viral protein but lacks in part of the genome e.g virus genome or, recombinant genome. Not being bound by any theory, in the invention disclosed herein, the population or plurality of “full” or, “recombinantly expressed AAV particle” can comprise an enriched population or plurality of “full” or, “recombinantly expressed AAV particle” over “partially full AAV particle”. As used herein, “partially full particle” also include particles containing DNA from the host cell or pDNA used in transfection. Empty particles do not include, e.g., an intact viral particle vector comprising a heterologous polynucleotide. It is noted that the terms “particle” and “capsid” can be used interchangeably herein.

The recombinant virus particles (rAAV) described herein include recombinant adeno associated virus (rAAV) particles. The rAAV particles can be AAV-1, AAV-2, AAV-2i8, AAV-3, AAV-4, AAV-5, AAV-6, AAV-7, AAV-8, AAV-9, AAV-10, AAV-11, AAV-12, AAV-13, AAV-14, AAV-15, AAV-16 or a chimera, derivative, modification, or pseudotype thereof. Accordingly, in some embodiments of any one of the aspects, the rAAV particle comprises a capsid protein from serotype AAV-1, AAV-2, AAV-2i8, AAV-3, AAV-4, AAV-5, AAV-6, AAV-7, AAV-8, AAV-9, AAV-10, AAV-11, AAV-12, AAV-13, or a chimera, derivative, modification, or pseudotype thereof. In some embodiments, the rAAV particle comprises capsids from polyploid (also referred to as haploid) in that they can comprise different combinations of VP1, VP2, and VP3 AAV serotypes in a single AAV capsid as described in PCT/US18/22725 and U.S. Pat. No. 10,550,405, which is incorporated by reference herein. In some embodiments rAAVs can comprise rAAV virion. In certain embodiments rAAV capsids comprise substitution, addition and/or deletion of one or more amino acids; for example, capsids comprising inserted peptides for targeting.

It is noted that virus particle and capsid are used interchangeably herein.

As used herein, the terms “recombinant AAV (rAAV) vector” or “gene delivery vector” refer to a virus particle that functions as a nucleic acid delivery vehicle, and which comprises the vector genome (e.g., a therapeutic payload encapsidated as a viral genome) packaged within an AAV capsid. Alternatively, in some contexts, the term “vector” may be used to refer to the vector genome/therapeutic payload alone

A “rAAV vector genome” or “rAAV genome” is an AAV genome (i.e., vDNA) that comprises one or more heterologous nucleotide sequences. The manufacture of rAAV vectors generally require only the 145 base terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the minimal TR sequence(s) so as to maximize the size of the transgene that can be efficiently packaged by the vector. The structural and non-structural protein coding sequences may be provided in trans (e.g., from a vector, such as a plasmid, or by stably integrating the sequences into a packaging cell). The rAAV vector genome comprises at least one TR sequence (e.g., AAV TR sequence, synthetic, or other parvovirus TR sequence), optionally two TRs (e.g., two AAV TRs), which typically will be at the 5′ and 3′ ends of the heterologous nucleotide sequence(s), but need not be contiguous thereto. The TRs can be the same or different from each other.

The term “terminal repeat” or “TR” includes any viral terminal repeat and synthetic sequences that form hairpin structures and function as an inverted terminal repeat (ITR), such as the “double-D sequence” as described in U.S. Pat. No. 5,478,745 to Samulski et al. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99: 10405-10), AAV4 (Padron et al., (2005) I. Virol. 79: 5047-58), AAVS (Walters et al., (2004) I. Virol. 78: 3361-71) and CPV(Xie et al., (1996) I. Mol. Biol. 6:497-520 and Tsao et al., (1991) Science 251: 1456-64).

An “AAV terminal repeat” or “AAV TR” may be from any AAV, including but not limited to serotypes AAV-1, AAV-2, AAV-2i8, AAV-3, AAV-4, AAV-5, AAV-6, AAV-7, AAV-8, AAV-9, AAV-10, AAV-11, AAV-12, AAV-13, or any other AAV now known or later discovered. The AAV terminal repeats need not have a wild-type terminal repeat sequence (e.g., a wild-type sequence may be altered by insertion, deletion, truncation or missense mutations), as long as at least one of the terminal repeat mediates the desired functions, a functional TR, e.g., replication, virus packaging, integration, and/or provirus rescue, and the like. One of skill in the art understands to choose a Rep protein that is functional for replication of the functional TR. In some embodiments, the method described herein produces recombinant AAV with 145 bp ITR (inverted terminal repeat) sequences or, smaller ITR sequences. In some embodiments, the recombinant AAV produced by this method comprises 130 bp ITR. In some embodiments, the recombinant AAV produced by this method comprises smaller than 130 bp ITR. In some embodiments, the recombinant AAV produced by the instant method, comprises synthetic or, mutant ITR or, restrictive ITR for example as described in WO2014143932, U.S. Pat. No. 9,447,433; WO2011088081, U.S. Pat. No. 9,169,494; WO 2019143950, all of which are incorporated in entirety by reference herein.

In some embodiments of any one of the aspects, the viral particle, e.g. rAAV comprises a transgene. The transgene can be any transgene. Herein, the transgene is used interchangeably with heterologous transgene. As used herein, a “transgene” refers to a polynucleotide or a nucleic acid that is intended or has been introduced into a cell or organism. Transgenes include any nucleic acid, such as a gene that encodes a polypeptide or protein. Suitable transgenes, for example, for use in gene therapy are well known to those of skill in the art. For example, the rAAVs described herein can include transgenes and uses that include, but are not limited to, those described in U.S. Pat. Nos. 6,547,099; 6,506,559; and 4,766,072; Published U.S. Application No. 20020006664; 20030153519; 20030139363; and published PCT applications of WO01/68836 and WO03/010180, and e.g. miRNAs and other transgenes of WO2017/152149; each of which are hereby incorporated herein by reference in their entirety.

The terms “host cell” and “producer cell” are used interchangeably herein and refer to any cell or cells capable of producing a recombinant virus, e.g., recombinant adeno-associated virus vector. For example, the host cells can be transfected with one or more nucleic acids to produce the recombinant virus, e.g., recombinant adeno-associated virus particles. It is noted that cells can be transfected at density, e.g., from about 1×10⁶ to about 8×10⁷ viable cells/ml. The cells can be cultured in suspension.

Host cell can be a mammalian, bacterial, or yeast cell. In some embodiments of any one of the aspect, the host cell can be HeLa cell, COS cell, COS-1 cell, COS-7 cell, HEK293 cell, A549 cell, BHK cell, BSC-1 cell, BSC-40 cell, Vero cell, Sf′c9 cell, Sf-21 cell, Tn-368 cell, BTI-Tn-5B1-4 (High-Five) cell, Saos cell, C2C12 cell, L cell, HT1080 cell, HepG2 cell, WEHI cell, 3T3 cell, 10T1/2 cell, MDCK cell, BMT-10 cell, WI38 cell, or primary fibroblast, hepatocyte or myoblast cells derived from mammals. Unless otherwise indicated, the terms “cell” or “cell line” are understood to include modified or engineered variants of the indicated cell or cell line. A recombinant viral particle, e.g., aAAV can be produced from a host cell using any suitable method known in the art.

The titer of the recombinant viral particles, e.g., rAAV vector particles is from about 1×10⁹ to about 1×10¹⁴ vector genomes/ml, or, from about 1×10¹⁰ to about 5×10¹³ vector genomes/ml, or, from about 1×10¹⁰ to about 1×10¹³ vector genomes/ml, or from about 1×10¹¹ to about 5×10¹² viral particles/ml. In certain embodiments, the titer of recombinant viral particles, e.g., rAAV vector particles is from about 1×10¹¹ to about 1×10¹² vector genomes/ml or viral particles/ml. In some embodiments, the titer can range from about 1×10⁹ to about 1×10¹¹ TCID50/ml. In some embodiments, the particle to infectivity ratio of recombinant viral particles (e.g rAAV) can range from about 10² to about 10⁵ vg/TCID50 or, from about 10² to about 5×10⁴ vg/TCID50 or, from about 10² to about 10⁴ vg/TCID50. In certain embodiments, the particle to infectivity ratio is from 10² to about 10³ vg/TCID50. In yet another embodiment, the particle to infectivity ratio is less than 10² vg/TCID50. It is noted that “vector genome” can interchangeably be used as “viral genome”.

TCID50 assay: The infectious titer (TCID50) method is used to evaluate the in vitro AAV infectivity of drug product in HeLa RC32 cells. In this assay, HeLa RC32 cells are transduced with adenovirus type 5 helper virus and serial dilutions of drug product. After three days of infection the cells are treated with proteinase K to digest protein and the replicated AAV vector DNA is quantitated with qPCR technology. This method utilizes a DNA primer and fluorescent dye-based detection system. The absolute quantity of the ITR target sequence from the vector DNA is interpolated from a standard curve prepared with a plasmid. Containing ITR is prepared as a test sample and is used as an assay control. Results are expressed as infectious units per milliliter (IU/mL). It is noted that for comparing TCID50/ml among different preparations, TCID50/ml is preferably normalized to vg/ml.

“Partitioning” or. “Partitioning Effect”, or, ‘partition effect” is used in the instant application as a separation of the “full” or recombinantly expressed virus particle from the empty virus particle. In the present invention, the partitioning effect is achieved with anion exchange (AEX) modalities where adjusting the buffers e.g., dilution buffer and/or equilibration buffer, empty particles (e.g empty AAV particles) are shifted towards the unbound fraction and thus results in an enriched population of full viral particles (e.g full AAV particles) in the bound fraction of AEX chromatography. It is noted, that enriched population of full or, packaged viral particles may comprise partially packaged or, partially full viral particles. Without limiting to any theory, the operating window to achieve optimum partitioning effect can be established with a high throughput screening method for example, as depicted in Example 1. As such it likely provides a valuable alternative to bind-elute or standard flow-through modes of chromatography. It is not meant to refer specifically to any one type of liquid chromatography, such as “partition” chromatography, a term that is used when exploiting relative solubilities in the stationary and mobile phases.

### Compositions Comprising Recombinant AAV Vector Particles

The rAAV vectors as disclosed herein can be formulated in a composition. For example, the rAAV vectors as disclosed herein can be formulated in a pharmaceutical composition with a pharmaceutically acceptable excipient, i.e., one or more pharmaceutically acceptable carrier substances and/or additives, e.g., buffers, carriers, excipients, stabilisers, etc. The composition, e.g., the pharmaceutical composition may be provided in the form of a kit.

Accordingly, in one aspect, provided herein is a composition comprising the recombinant AAV vector particles described herein. Generally, the composition comprises the recombinant AAV vector particles described herein at a concentration from about 1e⁹ vg/ml to about 1e¹⁵ vg/ml. In some embodiments, the composition comprises the recombinant AAV vector particles described herein at a concentration from about 1e¹⁰ vg/ml to about 1e¹⁴ vg/ml. In some embodiments, the composition comprises the recombinant AAV vector particles described herein at a concentration from about 1e¹² vg/ml to about 1e¹⁴ vg/ml. In some embodiments, the composition comprises the recombinant AAV vector particles described herein at a concentration from about 1e¹² vg/ml to about 1e¹⁵ is vg/ml. For example, the composition comprises the recombinant AAV vector particles described herein at a concentration from about 3e¹² vg/ml to about 3e¹³ vg/ml, from about 2.5e¹² vg/ml to about 1e¹⁴ vg/ml, from about 3e¹³ vg/ml to about 1e¹⁴ vg/ml, or from 1e¹³ vg/ml to about 1e¹⁴ vg/ml.

In some embodiments, the composition comprises the recombinant AAV vector particles described herein at a concentration of about 1e¹² vg/ml, or about 1.5e¹² vg/ml, or about 2e¹² vg/ml, or about 2.5e¹² vg/ml, or about 3e¹² vg/ml, or about 3.5e¹² vg/ml, or about 4e¹² vg/ml, or about 4.5e¹² vg/ml, or about 5e¹² vg/ml, or about 5.5e¹² vg/ml, or about 6e¹² vg/ml, or about 6.5e¹² vg/ml, or about 7e¹² vg/ml, or about 7.5e¹² vg/ml, or about 8e¹² vg/ml, or about 8.5e¹² vg/ml, or about 9e¹² vg/ml, or about 9.5e¹³ vg/ml, or about 1e¹³ vg/ml, or about 1.5e¹³ vg/ml, or about 2e¹³ vg/ml, or about 2.5e¹³ vg/ml, or about 3e¹³ vg/ml, or about 3.5e¹³ vg/ml, or about 4e¹³ vg/ml, or about 4.5e¹³ vg/ml, or about 5e¹³ vg/ml, or about 5.5e¹³ vg/ml, or about 6e¹³ vg/ml, or about 6.5e¹³ vg/ml, or about 7e¹³ vg/ml, or about 7.5e¹³ vg/ml, or about 8e¹³ vg/ml, or about 8.5e¹³ vg/ml, or about 9e¹³ vg/ml, or about 9.5e¹³ vg/ml, or about 1e¹⁴ vg/ml.

In some embodiments, the composition is substantially free of glycine.

In some embodiments, the composition is substantially free of trehalose. For example, the composition comprises a bulking agent and the bulking agent is not trehalose, such as trehalose dehydrate.

In some embodiments, the composition is substantially free of sodium chloride.

In some embodiments, the composition is substantially free of a non-ionic surfactant. For example, the composition is substantially free of polysorbate 80 (PS80). In some embodiments, the composition comprises a non-ionic surfactant and wherein the non-ionic surfactant is not a polysorbate, such as PS80.

In some embodiments, the composition is substantially free of pharmaceutically acceptable salts (sodium salts, ammonium salts or potassium salts, e.g., NaCl).

Generally, the composition has a pH of about 6.5 to about 8.0. For example, the composition has a pH of about 6.5 to about 7.5. In some embodiments, the composition has a pH of from about 7 to about 8. For example, the composition has a pH of from about 7.3 to about 7.9. In some other non-limiting example, the composition has a pH of from about 7.4 to about 7.8 or from about 7.4 to about 7.7. In some embodiments, the composition has a pH of from about 7.3 to about 7.6, e.g., from about 7.3 to about 7.55. In some preferred embodiments, the composition has a pH less than about 7.5. For example, the composition has a pH about 7.4 or lower, about 7.3 or lower, about 7.2 or lower, about 7.1 or lower, about 7.0 or lower, about 6.9 or lower, about 6.8 or lower, about 6.7 or lower, about 6.6 or lower, or about 6.5 or lower. For example, the pH of the composition is about 7.4 or lower, about 7.3 or lower, about 7.2 or lower, about 7.1 or lower, or about 7.0 or lower. In some embodiments, the composition has a pH of about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8. In some embodiments, the composition has a pH of at least about 7.0, at least about 7.2, at least about 7.3, at least about 7.4, at least about 7.5, at least about 7.6, at least about 7.7, as least about 7.7 or at least about.

In some embodiments, the composition comprises a buffer. It is noted that any physiological buffer can be used. Non-limiting examples of buffers include, but are not limited to, PBS, Tris.HCl, phosphate, citric acid, histidine, tromethamine, succinic acid, malic acid, α-ketoglutaric acid, carbonate (bicarbonate-carbonic acid buffer), and protein buffers. In some embodiments, the buffer is PBS. In some embodiments, the buffer comprises Tris. In some embodiments, buffer is Tris.HCl. In some embodiments, the buffer is histidine buffer.

Generally, the buffer has a salt concentration of from about 50 mM to about 750 mM. For example, the buffer has a salt concentration from about 75 mM to about 700 mM, from about 100 mM to about 650 mM, from about 120 mM to about 600 mM, or from about 140 mM to about 550 mM. In some embodiments, the buffer has a salt concentration from about 150 mM to about 400 mM. In some embodiments, the buffer has a salt concentration of about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, or about 475 mM. In some preferred embodiments, the buffer has a salt concentration of about 150 mM, about 200 mM or about 365 mM.

In some embodiments, the ionic strength of the composition is at least about 100 mM. For example, the ionic strength of the composition is from about 125 mM to about 750 mM, or from about 150 mM to about 500 mM, or from about 175 mM to about 700 mM, from about 200 mM to about 600 mM, or from about 225 mM to about 550 mM, or from about 250 mM to about 500 mM, or from about 275 mM to about 450 mM, or from about 300 mM to about 400 mM. In some embodiments, the ionic strength of the composition is at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM or at least about 500 mM. In some preferred embodiments, the ionic strength of the composition is about 170 mM, about 210 mM or about 380 mM. In some embodiments, the ionic strength of the composition is less than 100 mM, for example about 95 mM, about 90 mM, about 85 mM, about 80 mM, about 75 mM, about 70 mM, about 65 mM, about 60 mM, about 55 mM, about 50 mM, or, even less.

Generally, the osmolarity of the composition is maintained at near isotonic levels. For example, the osmolarity of the composition can be from about 100 mOsm to about 600 mOsm, such as from about 125 mOsm to about 500 mOsm, or, from about 130 mOsm to about 350 mOsm, or, from about 140 mOsm to about 400 mOsm, or, from about 140 mOsm to about 350 mOsm, or from about 200 mOsm to about 400 mOsm, or from about 500 mOsm to about 600 mOsm, or from about 200 mOsm to about 600 mOsm, or from about 300 mOsm to about 600 mOsm, or from about 200 mOsm to about 500 mOsm, or from about 300 mOsm to about 400 mOsm, or from about 150 mOsm to about 350 mOsm, or from about 175 mOsm to about 300 mOsm. In some embodiments, the osmolarity of the composition is from about 300 mOsm to about 375 mOsm, or from about 200 mOsm to about 350 mOsm, or from about 225 mOsm to about 325 mOsm, or from about 525 mOsm to about 590 mOsm. In some embodiments, the osmolarity of the composition is about 125 mOsm, about 126 mOsm, about 127 mOsm, about 128 mOsm, about 129 mOsm, about 130 mOsm, about 131 mOsm, about 132 mOsm, about 133 mOsm, about 134 mOsm, about 135 mOsm, about 136 mOsm, about 137 mOsm, about 138 mOsm, about 139 mOsm, about 140 mOsm, about 141 mOsm, about 142 mOsm, about 143 mOsm, about 144 mOsm, about 145 mOsm, about 146 mOsm, about 147 mOsm, about 148 mOsm, about 149 mOsm, about 150 mOsm, about 151 mOsm, about 152 mOsm, about 153 mOsm, about 154 mOsm, about 155 mOsm, about 160 mOsm, about 165 mOsm, about 170 mOsm, about 175 mOsm, about 180 mOsm, about 185 mOsm, about 190 mOsm, about 191 mOsm, about 192 mOsm, about 193 mOsm, about 194 mOsm, about 195 mOsm, about 200 mOsm, about 205 mOsm, about 210, mOsm, about 215 mOsm, about 220 mOsm, about 225 mOsm, about 250 mOsm, about 275 mOsm, about 280 mOsm, about 285 mOsm, about 290 mOsm, about 295 mOsm, about 300 mOsm, about 301 mOsm, about 302 mOsm, about 303 mOsm, about 304 mOsm, about 305 mOsm, about 306 mOsm, about 307 mOsm, about 308 mOsm, about 309 mOsm, about 310 mOsm, about 311 mOsm, about 312 mOsm, about 313 mOsm, about 314 mOsm, about 315 mOsm, about 316 mOsm, about 317 mOsm, about 318 mOsm, about 319 mOsm, about 320 mOsm, about 321 mOsm, about 322 mOsm, about 323 mOsm, about 324 mOsm, about 325 mOsm, about 326 mOsm, about 327 mOsm, about 328 mOsm, about 329 mOsm, about 330 mOsm, about 331 mOsm, about 332 mOsm, about 333 mOsm, about 334 mOsm, about 335 mOsm, about 336 mOsm, about 337 mOsm, about 338 mOsm, about 339 mOsm, about 340 mOsm, about 341 mOsm, about 342 mOsm, about 343 mOsm, about 344 mOsm, about 345 mOsm, about 346 mOsm, about 347 mOsm, about 348 mOsm, about 349 mOsm, about 350 mOsm, about 355 mOsm, about 360 mOsm, about 365 mOsm, about 370 mOsm, about 375 mOsm, about 400 mOsm, about 425 mOsm, about 450 mOsm, about 475 mOsm, about 500 mOsm, about 525 mOsm, about 530 mOsm, about 540 mOsm, about 550 mOsm, about 560 mOsm, about 570 mOsm, about 580 mOsm, or about 590 mOsm. In some embodiments, the composition comprises isotonic solution.

In some embodiments, the composition has an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or, about 340 mOsm or, lower, or, about 335 mOsm or lower, or, about 330 mOsm or, lower, or about 325 mOsm or lower, or about 300 mOsm or lower, or, about 280 mOsm or, lower, or, about 260 mOsm, or lower, or, about 250 mOsm or, lower, or, 240 mOsm, or, lower, or, about 230 mOsm or, lower, or, about 225 mOsm or, lower, or, about 220 mOsm or, lower, or about 215 mOsm or, lower, or about, 210 mOsm or, lower, or about 200 mOsm or, lower, or, about 195 mOsm or, lower, or, about 190 mOsm, or, lower, or, about 185 moOsm or lower, or, about 180 mOsm or lower, or, about 175 moOsm or lower, or, about 170 moOsm or lower, or, about 165 moOsm or lower, or, about 160 moOsm or lower, or, about 155 moOsm or lower, or, about 150 moOsm or lower, or, about 145 moOsm or lower, or, about 140 moOsm or lower, or, about 135 moOsm or lower, or, about 130 moOsm or lower, or, about 125 moOsm or lower. In some embodiments, the composition comprises one or more ions and/or salts thereof. Exemplary ions include, but are not limited to sodium, potassium, chloride, magnesium ammonium, carbonate, nitrate, chlorate, chlorite, and calcium. The ions can be provided as a salt, such as a halide (F, Cl, Br, I) salt of sodium, potassium, magnesium, and/or calcium, non-limiting examples of which include NaCl, KCl, MgCl₂, CaCl₂, and combinations thereof. Additional exemplary salts that can be used include, but are not limited to, carboxylic acid salts, such as acetates, propionates, pyrrol idonecarboxylates (or pidolates) or sorbates; poly hydroxylated carboxylic acid salts, such as gluconates, heptagluconates, ketogluconates, lactate gluconates, ascorbates or pantothenates; mono- or polycarboxyl hydroxy acid salts, such as citrates or lactates; amino acid salts, such as aspartates or glutamates; and fulvate salts. The salts are individually included at a concentration of from about 500 NM to about 500 mM, for example, at a concentration of about 500 μM, about 750 NM, about 1 mM, about 1.3 mM, about 1.5 mM, about 1.7 mM, about 2.3 mM, about 2.5 mM, about 2.7 mM, about 3.3 mM, about 3.5 mM, about 3.7 mM, about 4.3 mM, about 4.5 mM, about 4.7 mM, about 5 mM, about 10 mM, about 25 mM, about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM.

In some embodiments, the composition comprises one or more multivalent ions and/or salts thereof. Exemplary multivalent ions include, but are not limited to, calcium, citrate, sulfate, magnesium, and phosphate. Multivalent ions and/or salts thereof can be individually included in the composition at a concentration of from about 500 NM to about 500 mM, for example, at a concentration of about 500 μM, about 750 μM, about 1 mM, about 1.3 mM, about 1.5 mM, about 1.7 mM, about 2.3 mM, about 2.5 mM, about 2.7 mM, about 3.3 mM, about 3.5 mM, about 3.7 mM, about 4.3 mM, about 4.5 mM, about 4.7 mM, about 5 mM, about 10 mM, about 25 mM, about 50 mM, about 75 mM, about 80 mM, about 85 mM, about 90 mM, about 95 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM. Non limiting examples of salts are NaCl, KCl, CaCl₂, CaSO₄, MgSO₄, Na₃PO₄, CaCO₃, NaNO₃, Al₂(SO₄)₃.

In some embodiments, the composition comprises NaCl. When present, NaCl can be at a concentration from about 100 mM to about 500 mM, or from about 125 mM to about 450 mM, or from about 100 mM to about 200 mM, or from about 150 mM to about 200 mM. For example, the composition can comprise NaCl at a concentration from about 150 mM to about 425 mM, from about 175 mM to about 400 mM, or from about 175 mM to about 375 mM. In some embodiments, the composition comprises NaCl at a concentration from about 200 mM to about 375 mM. For example, the composition can comprise NaCl at a concentration of about 100 mM, about 125 mM, about 130 mM, about 135 mM, about 137 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM, about 205 mM, about 210 mM, about 215 mM, about 220 mM, about 225 mM, about 230 mM, about 235 mM, about 240 mM, about 245 mM, about 250 mM, about 255 mM, about 260 mM, about 265 mM, about 270 mM, about 275 mM, about 300 mM, about 325 mM, about 350 mM, about 375 mM or about 400 mM. In some preferred embodiments, the composition comprises NaCl at a concentration of about 140 mM, about 175 mM, about 200 mM, about 300 mM, about 350 mM or about 375 mM. In some embodiments, the composition is substantially free of NaCl.

In some embodiments, the composition comprises KCl. When present, KCl can be at a concentration from about 1 mM to about 10 mM. For example, the composition can comprise KCl at a concentration from about 1.5 mM to about 7.5 mM. In some embodiments, the composition comprises KCl at a concentration from about 2 mM to about 5.5 mM. For example, the composition can comprise KCl at a concentration of about 2 mM, about 2.25 mM, about 2.5 mM, about 2.7 mM, about 2.75 mM, about 3 mM, about 3.25 mM, about 3.5 mM, about 3.75 mM, about 4 mM, about 4.25 mM, about 4.5 mM, about 4.75 mM, about 5 mM, or about 5.25 mM. In some preferred embodiments, the composition comprises KCl at a concentration of about 2.7 mM, about 3 mM, about 3.5 mM or about 5 mM. In some embodiments, the composition is substantially free of KCl.

In some embodiments, the composition comprises CaCl₂. When present, CaCl₂ can be at a concentration from about 0.1 mM to about 2 mM. For example, the composition can comprise CaCl₂ at a concentration from about 0.5 mM to about 1.5 mM. In some embodiments, the composition comprises CaCl₂ at a concentration from about 0.75 mM to about 1.25 mM. For example, the composition can comprise CaCl₂ at a concentration of about 0.1 mM, about 0.15 mM, about 0.2 mM, about 0.25 mM, about 0.3 mM, about 0.35 mM, about 0.4 mM, about 0.45 mM, about 0.5 mM, about 0.55 mM, about 0.6 mM, about 0.65 mM, about 0.7 mM, about 0.75 mM, about 0.8 mM, about 0.85 mM, about 0.9 mM, about 0.95 mM, about 1 mM, about 1.1 mM, about 1.15 mM, about 1.2 mM, about 1.25 mM, about 1.3 mM, about 1.35 mM, about 1.4 mM, about 1.45 mM, about 1.5 mM, about 1.55 mM, about 1.6 mM, about 1.65 mM, about 1.7 mM, about 1.75 mM, about 1.8 mM, about 1.85 mM, about 1.9 mM, about 1.95 mM, or about 2 mM. In some preferred embodiments, the composition comprises CaCl₂ at a concentration of about 0.9 mM. In some embodiments, the composition is substantially free of CaCl₂.

In some embodiments, the composition comprises MgCl₂. When present, MgCl₂ can be at a concentration from about 0.1 mM to about 1.5 mM. For example, the composition can comprise MgCl₂ at a concentration from about 0.25 mM to about 1 mM. In some embodiments, the composition comprises MgCl₂ at a concentration from about 0.25 mM to about 0.75 mM. For example, the composition can comprise MgCl₂ at a concentration of about 0.1 mM, about 0.15 mM, about 0.2 mM, about 0.25 mM, about 0.3 mM, about 0.35 mM, about 0.4 mM, about 0.45 mM, about 0.5 mM, about 0.55 mM, about 0.6 mM, about 0.65 mM, about 0.7 mM, about 0.75 mM, about 0.8 mM, about 0.85 mM, about 0.9 mM, about 0.95 mM, about 1 mM, about 1.1 mM, about 1.15 mM, about 1.2 mM, about 1.25 mM, about 1.3 mM, about 1.35 mM, about 1.4 mM, about 1.45 mM, or about 1.5 mM. In some preferred embodiments, the composition comprises MgCl₂ at a concentration of about 0.5 mM. In some embodiments, the composition is substantially free of MgCl₂.

In some embodiments, the composition comprises MgSO₄. When present, MgSO₄ can be at a concentration from about 5 mM to about 150 mM. For example, the composition can comprise MgSO₄ at a concentration from about 10 mM to about 120 mM, or from about 10 mM to about 50 mM, or from about 15 mM to about 45 mM, or about 75 mM to about 125 mM, or from about 80 mM to about 100 mM, or from about 85 mM to about 95 mM. In some embodiments, the composition comprises MgSO₄ at a concentration from about 15 mM to about 100 mM. For example, the composition can comprise MgSO₄ at a concentration of about 5 mM, about 10 mM, about 15 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 115 mM or about 120 mM. In some embodiments, the composition is substantially free of MgSO₄.

In some embodiments, the composition comprises heptagluconate or a salt thereof. For example, the composition comprises calcium heptagluconate such as calcium α-d-heptagluconate. When present, heptagluconate or a salt thereof, e.g., calcium α-d-heptagluconate can be present at a concentration of from about 1% (w/v) to about 20% (w/v). For example, the composition can comprise heptagluconate or a salt thereof, e.g., calcium α-d-heptagluconate at a concentration of from about 2.5% (w/v) to about 17.5% (w/v), from about 5% (w/v) to about 15% (w/v), or from about 7.5% (w/v) to about 12.5% (w/v). In some embodiments, the composition comprises heptagluconate or a salt thereof, e.g., calcium α-d-heptagluconate at a concentration of about 1% (w/v), about 2.5% (w/v), about 5% (w/v), about 7.5% (w/v), about 10% (w/v), about 12.5% (w/v), about 15% (w/v), about 17.5% (w/v) or about 20% (w/v). In some embodiments, the composition is substantially free of heptagluconate or a salt thereof, e.g., calcium heptagluconate such as calcium α-d-heptagluconate.

In some embodiments, the composition comprises phosphate, e.g., mono basic or dibasic phosphate or a salt thereof. When present, the phosphate, e.g., mono basic or dibasic phosphate or a salt thereof can be at a concentration from about 5 mM to about 30 mM. For example, the composition can comprise phosphate, e.g., mono basic or dibasic phosphate or a salt thereof at a concentration from about 7.5 mM to about 25 mM. In some embodiments, the composition comprises phosphate, e.g., mono basic or dibasic phosphate or a salt thereof at a concentration from about 10 mM to about 20 mM. For example, the composition can comprise phosphate, e.g., mono basic or dibasic phosphate or a salt thereof at a concentration of about 5 mM, about 7.5 mM, about 10 mM, about 12.5 mM, about 15 mM, about 17.5 mM, about 20 mM, about 22.5 mM, about 25 mM, about 25.75 mM or about 30 mM. In some preferred embodiments, the composition comprises phosphate, e.g., mono basic or dibasic phosphate or a salt thereof at a concentration of about 10 mM, 15 mM or about 20 mM.

In some embodiments, the composition comprises a mono basic phosphate or a salt thereof at a concentration from about 0.25 mM to about 3 mM. For example, the composition comprises a mono basic phosphate or a salt thereof at a concentration from about 0.5 mM to about 2.75 mM, from about 0.75 mM to about 2.5 mM or from about 1 mM to about 2.25 mM. In some embodiments, the composition comprises a mono basic phosphate or a salt thereof at a concentration from about 1.25 mM to about 2.25 mM. For example, the composition comprises a mono basic phosphate or a salt thereof at a concentration of about 0.25 mM, about 0.5 mM, about 0.75 mM, about 1 mM, about 1.25 mM, about 1.5 mM, about 1.75 mM, about 2 mM, about 2.25 mM, about 2.5 mM, about 2.75 mM, or about 3 mM. In some preferred embodiments, the composition comprises a mono basic phosphate or a salt thereof at a concentration of about 1 mM, about 1.5 mM or about 2 mM. In some embodiments, the mono basic phosphate or salt thereof is potassium phosphate monobasic. In some embodiments, the composition is substantially free of mono basic phosphate, e.g., potassium phosphate monobasic.

In some embodiments, the composition comprises a dibasic phosphate or a salt thereof at a concentration from about 5 mM to about 15 mM. For example, the composition comprises a dibasic phosphate or a salt thereof at a concentration from about 7.5 mM to about 12.5 mM or from about 8 mM to about 10 mM. In some embodiments, the composition comprises a dibasic phosphate or a salt thereof at a concentration of about 5 mM, about 5.5 mM, about 6 mM, about 6.5 mM, about 7 mM, about 7.5 mM, about 8 mM, about 8.5 mM, about 9 mM, about 9.5 mM, about 10 mM, about 10.5 mM, about 11 mM, about 11.5 mM, about 12 mM, about 12.5 mM, about 13 mM, about 13.5 mM, about 14 mM, about 14.5 mM, or about 15 mM. In some preferred embodiments, the composition comprises a dibasic phosphate or a salt thereof at a concentration of about 8 mM or about 9 mM. In some embodiments, the dibasic phosphate or a salt thereof is sodium phosphate dibasic. In some embodiments, the composition is substantially free of dibasic phosphate, e.g., sodium phosphate di basic.

In some embodiments, the composition comprises Tris (e.g., Tris.HCl) or a salt thereof at a concentration from about 1 mM to about 50 mM. For example, the composition comprises Tris (e.g., Tris.HCl) or a salt thereof at a concentration of from about 5 mM to about 40 mM, from about 7.5 mM to about 35 mM, from about 10 mM to about 30 mM or from about 15 mM to about 25 mM. In some embodiments, the composition comprises Tris (e.g., Tris.HCl) or a salt thereof at a concentration of about 5 mM, about 7.5 mM, about 10 mM, about 12.5 mM, about 15 mM, about 17.5 mM, about 20 mM, about 22.5 mM, about 25 mM, about 27.5 mM, about 30 mM, about 32.5 mM, about 35 mM, about 37.5 mM, about 40 mM, about 42.5 mM, about 45 mM, about 47.5 mM, or about 50 mM

In some embodiments, the composition comprises histidine or a salt thereof at a concentration from about 1 mM to about 50 mM. For example, the composition comprises histidine or a salt thereof at a concentration of from about 5 mM to about 40 mM, from about 7.5 mM to about 35 mM, from about 10 mM to about 30 mM or from about 15 mM to about 25 mM. In some embodiments, the composition comprises histidine or a salt thereof at a concentration of about 5 mM, about 7.5 mM, about 10 mM, about 12.5 mM, about 15 mM, about 17.5 mM, about 20 mM, about 22.5 mM, about 25 mM, about 27.5 mM, about 30 mM, about 32.5 mM, about 35 mM, about 37.5 mM, about 40 mM, about 42.5 mM, about 45 mM, about 47.5 mM, or about 50 mM.

In some embodiments, the composition further comprises a bulking agent. In some embodiments, the bulking agent is a polyol or providone (PVP K24). Exemplary polyols include, but are not limited to, polyhydroxy hydrocarbons, monosaccharides, disaccharides, and trisaccharides. Some exemplary polyols include but are not limited to, sorbitol, mannitol, glycerol, propylene glycol, polyethylene glycol, dulcitol, sucrose, lactose, maltose, trehalose and dextran. In some embodiments, polyol is sorbitol, sucrose or mannitol. In some embodiments, the bulking agent is sorbitol. In some embodiments, the bulking agent is sucrose. In some embodiments, the bulking agent is mannitol. In some embodiments, the bulking agent is not trehalose, e.g., trehalose dehydrate. In some embodiments, the bulking agent is not a dextran, e.g., Dextran T40 and/or Dextran T10.

When present, the bulking agent, e.g., a polyol or providone (PVP K24) can be present at a concentration of from about 0.5% (w/v) to about 10% (w/v). For example, the composition can comprise a bulking agent, e.g., a polyol or providone (PVP K24) at a concentration from about from about 1% (w/v) to about 7.5% (w/v), e.g., from about 3% (w/v) to about 6% (w/v). In some embodiments, the composition comprises a bulking agent, e.g., a polyol or providone (PVP K24) at a concentration of about 1% (w/v), about 1.5% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), about 8% (w/v), about 8.5% (w/v), about 39% (w/v), about 9.5% (w/v) or about 10% (w/v). In some preferred embodiments, the composition comprises a bulking agent, e.g., a polyol or providone (PVP K24) at a concentration of about 1% (w/v), about 3% (w/v), or about 5% (w/v).

In some embodiments, the composition comprises glycerol, sorbitol, sucrose, or mannitol at a concentration from about 1% (w/v) to about 10% (w/v). In some embodiments, the composition comprises glycerol, sorbitol, sucrose, or mannitol at a concentration from about 1% (w/v) to about 10% (w/v). In some embodiments, the composition comprises sorbitol at concentration from about 3% (w/v) to about 6% (w/v). In some embodiments, the composition comprises sorbitol at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, the composition comprises sucrose at concentration from about 3% (w/v) to about 6% (w/v). In some embodiments, the composition comprises sucrose at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, the composition comprises mannitol at concentration from about 3% (w/v) to about 6% (w/v). In some embodiments, the composition comprises mannitol at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v).

In some embodiments, the composition further comprises a non-ionic surfactant. The non-ionic surfactant can be selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof. Non-limiting examples of suitable non-ionic surfactants include polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and combinations thereof. In some embodiments, the non-ionic surfactant is selected from the group consisting of TWEEN 60 nonionic detergent, PPG-PEG-PPG Pluronic 10R5, Pluronic F-68 (PF 68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof. In some embodiments, the non-ionic surfactant is Poloxamer P 188, Poloxamer P407, Pluronic F-68, Ecosurf SA-15, Brij S20, Tergitol NP-10, IGEPAL CA 720 or Tween 80. In some embodiments, the composition is substantially free of a non-ionic surfactant. In some embodiments, the non-ionic surfactant is not a polysorbate, e.g., Tween 80 (also referred to as polysorbate 80 or PS80).

When present, the non-ionic surfactant can be present at a concentration from about 0.0001% (w/v) to about 0.01% (w/v). For example, the composition can comprise a non-ionic surfactant at a concentration from about 0.0005% (w/v) to about 0.0015% (w/v). In some embodiments, the composition can comprise a non-ionic surfactant at a concentration of about 0.0001% (w/v), about 0.0002% (w/v), about 0.0003% (w/v), about 0.0004% (w/v), about 0.0005% (w/v), about 0.0006% (w/v), about 0.0007% (w/v), about 0.0008% (w/v), about 0.0009% (w/v), about 0.001% (w/v), about 0.002% (w/v), about 0.003% (w/v), about 0.004% (w/v), about 0.005% (w/v), about 0.006% (w/v), about 0.007% (w/v), about 0.008% (w/v), about 0.009% (w/v), or about 0.01%. (w/v). In some preferred embodiments, the composition comprises a non-ionic surfactant at a concentration of about 0.0005% (w/v) or about 0.001% (w/v).

In certain embodiments, the composition comprises Poloxamer P 188, Poloxamer P407, Pluronic F-68 or Ecosurf SA-15 at a concentration of about 0.0005% (w/v), about 0.0008% (w/v), about 0.0009% (w/v), 0.001% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.006% (w/v), 0.007% (w/v), about 0.008% (w/v), about 0.009% (w/v), or about 0.01% (w/v).

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran) and a non-ionic surfactant (e.g., Poloxamer P 188, Poloxamer P407, Pluronic F-68, Ecosurf SA-15, Brij S20, Tergitol NP-10, IGEPAL CA 720 or Tween 80), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), a non-ionic surfactant (e.g., Poloxamer P 188, Poloxamer P407, Pluronic F-68, Ecosurf SA-15, Brij S20, Tergitol NP-10, IGEPAL CA 720 or Tween 80), and a multivalent ion selected from the group consisting of calcium, citrate, sulfate, and magnesium, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran) and a non-ionic surfactant (e.g., Poloxamer P 188, Poloxamer P407, Pluronic F-68, Ecosurf SA-15, Brij S20, Tergitol NP-10, IGEPAL CA 720 or Tween 80), and wherein the composition is substantially free of magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), and a multivalent ion (e.g., calcium, citrate, sulfate, or magnesium), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), a multivalent ion (e.g., calcium, citrate, sulfate, or magnesium), and a non-ionic surfactant (e.g., Poloxamer P 188, Poloxamer P407, Pluronic F-68, Ecosurf SA-15, Brij S20, Tergitol NP-10, IGEPAL CA 720 or Tween 80), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris or histidine), a bulking agent (e.g., sucrose, sorbitol or mannitol), and a non-ionic surfactant (e.g., Pluronic F-68), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris or histidine), a bulking agent (e.g., sucrose, sorbitol or mannitol), a non-ionic surfactant (e.g., Pluronic F-68), and a multivalent ion or salt thereof (e.g., magnesium sulfate), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris or histidine), a bulking agent (e.g., sucrose, sorbitol or mannitol), and a non-ionic surfactant (e.g., Pluronic F-68), and the composition is substantially free of magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris or histidine), a bulking agent (e.g., sucrose, sorbitol or mannitol), and a multivalent ion or salt thereof (e.g., magnesium sulfate), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., PBS, Tris or histidine), a bulking agent (e.g., sucrose, sorbitol or mannitol), and a multivalent ion or salt thereof (e.g., magnesium sulfate), and the composition is substantially free of non-ionic surfactant (e.g., Pluronic F-68), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition has a pH of from about 7 to about 8, e.g., the composition has a pH of from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph. In other words, all individual specific combinations of buffers, buffer groups, bulking agents, bulking agent groups, non-ionic surfactants and non-ionic surfactant groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and a multivalent ion or salt thereof (e.g., a multivalent ion selected from the group consisting of calcium, citrate, sulfate, magnesium, and phosphate, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has a pH of from about 7 to about 8, e.g., the composition has a pH of from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants and non-ionic surfactant groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition is substantially free of a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has a pH of from about 7 to about 8, e.g., the composition has a pH of from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants, non-ionic surfactant groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has a pH of from about 7 to about 8, e.g., a pH of from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific multivalent ions and multivalent ion group listed above in this paragraph. Likewise, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer groups, bulking agents, bulking agent groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), and the composition is substantially free of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition has a pH of from about 7 to about 8, e.g., a pH of from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants, non-ionic surfactant groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph. In other words, all individual specific combinations of buffers, buffer groups, bulking agents, bulking agent groups, non-ionic surfactants and non-ionic surfactant groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and a multivalent ion or salt thereof (e.g., a multivalent ion selected from the group consisting of calcium, citrate, sulfate, magnesium, and phosphate, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants and non-ionic surfactant groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition is substantially free of a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants, non-ionic surfactant groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific multivalent ions and multivalent ion group listed above in this paragraph. Likewise, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer groups, bulking agents, bulking agent groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), and the composition is substantially free of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants, non-ionic surfactant groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition has a pH of from about 7 to about 8 (e.g., from about 7.3 to about 7.8 or from about 7.4 to about 7.7 or from about 7.3 to about 7.55), and wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph. In other words, all individual specific combinations of buffers, buffer groups, bulking agents, bulking agent groups, non-ionic surfactants and non-ionic surfactant groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), and a multivalent ion or salt thereof (e.g., a multivalent ion selected from the group consisting of calcium, citrate, sulfate, magnesium, and phosphate, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has a pH of from about 7 to about 8 (e.g., a pH of from about 7.3 to about 7.8 or from about 7.4 to about 7.7 or from about 7.3 to about 7.55), and wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants, non-ionic surfactant groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition is substantially free of a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has a pH of from about 7 to about 8 (e.g., from about 7.3 to about 7.8 or from about 7.4 to about 7.7 or from about 7.3 to about 7.55), and wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants, non-ionic surfactant groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), wherein the composition has a pH of from about 7 to about 8 (e.g., from about 7.3 to about 7.8 or from about 7.4 to about 7.7 or from about 7.3 to about 7.55), and wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific multivalent ions and multivalent ion group listed above in this paragraph. Likewise, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer groups, bulking agents, bulking agent groups, multivalent ions and multivalent ion groups listed above in this paragraph are specifically contemplated and claimed.

In some embodiments, the composition comprises, in addition to the rAAV, a buffer (e.g., a buffer selected from the group consisting of PBS, Tris.HCl, phosphate, citric acid, histidine, tromethamine, succinic acid, malic acid, α-ketoglutaric acid, carbonate, protein buffers and any combinations thereof, or a buffer selected from the group consisting of PBS, Tris.HCl, phosphate, citric acid and any combinations thereof, or a buffer selected from the group consisting of citric acid, histidine, succinic acid, malic acid, α-ketoglutaric acid and any combinations thereof, or a buffer selected from the group consisting of PBS, Tris.HCl, histidine, and any combinations thereof, or a buffer selected from the group consisting of Tris.HCl, phosphate, citric acid, carbonate and any combinations thereof), a bulking agent (e.g., a polyol or providone (PVP K24), or a polyol selected from the group consisting of hydrocarbons, monosaccharides, disaccharides, trisaccharides and any combinations thereof, or a polyol selected from the group consisting of sorbitol, mannitol, glycerol, propylene glycol, polyethylene glycol, dulcitol, sucrose, lactose, maltose, trehalose, dextran and any combinations thereof, or a polyol selected from the group consisting of sorbitol, mannitol, dulcitol, sucrose, lactose, maltose, trehalose and any combinations thereof, or polyol selected from the group consisting of sucrose, mannitol, sorbitol and any combinations thereof, or a polyol selected from the group consisting of propylene glycol, polyethylene glycol, dextran and any combinations thereof, or a polyol selected from the group sucrose, mannitol, sorbitol, glycerol, propylene glycol, polyethylene glycol, dextran and any combinations thereof), and a multivalent ion or salt thereof (e.g., a multivalent ion selected from the group consisting of calcium, citrate, sulfate, and magnesium, or a multivalent ion selected from the group consisting of calcium, citrate, sulfate and magnesium, or a multivalent ion selected from the group consisting of calcium, sulfate and magnesium or a multivalent ion selected from the group consisting of citrate, sulfate and magnesium, or magnesium sulfate), and the composition is substantially free of a non-ionic surfactant (e.g., a non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof, or non-ionic surfactant selected from the group consisting of polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and any combinations thereof, or a non-ionic surfactant selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof, or a non-ionic surfactant selected from the group consisting of Poloxamer P 188, Poloxamer P407, Pluronic 10R5, Pluronic F-68, Ecosurf SA-15, Brij S20, Tergitol NP-10, IGEPAL CA 720, Tween 80 and any combinations thereof, or a non-ionic surfactant selected from the group consisting of Pluronic 10R5 and Pluronic F-68, or a non-ionic surfactant selected from the group consisting of Poloxamer P188, Poloxamer P407, Poloxamer P 338 and any combinations thereof, or a non-ionic surfactant selected from the group consisting of Brij S20, Brij S10, Brij 010, Brij C10, BRIJ 020 and any combinations thereof, or a non-ionic surfactant selected from the group consisting of ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13 and any combinations thereof), wherein the composition has a pH of from about 7 to about 8 (e.g., from about 7.3 to about 7.8 or from about 7.4 to about 7.7 or from about 7.3 to about 7.55), and wherein the composition has an osmolarity from about 100 mOsm to about 500 mOsm, e.g., an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, such as an osmolarity of about 500 mOsm or lower, or about 475 mOsm or lower, about 450 mOsm or lower, or about 425 mOsm or lower, or about 400 mOsm or lower, or about 375 mOsm or lower, about 350 mOsm or lower, or about 325 mOsm or lower, or about 300 mOsm or lower, or about 375 mOsm or lower, or about 350 mOsm or lower, or about 325 mOsm or lower, or 300 mOsm or lower, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. It is noted that any one of the specific buffers or group of buffers listed above in this paragraph can be used with any one of the specific bulking agents or group of bulking agents listed above in this paragraph and with any of the specific non-ionic surfactants or group of surfactants listed above in this paragraph and with any of the specific multivalent ions and multivalent ion group listed above in this paragraph. Similarly, any one of the specific bulking agents or group of bulking agents listed above in this paragraph can be used with any one of the specific buffers or group of buffers listed above in this paragraph and with any of the specific non-ionic surfactants or group of surfactants listed above in this paragraph and with any of the specific multivalent ions and multivalent ion group listed above in this paragraph. Likewise, any of the specific non-ionic surfactants or group of surfactants listed above in this paragraph can be used with any one of the specific buffers or group of buffers listed above in this paragraph and with any one of the specific bulking agents or group of bulking agents listed above in this paragraph and with any of the specific multivalent ions and multivalent ion group listed above in this paragraph. As well, any of the specific multivalent ions and multivalent ion group listed above in this paragraph can be used with any one of the specific buffers or group of buffers listed above in this paragraph and with any one of the specific bulking agents or group of bulking agents listed above in this paragraph and with any of the specific non-ionic surfactants or group of surfactants listed above in this paragraph. In other words, all individual specific combinations of buffers, buffer group, bulking agents, bulking agent groups, non-ionic surfactants, non-ionic surfactant groups, multivalent ions and multivalent ion groups listed above in this paragraph are specifically contemplated and claimed.

In some embodiments, the buffer is PBS, the bulking agent is mannitol and the non-ionic surfactant is Pluronic-F68. For example, the buffer is PBS, the bulking agent is mannitol, the non-ionic surfactant is Pluronic-F68, and the composition has a pH of from about 7 to about 8, e.g., from about 7.3 to about 7.8 or from about 7.4 to about 7.7 or from about 7.3 to about 7.55.

In some embodiments, the buffer is PBS, the bulking agent is sorbitol and the non-ionic surfactant is Pluronic-F68. For example, the buffer is PBS, the bulking agent is sorbitol, the non-ionic surfactant is Pluronic-F68, and the composition has a pH of from about 7 to about 8, e.g., from about 7.3 to about 7.8 or from about 7.4 to about 7.7 or from about 7.3 to about 7.55.

In some embodiments, the buffer is PBS, the bulking agent is sorbitol, the non-ionic surfactant is Pluronic-F68, and the multivalent ion or salt thereof is magnesium sulfate. For example, the buffer is PBS, the bulking agent is sorbitol, the non-ionic surfactant is Pluronic-F68, the multivalent ion or salt thereof is magnesium sulfate and the composition has a pH of from about 7 to about 8, e.g., from about 7.3 to about 7.8 or from about 7.4 to about 7.7 or from about 7.3 to about 7.55.

In some embodiments, the buffer is Tris the bulking agent is mannitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is mannitol, the non-ionic surfactant is Pluronic-F68, and the composition has a pH of from about 7 to about 8, e.g., from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is Tris, the bulking agent is sorbitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, and the composition has a pH of from about 7 to about 8, e.g., from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is Tris, the bulking agent is sorbitol, the non-ionic surfactant is Pluronic-F68, and the multivalent ion or salt thereof is magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, the multivalent ion or salt thereof is magnesium sulfate and the composition has a pH of from about 7 to about 8, e.g., from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is mannitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is mannitol, the non-ionic surfactant is Pluronic-F68, and the composition has a pH of from about 7 to about 8, e.g., from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is sorbitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, and the composition has a pH of from about 7 to about 8, e.g., from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is sorbitol, the non-ionic surfactant is Pluronic-F68, and the multivalent ion or salt thereof is magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, the multivalent ion or salt thereof is magnesium sulfate and the composition has a pH of from about 7 to about 8, e.g., from about 7.3 to about 7.8 or from about 7.4 to about 7.7 or from about 7.3 to about 7.55, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is PBS, the bulking agent is mannitol and the non-ionic surfactant is Pluronic-F68. For example, the buffer is PBS, the bulking agent is mannitol, the non-ionic surfactant is Pluronic-F68, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm.

In some embodiments, the buffer is PBS, the bulking agent is sorbitol and the non-ionic surfactant is Pluronic-F68. For example, the buffer is PBS, the bulking agent is sorbitol, the non-ionic surfactant is Pluronic-F68, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm.

In some embodiments, the buffer is PBS, the bulking agent is sorbitol, the non-ionic surfactant is Pluronic-F68, and the multivalent ion or salt thereof is magnesium sulfate. For example, the buffer is PBS, the bulking agent is sorbitol, the non-ionic surfactant is Pluronic-F68, the multivalent ion or salt thereof is magnesium sulfate, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm.

In some embodiments, the buffer is Tris the bulking agent is mannitol and the non-ionic surfactant is Pluronic-F68. For example, the buffer is PBS, the bulking agent is mannitol, the non-ionic surfactant is Pluronic-F68, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm.

In some embodiments, the buffer is Tris, the bulking agent is sorbitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is Tris, the bulking agent is sorbitol, the non-ionic surfactant is Pluronic-F68, and the multivalent ion or salt thereof is magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, the multivalent ion or salt thereof is magnesium sulfate, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is mannitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is mannitol, the non-ionic surfactant is Pluronic-F68, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is sorbitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is sorbitol, the non-ionic surfactant is Pluronic-F68, and the multivalent ion or salt thereof is magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, the multivalent ion or salt thereof is magnesium sulfate, and the composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is PBS, the bulking agent is mannitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is mannitol, the non-ionic surfactant is Pluronic-F68, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is PBS, the bulking agent is sorbitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is PBS, the bulking agent is sorbitol, the non-ionic surfactant is Pluronic-F68, and the multivalent ion or salt thereof is magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, the multivalent ion or salt thereof is magnesium sulfate, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is Tris the bulking agent is mannitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is mannitol, the non-ionic surfactant is Pluronic-F68, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is Tris, the bulking agent is sorbitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is Tris, the bulking agent is sorbitol, the non-ionic surfactant is Pluronic-F68, and the multivalent ion or salt thereof is magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, the multivalent ion or salt thereof is magnesium sulfate, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is mannitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is mannitol, the non-ionic surfactant is Pluronic-F68, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is sorbitol and the non-ionic surfactant is Pluronic-F68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the buffer is histidine buffer, the bulking agent is sorbitol, the non-ionic surfactant is Pluronic-F68, and the multivalent ion or salt thereof is magnesium sulfate, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80. For example, the buffer is PBS, the bulking agent is sorbitol, the non-ionic surfactant is Pluronic-F68, the multivalent ion or salt thereof is magnesium sulfate, and the composition has a pH of from about 7 to about 8 (e.g., from about 7.3 to about 7.8 or from about 7.4 to about 7.7 or from about 7.3 to about 7.55) and an osmolarity from about 125 mOsm to about 500 mOsm (e.g., from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm), and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, a composition described herein has a pH of from about 7 to about 8. For example, any one of the above described composition can have a pH of about 7.3 to about 7.8 or about 7.4 to about 7.5. In some embodiments, any of the above described composition can have a pH of about 7.3, or about 7.4, or about 7.5, or about 7.6, or about 7.7, or about 7.8.

In some embodiments, a composition described herein has an osmolarity from about 100 mOsm to about 500 mOsm. For example, any of the above described composition has an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm.

In some embodiments, a composition described herein has a pH of from about 7 to about 8 and an osmolarity from about 100 mOsm to about 500 mOsm. For example, any one of the above described composition can have a pH of about 7.3 to about 7.8 or about 7.4 to about 7.5, and an osmolarity from about 125 mOsm to about 500 mOsm, or from about 200 mOsm to about 400 mOsm, or from about 200 mOsm to about 500 mOsm, or from about 300 mOsm to about 400 mOsm, or from about 150 mOsm to about 350 mOsm, or from about 175 mOsm to about 300 mOsm.

Without wishing to be bound by a theory, the AAV vector particles are stored in the composition without significant aggregation. Aggregation can be assessed by dynamic light scattering (DLS), photon correlation spectroscopy and visual appearance. In some embodiments, the AAV vector particles stored using the compositions described herein do not exhibit significant aggregation when stored at 4° C. for one, two, three, four, five, six, seven, eight, nine, ten or more days. In some embodiments, the AAV vector particles that are stored as such compositions do not exhibit significant aggregation after one, two, three, four, five, six, seven, eight, nine, ten or more freeze-thaw cycles at −20° C. or at −80° C. In some embodiments, the compositions described herein are used for multiple AAV serotypes e.g., AAV2, AAV9, AAVrh10. In some embodiments, the compositions described herein are used for multiple AAV serotypes e.g. for heparin binding and non heparin binding AAV serotypes. In some embodiments, the compositions described herein are used for multiple AAV serotypes e.g. for heparin binding and non heparin binding AAV serotypes, wherein the composition is maintained at isotonic or, near isotonic strength. In some embodiments, the compositions described herein are used for multiple AAV serotypes e.g. for heparin binding and non heparin binding AAV serotypes, wherein the composition has low osmolarity e.g less than about 400 mOsm, or, less than about 350 mOsm, or, less than about 300 mOsm, or, less than about 250 mOsm, or, less than about 225 mOsm, or, less than about 200 mOsm, or, less than about 180 mOsm, or, less than about 160 mOsm, or less than about 150 mOsm, or, less than about 145 mOsm. Or, less than about 140 mOsm, or, even less.

In some embodiments, the AAV vectors or, the purified recombinant AAV particles (rAAV) stored according to the compositions described herein exhibit a low polydispersity index (PDI), as measured by dynamic light scattering, indicating that no significant aggregation of the AAV vectors has taken place. In some embodiments, the AAV vectors or, the purified rAAV particles stored according to the compositions described herein exhibit a PDI less than 0.1, for example about 0.099, about 0.098, about 0.097, about, 0.095, about 0.09, about 0.085, about 0.08, about 0.075, about 0.073, about 0.07, about 0.069, about, about 0.065, about 0.060, about 0.058, about 0.057, about 0.055, about 0.05, about 0.045, about 0.040, about 0.035 or, even less. In some embodiments, the AAV vectors or, the purified rAAV particles stored according to the compositions described herein exhibit a PDI less than 0.1, for example about 0.099, about 0.098, about 0.097, about, 0.095, about 0.09, about 0.085, about 0.08, about 0.075, about 0.073, about 0.07, about 0.069, about, about 0.065, about 0.060, about 0.058, about 0.057, about 0.055, about 0.05, about 0.045, about 0.040, about 0.035 or, even less when stored at 4° C. for one, two, three, four, five, six, seven, eight, nine, ten or more days. In some embodiments, the AAV vectors or, purified recombinant AAV particles stored according to the compositions described herein exhibit substantially no aggregation. In some embodiments, the AAV vectors or, purified rAAV particles stored according to the compositions described herein exhibit substantially no aggregation with PDI values less than about 0.1. In some embodiments, the AAV vectors or, purified rAAV particles stored according to the compositions described herein exhibit a Polydispersity (PD) value of less than about 30% PD, or, less than about 25% PD, or, less than about 20% PD, or, less than about 15% PD, or, even less.

In some embodiments, the AAV vectors or, purified rAAV particles stored according to the compositions described herein exhibit a low polydispersity index (PDI), as measured by dynamic light scattering, indicating that no significant aggregation of the AAV vectors has taken place after one, two, three, four, five, six, seven, eight, nine, ten or more freeze-thaw cycles wherein each freeze thaw cycle comprises 24 hr at −80° C. followed by 24 hr at room temperature.

In some embodiments, the AAV vectors or, purified rAAV particles stored according to the compositions described herein exhibit PDI less than 0.1, for example about 0.099, about 0.098, about 0.097, about, 0.095, about 0.09, about 0.085, about 0.08, about 0.075, about 0.073, about 0.07, about 0.069, about, about 0.065, about 0.060, about 0.058, about 0.057, about 0.055, about 0.05, about 0.045, about 0.040, about 0.035 or, even less one, two, three, four, five, six, seven, eight, nine, ten or more freeze-thaw cycles, wherein each freeze thaw cycle comprises 24 hr at −80° C. followed by 24 hr at room temperature.

In some embodiments, the AAV vectors or, purified rAAV particles stored according to the compositions described herein exhibit substantially no aggregation. In some embodiments, the AAV vectors or, purified rAAV particles stored according to the compositions described herein exhibit substantially no aggregation with PDI values less than about 0.1. In some embodiments, the compositions described herein can be used to store AAV vectors wherein the TCID50/ml of the AAV vector is retained by at least 50% or, more e.g, at least about 55%, or, at least about 60%, or at least about 65%, or, at least about 70%, or, at least about 75%, or, at least about 80%, or, at least about 85%, or, at least about 90%, or, at least about 95%, or, at least about 96%, or, at least about 97%, or, at least about 98%, or, at least about 99%, after one, two, three, four, five, six, seven, eight, nine, ten or, more freeze thaw cycles than that measured before starting the freeze thaw cycle, wherein each freeze thaw cycle comprises 24 hr at −80° C. followed by 24 hr at room temperature. In some embodiments, the compositions described herein can be used to store AAV vectors wherein the TCID50/ml of the AAV vector is substantially unchanged after one, two, three, four, five, six, seven, eight, nine, ten or, more freeze thaw cycles than that measured before starting the freeze thaw cycle, wherein each freeze thaw cycle comprises 24 hr at −80° C. followed by 24 hr at room temperature.

In some embodiments, the AAV vectors stored according to the compositions described herein exhibit an average aggregate particle radius (Rh), as measured by dynamic light scattering, indicating that no significant aggregation of the AAV vectors has taken place. In some embodiments, the AAV vectors stored according to the compositions described herein exhibit an average aggregate particle radius of less than about 35 nm when stored at 4° C. for one, two, three, four, five, six, seven, eight, nine, ten or more days. For example, the AAV vectors stored according to the compositions described herein exhibit an average aggregate particle radius of less than about 30 nm, less than about 25 nm, less than about 20 nm, less than about 15 nm, less than about 10 nm, or less than about 5 nm when stored at 4° C. for one, two, three, four, five, six, seven, eight, nine, ten or more days.

In some embodiments, the AAV vectors stored according to the compositions described herein exhibit an average aggregate particle radius, as measured by dynamic light scattering, indicating that no significant aggregation of the AAV vectors has taken place after one, two, three, four, five, six, seven, eight, nine, ten or more freeze-thaw cycles at −20° C. or at −80° C. In some embodiments, the AAV vectors stored according to the compositions described herein exhibit an average aggregate particle radius of less than 35 nm after one, two, three, four, five, six, seven, eight, nine, ten or more freeze-thaw cycles at −20° C. or at −80° C. For example, the AAV vectors stored according to the compositions described herein exhibit an average aggregate particle radius of less than about 30 nm, less than about 25 nm, less than about 20 nm, less than about 15 nm, less than about 10 nm, or less than about 5 nm after one, two, three, four, five, six, seven, eight, nine, ten or more freeze-thaw cycles at −20° C. or at −80° C.

In some embodiments, the composition comprises, in addition to the rAAV, about 10 mM Phosphate pH 7.4, about 200 mM NaCl, about 5 mM KCl, about 1% (w/v) mannitol, and about 0.0005% (w/v) IGEPAL CA 720.

In some embodiments, the composition comprises, in addition to the rAAV, about 20 mM Phosphate pH 7.4, about 300 mM NaCl, about 3 mM KCl, about 3% (w/v) mannitol, and about 0.001% (w/v) Brij S20.

In some embodiments, the composition comprises, in addition to the rAAV, about 20 mM Phosphate pH 7.4, about 300 mM NaCl, about 3 mM KCl, about 3% (w/v) sorbitol, and about 0.001% (w/v) Ecosurf SA-15.

In some embodiments, the composition comprises, in addition to the rAAV, about 10 mM Phosphate pH 7.4, about 350 mM NaCl, about 2.7 mM KCl, about 5% (w/v) sorbitol, and about 0.001% (w/v) poloxamer 188.

In some embodiments, the composition comprises, in addition to the rAAV, about 10 mM Phosphate pH 6.95-7.2, about 137 mM NaCl, about 2.7 mM KCl, about 0.9 mM CaCl₂, about 0.5 mM MgCl₂, and about 0.001% (w/v) Pluronic F-68.

In some embodiments, the composition comprises, in addition to the rAAV, about 10 mM Phosphate pH 7.3, about 180 mM NaCl, about 2.7 mM KCl, about 5% (w/v) sorbitol, and about 0.001% (w/v) Poloxamer 188.

In some embodiments, the composition comprises, in addition to the rAAV, about 15 mM Phosphate pH 7.4, about 375 mM NaCl, about 3.5 mM KCl, about 5% (w/v) sorbitol, and about 0.0005% (w/v) Tergitol NP-10.

In some embodiments, the composition comprises, in addition to the rAAV, about 15 mM Phosphate pH 7.4, about 375 mM NaCl, about 3.5 mM KCl, about 3% (w/v) glycerol, and about 0.0005% (w/v) Tween 80.

In some embodiments, the composition comprises, in addition to the rAAV, about 10 mM Phosphate pH 7.6, about 137 mM NaCl, about 2.7 mM KCl, about 5% (w/v) sorbitol, and about 0.01% Pluronic F-68.

In some embodiments, the composition comprises, in addition to the rAAV, about 10 mM Phosphate pH 7.4, about 137 mM NaCl, about 2.7 mM KCl, about 5% (w/v) sorbitol, about 0.01% Pluronic F-68, and about 20 mM MgSO₄.

In some embodiments, the composition comprises, in addition to the rAAV, about 10 mM Phosphate pH 7.6, about 137 mM NaCl, about 2.7 mM KCl, about 5% (w/v) mannitol, and about 0.01% Pluronic F-68.

In some embodiments, the composition comprises, in addition to the rAAV, about 10 mM Phosphate pH 7.3, about 137 mM NaCl, about 2.7 mM KCl, about 5% (w/v) mannitol, about 0.01% Pluronic F-68, and about 20 mM MgSO₄.

In some embodiments, the composition comprises, in addition to the rAAV, about 10 mM Phosphate pH 7.4, about 137 mM NaCl, about 2.7 mM KCl, about 5% (w/v) sorbitol, and about 20 mM MgSO₄.

In some embodiments, the composition comprises, in addition to the rAAV, about 10 mM Phosphate pH 7.4, about 137 mM NaCl, about 2.7 mM KCl, about 5% (w/v) mannitol, and about 20 mM MgSO₄.

In some embodiments, the composition comprises, in addition to the rAAV, about 10 mM Phosphate pH 6.2, about 137 mM NaCl, about 2.7 mM KCl, about 5% (w/v) sorbitol, and about 10% (w/v) calcium α-d-heptagluconate.

In some embodiments, the composition comprises, in addition to the rAAV, about 10 mM Phosphate pH 6.2, about 137 mM NaCl, about 2.7 mM KCl, about 5% (w/v) mannitol, and about 10% (w/v) calcium α-d-heptagluconate.

In some embodiments, the composition comprises, in addition to the rAAV, about 20 mM Tris pH 7.5, about 5% (w/v) sorbitol, and about 0.01% Pluronic F-68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 20 mM Tris pH 7.4, about 5% (w/v) sorbitol, about 0.01% Pluronic F-68, and about 20 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 20 mM Tris pH 7.5, about 5% (w/v) mannitol, and about 0.01% Pluronic F-68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 20 mM Tris pH 7.4, about 5% (w/v) mannitol, about 0.01% Pluronic F-68, and about 20 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 20 mM Tris pH 7.5, about 5% (w/v) sorbitol, and about 20 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 20 mM Tris pH 7.5, about 5% (w/v) mannitol, and about 20 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine pH 7.69, about 5% (w/v) sorbitol, and about 0.01% Pluronic F-68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine pH 7.46, about 5% (w/v) sorbitol, about 0.01% Pluronic F-68, and about 20 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine pH 7.62, about 5% (w/v) mannitol, and about 0.01% Pluronic F-68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine pH 7.49, about 5% (w/v) mannitol, about 0.01% Pluronic F-68, and about 20 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine pH 7.53, about 5% (w/v) sorbitol, and about 20 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80

In some embodiments, the composition comprises, in addition to the rAAV, about 25 mM histidine pH 7.55, about 5% (w/v) mannitol, and about 20 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine and about 90 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine, about 90 mM MgSO₄, and about 0.01% Pluronic F-68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine, about 90 mM MgSO₄, and about 5% (w/v) sucrose, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 25 mM histidine, about 90 mM MgSO₄, about 5% (w/v) sucrose, and about 0.01% Pluronic F-68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 20 mM Tris and about 90 mM MgSO₄, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 20 mM Tris, about 90 mM MgSO₄, and about 0.01% Pluronic F-68, and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

In some embodiments, the composition comprises, in addition to the rAAV, about 20 mM Tris, about 90 mM MgSO₄, and about 5% (w/v) sucrose and optionally: (i) the composition is substantially free of glycine; and/or (ii) the composition is substantially free of trehalose, e.g., trehalose dehydrate or dextran, e.g., Dextran T10 or T40; and/or (iii) the composition is substantially free of pharmaceutically acceptable salts (sodium salts, ammonium salts or potassium salts, e.g., NaCl); and/or (iv) the composition is substantially free of polysorbate, such as PS80.

### Exemplary Compositions:

In some embodiments, the composition, e.g., the pharmaceutical composition comprises recombinant AAV vector (rAAV), in 10 mM Phosphate pH 7.4, 200 mM NaCl, 5 mM KCl, 1% (w/v) mannitol, 0.0005% (w/v) IGEPAL CA 720 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 20 mM Phosphate pH 7.4, 300 mM NaCl, 3 mM KCl, 3% (w/v) mannitol, 0.001% (w/v) Brij S20 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 20 mM Phosphate pH 7.4, 300 mM NaCl, 3 mM KCl, 3% (w/v) sorbitol, 0.001% (w/v) Ecosurf SA-15 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 10 mM Phosphate pH 7.4, 350 mM NaCl, 2.7 mM KCl, 5% (w/v) sorbitol, 0.001% (w/v) poloxamer 188 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 15 mM Phosphate pH 7.4, 375 mM NaCl, 3.5 mM KCl, 5% (w/v) sorbitol, 0.0005% (w/v) Tergitol NP-10 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In some embodiments, the composition, e.g., the pharmaceutical composition comprises recombinant AAV vector (rAAV), in 15 mM Phosphate pH 7.4, 375 mM NaCl, 3.5 mM KCl, 3% (w/v) glycerol, 0.0005% (w/v) Tween 80 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

### HTS Assay and Kits

In another aspect, provided herein is a high throughput screening (HTS) assay for determining conditions for purifying or isolating viral particles from a sample, e.g., a harvesting media. For example, provided herein is a HTS method for determining conditions (e.g., buffer component concentrations, chromatography media, chromatography size, and the like) for purifying or isolating viral particles (e.g., different AAV serotypes) from a sample, e.g., a harvesting media using anion exchange chromatography by HTS.

Generally, the HTS method comprises subjecting a sample, e.g., a harvest media such as an affinity eluate from obtained from a harvest media comprising the viral particles (e.g., rAAV particles) to a high throughput AEX method. For example, a harvest media such as an affinity eluate from obtained from a harvest media comprising the viral particles (e.g., rAAV particles) is subjected to a high throughput AEX method (e.g., 0.1 mL) using Tecan or FPLC in a scouting mode. The buffers used for the AEX comprise varying amounts of one or more components. For example, the sample dilution buffer, and/or the column equilibration buffer comprises a weak acid or a salt thereof in a varying amount. In some embodiments, the AEX method evaluation comprises addition of 0-20 mM citric acid (or, 0-30 mM succinic acid or, 0-60 mM acetic acid) in the sample dilution/column equilibration phases, and optionally, a conductivity mediated isocratic step elution at the pH greater than 8.

In some embodiments, the sample dilution buffer, and/or the column equilibration buffer is substantially free of a weak acid or salt thereof and the sample dilution buffer, and/or the column equilibration buffer comprises an amino acid in a varying amount. For example, the AEX method evaluation comprises addition of 0-150 mM of an amino acid such as histidine in the sample dilution/column equilibration phases, and optionally, a conductivity mediated isocratic step elution at the pH greater than 8

The AEX flowthrough and eluate fractions are then analyzed to assess the separation of empty capsids from full capsids as well as capsids recovery. For example, the AEX flowthrough and eluate fractions are analyzed using Tecan plate reader and/or SEC-HPLC to assess the separation of empty capsids from full capsids as well as capsids recovery.

In some embodiments, the sample used in the HTS for high throughput AEX is an affinity eluate from a harvesting media. Thus, in some embodiments, the method further comprises a step of purifying/isolating a plurality of recombinantly expressed virus particles from the harvesting media via affinity chromatography to produce an affinity eluate comprising the plurality of recombinantly expressed virus particles for use in the HTS method described herein. For example, a harvesting media is subjected to affinity chromatography purification and the affinity purified material is then carried over the high throughput AEX method described herein.

In some embodiments, the HTS method comprises subjecting a sample comprising a plurality of recombinantly expressed virus particles to a high throughput AEX method (0.1 mL) using Tecan or FPLC in a scouting mode. AEX method evaluation contains addition of 0-20 mM citric acid (or, 0-30 mM succinic acid or, 0-60 mM acetic acid) in the sample dilution/column equilibration phases and a conductivity mediated isocratic step elution at the pH greater than 8. The AEX flowthrough and eluate fractions are then analyzed using Tecan plate reader or SEC-HPLC to assess the separation of empty capsids from full capsids as well as capsids recovery.

In some embodiments, the HTS method comprises purifying/isolating a plurality of recombinantly expressed virus particles from a culture media (with or without lysis) via affinity chromatography to produce an affinity eluate comprising the plurality of recombinantly expressed virus particles and subjecting the affinity purified material to a high throughput AEX method (0.1 mL) using Tecan or FPLC in a scouting mode. AEX method evaluation contains addition of 0-20 mIVI citric acid (or, 0-30 mM succinic acid or, 0-60 mM acetic acid) in the sample dilution/column equilibration phases and a conductivity mediated isocratic step elution at the pH greater than 8. The AEX flowthrough and eluate fractions are then analyzed using Tecan plate reader or SEC-HPLC to assess the separation of empty capsids from full capsids as well as capsids recovery.

In some embodiments, the HTS method comprises purifying/isolating a plurality of recombinantly expressed virus particles from a culture media (with or without lysis) via affinity chromatography using different affinity elution buffers to produce different affinity eluates comprising the plurality of recombinantly expressed virus particles and subjecting the affinity purified materials to a high throughput AEX method (0.1 mL) using Tecan or FPLC in a scouting mode. AEX method evaluation contains addition of 0-20 mM citric acid (or, 0-30 mM succinic acid or, 0-60 mM acetic acid) in the sample dilution/column equilibration phases and a conductivity mediated isocratic step elution at the pH greater than 8. The AEX flowthrough and eluate fractions are then analyzed using Tecan plate reader or SEC-HPLC to assess the separation of empty capsids from full capsids as well as capsids recovery.

Without wishing to be bound by a theory, the HTS methods described herein enable identification of various chromatography conditions, e.g., buffer components and concentrations, chromatography media, sample preparations and the like to remove empty viral particles from a sample in a high throughput manner. It is noted that the method can be scaled down or, scaled up according to the production need.

In another aspect, provided herein is a kit for determining conditions (e.g., buffer component concentrations, chromatography media, chromatography column size, and the like) for purifying or isolating viral particles (e.g., different AAV serotypes) from a harvesting media. For example, provided herein is a kit for determining conditions (e.g., buffer component concentrations, chromatography media, chromatography size, and the like) for purifying or isolating viral particles (e.g., different AAV serotypes) from a harvesting media using anion exchange chromatography by HTS. Generally, the kit comprises one or more of, for example, buffers, chromatography media, systems for HTS, and the like such that the skilled artisan may carry out the methods described herein. Additionally, the kit can include instructions for carrying out the methods described herein.

In some embodiments, the kit comprises a buffer described herein. For example, the kit comprises a buffer described herein, e.g., anion exchange dilution buffer, anion exchange equilibration buffer, anion exchange elution buffer, and/or affinity elution buffer. In some embodiments, a buffer in the kit can have a varying amount of a component of the buffer. For example, the buffer comprises a weak acid in a varying amount.

In some embodiments, the kit comprises an anion exchange dilution buffer described herein, where at least one component of the buffer is in a varying amount. In some embodiments, the kit comprises an anion exchange dilution buffer described herein, where the buffer comprises a weak acid or a salt thereof and where the weak acid or a salt thereof is present in a varying amount in the buffer. In some embodiments, the kit comprises an anion exchange dilution buffer described herein, where the buffer does not comprise a weak acid or salt thereof and at least one component of the buffer, e.g., an amino acid, is in a varying amount.

In some embodiments, the kit comprises an anion exchange equilibration buffer described herein, where at least one component of the buffer is in a varying amount. In some embodiments, the kit comprises an anion exchange equilibration buffer described herein, where the buffer comprises a weak acid or a salt thereof and where the weak acid or a salt thereof is present in a varying amount in the buffer. In some embodiments, the kit comprises an anion exchange equilibration buffer described herein, where the buffer does not comprise a weak acid or salt thereof and at least one component of the buffer, e.g., an amino acid, is in a varying amount.

The kit can also include chromatography media. For example, the kit can comprise an anion exchange chromatography media and/or affinity chromatography media. It is noted that the chromatography media can be in a column for use in a chromatography system, e.g., a HTS system. Further, the column can be any desired size for use in a HTS assay. In some embodiments, the kit comprises an anion exchange chromatography media. In some embodiments, the kit comprises an affinity chromatography media.

In some embodiments, the kit comprises a high-throughput liquid handler.

In some embodiments, the kit comprises one or more multi-well plates. For example, the kit comprises one or more multi-well plates and where one or more wells comprise a buffer, e.g., a buffer described herein.

The kit can also include instructions for use. For example, the kit can include instructions for practicing the methods described herein. In some embodiments, the kit includes instructions for determining conditions (e.g., buffer component concentrations, chromatography media, chromatography size, and the like) for purifying or isolating viral particles (e.g., different AAV serotypes) from a harvesting media. It is noted that the instructions can be present in the kit in a variety of forms, one or more of which can be present in or on the kit. One form in which these instructions may be present is as printed information on a suitable medium or substrate, e.g., a piece or pieces of paper on which the information is printed, in or on the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

In some embodiments, the kit comprises one or more multi-well plates and at least one of an anion exchange dilution buffer, an anion exchange equilibration buffer, an anion exchange elution buffer or an affinity elution buffer.

In some embodiments, the kit comprises one or more multi-well plates; at least one of an anion exchange dilution buffer, an anion exchange equilibration buffer, an anion exchange elution buffer or an affinity elution buffer; and a chromatography media, e.g., AEX chromatography media.

In some embodiments, the kit comprises at least two of an anion exchange dilution buffer, an anion exchange equilibration buffer, an anion exchange elution buffer and an affinity elution buffer.

In some embodiments, the kit comprises a chromatography media, e.g., AEX chromatography media and at least two of an anion exchange dilution buffer, an anion exchange equilibration buffer, an anion exchange elution buffer and an affinity elution buffer.

Exemplary embodiments of the various aspects described herein can be described with the following numbered embodiments:

Embodiment 1: A process for purifying or isolating recombinantly expressed adeno associated virus particles from a harvesting media, the method comprising: (a) purifying/isolating a plurality of recombinantly expressed virus particles from the harvesting media via affinity chromatography to produce an eluate comprising the plurality of recombinantly expressed virus particles, wherein an elution buffer for affinity chromatography (affinity elution buffer) comprises a predetermined amount of glycine, optionally, the affinity elution buffer is substantially free of weak acids or salts thereof; and optionally, the affinity elution buffer comprises an amino acid; (b) adjusting the affinity chromatography eluate for subsequent purification through anion exchange chromatography, wherein the adjusted eluate comprises a predetermined amount of an anionic compound; and (c) purifying/isolating the plurality of recombinantly expressed virus particles from the adjusted eluate of affinity chromatography by anion exchange chromatography to produce a solution comprising a plurality of purified/isolated recombinantly expressed virus particles, wherein an equilibration buffer for anion exchange chromatography comprises a predetermined amount of a weak acid or a salt thereof, optionally the weak acid is citric acid, acetic acid or succinic acid.

Embodiment 2: The process of Embodiment 1, wherein the anionic compound of 1 (b) is an acid or a salt thereof, optionally the acid is citric acid, citrate, acetic acid, or succinic acid.

Embodiment 3: The process of any one Embodiments 1-2, wherein the affinity elution buffer comprises histidine at a concentration of at least about 1 mM, 5 mM, 10 mM, 15 mM, 20 mM, 25 mM, 30 mM, 35 mM, 40 mM, 45 mM, 50 mM or more.

Embodiment 4: The process of any one Embodiments 1-3, wherein the affinity elution buffer comprises histidine at a concentration from about 1 mM to about 50 mM, from about 5 mM to about 45 mM, from about 10 mM to about 40 mM, from about 15 mM to about 35 mM or from about 20 mM to about 30 mM.

Embodiment 5: The process of any one Embodiments 1-4, wherein the affinity elution buffer comprises histidine at a concentration of about 1 mM, about 5 mM, about 10 mM, about 15 mM, about 20 mM, about 25 mM, about 30 mM, about 35 mM, about 40 mM, about 45 mM or about 50 mM.

Embodiment 6: The process of any one Embodiments 1-5, wherein the affinity elution buffer comprises histidine at a concentration of about 25 mM.

Embodiment 7: The process of any one of Embodiments 1-6, wherein the affinity elution buffer comprises glycine at a concentration of at least about 20 mM, 25 mM, 30 mM, 35 mM, 40 mM, 45 mM, 50 mM, 55 mM, 60 mM, 65 mM, 70 mM, 75 mM, 80 mM, 90 mM, 95 mM, 100 mM or more.

Embodiment 8: The process of any one of Embodiments 1-7, wherein the affinity elution buffer comprises glycine at a concentration of from about 25 mM to about 100 mM, from about 30 mM to about 95 mM, from about 35 mM to about 90 mM, from about 40 mM to about 80 mM, or from about 45 mM to about 75 mM.

Embodiment 9: The process of any one of Embodiments 1-8, wherein the affinity elution buffer comprises glycine at a concentration of about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 90 mM, about 95 mM or about 100 mM.

Embodiment 10: The process of any one of Embodiments 1-9, wherein the affinity elution buffer comprises a salt.

Embodiment 11: The process of any one of Embodiments 1-10, wherein the affinity elution buffer comprises a salt at concentration of at least about 5 mM, 6 mM, 7 mM, 8 mM, 9 mM, 10 mM, 11 mM, 12 mM, 13 mM, 14 mM, 15 mM or more.

Embodiment 12: The process of any one of Embodiments 1-11, wherein the affinity elution buffer comprises a salt concentration of from about 5 mM to about 15 mM, from about 6 mM to about 14 mM, from about 7 mM to about 13 mM, from about 8 mM to about 12 mM or from about 9 mM to about 11 mM.

Embodiment 13: The process of any one of Embodiments 1-12, wherein the affinity elution buffer comprises a salt at concentration of about 5 mM, about 6 mM, about 7 mM, about 8 mM, about 9 mM, about 10 mM, about 11 mM, about 12 mM, about 13 mM, about 14 mM, or about 15 mM.

Embodiment 14: The process of any one of Embodiments 10-13, wherein the salt is MgCl₂.

Embodiment 15: The process of any one of Embodiments 1-14, wherein the affinity elution buffer comprises a polymer.

Embodiment 16: The process of any one of Embodiments 1-15, wherein the affinity elution buffer comprises a polymer at a concentration of at least about 0.1%, 0.15%, 0.2%, 0.25%, 0.3%, 0.35%, 0.4%, 0.45%, 0.5% or more.

Embodiment 17: The process of any one of Embodiments 1-16, wherein the affinity elution buffer comprises a polymer at a concentration of from about 0.1% to about 0.5%, from about 0.15% to about 0.45%, from about 0.2% to about 0.4%, or from about 0.25% to about 0.35%. 0.1%,

Embodiment 18: The process of any one of Embodiments 1-17, wherein the affinity elution buffer comprises a polymer at a concentration of about 0.1%, about 0.15%, about 0.2%, about 0.25%, about 0.3%, about 0.35%, about 0.4%, about 0.45%, or about 0.5%.

Embodiment 19: The process of any one of Embodiments 15-18, the polymer is a non-ionic polymer.

Embodiment 20: The process of any one of Embodiments 15-19, wherein the polymers is a poloxomer.

Embodiment 21: The process of any one of Embodiments 1-20, wherein the affinity elution buffer has a low pH.

Embodiment 22: The process of any one of Embodiments 1-21, wherein the affinity elution buffer has a pH lower than or equal to about 6.5, 6.0, 5.5, 5.0, 4.5, 4.0, 3.5, 3.0, 2.5, 2.2, 2.0, 1.5 or lower.

Embodiment 23: The process of any one of Embodiments 1-22, wherein the affinity elution buffer has a pH of from about 2.0 to about 3.0.

Embodiment 24: The process of any one of Embodiments 1-23, wherein the affinity elution buffer comprises: about 75 mM glycine, about 25 mM histidine, about 10 mM MgCl₂, about 0.3% (w/v) P188 and has a pH of about 3.0.

Embodiment 25: The process of any one of Embodiments 1, 2 or 7-23, wherein the affinity elution buffer comprises citric acid or a salt thereof.

Embodiment 26: The process of Embodiment 25, wherein the affinity elution buffer comprises citric acid or a salt thereof at a concentration of at least about 50 mM, 55 mM, 60 mM, 65 mM, 70 mM, 75 mM, 80 mM, 85 mM, 90 mM, 95 mM, 100 mM or more.

Embodiment 27: The process of Embodiment 25 or 26, wherein the affinity elution buffer comprises citric acid or a salt thereof at a concentration of from about 50 mM to about 100 mM, from about 55 mM to about 95 mM, from about 60 mM to about 90 mM, from about 65 mM to about 85 mM or from about 70 mM to about 75 mM.

Embodiment 28: The process of any one of Embodiments 25-27, wherein the affinity elution buffer comprises citric acid or a salt thereof at a concentration of about 50 mM, about 55 mM, about 60 mM, about 65 mM, about 70 mM, about 75 mM, about 80 mM, about 85 mM, about 90 mM, about 95 mM, or about 100 mM.

Embodiment 29: The process of any one of Embodiments 25-28, wherein the affinity elution buffer comprises citric acid or a salt thereof at a concentration of about 75 mM.

Embodiment 30: The process of any one of Embodiments 1, 2, 7-23 or 25-29, wherein the affinity elution buffer comprises: about 50 mM glycine, about 75 mM citrate, about 10 mM MgCl₂, about 0.3% (w/v) P188 and has a pH of about 3.0.

Embodiment 31: The process of any one of Embodiments 1-30, wherein the affinity elution buffer has conductivity in a range from about 5 mS/cm to about 8 mS/cm, optionally the affinity elution buffer has conductivity in a range from about 5.5 mS/cm to about 7 mS/cm.

Embodiment 32: The process of any one of Embodiments 1-31, wherein the affinity elution buffer has conductivity in a range from about 5.75 mS/cm to about 6.75 mS/cm, optionally the affinity elution buffer has conductivity in a range from about 6.15 mS/cm to about 6.25 mS/cm.

Embodiment 33: The process of any one of Embodiments 1-32, wherein the affinity elution buffer has an osmolarity in a range from about 100 mOms to about 225 mOms, optionally the affinity elution buffer has an osmolarity in a range from about 125 mOms to about 120 mOms.

Embodiment 34: The process of any one of Embodiments 1-33, wherein the affinity elution buffer has an osmolarity in a range from about 150 mOms to about 175 mOms, optionally the affinity elution buffer has an osmolarity in a range from about 155 mOms to about 165 mOms.

Embodiment 35: The process of any one of Embodiments 1-34, wherein the equilibration buffer for anion exchange chromatography comprises the acid or a salt thereof in a concentration of at least about 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM or higher.

Embodiment 36: The process of any one of Embodiments 1-35, wherein the equilibration buffer comprises the acid at a concentration from about 0.5 mM to about 15 mM, from about 1 mM to about 10 mM, from about 1.5 mM to about 7.5 mM, or about 2 mM to about 7 mM.

Embodiment 37: The process of any one of Embodiments 1-36, wherein the wherein the equilibration buffer comprises the acid at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM.

Embodiment 38: The process of any one of Embodiments 1-37, wherein the acid is citric acid or citrate.

Embodiment 39: The process of any one of Embodiments Embodiment 1-38, wherein adjusting the affinity chromatography eluate for subsequent purification through anion exchange chromatography comprises adding an acid or salt thereof to the eluate, optionally the acid or salt thereof is citric acid, citrate, acetic acid or succinic acid.

Embodiment 40: The process of Embodiment 39, wherein the acid or a salt thereof is added to the eluate to a final a concentration of at least about 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM or higher.

Embodiment 41: The process of Embodiment 39 or 40, wherein the acid or a salt thereof is added to the eluate to a final concentration from about 0.5 mM to about 15 mM, from about 1 mM to about 10 mM, from about 1.5 mM to about 7.5 mM, or about 2 mM to about 7 mM.

Embodiment 42: The process of any one of Embodiments 39-41, wherein the acid or a salt thereof is added to the eluate to a final concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM.

Embodiment 43: The process of any one of Embodiments 39-42, wherein the acid is citric acid or a salt thereof (e.g., citrate).

Embodiment 44: The process of any one of Embodiments 1-43, wherein adjusting the affinity chromatography eluate for subsequent purification through anion exchange chromatography comprises diluting the eluate.

Embodiment 45: The process of any one of Embodiments 1-44, wherein adjusting the affinity eluate for anion exchange chromatography comprises diluting the eluate by at least 2×, 3×, 4×, 5×, 6×, 7×, 8×, 9×, 10×, 11×, 12×, 13×, 14×, 15×, 16×, 17×, 18×, 19×, 20× or more.

Embodiment 46: The process of any one of Embodiments 1-41, wherein adjusting the affinity eluate for anion exchange chromatography comprises diluting the eluate with a dilution buffer.

Embodiment 47: The process of Embodiment 46, wherein the dilution buffer comprises an acid or salt thereof.

Embodiment 48: The process of Embodiment 46 or 47, wherein the dilution buffer comprises an acid or salt thereof at a concentration of about at least about 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM, 15 mM, 20 mM, 25 mM, 30 mM, 35 mM, 40 mM, 45 mM, 50 mM or higher.

Embodiment 49: The process of any one of Embodiments 46-48, wherein the dilution buffer comprises an acid or salt thereof at a concentration from about 0.5 mM to about 15 mM, from about 1 mM to about 10 mM, from about 1.5 mM to about 7.5 mM, or about 2 mM to about 7 mM.

Embodiment 50: The process of any one of Embodiments 46-49, wherein the dilution buffer comprises an acid or salt thereof at a concentration of about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 8 mM, about 8.5 mM, about 9 mM, about 9.5 mM or about 10 mM.

Embodiment 51: The process of any one of Embodiments 46-50, wherein the acid is citric acid or a salt thereof, acetic acid or a salt thereof, or succinic acid or a salt thereof.

Embodiment 52: The process of any one of Embodiments 46-51, wherein the dilution buffer comprises bis-tris propane (BTP).

Embodiment 53: The process of any one of Embodiments 46-52, wherein the dilution buffer comprises BTP at a concentration of at least about 25 mM, 50 mM, 75 mM, 100 mM, 125 mM, 150 mM or higher.

Embodiment 54: The process of any one of Embodiments 46-53, wherein the dilution buffer comprises BTP at a concentration of from about 25 mM to about 175 mM, from about 50 mM to about 150 mM, from about 75 mM to about 125 mM, from about 80 mM to about 120 mM, from about 85 mM to about 115 mM, from about 90 mM to about 110 mM or from about 95 mM to about 105 mM.

Embodiment 55: The process of any one of Embodiments 46-54, wherein the dilution buffer comprises BTP at a concentration of about 50 mM, about 75 mM, about 80 mM, about 85 mM, about 90 mM, about 95 mM, about 100 mM, about 105 mM, about 110 mM, about 115 mM, about 120 mM, about 125 mM, about 150 mM or about 175 mM.

Embodiment 56: The process of any one of Embodiments 46-55, wherein the dilution buffer comprises BTP at a concentration of about 100 mM.

Embodiment 57: The process of any one of Embodiments 46-56, wherein the dilution buffer comprises an amino acid.

Embodiment 58: The process of any one of Embodiments 46-57, wherein the dilution buffer comprises an amino acid at a concentration of at least about 25 mM, 50 mM, 75 mM, 100 mM, 125 mM, 150 mM or higher.

Embodiment 59: The process of any one of Embodiments 46-58, wherein the dilution buffer comprises an amino acid at a concentration of from about 25 mM to about 175 mM, from about 50 mM to about 150 mM, from about 75 mM to about 125 mM, from about 80 mM to about 120 mM, from about 85 mM to about 115 mM, from about 90 mM to about 110 mM or from about 95 mM to about 105 mM.

Embodiment 60: The process of any one of Embodiments 46-59, wherein the dilution buffer comprises an amino acid at a concentration of about 50 mM, about 75 mM, about 80 mM, about 85 mM, about 90 mM, about 95 mM, about 100 mM, about 105 mM, about 110 mM, about 115 mM, about 120 mM, about 125 mM, about 150 mM or about 175 mM.

Embodiment 61: The process of any one of Embodiments 46-60, wherein the dilution buffer comprises an amino acid at a concentration of about 100 mM.

Embodiment 62: The process of any one of Embodiments 46-61, wherein the amino acid is histidine.

Embodiment 63: The process of any one of Embodiments 46-62, wherein the dilution buffer comprises glycerol.

Embodiment 64: The process any one of Embodiments 46-63, wherein the dilution buffer comprises glycerol at a concentration of at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% (v/v or w/v) or higher.

Embodiment 65: The process of any one of Embodiments 46-64, wherein the dilution buffer comprises glycerol at a concentration of from about 0.5% to about 9.5%, from about 1% to about 9%, from about 2% to about 8.5%, from about 2.5% to about 8% from about 3% to about 7.5%, from about 3.5% to about 7%, from about 4% to about 6.5% or from about 4.5% to about 5.5% (v/v or w/v).

Embodiment 66: The process of any one of Embodiments 42-65, wherein the dilution buffer comprises glycerol at a concentration of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9% or about 9.5% (v/v or w/v).

Embodiment 67: The process of any one of Embodiments 42-66, wherein the dilution buffer comprises glycerol at a concentration of about 5% (v/v or w/v).

Embodiment 68: The process of any one of Embodiments 46-67, wherein the dilution buffer comprises a non-ionic surfactant.

Embodiment 69: The process of any one of Embodiments 46-68, wherein the dilution buffer comprises a non-ionic surfactant at a concentration of at least about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, about 0.9%, 0.9.5% (v/v or w/v) or higher.

Embodiment 70: The process of any one Embodiments 46-69, wherein the dilution buffer comprises a non-ionic surfactant at a concentration of about 0.05% to about 0.95%, from about 0.1% to about 0.9%, from about 0.15% to about 0.85%, from about 0.2% to about 0.8%, from about 0.25% to about 0.75%, from about 0.3% to about 7%, from about 0.35% to about 0.65% from about 0.4% to about 0.6% or from about 0.45% to about 0.55% (w/v).

Embodiment 71: The process of any one of Embodiments 46-70, wherein the dilution buffer comprises a non-ionic surfactant at a concentration of about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9% or about 0.95% (w/v).

Embodiment 72: The process of any one of Embodiments 46-71, wherein the dilution buffer comprises a non-ionic surfactant at a concentration of about 0.5% (w/v).

Embodiment 73: The process of any one of Embodiments 46-72, wherein the non-ionic surfactant is selected from the group consisting of polyoxyethylene fatty alcohol ethers, polyoxyethylene alkylphenyl ethers, polyoxyethylene-polyoxypropylene block copolymers, alkylglucosides, alkylphenol ethoxylates, preferably polysorbates, polyoxyethylene alkyl phenyl ethers, and any combinations thereof.

Embodiment 74: The process of any one of Embodiments 46-73, wherein the dilution buffer comprises a salt.

Embodiment 75: The process of any one of Embodiments 46-74, wherein the dilution buffer comprises a salt at a concentration of at least about 0.1 mM, 0.25 mM, 0.5 mM, 0.75 mM, 1 mM, 1.25 mM, 1.5 mM, 1.75 mM, 2 mM or higher.

Embodiment 76: The process of any one of Embodiments 46-75, wherein the dilution buffer comprises a salt at a concentration of from about 0.1 mM to about 2 mM, from about 0.25 mM to about 1.75 mM, from about 0.5 mM to about 1.5 mM, or from about 0.75 mM to about 1.25 mM.

Embodiment 77: The process of any one of Embodiments 46-76, wherein the dilution buffer comprises a salt at a concentration of about 0.1 mM, about 0.25 mM, about 0.5 mM, about 0.75 mM, about 1 mM, about 1.25 mM, about 1.5 mM, about 1.75 mM or about 2 mM.

Embodiment 78: The process of any one of Embodiments 46-77, wherein the dilution buffer comprises a salt at a concentration of about 1 mM.

Embodiment 79: The process of any one of Embodiments 46-78, wherein the salt comprises MgCl₂.

Embodiment 80: The process of any one of Embodiments 46-79, wherein the dilution buffer has a high pH.

Embodiment 81: The process of any one of Embodiments 46-80, wherein the dilution buffer has a pH greater than or equal to about 8, about 8.5, about 9, about 9.5 or about 10.

Embodiment 82: The process of any one of Embodiments 46-81, wherein the dilution buffer has a pH of about 9.

Embodiment 83: The process of any one of Embodiments 46-82, wherein the dilution buffer comprises: BTP, histidine, glycerol, PF68, MgCl₂ and has a high pH.

Embodiment 84: The process of any one of Embodiments 14-30 or 35-50, wherein the dilution buffer comprises: BTP, histidine, PF68, MgCl₂ and has a high pH.

Embodiment 85: The process of any one of Embodiments 1-84, wherein the dilution buffer has conductivity in a range from about 0.5 mS/cm to about 3 mS/cm, optionally the dilution Embodiment 86: buffer has conductivity in a range from about 1 mS/cm to about 2.5 mS/cm.

Embodiment 86: The process of any one of Embodiments 1-85, wherein the dilution buffer has conductivity in a range from about 1.25 mS/cm to about 2.25 mS/cm, optionally the dilution buffer has conductivity in a range from about 1.5 mS/cm to about 1.75 mS/cm.

Embodiment 87: The process of any one of Embodiments 1-86, wherein the dilution buffer has an osmolarity of less than 900 mOsm.

Embodiment 88: The process of any one of Embodiments 1-87, further comprising a step of removing or reducing amount of impurities (e.g., host cell DNA (hcDNA)) from the harvest media prior to affinity purification.

Embodiment 89: The process of Embodiment 88, wherein said removing or reducing the amount of impurities comprises adding a cationic amine or nuclease to the harvest media.

Embodiment 90: The process of Embodiment 88 or 89, wherein said removing or reducing the amount of impurities comprises adding a selective precipitation agent to harvest media.

Embodiment 91: The process of any one of Embodiments 1-90 further comprising a step of lysing a host cell in the harvest media with a non-ionic surfactant prior purifying/isolating by affinity chromatography.

Embodiment 92: The process of Embodiment 91, wherein the non-ionic surfactant is added to the harvest media to a final concentration of at least about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1% or higher.

Embodiment 93: The process of Embodiment 91 or 92, wherein the non-ionic surfactant is added to the harvest media to a final concentration of from about 0.05% to about 1%, from about 0.1% to about 0.95%, from about 0.15% to about 0.9%, from about 0.2% to about 0.85%, from about 0.25% to about 0.8%, from about 0.3% to about 0.75%, from about 0.35% to about 0.65% from about 0.4% to about 0.6% or from 0.45% to about 0.55%.

Embodiment 94: The process of any one of Embodiments 91-93, wherein the non-ionic surfactant is added to the harvest media to a final concentration of about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 7%, about 0.75%, about 0.8%, about 0.85%, about 0.9%, about 0.95%, or about 1%.

Embodiment 95: The process of any one of Embodiments 91-94, wherein the non-ionic surfactant is added to the harvest media to a final concentration of about 0.5%.

Embodiment 96: The process of any one of Embodiments 91-95, wherein the non-ionic surfactant is mixed with the harvest media for a period of from about 15 minutes to about 2 hours.

Embodiment 97: The process of any one of Embodiments 91-96, wherein the non-ionic surfactant is mixed with the harvest media for a period of from about 30 minutes to about 60 minutes.

Embodiment 98: The process of any one of Embodiments 91-97, wherein the non-ionic surfactant is not Triton X-100.

Embodiment 99: The process of any one of Embodiments 91-98, wherein the non-ionic surfactant is selected from the group consisting of polyoxyethylene fatty alcohol ethers, polyoxyethylene alkylphenyl ethers, polyoxyethylene-polyoxypropylene block copolymers, alkylglucosides, alkylphenol ethoxylates, preferably polysorbates, polyoxyethylene alkyl phenyl ethers, and any combinations thereof.

Embodiment 100: The process of any one of Embodiments 1-99, wherein less than 5%, less than 4%, less than 3%, less than 2%, or less than 1% of empty virus particles in the affinity eluate bind to anion exchange chromatography media.

Embodiment 101: The process of any one of Embodiments 1-100, wherein substantially no empty virus particles in the affinity eluate bind to anion exchange chromatography media.

Embodiment 102: The process of any one of Embodiments 1-101, wherein less than 10%, less than 8%, less than 5%, or less than 2%, or preferably even less, of the virus particles in the eluate from the anion exchange are empty viral particles.

Embodiment 103: The process of any one of Embodiments 1-102, wherein the eluate from the anion exchange is substantially free of empty virus particles.

Embodiment 104: The process of any one of Embodiments 1-103, wherein the recombinantly expressed virus particles are recombinant adeno associated virus (rAAV) particle.

Embodiment 105: The process of any one of Embodiments 1-104, wherein the recombinant adeno associated virus (rAAV) particle comprise rAAV virion.

Embodiment 106: A population of recombinantly expressed virus particles purified or isolated by a method of any one of Embodiments 1-105.

Embodiment 107: A population of purified recombinant adeno-associated virus (rAAV) lacking prokaryotic sequences, wherein the purified rAAV has a particle to infectivity ratio less than 2×10⁴ vg/TCID50, optionally the population of purified rAAV comprises less than about 10% empty viral capsids, wherein, the purified rAAV is obtained by a method comprising, transfecting a suspension mammalian cell line, and optionally the cells are transfected in suspension.

Embodiment 108: The population of purified recombinant adeno-associated virus (rAAV) of embodiment 107, wherein, the population comprises less than about 5% empty viral capsids.

Embodiment 109: The population of purified recombinant adeno-associated virus (rAAV) of any one of embodiments 107-108, wherein, the population comprises less than about 2% empty viral capsids.

Embodiment 110: The population of purified recombinant adeno-associated virus (rAAV) of any one of embodiments 107-109, wherein, the population comprises less than about 1% empty viral capsids.

Embodiment 111: The population of purified recombinant adeno-associated virus (rAAV) of any one of embodiments 107-110, wherein, the population comprises less than about 0.5% empty viral capsids.

Embodiment 112: The population of purified recombinant adeno-associated virus (rAAV) of any one of embodiments 107-111, wherein, the population comprises less than about 0.2% empty viral capsids.

Embodiment 113: The population of purified recombinant adeno-associated virus (rAAV) of any one of embodiments 107-112, wherein, the population comprises less than about 0.05% empty viral capsids.

Embodiment 114: The population of purified recombinant adeno-associated virus (rAAV) of any one of embodiments 107-113, wherein, the population comprises less than about 0.03% empty viral capsids.

Embodiment 115: The population of purified recombinant adeno-associated virus (rAAV) of any one of embodiments 107-114, wherein, the population is substantially devoid of empty viral capsids.

Embodiment 116: The population of purified recombinant adeno-associated virus (rAAV) any one of embodiments 107-115, wherein the mammalian cell line is derived from a human embryonic cell line.

Embodiment 117: The population of purified recombinant adeno-associated virus (rAAV) any one of embodiments 107-116, wherein the human embryonic cell line is suspension adapted, serum free cell line derived from a human embryonic kidney cell line.

Embodiment 118: A population of purified recombinant adeno-associated virus (rAAV) lacking prokaryotic sequences, wherein, the purified rAAV has a particle to infectivity ratio less than 2×10⁴ vg/TCID50, optionally, the purified rAAV is obtained by a method comprising transfecting a suspension mammalian cell line.

Embodiment 119: The population of purified recombinant adeno-associated virus (rAAV) of Embodiment 118, wherein, the mammalian cell line is transfected in suspension with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements.

Embodiment 120: The population of purified recombinant adeno-associated virus (rAAV) of any one of Embodiments 118-119, wherein the mammalian cell line is derived from a human embryonic cell line.

Embodiment 121: The population of purified recombinant adeno-associated virus (rAAV) of any one of Embodiments 118-120, wherein the human embryonic cell line is suspension adapted, serum free cell line derived from a human embryonic kidney cell line.

Embodiment 122: A population of purified recombinant adeno-associated virus (rAAV), wherein the population of purified rAAV comprises less than about 10% empty viral capsids, optionally, the population of rAAV is purified by a process comprising:
a. purifying/isolating a plurality of recombinantly expressed virus particles from a harvesting media via affinity chromatography to produce an eluate (affinity chromatography eluate) comprising the plurality of recombinantly expressed virus particles, wherein an elution buffer for affinity chromatography (affinity elution buffer) comprises a predetermined amount of glycine, optionally, the affinity elution buffer is substantially free of weak acids or salts thereof; and optionally, the affinity elution buffer comprises an amino acid that is not glycine
b. adjusting the affinity chromatography eluate for subsequent purification through anion exchange chromatography, wherein the adjusted eluate comprises a predetermined amount of an anionic compound;
c. purifying/isolating the plurality of recombinantly expressed virus particles from the adjusted eluate of affinity chromatography by anion exchange chromatography to produce a solution comprising a plurality of purified/isolated recombinantly expressed virus particles, wherein an equilibration buffer for anion exchange chromatography comprises a predetermined amount of a weak acid or a salt thereof, optionally the weak acid is citric acid, acetic acid or succinic acid, thereby generating said population of purified recombinant adeno associated virus (rAAV).

Embodiment 123: The population of purified recombinant adeno-associated virus (rAAV) of embodiment 122, wherein, the population comprises less than about 5% empty viral capsids.

Embodiment 124: The population of purified recombinant adeno-associated virus (rAAV) of any one of embodiments 122-123, wherein, the population comprises less than about 2% empty viral capsids.

Embodiment 125: The population of purified recombinant adeno-associated virus (rAAV) of any one of embodiments 122-124, wherein, the population comprises less than about 1% empty viral capsids.

Embodiment 126: The population of purified recombinant adeno-associated virus (rAAV) of any one of embodiments 122-125, wherein, the population comprises less than about 0.5% empty viral capsids.

Embodiment 127: The population of purified recombinant adeno-associated virus (rAAV) of any one of embodiments 122-126, wherein, the population comprises less than about 0.2% empty viral capsids.

Embodiment 128: The population of purified recombinant adeno-associated virus (rAAV) of any one of embodiments 122-127, wherein, the population comprises less than about 0.05% empty viral capsids.

Embodiment 129: The population of purified recombinant adeno-associated virus (rAAV) of any one of embodiments 122-128, wherein, the population comprises less than about 0.03% empty viral capsids.

Embodiment 130: The population of purified recombinant adeno-associated virus (rAAV) of any one of embodiments 122-129, wherein, the population is substantially devoid of empty viral capsids.

Embodiment 131: A composition comprising a population of purified recombinant adeno-associated virus particles and a pH from about 6.5 to about 8.0, and wherein: (i) the purified rAAV has a particle to infectivity ratio less than 2×104 vg/TCID50; and/or (ii) the population of purified rAAV comprises less than about 10% empty viral capsids.

Embodiment 132: The composition of Embodiment 131, wherein the composition comprises the purified rAAV are at a concentration of from about 1e9 vg/ml to about 1e15 vg/ml.

Embodiment 133: The composition of Embodiment 131 or 132, wherein the composition comprises the purified rAAV are at a concentration of from about 1e12 vg/ml to about 1e14 vg/ml.

Embodiment 134: The composition of any one of Embodiments 131-133, wherein the composition comprises the purified rAAV are at a concentration of from about 1e12 vg/ml to about 1e14 vg/ml, optionally, the purified rAAV are at a concentration of from about 1e13 vg/ml to about 1e14 vg/ml.

Embodiment 135: The composition of any one of Embodiments 131-134, wherein the composition has a pH of from about 6.5 to about 8.0.

Embodiment 136: The composition of any one of Embodiments 131-135, wherein the composition has a pH of from about 6.5 to about 7.5.

Embodiment 137: The composition of any one of Embodiments 131-136, wherein the composition has a pH of from about 7.0 to about 7.4.

Embodiment 138: The composition of any one of Embodiments 131-135, wherein the composition has a pH of about 7.0, about 7.1, about 7.2, about 7.3, about 7.4, about 7.5, about 7.6, about 7.7, about 7.8 or about 7.9.

Embodiment 139: The composition of any one of Embodiments 131-138, wherein the composition comprises a buffer.

Embodiment 140: The composition of Embodiment 139, wherein the buffer has a salt concentration of from about 50 mM to about 750 mM.

Embodiment 141: The composition of Embodiment 139 or 140, wherein the buffer has a salt concentration of from about 100 mM to about 650 mM.

Embodiment 142: The composition of any one of Embodiments 139-141, wherein the buffer has a salt concentration of from about 150 mM to about 400 mM.

Embodiment 143: The composition of any one of Embodiments 139-142, wherein the buffer has a salt concentration of about 150 mM, about 200 mM or about 365 mM.

Embodiment 144: The composition of any one of Embodiments 131-143, wherein the composition has an ionic strength of at least 100 mM.

Embodiment 145: The composition of Embodiment 144, wherein the composition has an ionic strength from about 125 mM to about 750 mM.

Embodiment 146: The composition of Embodiment 144 or 145, wherein the composition has an ionic strength from about 150 mM to about 550 mM.

Embodiment 147: The composition of any one of Embodiments 144-146, wherein the composition has an ionic strength of about 170 mM, about 210 mM or about 380 mM.

Embodiment 148: The composition of any one of Embodiments 131-147, wherein the composition has an osmolarity from about 100 mOsm to about 600 mOsm.

Embodiment 149: The composition of Embodiment 148, wherein the composition has an osmolarity from about 125 mOsm to about 500 mOsm.

Embodiment 150: The composition of Embodiment 148, wherein the composition has an osmolarity from about 200 mOsm to about 400 mOsm.

Embodiment 151: The composition of Embodiment 149, wherein the composition has an osmolarity from about 140 mOsm to about 315 mOsm.

Embodiment 152: The composition of any one of Embodiments 131-151, wherein the composition comprises one or more ions and/or salts thereof.

Embodiment 153: The composition of Embodiment 152, wherein the ion is selected from the group consisting of sodium, potassium, chloride, ammonium, carbonate, nitrate, chlorate, chlorite, and calcium.

Embodiment 154: The composition of any one of Embodiments 131-153, wherein the composition comprises NaCl at a concentration from about 125 mM to about 450 mM.

Embodiment 155: The composition of Embodiment 154, wherein the composition comprises NaCl at a concentration from about 150 mM to about 400 mM.

Embodiment 156: The composition of Embodiment 155, wherein the composition comprises NaCl at a concentration from about 175 mM to about 375 mM.

Embodiment 157: The composition of any one of Embodiments 131-156, wherein the composition comprises KCl at a concentration from about 1 mM to about 10 mM.

Embodiment 158: The composition of Embodiment 157, wherein the composition comprises KCl at a concentration from about 2 mM to about 5.5 mM.

Embodiment 159: The composition of any one of Embodiments 131-158, wherein the composition comprises CaCl₂ at a concentration of from about 0.1 mM to about 2 mM.

Embodiment 160: The composition of Embodiment 159, wherein the composition comprises CaCl₂ at a concentration of from about 0.75 mM to about 1.25 mM.

Embodiment 161: The composition of any one of Embodiments 131-160, wherein the composition comprises MgCl₂ at a concentration from about 0.1 mM to about 1.5 mM.

Embodiment 162: The composition of Embodiment 161, wherein the composition comprises MgCl₂ at a concentration from about 0.25 mM to about 0.75 mM.

Embodiment 163: The composition of any one of Embodiments 131-162, wherein the composition comprises a monobasic phosphate or a salt thereof at a concentration from about 0.25 mM to about 3 mM.

Embodiment 164: The composition of Embodiment 163, wherein the composition comprises a mono basic phosphate or a salt thereof at a concentration from about 1 mM to about 2.25 mM.

Embodiment 165: The composition of Embodiment 163 or 164, wherein the monobasic phosphate is potassium phosphate monobasic.

Embodiment 166: The composition of any one of Embodiments 131-165, wherein the composition comprises a dibasic phosphate or a salt thereof at a concentration from about 5 mM to about 15 mM.

Embodiment 167: The composition of Embodiment 166, wherein the composition comprises a dibasic phosphate or a salt thereof at a concentration from about 8 mM to about 10 mM.

Embodiment 168: The composition of Embodiment 166 or 167, wherein the dibasic phosphate or a salt thereof is sodium phosphate dibasic.

Embodiment 169: The composition of any one of Embodiments 131-168, wherein the composition comprises a bulking agent, e.g., a polyol or providone (PVP K24).

Embodiment 170: The composition of Embodiment 169, wherein the bulking agent is selected from the group consisting of polyhydroxy hydrocarbons, monosaccharides, disaccharides, and trisaccharides.

Embodiment 171: The composition of Embodiment 169 or 170, wherein the bulking agent is selected from the group consisting of sorbitol, mannitol, glycerol, propylene glycol, polyethylene glycol, dulcitol, sucrose, lactose, maltose, trehalose, and dextran.

Embodiment 172: The composition of any one of Embodiments 166-171, wherein the composition comprises the bulking agent, e.g., a polyol or providone (PVP K24) at a concentration from about 0.5% (w/v) to about 10% (w/v).

Embodiment 173: The composition of any one of Embodiments 166-172, wherein composition comprises the bulking agent, e.g., a polyol or providone (PVP K24) at a concentration from about 1% (w/v) to about 7.5% (w/v).

Embodiment 174: The composition of any one of Embodiments 166-173, wherein composition comprises the bulking agent, e.g., a polyol or providone (PVP K24) at a concentration of about 1% (w/v), about 3% (w/v), or about 5% (w/v).

Embodiment 175: The composition of any one of Embodiments 131-174, wherein the composition comprises a non-ionic surfactant.

Embodiment 176: The composition of Embodiment 175, wherein the non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof.

Embodiment 177: The composition of Embodiment 175 or 176, wherein the non-ionic surfactant is selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof.

Embodiment 178: The composition of any one of Embodiments 175-177, wherein the composition comprises the non-ionic surfactant at a concentration from about 0.0001% (w/v) to about 0.01% (w/v).

Embodiment 179: The composition of any one of Embodiments 175-178, wherein the composition comprises the non-ionic surfactant at a concentration from about 0.0005% (w/v) to about 0.0015% (w/v).

Embodiment 180: The composition of any one of Embodiments 175-179, wherein the composition comprises the non-ionic surfactant at a concentration of about 0.001% (w/v).

Embodiment 181: The composition of any one of Embodiments 131-180, wherein the composition comprises one or more multivalent ions or salts thereof.

Embodiment 182: The composition of Embodiment 181, wherein the multivalent ions are selected from the group consisting of citrate, sulfate, magnesium and phosphate.

Embodiment 183: The composition of any one of Embodiments 131-182, wherein the composition comprises MgSO₄ at a concentration from about 5 mM to about 150 mM.

Embodiment 184: The composition of Embodiment 183, wherein the composition comprises MgSO₄ at a concentration from about 15 mM to about 100 mM.

Embodiment 185: The composition of any one of Embodiments 131-184, wherein the composition comprises calcium α-d-heptagluconate at a concentration from about 1% (w/v) to about 20% (w/v).

Embodiment 186: The composition of any one of Embodiments 131-185, wherein the population of purified rAAV comprises less than about 5%, less than about 2%, less than about 1%, less than about 0.5%, less than about 0.2%, less than about 0.15%, less than about 0.1%, less than about 0.05%, less than 0.04%, less than 0.03% or less than about 0.025% empty viral capsids.

Embodiment 187: The composition of any one of Embodiments 131-186, wherein the population of purified rAAV is substantially devoid of empty viral capsids.

Embodiment 188: The composition of any one of Embodiments 131-187, wherein the purified rAAV has a particle to infectivity ratio less than 1.5×104 vg/TCID50, less than 1×104 vg/TCID50, less than 9×103 vg/TCID50, less than 8×103 vg/TCID50, less than 6×103 vg/TCID50, less than 5×103 vg/TCID50, less than 4×103 vg/TCID50, less than 3×103 vg/TCID50, less than 2×103 vg/TCID50, less than 9×102 vg/TCID50, less than 8×102 vg/TCID50, less than 7×102 vg/TCID50, less than 6×102 vg/TCID50, less than 5×102 vg/TCID50, less than 4×102 vg/TCID50, less than 3×102 vg/TCID50, less than 2×102 vg/TCID50, or, less than 1×102 vg/TCID50, or less than 0.5×102 vg/TCID50.

Embodiment 189: The composition of any one of Embodiments 131-188, wherein the population of purified recombinant adeno-associated virus particles is the population of purified rAAV of any one of Embodiments 107-130.

Embodiment 190: A pharmaceutical composition comprising the population of purified recombinant adeno-associated virus (rAAV) particles of any of the preceding Embodiments.

Embodiment 191: The composition of any of the preceeding embodiments, wherein the composition exhibits substantially no aggregation of the rAAV particles after two or, more freeze thaw cycles.

Embodiment 192: The composition of any of the preceeding embodiments, wherein the purified rAAV particle retains its TCID50/ml by at least about 80% after two or, more freeze thaw cycles.

### Some Selected Definitions

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “cell culture,” refers to cells grown adherent or in suspension, bioreactors, roller bottles, hyperstacks, microspheres, macrospheres, flasks and the like, as well as the components of the supernatant or suspension itself, including but not limited to viral particles (e.g., rAAV particles), cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure.

The terms “cell supernatant” or “cell culture supernatant” refer to the liquid media and extracellular components in the media suspension which may be separated from cellular material of an adherent cell culture or cell suspension culture. Generally, material secreted form the cells in culture can be purified from the cell supernatant. In some instances, viral particles can be secreted into the supernatant and purified therefrom, with or without lysing cells.

The terms “purifying”, “purification”, “separate”, “separating”, “separation”, “isolate”, “isolating”, or “isolation”, as used herein, refer to increasing the degree of purity of recombinant virus particles, e.g., rAAV particles from a sample comprising the target recombinant virus particles, e.g., rAAV particles and one or more impurities. Typically, the degree of purity of the target product is increased by removing (completely or partially) at least one impurity from the sample. In some embodiments, the degree of purity of the recombinant virus particles, e.g., rAAV particles in a sample is increased by removing (completely or partially) one or more impurities from the sample by using a method described herein.

The term “impurity” refers to any foreign or objectionable molecule, including a biological macromolecule such as DNA, RNA, one or more host cell proteins, endotoxins, lipids and one or more additives which can be present in a sample containing the recombinant viral particles. The term “impurity” further encompasses product-related impurities, for example, inactive vector forms, empty viral capsids, aggregated viral particles or capsids, misfolded viral capsids, degraded viral particles. In some embodiments, an impurity comprises an empty viral capsid or a viral aggregate. Additionally, such impurity can include any reagent which is used in a step which may occur prior to one or more of the disclosed methods. An impurity can be soluble or insoluble in nature. Insoluble impurities include any undesirable or objectionable entity present in a sample containing recombinant viral particles, where the entity is a suspended particle or a solid. Exemplary insoluble impurities include without limitation, aggregated viral particles or capsids, whole cells, cell fragments and cell debris. Soluble impurities include any undesirable or objectionable entity present in a sample containing recombinant viral particles where the entity is not an insoluble impurity. Exemplary soluble impurities include without limitation, host cell proteins, DNA, RNA, lipids viruses, endotoxins, and cell culture media components.

As used herein, the term “helper virus” or “contaminating helper virus” refers to a virus used when producing copies of a helper virus-dependent viral vector, such as adeno-associated virus, which does not have the ability to replicate on its own. The helper virus is used to co-infect ells alongside the viral vector and provides the necessary proteins for replication of the genome of the viral vector. The term encompasses intact viral particles, empty capsids, viral DNA and the like. Helper viruses commonly used to produce rAAV particles include adenovirus, herpes simplex virus, cytomegalovirus, Epstein-Barr virus, and vaccinia virus.

Helper viruses include Adenovirus (AV), and herpes simplex virus (HSV), and systems exist for producing AAV in insect cells using baculovirus and mammalian cells. It has also been proposed that papilloma viruses may also provide a helper function for AAV (See, e.g., Hermonat et al., Molecular Therapy 9, 5289-S290(2004)). Helper viruses include any virus capable of creating an allowing AAV replication. AV is a nonenveloped nuclear DNA virus with a double-stranded DNA genome of approximately 36 kb. AV is capable of rescuing latent AAV provirus in a cell by providing E1a, E1b55K, E2a, E4orf6, and VA genes, allowing AAV replication and encapsidation. HSV is a family of viruses that have a relatively large double-stranded linear DNA genome encapsidated in an icosahedral capsid, which is wrapped in a lipid bilayer envelope. HSV are infectious and highly transmissible. The following HSV-1 replication UL8, and UL52) and the DNA binding protein ICP8 encoded by the UL29 gene, with other proteins enhancing the helper function.

The term “non-adherent cell line” or “suspension cell line”, as used herein, refers to a cell line that is able to survive in a suspension culture without being attached to a surface (e.g. tissue culture plastic carrier or micro-carrier). The adaptation to a non-adherent cell line is a prolonged process requiring passaging with diminishing amounts of serum, thereby selecting an irreversibly modified cell population. The cell line can be grown to a higher density than adherent conditions would allow and is, thus, more suited for culturing in an industrial scale, e.g. in a bioreactor setting or in an agitated culture.

As used in this specification and the appended claims, the term “or” is generally employed in its sense including “and/or” unless the content clearly dictates otherwise.

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

The term “promoter” as used herein is defined as a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A ‘regulatory element’ is any transcriptional or, non-transcriptional regulatory element that works at the transcriptional level or, post-transcriptional level. A regulatory element can be a cis regulatory element or cis-regulatory module that are regions of non-coding DNA that regulate the transcription of neighboring genes. The non-limiting examples of regulatory element or, cis regulatory elements are promoters, enhancers, silencers, operators. The regulatory element can be synthetic. One example of synthetic regulatory element is combining two or, more of the cis regulatory elements. In some examples, a regulatory element can be trans regulatory elements. Trans regulatory elements are typically DNA sequences encoding upstream regulators (i.e trans acting factors) that may modify or, regulate expression of distant genes. The non-limiting examples of trans regulatory elements are nucleic acid encoding transcription factors or, fragments thereof; nucleic acid encoding DNA editing proteins or, fragments thereof—non limiting examples include nucleic acid encoding RAG1/RAG2, TdT, Cas1/Cas2; nucleic acid encoding mRNA processing proteins or fragments thereof,—non-limiting examples include nucleic acid encoding SR proteins, Ribonucleoproteins e.g hnRNP, snRNP; nucleic acid encoding mRNA binding proteins and/or, non-coding RNA sequences-non limiting examples include, nucleic acid sequence encoding RNA binding protein, or, siRNA, shRNA, miRNA, or, piRNA sequences.

A “protelomerase” target sequence is any DNA sequence whose presence in a DNA template allows for its conversion into a closed linear DNA by the enzymatic activity of protelomerase. In other words, the protelomerase target sequence is required for the cleavage and religation of double stranded DNA by protelomerase to form covalently closed linear DNA. Typically, a protelomerase target sequence comprises any perfect palindromic sequence i.e any double-stranded DNA sequence having two-fold rotational symmetry, also described herein as a perfect inverted repeat. The length of the perfect inverted repeat differs depending on the specific organism. In Borrelia burgdorferi, the perfect inverted repeat is 14 base pairs in length. In various mesophilic bacteriophages, the perfect inverted repeat is 22 base pairs or greater in length. Also, in some cases, e.g. E. coli N15, the central perfect inverted palindrome is flanked by inverted repeat sequences, i.e. forming part of a larger imperfect inverted palindrome.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.1, 2.2, 2.7, 3, 4, 5, 5.5, 5.75, 5.8, 5.85, 5.9, 5.95, 5.99, and 6. This applies regardless of the breadth of the range.

As used herein, “substantially free” means that no significant amount of the indicated component is present. Generally, the indicated component is not present or only present in a minimal amount without any substantial effect on the properties of the composition. For example, the indicated component is present in an amount (mol/mol) of about 4.5% or lower, about 4% or lower, about 3.5% or lower, about 3% or lower, about 2.5% or lower, about 2% or lower, about 1.5% or lower, about 1% or lower, about 0.5% or lower, 0.25% or lower, 0.20% or lower, 0.15% or lower, 0.05% or lower, or 0.01% or lower. In some embodiments, the indicated component is present in an amount (v/v, w/v, or w/w) of about 4.5% or lower, about 4% or lower, about 3.5% or lower, about 3% or lower, about 2.5% or lower, about 2% or lower, about 1.5% or lower, about 1% or lower, about 0.5% or lower, 0.25% or lower, 0.20% or lower, 0.15% or lower, 0.05% or lower, 0.01% or lower. In some embodiments, the indicated component is present in an amount of about 10,000 ppm or lower, 7,500 ppm or lower, 5,000 ppm or lower, 2,500 ppm or lower, 2,000 ppm or lower, 1,500 ppm or lower, 1,000 ppm or lower, 750 ppm or lower, 500 ppm or lower, 400 ppm or lower, 300 ppm or lower, 200 ppm or lower, 100 ppm or lower, 75 ppm or lower, 50 ppm or lower, 40 ppm or lower, 30 ppm or lower, 20 ppm or lower, 10 ppm or lower, or 5 ppm or lower. In some embodiments, the indicated component is present in an undetectable amount. For example, the indicated component is present in an amount that is undetectable by HPLC, gas chromatography, mass spectrometry or other means used for detecting the indicated component.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The description of embodiments of the disclosure is not intended to be exhaustive or to limit the disclosure to the precise form disclosed. While specific embodiments of, and examples for, the disclosure are disclosed herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments disclosed herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

### Example 1: High Throughput Screening to Purify Multiple AAV Serotypes

In the present invention, using a high throughput Ambr system in tandem with a Tecan system, several AAV serotypes can be purified simultaneously. Using a high throughput Ambr system, 24 small scale bioreactors containing 15 ml of transfected Pro 10 cells are grown for 3 days. In this exemplary method, 12 different AAV serotypes are purified e.g., AAV2, AAV3b, AAV4, AAV5, AAV6, AAV8, AAV9, AAV2i8, AAVrh10 and AAVrh74. For each serotype, amount of DNA transfected varied based on different cell densities used for transfection. After growing the cells for 3 days, the culture media from 24 different bioreactors are purified (with or, without lysis) over 48 small scale (0.1 ml) Affinity RoboColumns. Culture media (with or, without lysis) from each bioreactor is eluted with two different elution buffers comprising either glycine-histidine or glycine-citrate. The eluates are then analyzed using a plate reader on a Tecan system and a high-throughput SEC-HPLC method to determine the specific productivity (vp/ml culture) as well as packaging of % full capsids. Each sample is evaluated on a DLS for vector stability. The best candidates from the screen are identified and scaled up Affinity Column (range: 1-2000 mL). Affinity purified material is then carried over a high throughput AEX method (0.1 mL) using Tecan or FPLC in a scouting mode. AEX method evaluation contains addition of 0-20 mM citric acid (or, 0-30 mM succinic acid or, 0-60 mM acetic acid) in the sample dilution/column equilibration phases and a 3 CV conductivity mediated isocratic step elution at the pH greater than 8. The AEX flowthrough and eluate fractions are then analyzed using Tecan plate reader or SEC-HPLC to assess the separation of empty capsids from full capsids as well as capsids recovery. The instant method enables identification of elution buffer conditions and weak acid concentration to purify recombinantly expressed (full) AAV serotypes in a high throughput manner. This method can be scaled down or, scaled up according to the production need.

### Example 2: Glycine-Citrate AEX Stream

AEX experiments used 1 mL CIMmultus QA monolith (BIA Separations, catalogue #311.5113-2) with a 2.0 μm pore size. Chromatography experiments were performed using an AKTA Avant 25 system (Cytiva, formerly GE Healthcare Life Sciences). Chromatography experiments were performed in the pH range of 8.5 to 9.5 using bis-tris propane as the buffering systems. The CIMmultus QA was first equilibrated using 15 column volumes of 100 mM BTP 100 mM Histidine 1 mM MgCl2 0.4% F68 and a variable amount of Citric Acid. The affinity-purified pools comprising of glycine-citrate were diluted 12-15-fold into 100 mM BTP 100 mM Histidine 1 mM MgCl2 0.4% PF68 and a variable amount of Citric Acid, and loaded onto the CIMmultus QA column at a loading ratio of 5e13 vp/mL to 2e14 vp/mL. The column was then chased with 25 column volumes of the equilibration solution, and the empty and/or full particles were eluted from the column by applying a linear gradient elution. All chromatography steps were performed at ambient temperature in a downflow direction.

### Example 3: Glycine-Histidine AEX Stream

AEX experiments used 1 mL CIMmultus QA monolith (BIA Separations, catalogue #311.5113-2) with a 2.0 nm pore size. Chromatography experiments were performed using an AKTA Avant 25 system (Cytiva, formerly GE Healthcare Life Sciences). Chromatography experiments were performed in the pH range of 8.5 to 9.5 using bis-tris propane as the buffering systems. The CIMmultus QA was first equilibrated using 15 column volumes of 100 mM BTP 100 mM Histidine 1 mM MgCl2 5% Glycerol 0.5% F68 and a variable amount of Citric Acid. The affinity-purified pools were diluted 5-fold into 100 mM BTP 100 mM Histidine 1 mM MgCl2 5% Glycerol 0.5% PF68 and a variable amount of Citric Acid, and loaded onto the CIMmultus QA column at a loading ratio of 5e13 vp/mL to 2e14 vp/mL. The column was then chased with 25 column volumes of the equilibration solution, and the empty and/or full particles were eluted from the column by applying a linear gradient elution. All chromatography steps were performed at ambient temperature in a downflow direction.

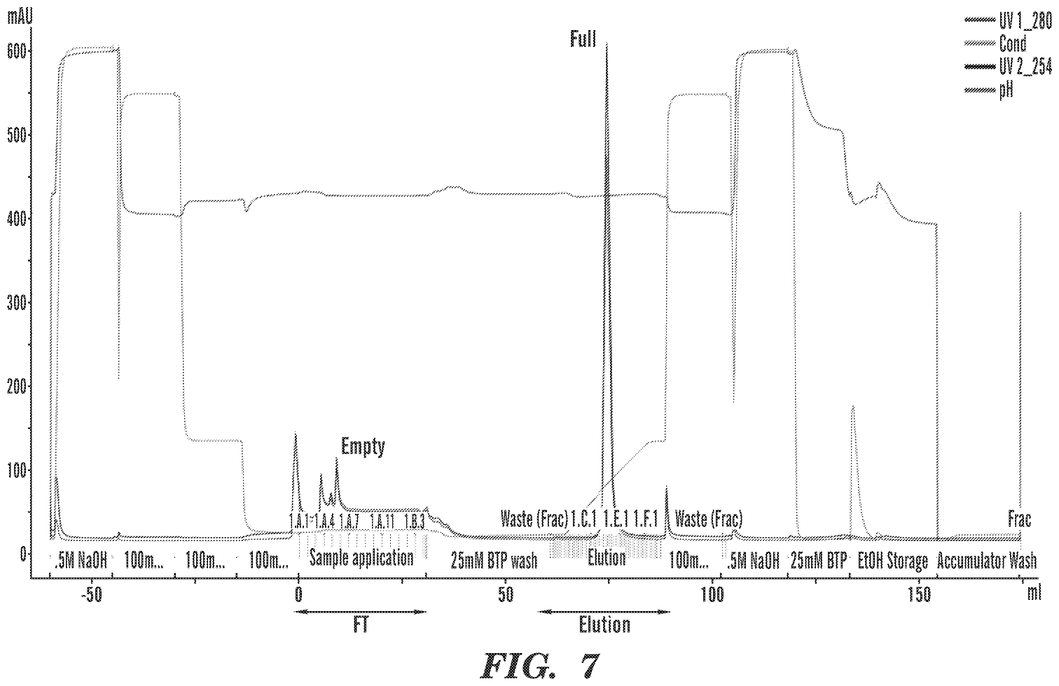

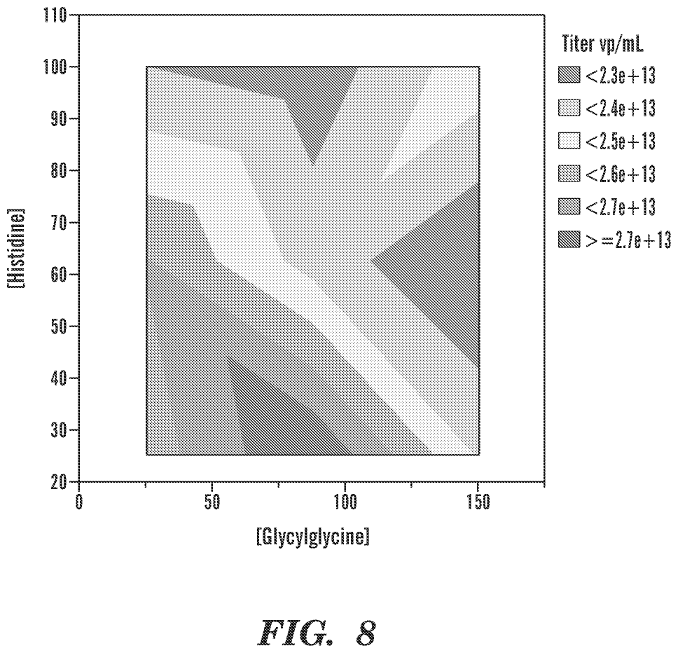

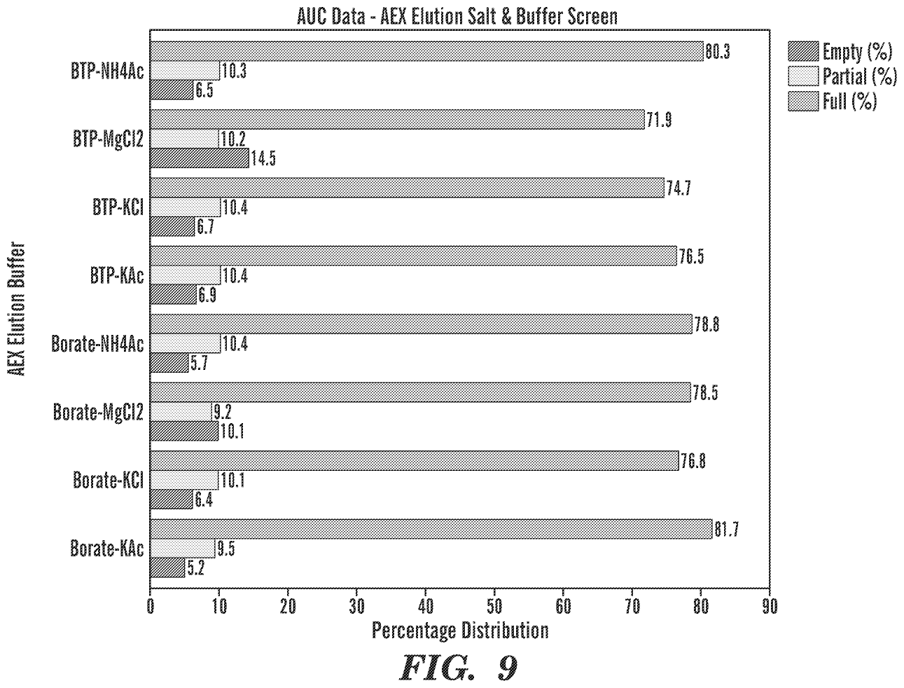

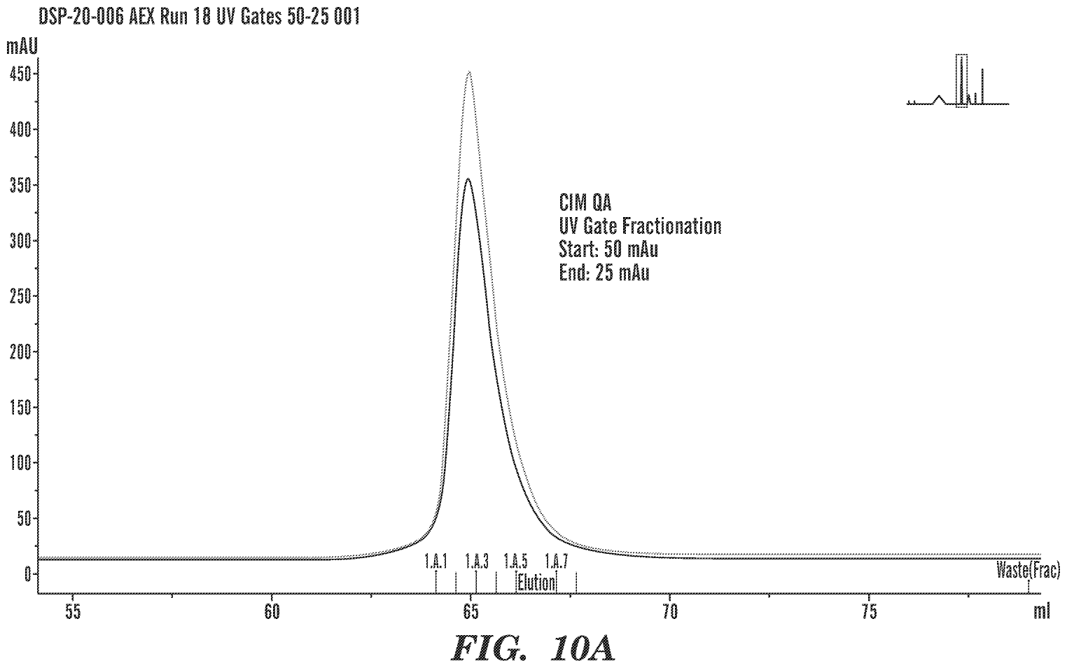

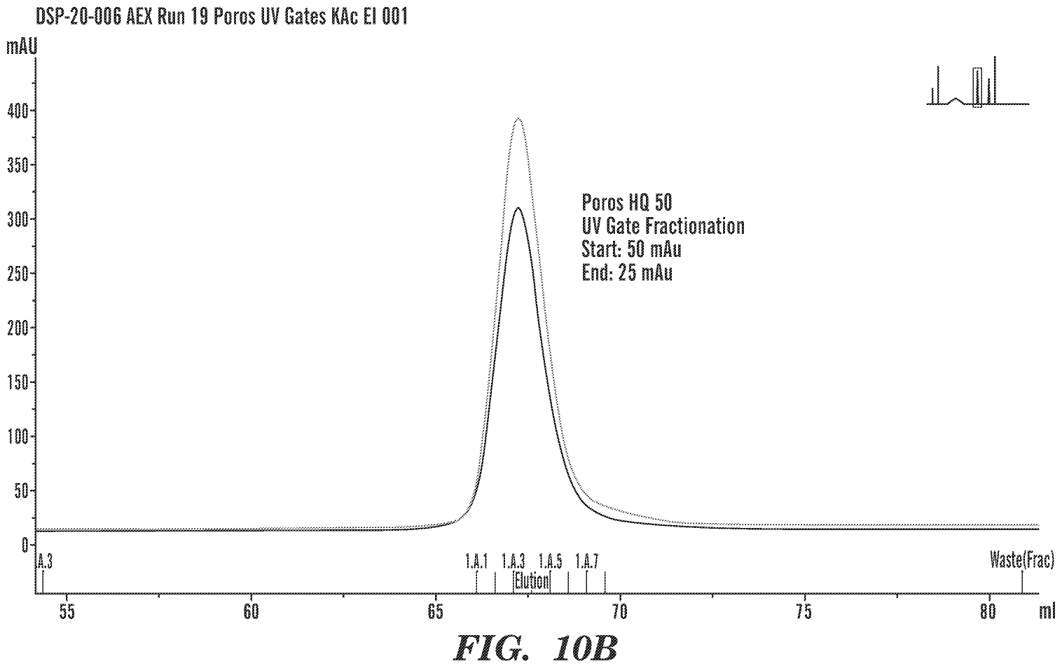

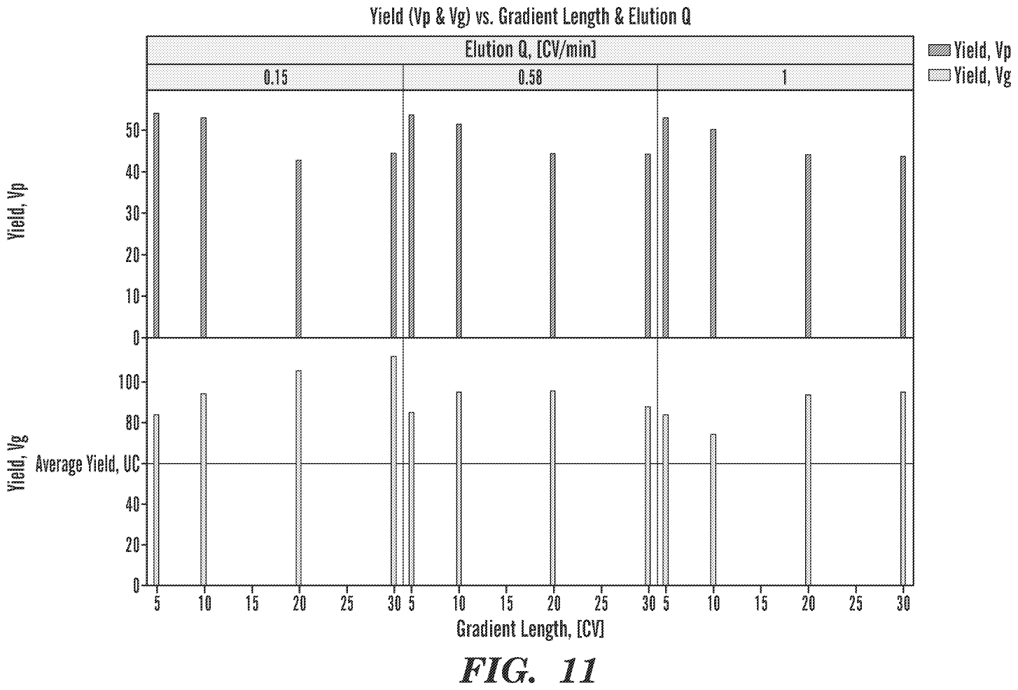

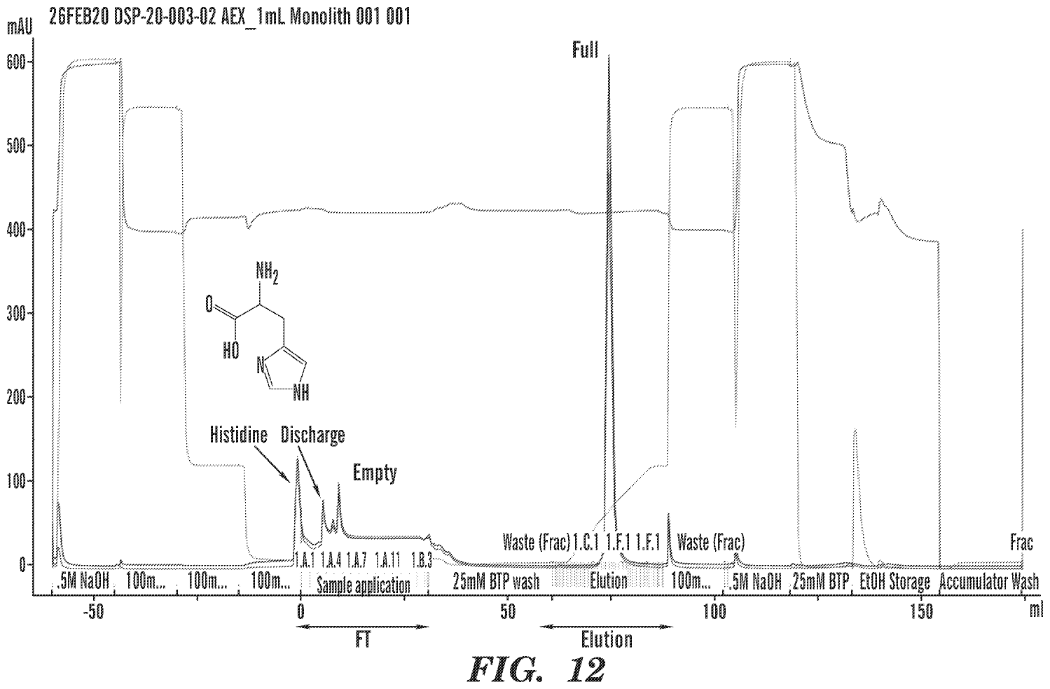

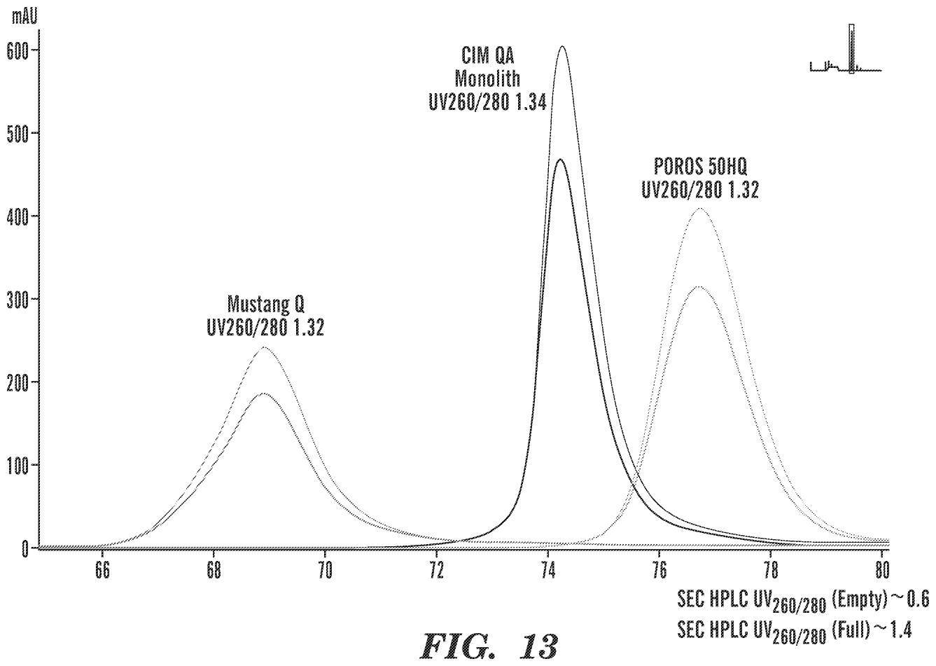

The AEX flowthrough and eluate fractions are then analyzed using Tecan plate reader or SEC-HPLC to assess the separation of empty capsids from full capsids as well as capsids recovery. The AEX eluate as described herein was substantially devoid of empty particles (for example as shown in FIG. 7 and FIG. 12). The recombinant AAV thus isolated from the AEX eluate, is substantially devoid of empty AAV particles.

### Example 4: AAV Production Using Closed Linear Duplexed (cl) DNA

TABLE 2
Description of analytical testing and specifications to
be completed for the 50 L scale vector productions.

| Assay | Specification |
|---|---|
| Western Blot | Presence of bands corresponding to VP1, VP2 and VP3 capsid |
| | proteins compared to molecular weight standard and control |
| Silver Stain | VP1, VP2 and VP3 capsid proteins are the dominant protein bands |
| | compared to molecular weight standard and control |
| Vector Genome | Sequence matches 100% |
| Sequencing | |
| Genome integrity | ≥75% full-length (densitometry) Co-migrates with Transgene clDNA |
| (alkaline gel) | or matches expected restriction size of the vector DNA from the |
| | plasmid pre-cursor. Size is confirmed versus a DNA marker. |
| Residual plasmid | None detected, results are reported as pg DNA/1 × 10⁹ vg |
| backbone | |
| (Multiplex qPCR) | |
| UV 260/280 | 1.20 to 1.40 |
| Replication Competent | <1 rcAAV/3.0 × 10¹⁰ vg |
| AAV | |

The PRO10™ cell line (AskBio, NC, USA), used to manufacture recombinant adeno-associated viral vectors (rAAV), is a suspension-adapted, serum-free cell line derived from the human embryonic kidney cell line 293 (HEK293). The PRO10™ Viral vector manufacture is a batch process carried out at mid- to high-range cell densities and employs a triple transfection method via condensation of the requisite plasmid (pDNA) or closed linear (cl) DNA substrate with linear Polyethylenimine MAX in a cocktail of production media. Both cell growth and production medias are chemically defined with no animal derived components. The triple transfection method comprises transfection with three DNA molecules or three DNA constructs; each DNA molecule provides a key element for the recombinant AAV production. The first provides Adenovirus helper (Ad helper) proteins for efficient replication and packaging of the vector but lacks essential Adenoviral structural and replication genes to generate an Adenovirus. The second is an AAV8 or, AAVrh10 Trans construct (packaging construct) containing the AAV2 rep gene and AAV8 capsid (cap) or, AAVrh10 capsid (cap) protein gene. The third construct is the therapeutic transgene encoding, AAV vector construct and contains the adeno-associated virus 2 inverted terminal repeat (ITR) sequences flanking (5′ to 3′) the gene of interest. The construct used for all experiments was the dual GFP and Luciferase reporter. Additionally, subsequent studies utilized two therapeutic transgene cassettes e.g., comprising CYP46A1 and GAA transgenes.

Initial experiments were conducted applying Design of Experiments (DoE) methodology in a traditional, non-block approach at bench scale (31.25 mL-2 L) to identify and optimize critical parameters relating to production by simultaneously examining the factors clDNA concentration, ratio of clDNA to transfection reagent. All small-scale experiments were controlled by side-by-side vector production using an optimized triple-plasmid transfection system. Additional factors that will be evaluated include, but are not limited to, media, cell density, time of transfection, transfection volume, temperature, and other cell-dependent or cell-independent factors.

Small-scale transfected cultures were incubated for approximately 72 hrs post-transfection (hpt) and then harvested by mechanical cell lysis. Total vector production was assessed via vector genome (vg) quantification using the in-house qPCR-based DNase Resistant Particle (DRP) method specific to the viral ITRs. Yields typically range from 4-6×10¹¹ vg/mL, as indicated by qPCR. Yields were further assessed by observing transgene-targeted qPCR as well as total viral particle (capsids) per mL (vp/mL) via ELISA. Relative packaging efficiency is also modeled by observing the A260/280 ratio at harvest of affinity-purified lysates via SEC-HPLC.

The primary aim of the small-scale screening experiments was to identify near-optimal transfection conditions for the 50 L scaled portion of the experimental plan. For both the pDNA and clDNA runs, cells were thawed, cultured and progressively expanded until inoculation into the 50 L production bioreactor. The cell culture expansion process continued in the production bioreactor prior to transient transfection being performed. The transfected cell culture was incubated in the production bioreactor for approximately 72-hpt. At harvest, the transfected cell culture was lysed and clarified via depth and membrane filtration followed by purification. Purification consists of capture chromatography, gradient ultracentrifugation, ion exchange chromatography, ultrafiltration/diafiltration (UF/DF), and a 0.2 μm filtration step.

### Detailed Process Description for SOL SUB Upstream Operations

To generate a 50 L batch, cells were thawed, cultured and progressively expanded until inoculation into the 50 L production bioreactor. The cell culture expansion process continued in the production bioreactor prior to transient transfection being performed. Currently, the seed train growth media is supplemented with L-Glutamine to a final concentration of 10 mM, which is used for recovery of frozen cell stocks as well as inoculum expansion up to 5 L suspensions using a 10 L WAVE bag bioreactor. The media used in the WAVE suspension was supplemented with 0.2% PLURONIC™ acid. The growth media used following seed of the ThermoFisher 50 L single-use, stirred-tank bioreactor (SUB, STR) is composed of the see train growth media supplemented with about 1 to 100 mM GLUTAMAX™, about 0.01% to 10% PLURONIC™ acid (ThermoFisher, Waltham, MA), and about 0.001% to 1% FOAMAWAY™ (Gibco, Waltham, MA). GLUTAMAX™ is a stabilized dipeptide source of L-glutamine designed to prevent degradation and reduce toxic buildup of excess ammonia.

Transient transfection to produce AAV was carried out at cell densities between 3.25-4.25×10⁶ viable cells/mL 3 via condensation of three clDNA and linear Polyethylenimine MAX (Polysciences Inc., Warrington, PA) (PEI Max). The transfection is performed under suspension condition. The transfection cocktail constitutes 10% (v/v) of the culture volume (5 L). Condensation was carried out in a custom 10 L WAVE Rocker bag equipped with tubing mated for the 50 L SUB. The transfection cocktail was prepared by first adding 4 L of media to the rocker bag at 25° C. with gentle rocking (8° angle, 25 RPM). To prevent the bag from deflating, an air overlay is applied at 0.2 LPM. The DNA (Table 3 shows clDNA) were then added, followed by a 1 L chase with media.

TABLE 3
Ratio of each clDNA used normalized to the
cell density at the time of transfection.

| Construct | Ratio |
|---|---|
| Ad helper | 0.15 to 0.32 μg DNA/1 × 10⁶ cells |
| Rep/Cap | 0.15 to 0.32 μg DNA/1 × 10⁶ cells |
| Transgene | 0.15 to 0.32 μg DNA/1 × 10⁶ cells |

Following the media chase, PEI was added over the course of 1 minute and chased with 1 L of media. The cocktail was incubated for 7 minutes, and then transferred to the SUB. The transfection-cell suspension is incubated for three hours and quenched by a 10% (v/v) volume of chemically defined, serum-free HEK293 media supplemented with 10 mM L-Glutamine.

### SUB Control Parameters

The current large-scale manufacturing platform utilized a Finesse G3Pro Universal Controller outfitted with a ThermoFisher jacketed 50 L SUB. The single-use vessels were equipped with a 3-blade, 45° pitch, axial impellor, dual-sparger (Frit-Drilled-Hole) design, along with primary Finesse TruFluor pH/DO single-use probe sheaths as well as secondary Pall Kleenpak connections for reusable pH/DO probe inserts. The day before media charge, the bag was installed and inflated with an air overlay at 10 LPM. The optical/reusable DO probe was connected to the transmitter. On the day of charge, the DO probe was calibrated using a 2-pt slope calibration. Following media addition, both single-use and reusable pH probes were standardized using an offline sample on a calibrated blood-gas analyzer.

The SUB temperature was ramped to 37° C. the day before inoculation. The media was then conditioned by saturating with a continuous drilled-hole air sparge at a flow rate of 0.5 LPM (0.025 VVM). Prior to inoculation, both single-use and reusable DO probes were standardized to 100% air saturation using a 1 pt calibration.

Following inoculation, the controller was set to administer a continuous drilled-hole air sparge at a rate of 0.5 LPM, and the headspace was swept with an air overlay of 1 LPM. DO was controlled via 02 gas cascade and designed to maintain the set point by increasing 02 flow rate to the frit sparger from 0.00 to 5.00 LPM (0-100% DO output/0-100% MFC-3 output). pH was controlled on the high end (7.0-14) by increasing CO₂ gas flow to the frit sparger from 0.00 to 2.00 LPM (0−(−100)%) output/0-100% MFC-4 output); however, a base supply was not used to control pH on the low end, but rather, it was allowed to drift naturally.

Results: Total vector production at harvest was evaluated via ITR-qPCR (data not shown). The data indicates 2-2.5 times increase in specific (vg/cell) productivity using the clDNA as starting material compared to the pDNA as starting material. The clDNA used to generate recombinant AAVrh10CYP46A1 suggest less than 1 ug DNA e.g between 0.6-0.7 ug required to achieve high titer of AAV. Furthermore, PEI:DNA ratio was 2.2 and 2.5 to generate recombinant AAVrh10CYP46A1 and AAV8GAA and the optimal clDNA was 0.6 ug considering both overall yield and packaging efficiency.

### Example 5: TCID50/Ml and Particle to Infectivity (Vg/TCID50) Ratio

TCID50 assay: The infectious titer (TCID50) method is used to evaluate the in vitro AAV infectivity of drug product in HeLa RC32 cells. In this assay, HeLa RC32 cells are transduced with adenovirus type 5 helper virus and serial dilutions of drug product. After three days of infection the cells are treated with proteinase K to digest protein and the replicated AAV vector DNA is quantitated with qPCR technology. This method utilizes a DNA primer and fluorescent dye-based detection system. The absolute quantity of the ITR target sequence from the vector DNA is interpolated from a standard curve prepared with a plasmid. Containing ITR is prepared as a test sample and is used as an assay control. Results are expressed as infectious units per milliliter (IU/mL). It is noted that for comparing TCID50/ml among different preparations, TCID50/ml is preferably normalized to vg/ml.

Without being construed to any limitation, the Table 4 below shows the TCID50/ml infectious titer and particle to infectivity (vg/TCID50) ratio results for plasmid DNA (pDNA) and close ended linear duplexed (clDNA)-derived AAV vector.

TABLE 4
TCID50/ml and particle to infectivity (vg/TCID50) ratio:

| | | Particle to infectivity ratio |
|---|---|---|
| | TCID50/ml | (vg/TCID50) |
| pDNA control | 6.32E+08 | 1106.8 |
| DoE clDNA | 1.36E+09 | 521.8 |
| DoE clDNA | 6.32E+08 | 983.47 |
| 50 L pDNA | 1.65E+10 | 9700 |
| 50 L clDNA | 2.94E+10 | 2425.4 |
| 50 L clDNA | 1.36E+10 | 6605.1 |
| 50 L clDNA | 3.56E+09 | 15000 |

As shown in the Table 4, in DoE experiments and in 50 L runs, clDNA derived vectors show increased infectivity compared to pDNA controls as indicated by lower vg/TCID50 ratio for clDNA compared to that of pDNA. In some aspects of the invention described herein, one example of the population of purified recombinant adeno-associated virus that lacks prokaryotic sequence, is derived from clDNA.

### Example 6: Use of Citric Acid for Empty and Full Capsids Separation Via AEX

This study demonstrates separation of empty (E) and full (F) capsids using partitioning mode with weak acids for multiple natural serotypes according to some exemplary embodiments of the invention. Without wishing to be bound by a theory, the chromatographic separation of empty (E) and full (F) capsids is based on the charge differences between the empty and full capsids—empty capsid isoelectric point 6.3 and full capsid isoelectric point 5.9. At high pH, full capsids are more negatively charged. The anion exchange is positively charge. As such, full capsids elute later during a salt gradient. Inclusion of a weak acid, such as citric acid, can reduce binding of the empty capsids to the anion exchange resin thereby partitioning the empty and full capsids.

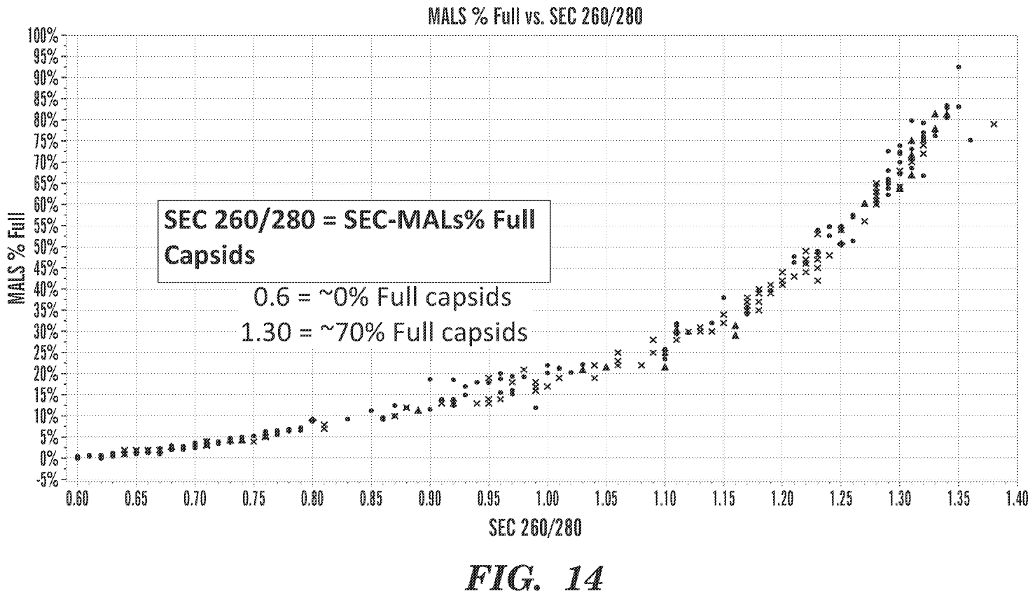

As shown in FIG. 14, UV_{260/280} ratio of a sample can be used to determine % full capsids in a sample. A UV_{260/280} ratio of about 0.6 indicates ˜0% full capsids in the sample and a UV_{260/280} ratio of about 1.30 indicates about 70% full capsids in the sample.

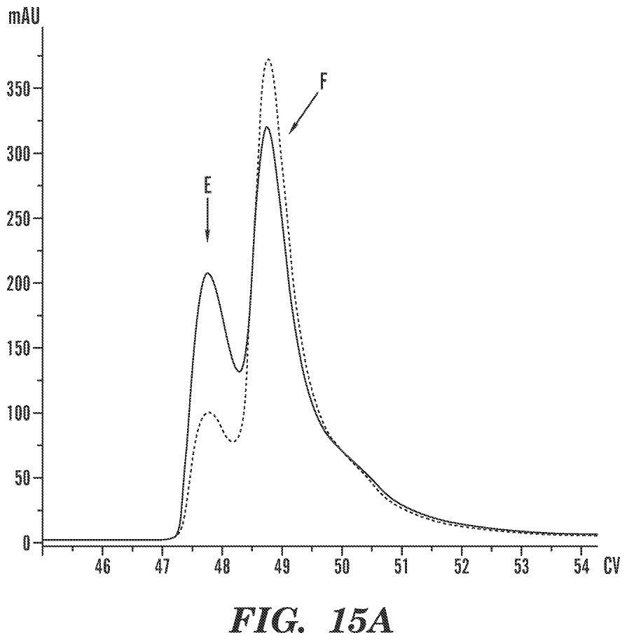

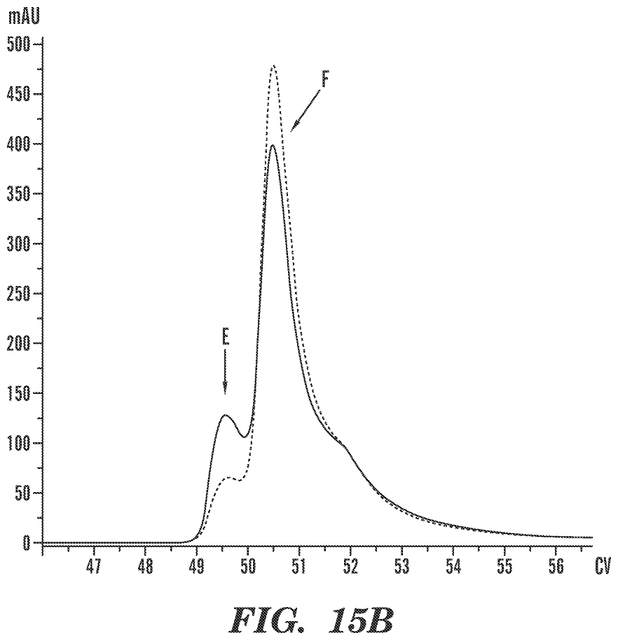

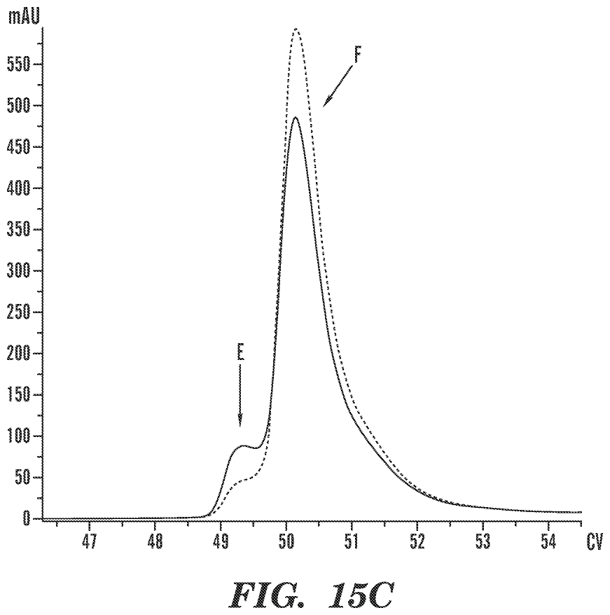

Sample preparation: The affinity eluate was diluted 5× with the AEX dilution buffer comprising different amounts of citric acid. Prior to applying the sample to the anion exchange column, the column was conditioned with a buffer similar to the dilution buffer except comprising a differing amount of the citric acid. Results are shown in FIGS. 15A-21D.

As seen from FIGS. 15A-15C, in the absence of a weak acid in the dilution buffer, there was complete binding and elution of full (F) and empty (E) capsids. Further, the UV_{260/280} ratio of the starting material (S/M) affects the “empty shoulder” and bioreactor consistency (Empty/Full distribution) has a large effect on the chromatographic separation.

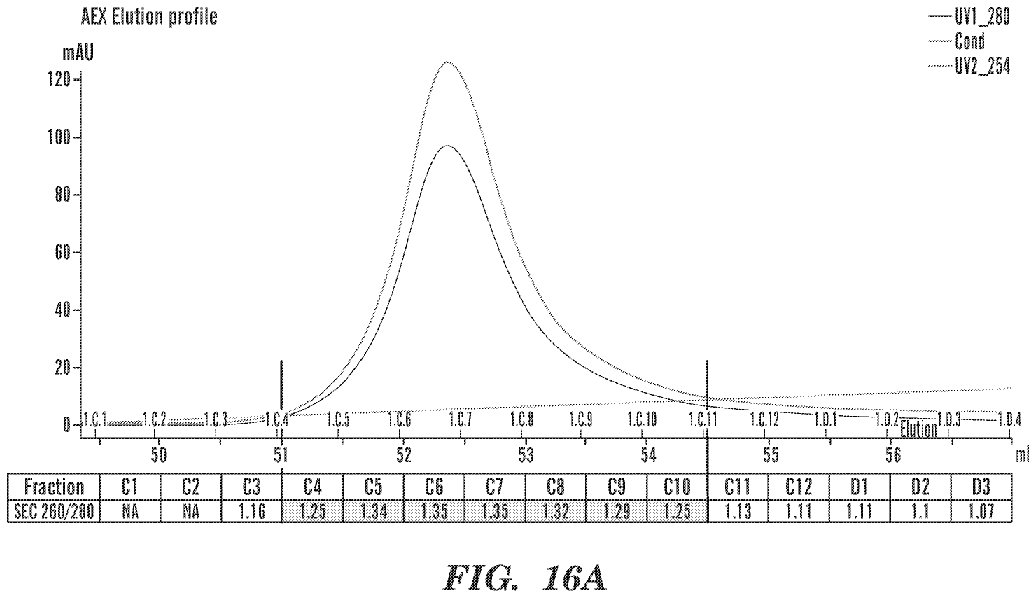

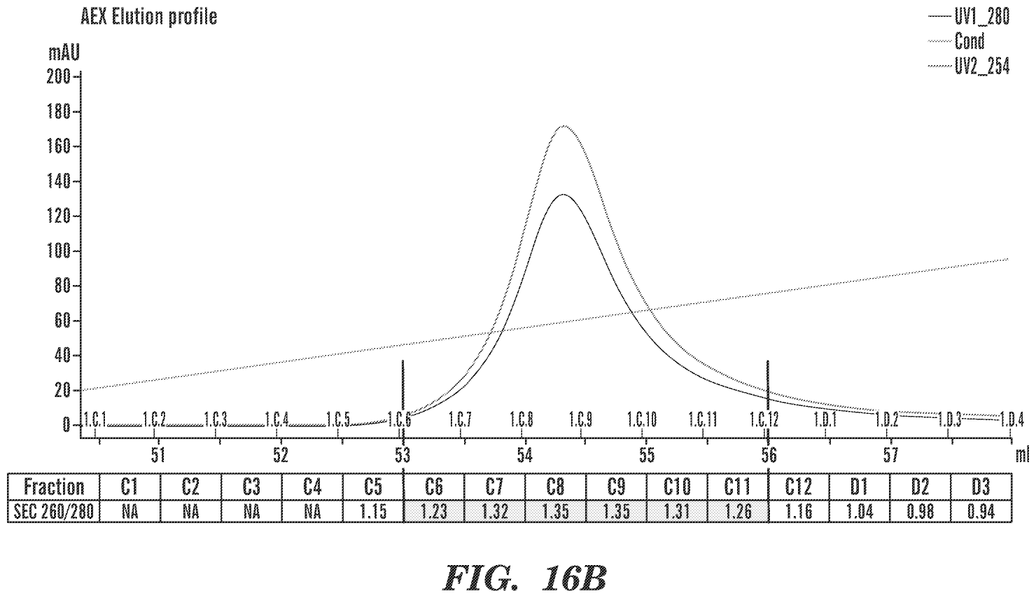

As seen from FIGS. 16A and 16B, inclusion of citric acid as a modulator shows partitioning of empty capsids and little or no empty capsids were observed with fraction enrichment up to 1.35 uv260/280 by SEC. As different starting materials were used for the elutions shown in FIGS. 16A and 16B, the results demonstrate reproducibility and robustness of the AEX method described herein.

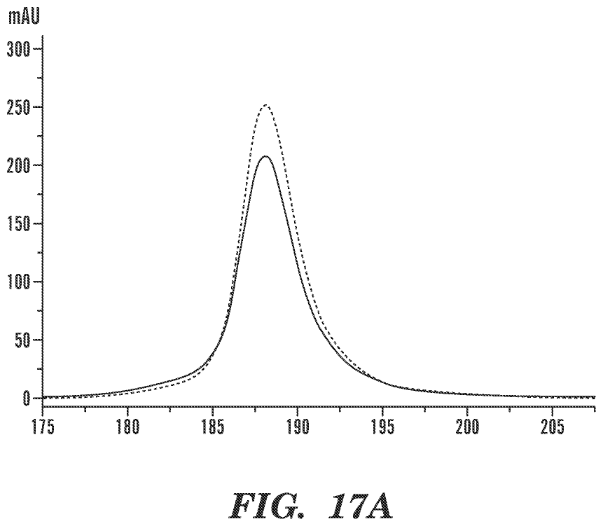

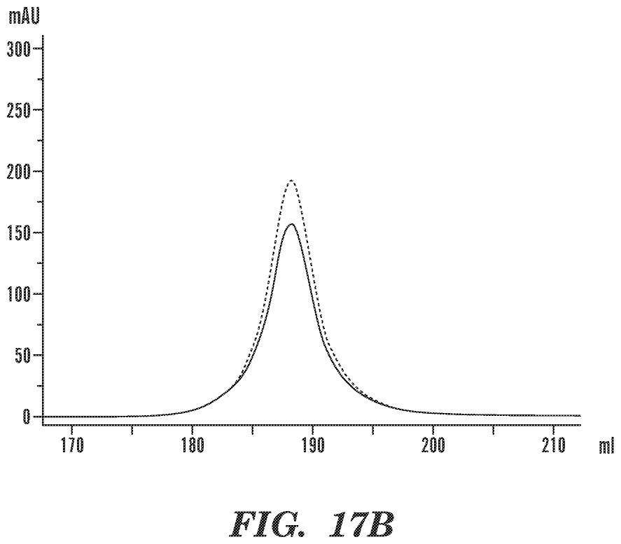

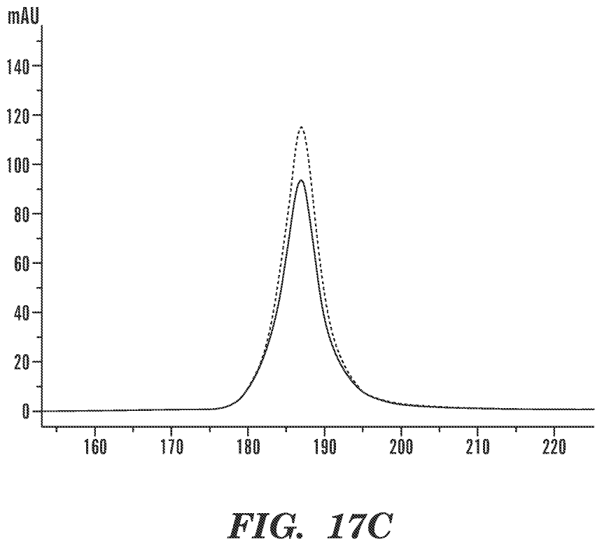

The effect of citric acid concentration in the AEX dilution was determined and results are summarized in Table 5 and exemplary elution profiles shown in FIGS. 17A-17C.

TABLE 5
Effect of citric acid on elution

| Citric Acid | | | | | | |
|---|---|---|---|---|---|---|
| Conditions | | | | AEX | | |
| (in dilution | | AEX | AEX | Elution | | |
| buffer, in | Inflection | Elution | Elution | Recovery | AEX | |
| equilibration | point of | Recovery | Recovery | ITR-qPCR | Elution | |
| buffer | empty shoulder | SEC (%) | ELISA (%) | (%) | UV_{260/280} | SEC-MALs_{(% Full)} |
| 8 mM, 3 mM | ~42 mAU | 18% | 18% | 56% | 1.31 | 60.6 |
| 9 mM, 3 mM | No Empty | 16% | 19% | 47% | 1.30 | 64.6 |
| | Shoulder | | | | | |
| 10 mM, 3 mM | No Empty | 11% | 12% | 34% | 1.30 | 66.1 |
| | Shoulder | | | | | |

As seen from Table 5 and FIGS. 17A-17C, modulation of citric acid in the AEX dilution buffer results in reduction of empty shoulder in AEX elution. As seen, slight changes in citric acid concentrations in the AEX dilution buffer can modulate extent of partitioning. HCP was below LOQ for all runs (<2 ng/mL).

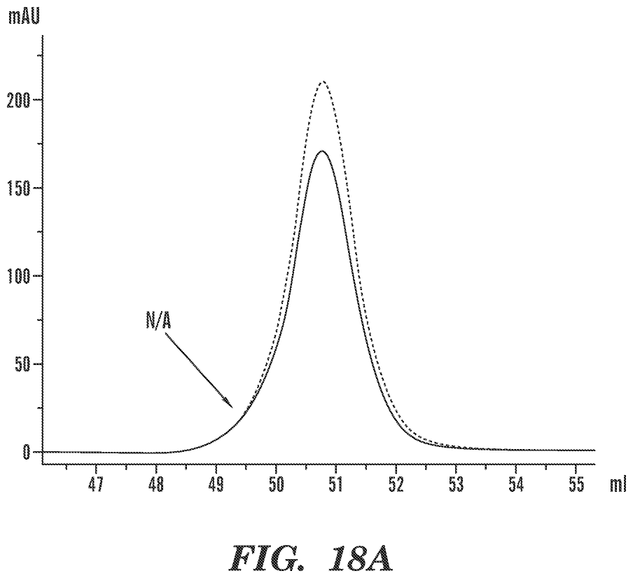

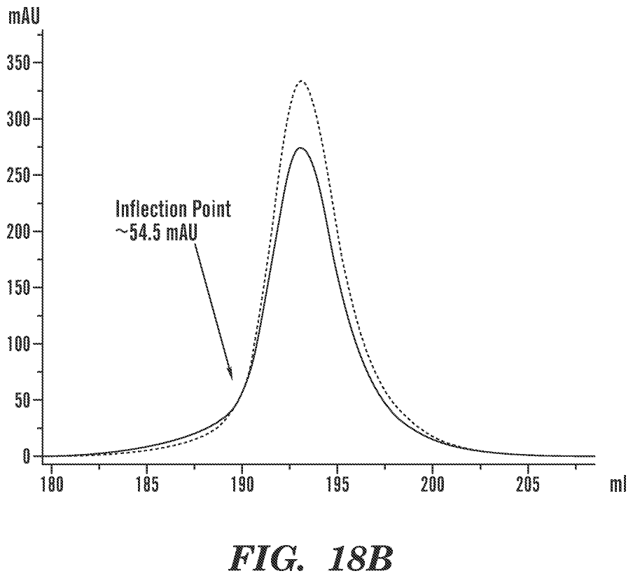

The effect of column size on AEX purification was studied and results are shown in Table 6 and FIGS. 18A-18C.

TABLE 6
Effect of column size on AEX purification

| | UV260/280 | AEX Recovery | AEX Recovery | SEC-MALs |
|---|---|---|---|---|
| Scale | AEX Pool | ITR-qPCR (%) | SEC (%) | (% Full) |
| 1 mL | 1.30 | 24% | 23% | 64.9 |
| 4 mL | 1.28 | 52% | 28% | 60.9 |
| 8 mL | 1.27 | 43% | 35% | 58.5 |

As seen from Table 6 and FIG. 18A-18C, the AEX method is reproducible between monolith scales for partitioning. While there was slight little variability in recovery, full capsids enrichment and capsid partitioning, this variability was related to monolith lot variability.

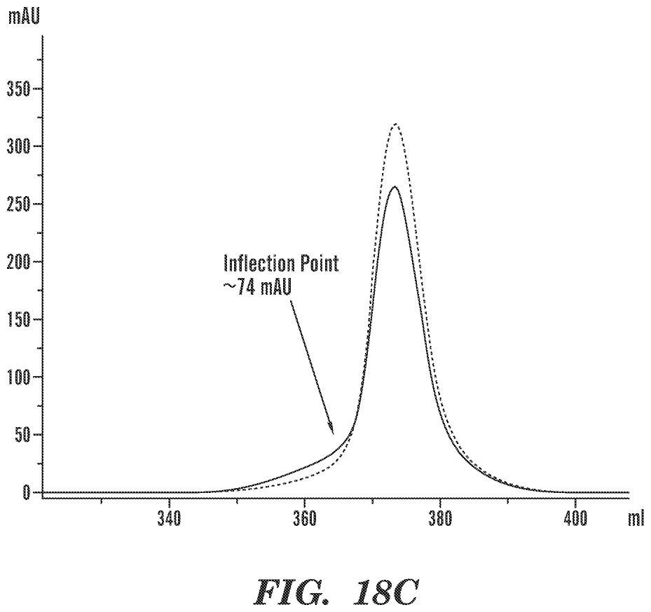

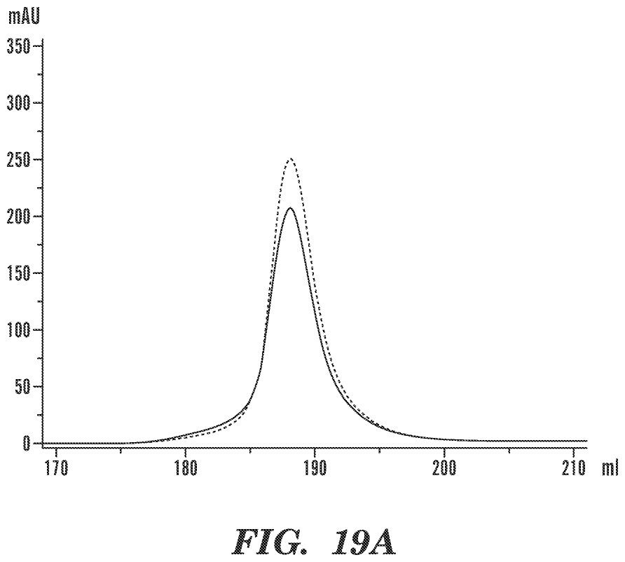

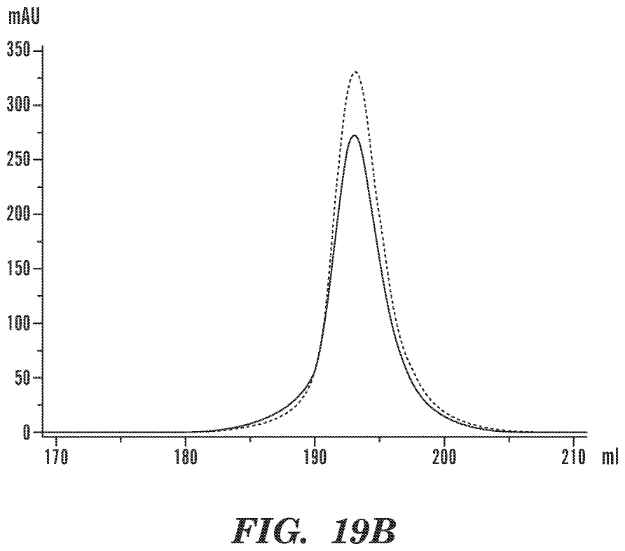

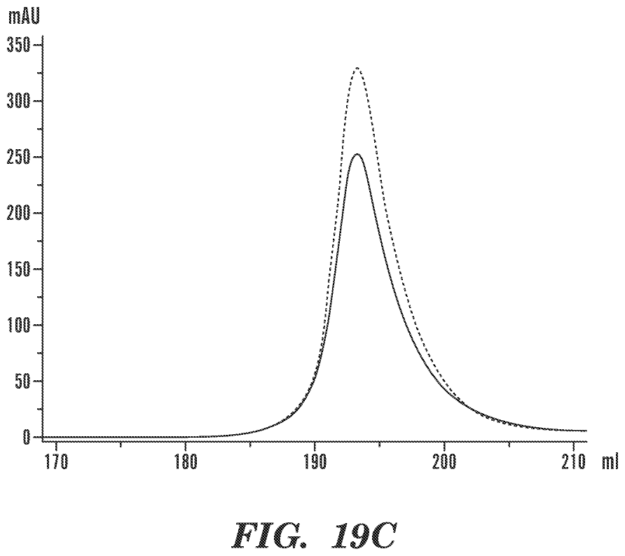

The effect of starting material packaging on AEX was also studied and the results are shown in Table 7 and FIGS. 19A-19C.

TABLE 7
Effect of starting material packaging on AEX separation

| Starting | | | | |
|---|---|---|---|---|
| material | | SEC | Recovery | Recovery |
| UV_{26/280} | Step | UV_{260/280} | SEC (%) | qPCR (%) |
| 0.87 | Affinity Eluate | 0.87 | 100 | 100 |
| | AEX flowthrough | 0.59 | 55 | 3 |
| | AEX Elution | 1.31 | 18 | 56 |
| 0.96 | Affinity Eluate | 0.96 | 100 | 100 |
| | AEX flowthrough | 0.60 | 45 | 3 |
| | AEX Elution | 1.28 | 28 | 86 |
| 1.04 | Affinity Eluate | 1.04 | 100 | 100 |
| | AEX flowthrough | 0.62 | 3 | 3 |
| | AEX Elution | 1.28 | 28 | 47 |

As seen from Table 7 and FIGS. 19A-19C, differences in UV_{260/280} ratio of starting material resulted in similar performance across the 4 mL monolith scale. The AEX flow through UV_{260/280} ratios and AEX eluate UV_{260/280} ratios were comparable between the different starting materials. Residual Host Cell Proteins (HCP) was below LOQ for all runs (<2 ng/mL).

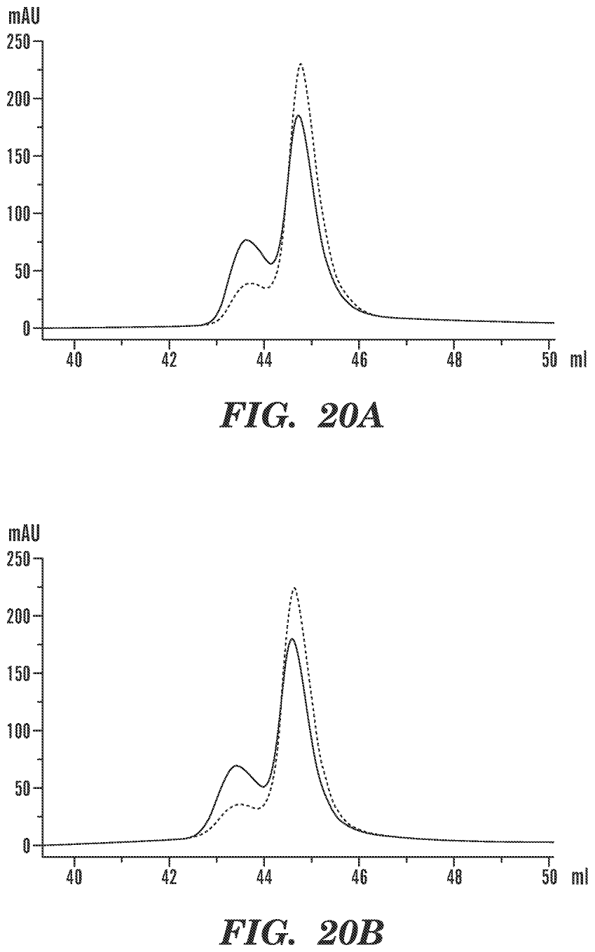

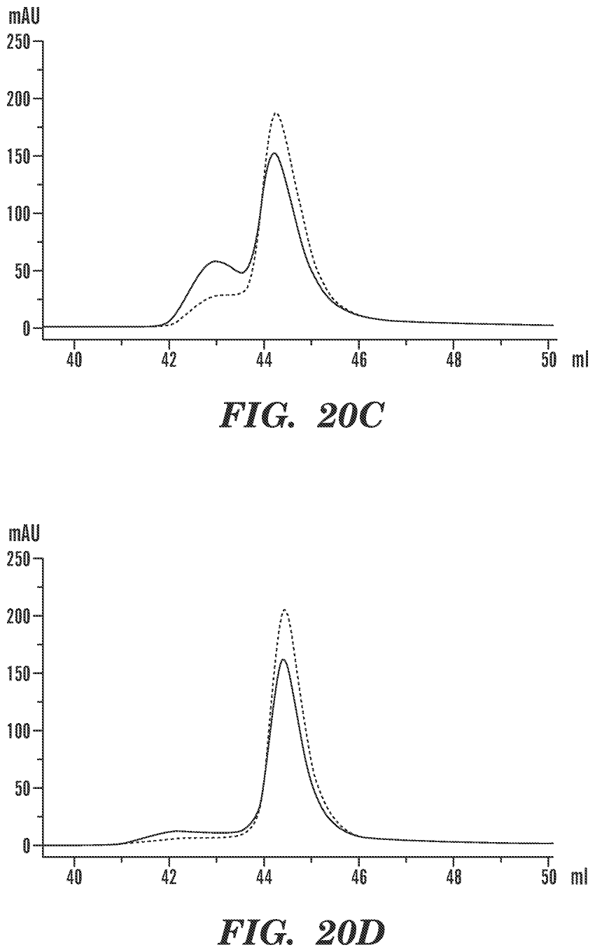

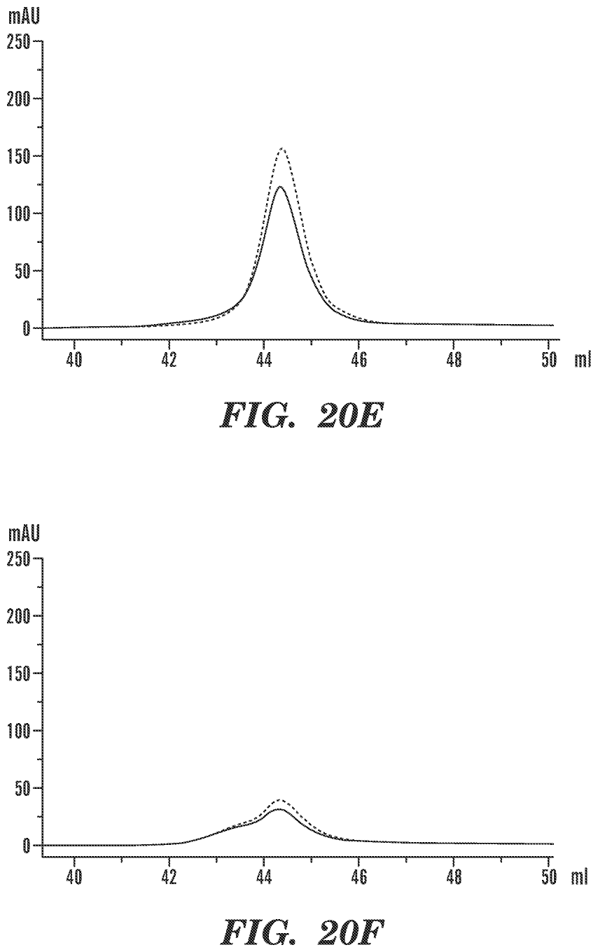

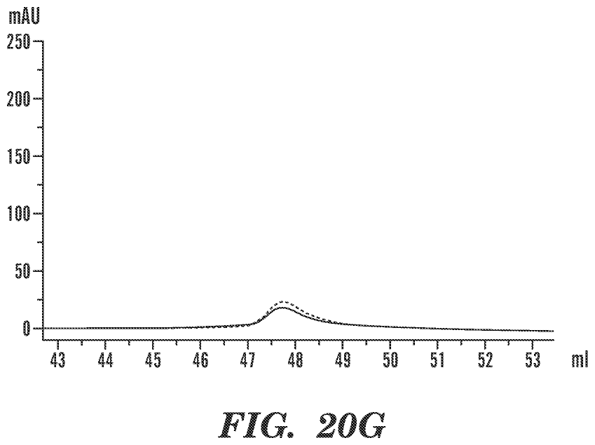

As seen from FIGS. 20A-20G, the amount of the weak acid, e.g., citric acid, in the AEX dilution buffer or AEX equilibration buffer affects the peak shape/purity/recover of AEX purification. In the elutions shown in FIGS. 20A-20G, peak shape was distorted at higher concentrations (>6 mM) of citric acid in the AEX dilution buffer but lower concentrations of citric acid in the AEX dilution buffer result in poor separation.

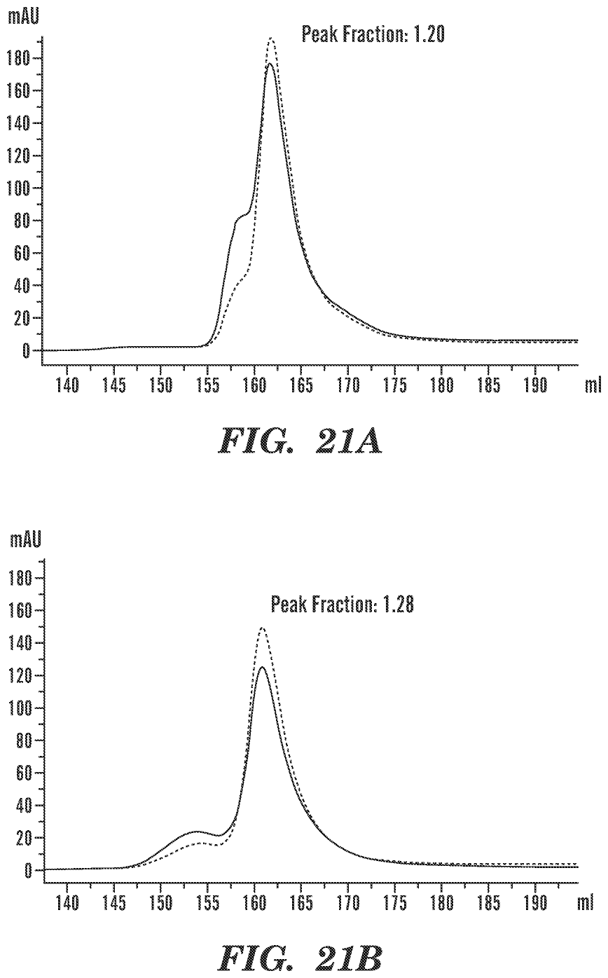

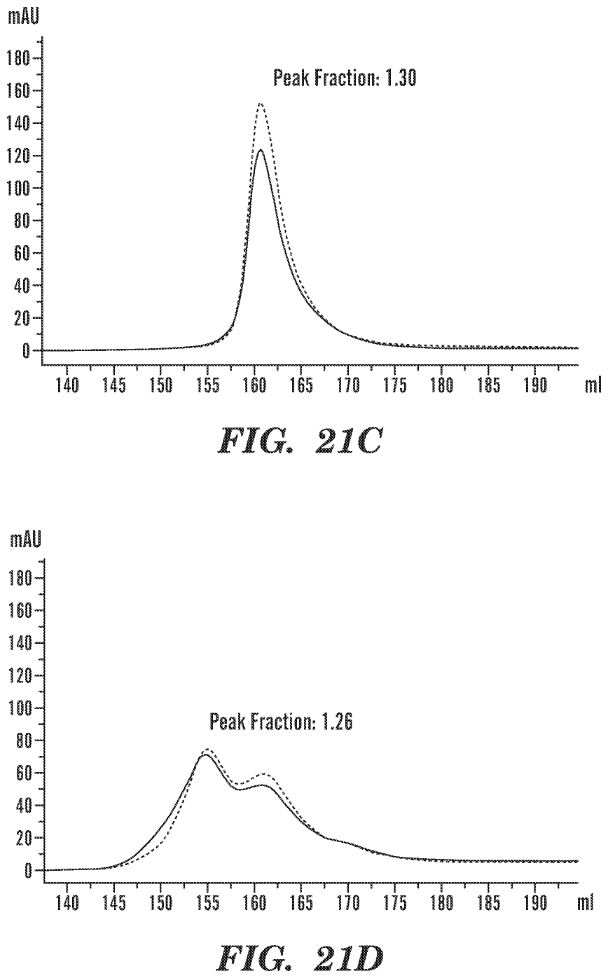

As seen from FIGS. 21A-21D, the partitioning was increased by equilibrated the column with a AEX equilibration buffer comprising a higher concentration of citric acid while maintaining the concentration of the citric acid in the AEX dilution buffer at 6 mM.

### Example 7: Use of Histidine for Modulating Separation of Empty and Full Capsids by AEX

While citric acid can serve as a modulator for many AAV serotypes (AAV2i8, AAV8, AAV rh10), it may not be amenable to some serotypes since it may readily outcompete both empty and full capsids of such AAV serotypes. Accordingly, the inventors tested histidine, which seems to be less harsh, for modulating separation of empty and full capsids similar to weak acids by AEX.

Sample preparation: The affinity eluate was diluted 5× with the AEX dilution buffer comprising different amounts of histidine. Prior to applying the sample to the anion exchange column (QA Monolith Column), the column was conditioned with a buffer comprised of similar histidine concentration to the diluted samples (+1-10 mM).

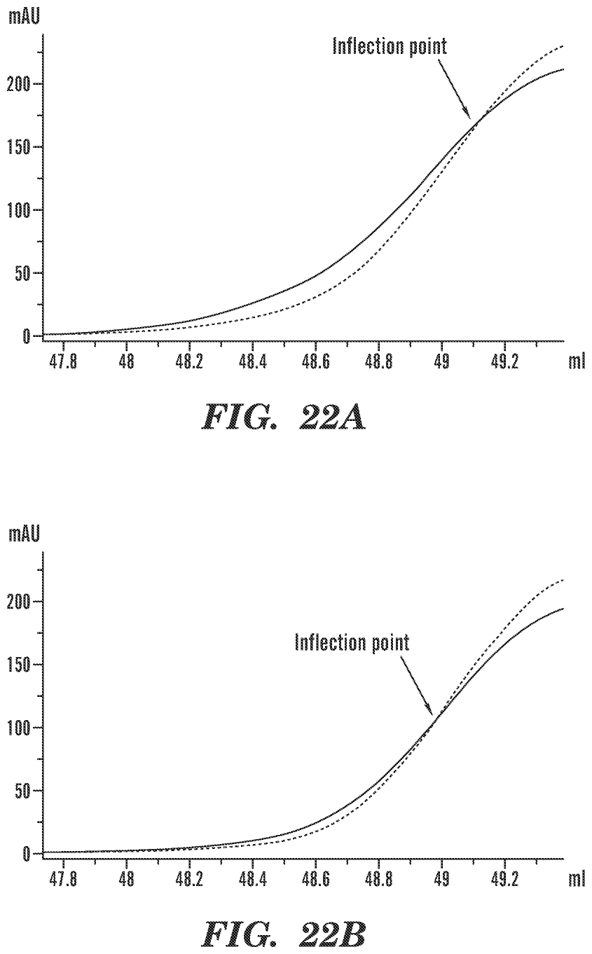

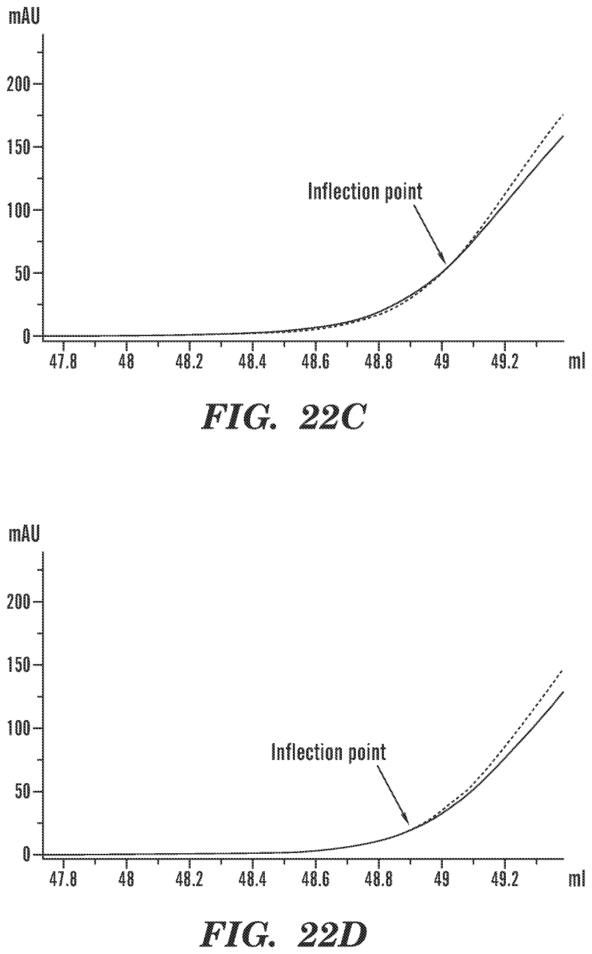

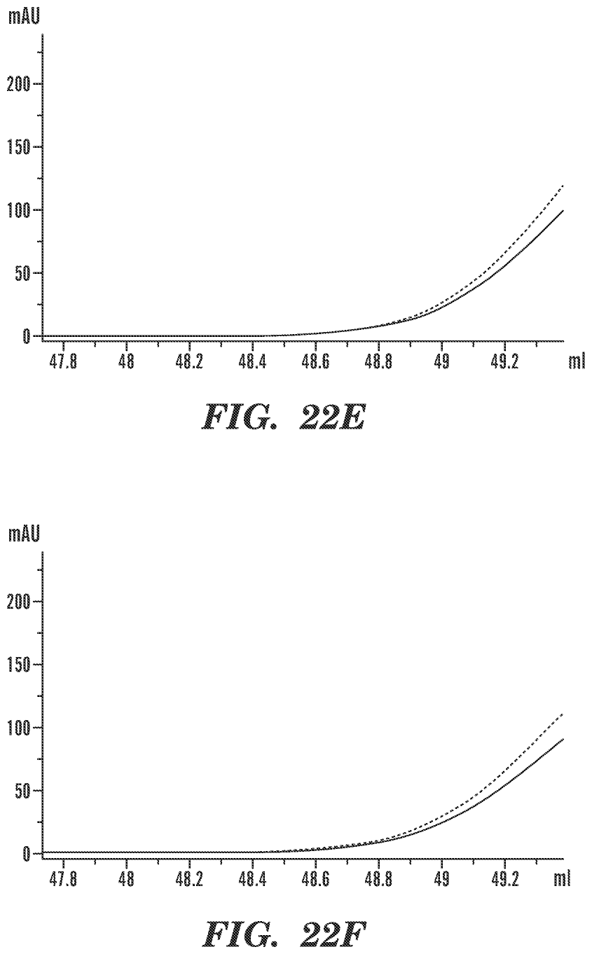

Results are shown in FIGS. 22A-22F and summarized in Tables 8 and 9.

TABLE 8

| | |
|---|---|
| AEX Elution | AEX Flow Through |

| Histidine | % Full | SEC | % Full | SEC |
|---|---|---|---|---|
| Conc. (mM) | Capsids | UV_{260/280} | Capsids | UV_{260/280} |
| 70 | 43.8 | 1.20 | 1.6 | 0.65 |
| 90 | 48.9 | 1.22 | 2.1 | 0.67 |
| 100 | 52.7 | 1.23 | 1.9 | 0.66 |
| 120 | 56.1 | 1.27 | 2.4 | 0.67 |
| 150 | 62.1 | 1.28 | 5.4 | 0.76 |
| 170 | 65.2 | 1.28 | 7.5 | 0.81 |
| 170 | 64.3 | 1.28 | 7.3 | 0.81 |
| 190 | 69.7 | 1.31 | 12.7 | 0.91 |
| 210 | 67.5 | 1.30 | 11.7 | 0.88 |

TABLE 9

| | AEX Elution | | | AEX Flow Through | |
|---|---|---|---|---|---|
| Histidine | Recovery | Recovery | | Recovery | Recovery |
| Conc. | by SEC | by ITR | HCP | by SEC | by ITR |
| (mM) | (%) | qPCR (%) | (ng/mL) | (%) | qPCR (%) |
| 70 | 26% | 37% | Below LOQ | 12% | 1% |
| 90 | 23% | 34% | Below LOQ | 20% | 2% |
| 100 | 23% | 29% | Below LOQ | 23% | 2% |
| 120 | 25% | 40% | Below LOQ | 27% | 4% |
| 150 | 23% | 42% | Below LOQ | 31% | 9% |
| 170 | 20% | 38% | Below LOQ | 35% | 13% |
| 170 | 20% | 33% | Below LOQ | 35% | 12% |
| 190 | 12% | 22% | Below LOQ | 42% | 22% |
| 210 | 14% | 26% | Below LOQ | 42% | 19% |

As seen, histidine concentration of about 120-170 mM in the AEX dilution buffer showed a good balance between recovery and purity.

### Example 8: High Throughput rAAV Production and Downstream Purification of Recombinant Viral Particles

The recombinant adeno-associated viral vector (rAAV) used in the development of this HTS AEX purification method was produced utilizing a transient triple transfection system in a suspension-adapted HEK293 cell line (Pro10™). The rAAV evaluated in the HTS AEX method include both natural and chimeric serotypes. For process demonstration, these include AAV8 and AAV2i8 serotypes. Harvested cell culture containing rAAV vector was clarified utilizing depth filtration and further purified by affinity chromatography.

Liquid Handling System Specifications: The automated liquid handling system used to conduct these experiments was a TECAN Freedom EVO 200 (TECAN, US). The Freedom Evo configuration included a liquid handling arm (LiHa) outfitted with fixed tips for liquid transfer and application of buffer and samples associated with the strong anion exchange chromatographic operation using POROS HQ50 RoboColumns from Repligen (Repligen GmbH, Germany) integrated to a TECAN TeChrom module (TECAN, US). The Freedom Evo 200 system was also equipped with a robotic manipulator arm (ROMA) for transfer of plates, activation and deactivation of the waste tray associated with the TeChrom module.

Modified HTS AEX RoboColumn Method: POROS 50 HQ RoboColumns (100 μL) were acquired from Repligen (Germany) and tested using TECAN's TeChrome module (TECAN, US). Buffers and the AEX load material were transferred from their respective deck locations and applied to RoboColumns using the fixed tips of the liquid handling arm (LiHa). A 96-deep well plate was incrementally passed, by means of a Te-Shuttle, underneath the base of the Te-Chrom-mounted RoboColumns to collect the fractionated flowthrough and AEX eluate samples. The number of anion exchange chromatography conditions (dilution buffer and column conditioning buffer combinations: 96 max conditions) were user specified at the beginning of the script and dictated the total number of columns that underwent the purification cycle and the number of plates needed for the experiment.

RoboColumn Preparation: The columns were cleaned in place (CIP), stripped, and conditioned with elution buffer. The robocolumns were then equilibrated in buffers with user specified weak acid concentrations (Ex. Column A1 was conditioned in buffer containing XmM weak acid while B1 was conditioned in buffer containing YmM weak acid.) It is noted that these variable buffers can be rearranged on the TECAN Worktable allowing for variable experimental design.

Sample Preparation: The affinity eluate obtained from bulk purification or obtained from the affinity chromatography purification on the TECAN, was subject to a 5× dilution (1 part affinity eluate and 4 parts dilution buffer) with a user specified dilution buffer containing a variable amount of weak acid. The number of samples (conditions) and the volume of the affinity eluate addition to vary resin challenge were selected. Dilution Buffers containing variable weak acid concentrations were transferred from Tecan troughs or tubes into sample prep containers. The affinity eluate was then transferred from a single tube on the TECAN worktable and applied to the desired amount of sample prep containers based on the specified number of experimental conditions.

Sample Application: A 96 deep well collection vessel was transferred from a holder “hotel” to the TeShuttle by the robotic manipulation arm (RoMA). The Te-Shuttle ferried the collection vessel underneath the appropriate set of RoboColumns in the Te-Chrome module to collect the resultant flowthrough upon sample application and AEX elution fractions upon sample elution. Sample was transferred from the sample prep containers to the RoboColumns by the liquid handling arm (LiHa) by fixed needle injection. Flowthrough was collected in 1 CV fractions by actuating the collection vessel via the Te-Shuttle. Post sample application, the RoboColumns undergwent a wash to elute any none specifically bound impurities and then the bound sample was subsequently eluted in a conductivity-mediated step elution in a 3 CV fraction to the respective 96 deep well collection vessel. The collection vessel was then returned to the holder “hotel” by the robotic manipulation arm (RoMA), and a new 96 deep well collective vessel was taken from the hotel and transferred to the TeShuttle for the subsequent collection of the next specified column conditioning experimental condition. This process of sample application, wash, and elution was repeated based on the user specified number of column conditioning experimental conditions. Upon completion of the last elution, the RoboColumns were subject to post elution cleaning in place procedures and are stored in a 20% ethanol solution.

UV Absorbance Reader: The UV absorbance of the AEX flowthrough and eluate fractions were measured by a TECAN Infinite 200 PRO microplate reader (TECAN, US). One hundred microliters of AEX flowthrough and eluate fractions were aliquoted by the liquid handler arm (LiHa) from 96 deep well collection vessel to a 96 well UV microplate (Greiner 96 Flat Transparent [GRE96ft_half area_UV-Star]). The UV microplate is then transferred by the robotic manipulation arm (RoMA) to the open tray of the TECAN Infinity 200 PRO microplate reader. The UV absorbance of each sample is measured at A260 nm and A280 nm to determine the UV_{260/280} ratio. The UV_{260/280} ratio serves as a rough estimation of the distribution of full and empty capsids within the sample.

### Example 9: Exemplary AAV Formulations

Materials: Trizma base [2-amino-2-(hydroxymethyl)-1,3-propanediol], potassium chloride, sodium chloride, sodium phosphate dibasic heptahydrate, potassium phosphate monobasic, d-sorbitol, d-mannitol, sucrose, magnesium sulfate anhydrous, calcium alpha-D-heptagluconate dihydrate, Proteinase K Solution, DNAse enzyme, and were purchased from Sigma-Aldrich (St. Louis, MO). Histidine Buffer 25 mM and 10×PBS, pH 6.0 was purchased from Bioworld (Dublin, Ohio). Pluronic™ F-68 Non-ionic Surfactant (100×), Low EDTA TE Buffer, Sheared Salmon Sperm DNA, Sodium Deoxycholate Detergent, Dulbecco's Modified Eagle Medium (DMEM), 2-propanol and TaqMan Fast Advanced MasterMix were purchased from Thermo Fisher Scientific (Pittsburgh, PA). Tween 20 was purchased from Millipore Sigma (Burlington, MA). All other chemicals were of analytical reagent grade and purchased from unless specified otherwise.

### Methods

Formulation preparation: Formulations were prepared by combining buffers, excipients, and/or surfactants in the appropriate concentrations and filtered 0.2 μm PES syringe filter (Corning Life Sciences, Corning, NY) for sterility. Stock vector, stored in AskBio Final formulation buffer, was added to each formulation and buffer exchange was then performed by using Amicon Ultra-0.5 Centrifugal Filter Units. A volume of 0.5 mL formulation was added into each filtration unit which was subsequently spun at 14,000 g for 15 minutes in a Heraeus Fresco 21 Centrifuge (Thermo Fisher Scientific, Waltham, MA). Following the spin, the permeate was discarded and the unit was refilled with formulation buffer and spun two more times. After the final spin, the permeate is discarded and the filter unit is inverted to retrieve the buffer exchanged vector, by now performing a 1,000 g spin for 2 minutes. Sample was then resuspended to starting volume of vector and analysis was immediately performed.

Freeze Thaw Evaluation: Samples were prepared in their respective buffers and stored in Eppendorf tubes. Four freeze-thaw cycles were performed, wherein 1 cycle consists of 24 hours at −80° C. followed by 24 hours at room temperature (20° C.). Analysis was performed following the fourth freeze-thaw cycle.

DLS: For DLS analysis, samples were spun at 21.1×g for 10 minutes in a Heraeus Fresco 21 Centrifuge (Thermo Fisher Scientific, Waltham, MA) and then placed in cuvettes and measured using a Zetasizer Ultra-Red (Malvern Instruments Ltd., Worcestershire, UK). Data was analyzed with ZS XPLORER 2.0.0.98 Software.

pH and Osmolarity: Formulation pH was evaluated in triplicate using the Mettler Toledo Seven Excellence™ pH Meter. Osmolarity values were collected using the PSI Multi Osmette 2430 (Precision Systems Inc (Washington, DC).

UNcle: Samples were centrifuged at 21.1×g for 10 minutes in a Heraeus Fresco 21 Centrifuge (Thermo Fisher Scientific, Waltham, MA). Then 8 uL was added to each well and samples were run in the UNcle (Unchained Labs, Pleasanton, CA) for thermal degradation analysis. The start temperature was set to 25° C. and a ramp rate of 1° C./min was used until the end temperature was reached (95° C.). Filter settings set to 266 nm and 473 nm. Data analysis was performed using the Uncle Analysis 5.03 software.

SEC-HPLC: Samples were vialed neat into polypropylene HPLC Vials (Mfg, Part No.) and placed in the multisampler at 6 C. The vial was then injected to target a load of 5e10 vp onto the column (Agilent Bio SEC-5 4.6 mm×150 mm 500 Å (Agilent P/N 5190-2534)). The sample was analyzed at 214 nm, 260 nm and 280 nm wavelengths at a flow rate of 0.4 mL/min for 10 minutes on the HPLC system equipped with pump, multisampler, column heater, and DAD detector (Agilent 1260 Prime II or equivalent). Data was analyzed with Empower 3 Data Acquisition Software.

qPCR: Samples were diluted 1:100 through 2 serial 1:10 dilutions in Sample Dilution Buffer (SDB; 100 mg/mL Sheared Salmon Sperm DNA, 0.1% Pluronic F68) and treated with DNAse enzyme for 30 minutes at 37° C. followed by treatment with Proteinase K (0.5 mg/mL) for 1 hour at 55° C. and 10 minutes at 95° C. Samples were then diluted again 1:10 in SDB. Samples were treated simultaneously alongside a positive control, which after DNAse and Proteinase K treatment was diluted to both 1:10 and 1:100 in SDB. Mastermix was prepared using Fast Taqman Advanced Mastermix (Applied Biosystems) with 0.05 μM forward (5′-GGAACCCCTAGTGATGGAGTT-3′ (SEQ ID NO: 1)) and reverse (5′-CGGCCTCAGTGAGCGA-3′ (SEQ ID NO: 2)) primers and 0.25 μM FAM probe (5′-/56-FAM/CACTCCCTCTCTGCGCGCTCG/3BHQ_1/-3′ (SEQ ID NO: 3)). 20 μL of mastermix was combined with 5 μL of treated and diluted samples and controls. Extracted viral vector single stranded DNA stock was heated at 95° C. for three minutes and then cooled at room temperature before being used to prepare a 7-point standard curve. This standard curve was plated also alongside samples, positive extraction control, negative extraction control (10 mM Phosphate, 350 mM NaCl, 2.7 mM KCl, 5% Sorbitol, 0.001% Pluronic, pH 7.4), and non-template controls. Mastermix containing samples was loaded and run on a QuantStudio Flex 6 (Thermo Fisher, Pittsburgh, PA) with a 5 minute 95° C. hold followed by 40 cycles consisting of 5 seconds at 95° C. and 30 seconds at 60° C. Titers of samples were determined using quantity interpolation from the extracted viral vector ssDNA standard and then adjusted for dilutions during the sample preparation and plating process.

TCID50: Samples were serially diluted in infection media, consisting of DMEM and wild type Adenovirus 5 (ATCC VR-1516) at a concentration of 4 E7 IU/mL, and used to infect HeLa RC32 cells (ATCC CRL-2972). Seventy-two hours after infection, cells are incubated with lysis buffer (Tween 20, Pro K Buffer, Pro K enzyme, sodium deoxycholate) and the resulting reaction is diluted in sample dilution buffer (Low EDTA buffer, 10% Pluronic, sheared salmon sperm DNA). The samples are analyzed via qPCR on the QuantStudio Flex 6 (Thermo Scientific, Pittsburgh, PA) to determine the copy number of the inverted terminal repeat (ITR) sequences in each well. The Spearman-Karber analytical method was then applied to determine the infectious titer.

Exemplary formulations and results are shown in Tables 10-24.

TABLE 10
Phosphate comprising formulations

| | Formulation | | | | | | | | | | | | | | | | | |
|---|---|---|---|---|---|---|---|---|---|---|---|---|---|---|---|---|---|---|
| Excipient | Control | | 1 | | 2 | | 3 | | 4 | | 5 | | 6 | | 7 | | 8 | |
| Phosphate | 10 | mM | 10 | mM | 10 | mM | 10 | mM | 10 | mM | 10 | mM | 10 | mM | 10 | mM | 10 | mM |
| NaCl | 350 | mM | 137 | mM | 137 | mM | 137 | mM | 137 | mM | 137 | mM | 137 | mM | 137 | mM | 137 | mM |
| KCL | 2.7 | mM | 2.7 | mM | 2.7 | mM | 2.7 | mM | 2.7 | mM | 2.7 | mM | 2.7 | mM | 2.7 | mM | 2.7 | mM |
| MgSO₄ | | | | | 20 | mM | | | 20 | mM | 20 | mM | 20 | mM | 20 | mM | 20 | mM |

| | | | | | | | | | |
|---|---|---|---|---|---|---|---|---|---|
| Sorbitol | 5% | 5% | 5% | | | 5% | | 5% | |
| Mannitol | | | | 5% | 5% | | 5% | | 5% |
| PF68 | 0.01% | 0.01% | 0.01% | 0.01% | 0.01% | | | | |
| Calcium α-d- | | | | | | | | 10% | 10% |
| heptagluconate | | | | | | | | | |
| pH | 7.4 ± 0.1 | 7.6 | 7.4 | 7.6 | 7.3 | 7.4 | 7.4 | 6.2 | 6.2 |
| Osmolarity | 900-1100 | 550 | 560 | 560 | 580 | 560 | 580 | 780 | 780 |
| (mOsm) | | | | | | | | | |
| Conductivity | 36 ± 5 | | | | | | | | |
| (mS/cm) | | | | | | | | | |
| AAV sero-type | AAV9 | AAV9 | AAV9 | AAV9 | AAV9 | AAV9 | AAV9 | AAV9 | AAV9 |

The phosphate comprising formulations were evaluated for vector stability (DLS), genome titer (ITR qPCR) and packaging of % full capsids (SEC-HPLC). Results are summarized in Table 11.

TABLE 11
Analysis of phosphate comprising formulations

| | | | ITR qPCR | SEC | |
|---|---|---|---|---|---|
| | DLS | | titer | | 260/ |
| | Aggregation | % PD | (vg/mL) | vp/mL | 280 |
| Process | Yes | 30 | 1.4 ± 0.03E14 | 8.6E13 | 1.31 |
| Control | | | | | |
| Control | Yes | 30 | 1.9 ± 0.04E14 | 1.14E14 | 1.33 |
| Formulation | | | | | |
| Formulation 1 | | 15 | 1.1 ± 0.03E14 | 8.6E13 | 1.32 |
| Formulation 2 | | 18 | 8.9 ± 0.6E13 | 5.29E13 | 1.32 |
| Formulation 3 | | 13 | 1.1 ± 0.04E14 | 7.5E13 | 1.33 |
| Formulation 4 | | 12 | 1.1 ± 0.04E14 | 7.01E13 | 1.32 |
| Formulation 5 | | 8 | 8.2 ± 0.7E13 | 6.61E13 | 1.33 |
| Formulation 6 | | 12 | 8.1E13 | 6.42E13 | 1.33 |
| Formulation 7^{a} | | 20% | 1.0 ± 1.1E14 | 7.94E13 | 1.31 |
| Formulation 8^{b} | | >50% | 9.1 ± 0.07E13 | 7.21E13 | 1.31 |

^{a}Formulation precipitated

As seen from the data summarized in Table 11, there was little change in qPCR titer for Formulations 1, 3 and 4. Further, aggregation (DLS) and capsid integrity (SEC) were comparable for Formulations 1-4.

TABLE 12
Tris comprising formulations

| | Formulation | | | | | |
|---|---|---|---|---|---|---|
| Excipient | 9 | 10 | 11 | 12 | 13 | 14 |
| Tris | 20 mM | 20 mM | 20 mM | 20 mM | 20 mM | 20 mM |
| MgSO₄ | | 20 mM | | 20 mM | 20 mM | 20 mM |
| Sorbitol | 5% | 5% | | | 5% | 5% |
| Mannitol | | | 5% | 5% | | Yes |
| PF68 | 0.01% | 0.01% | 0.01% | 0.01% | | |
| pH | 7.5 | 7.4 | 7.5 | 7.4 | 7.5 | 7.5 |
| Osmolarity (mOsm) | 332 ± 2 | | | | 356 ± 2 | 366 ± 4 |
| Conductivity (mS/cm) | | | | | | |
| AAV sero-type | AAV9 | AAV9 | AAV9 | AAV9 | AAV9 | AAV9 |

The Tris comprising formulations were evaluated for vector stability (DLS), genome titer (transgene qPCR) and packaging of % full capsids (SEC-HPLC). Results are summarized in Table 13.

TABLE 13
Analysis of Tris formulations

| | DLS | | | Transgene | | |
|---|---|---|---|---|---|---|
| | | Size | | qPCR titer | SEC | |
| | Aggregation | (nm) | PDI | (vg/mL) | vp/mL | 260/280 |
| Process Control | Yes | | 0.546 | 5.7 ± 0.14 | 2.87 | 1.35 |
| | | | | E12 | E13 | |
| Formulation 9 | No | 31 | 0.073 | 1.9 ± 0.02 | 2.7 ± 0.057 | 1.31 |
| | | | | E12 | E13 | |
| Formulation 10 | Yes | 41 | 0.25 | 2.1 ± 0.24 | 3.3 ± 0.0071 | 1.37 |
| | | | | E12 | E13 | |
| Formulation 11 | Yes | 39 | 0.21 | 2.1 ± 0.41 | 3.3 ± 0.014 | 1.33 |
| | | | | E12 | E13 | |
| Formulation 12 | Yes | 38 | 0.23 | 1.5 ± 0.59 | 2.7 | 1.37 |
| | | | | E12 | E13 | |
| Formulation 13 | No | 33 | 0.097 | 4.4 ± 0.57 | 3.4 ± 0.071 | 1.34 |
| | | | | E12 | E13 | |
| Formulation 14 | No | 32 | 0.66 | 3.3 ± 0.42 | 3.0 ± 0.085 | 1.32 |
| | | | | E12 | E13 | |

As seen from the analysis data summarized in Table 13, Tri buffer results in isotonic solutions. Use of MgSO₄, with or without PF68, inhibits aggregation while maintaining qPCR titer. Capsid integrity (SEC) were comparable for buffers comprising sorbitol or mannitol, with or without PF68.

TABLE 14
Histidine comprising formulations

| | Formulation | | | | | |
|---|---|---|---|---|---|---|
| Excipient | 15 | 16 | 17 | 18 | 19 | 20 |
| Histidine | 25 mM | 25 mM | 25 mM | 25 mM | 25 mM | 25 mM |
| MgSO₄ | | 20 mM | | 20 mM | 20 mM | 20 mM |
| Sorbitol | 5% | 5% | | | 5% | |
| Mannitol | | | 5% | 5% | | 5% |
| PF68 | 0.01% | 0.01% | 0.01% | 0.01% | | |
| pH | 7.69 | 7.46 | 7.62 | 7.49 | 7.53 | 7.55 |
| Osmolarity (mOsm) | 329 ± 1 | | 330 ± 10 | | | |
| Conductivity | | | | | | |
| (mS/cm) | | | | | | |
| AAV sero-type | AAV9 | AAV9 | AAV9 | AAV9 | AAV9 | AAV9 |

The histidine comprising formulations were evaluated for vector stability (DLS), genome titer (transgene qPCR) and packaging of % full capsids (SEC-HPLC). Results are summarized in Table 15.

TABLE 15
Analysis of histidine comprising formulations

| | DLS | | | Transgene | | |
|---|---|---|---|---|---|---|
| | | Size | | qPCR titer | SEC | SEC |
| | Aggregation | (nm) | PDI | (vg/mL) | (vp/mL) | 260/280 |
| Process Control | Yes | | 0.546 | 2.2 ± 0.14 | 4.4 | 1.36 |
| | | | | E13 | E13 | |
| Formulation 15 | No | 32 | 0.057 | 2.6 ± 0.18 | 5.7 ± 0.13 | 1.32 |
| | | | | E13 | E13 | |
| Formulation 16 | Yes | 35 | 0.13 | 2.0 ± 0.0.0071 | 5.0 ± 0.028 | 1.34 |
| | | | | E13 | E13 | |
| Formulation 17 | No | 32 | 0.040 | 2.1 ± 0.07 | 4.9 ± 0.05 | 1.35 |
| | | | | E13 | E13 | |
| Formulation 18 | Little bit | 34 | 0.12 | 1.8 ± 0.02 | 4.5 ± 0.02 | 1.33 |
| | | | | E13 | E13 | |
| Formulation 19 | Little bit | 34 | 0.12 | 1.7 ± 0.03 | 4.8 ± 0.014 | 1.30 |
| | | | | E13 | E13 | |
| Formulation 20 | No | 33 | 0.08 | 1.5 ± 0.12 | 4.0 ± 0.12 | 1.34 |
| | | | | E13 | E13 | |

As seen from the analysis data summarized in Table 15, histidine buffer results in isotonic solutions and PF68 inhibits aggregation. Transgene qPCR titers and capsid integrity (SEC) were comparable for buffers comprising sorbitol or mannitol, with or without PF68. No aggregation, change in SEC or change in qPCR was seen with Formulations 15 and 17.

Stability of some exemplary formulations after one or more freeze thaw cycles were determined. Results are summarized in Tables 16 and 17.

TABLE 16
Freeze thaw PBS Formulation results

| | | | | Transgene | | |
|---|---|---|---|---|---|---|
| | | Size | | qPCR titer | SEC | |
| | Aggregation | (nm) | % PD | (vg/mL) | vp/mL | 260/280 |
| Process Control | No | 31 | 6 | 5.7 ± 0.14 | 6.13 ± 0.2 | 1.25 |
| | | | | E12 | E13 | |
| Formulation 1 | No | 33 | 14 | 4.4 ± 0.36 | 2.22 ± 2.6 | 1.44 |
| | | | | E12 | E13 | |
| Formulation 2 | Yes | 36 | 22 | 3.8 ± 0.14 | 4.57 ± 0.02 | 1.34 |
| | | | | E12 | E13 | |
| Formulation 3 | Yes | 37 | 33 | 3.7 ± 0.11 | 3.6 ± 0.007 | 1.34 |
| | | | | E12 | E13 | |

TABLE 17
Freeze Thaw Study 2 Results

| | DLS | | | Transgene | | | | |
|---|---|---|---|---|---|---|---|---|
| | | Size | | qPCR titer | SEC | | Osmolarity | |
| | Aggregation | (nm) | PDI | (vg/mL) | vp/mL | 260/280 | (mOsm) | pH |
| Process Control | Yes | 50 | 0.34 | 2.6 ± 0.064 | 5.2 ± 0.198 | 1.32 | 900-1100 | 7.4 |
| | | | | E13 | E13 | | | |
| Formulation 1 | Yes | 52 | 0.29 | 2.6 ± 0.46 | 6.0 ± 0.032 | 1.36 | 550 | 7.60 |
| | | | | E13 | E13 | | | |
| Formulation 15 | No | 32 | 0.069 | 2.4 ± 0.07 | 5.6 ± 0.036 | 1.32 | 329 ± 1 | 7.51 |
| | | | | E13 | E13 | | | |
| Formulation 17 | Yes | 36 | 0.16 | 1.8 ± 0.035 | 4.1 ± 0.12 | 1.32 | 330 ± 10 | 7.62 |
| | | | | E13 | E13 | | | |
| Formulation 9 | No | 33 | 0.099 | 1.8 ± 0.069 | 3.7 ± 0.62 | 1.33 | 332 ± 2 | 7.53 |
| | | | | E13 | E13 | | | |
| Formulation 13 | Yes | 37 | 0.21 | 1.7 ± 0.12 | 3.7 ± 0.14 | 1.33 | 634 ± 200 | 7.55 |
| | | | | E13 | E13 | | | |
| Formulation 14 | No | 38 | 0.17 | 2.1 ± 0.007 | 4.9 ± 0.083 | 1.32 | 366 ± 4 | 7.57 |
| | | | | E13 | E13 | | | |

TABLE 18
TCID₅₀ Results

| | TCID₅₀/mL | vg/TCID₅₀ | % Recovery |
|---|---|---|---|
| Reference Formulation* | 1.12E10 | | |
| Formulation 1 | 5.22E9 | 5114.6 | 97 |
| Formulation 15 | 6.32E9 | 3826.4 | 98 |
| Formulation 9 | 1.12E10 | 1627.1 | 100 |

*no freeze thaw

As the data summarized in Table 18 show, activity after 4 freeze-thaw cycles is Tris>His>PBS.

### Thermal Stability Analysis

Some exemplary formulations were subjected to thermal stability analysis using the UNcle system (Unchained Labs, Pleasanton, CA). Results are summarized in Table 19.

TABLE 19
Stability Results

| | T_{agg} | Tₒₙₛₑₜ | Tm | DLS Pre Run | | DLS Post Run | |
|---|---|---|---|---|---|---|---|
| | (° C) | (° C.) | (° C.) | Size (nm) | PDI | Size (nm) | PDI |
| Process control | 76 | 76 | 79 | 35 | 0.149 | 346 | 0.249 |
| Formulation 1 | 76 | 80 | 80 | 39 | 0.237 | 333 | 0.201 |
| Formulation 15 | 78 | 78 | 81 | 33 | 0.118 | 95 | 0.177 |
| Formulation 9 | 78 | 78 | 81 | 33 | 0.146 | 190 | 0.185 |

The data in Table 19 shows impact of heating to 95° C. on the presence of aggregates in different formulations. As seen, Formulations 9 and 15 showed much less aggregation compared to the process control formulation or Formulation 1.

### Formulation Translatability

Formulations shown in Table 15 were prepared with different AAV serotypes (AAV2, AAV9 and AAVrh10) to determine translatability of the formulations to different serotypes.

TABLE 20
Exemplary Formulations

| | Formulation | | | | | | | |
|---|---|---|---|---|---|---|---|---|
| Excipient | 21 | 22 | 23 | 24 | 25 | 26 | 27 | 28 |
| Histidine | 25 mM | 25 mM | 25 mM | 25 mM | | | | |
| Tris | | | | | 20 mM | 20 mM | 20 mM | 20 mM |
| MgSO₄ | 90 mM | 90 mM | 90 mM | 90 mM | 90 mM | 90 mM | 90 mM | 90 mM |
| Sucrose | | | 5% | 5% | | | 5% | 5% |
| PF68 | | 0.01% | | 0.01% | | 0.01% | | 0.01% |
| Osmolarity (mOsm) | 144 | 143 | 295 | 298 | 174 | 193 | 310 | 314 |

The Formulations 21-28 comprising different AAV serotypes were evaluated for vector stability (DLS). Results are summarized in Table 21.

TABLE 21
DLS analysis

| | | Avg. DLS | | Major DLS Peak | |
|---|---|---|---|---|---|
| | | Size | | Size | % of |
| Formulation | Serotype | (nm) | PDI | (nm) | sample |
| Control (centrifuge) | AAV 2 | 28 | 0.030 | 26 | 100 |
| Control (buffer | AAV 2 | 516 | 0.788 | 26 | 79 |
| exchange) | | | | | |
| Control centrifuge | AAV2i8 | 33 | 0.01 | | |
| Control (buffer | AAV2i8 | 34 | 0.12 | | |
| exchange) | | | | | |
| Formulation 21 | AAV 2 | 318 | 0.440 | 501 | 71.8 |
| Formulation 21 | AAV 2i8 | 29 | 0.02 | | |
| Formulation 21 | AAV 9 | 27 | 0.426 | 27 | 100 |
| Formulation 21 | AAV rh10 | 34 | 0.242 | 27 | 99.2 |
| Formulation 22 | AAV 2 | 548 | 0.565 | 671 | 56.4 |
| Formulation 22 | AAV 2i8 | 30 | 0.03 | | |
| Formulation 22 | AAV 9 | 35 | 0.219 | 27 | 98.8 |
| Formulation 22 | AAV rh10 | 31 | 0.112 | 26 | 100 |
| Formulation 23 | AAV 2 | 235 | 0.405 | 43 | 66 |
| Formulation 23 | AAV 2i8 | 73 | 0.45 | 32 | 96 |
| Formulation 23 | AAV 9 | 44 | 0.253 | 31 | 98.2 |
| Formulation 23 | AAV rh10 | 36 | 0.133 | 31 | 98.9 |
| Formulation 24 | AAV 2 | 268 | 0.533 | 59 | 51.4 |
| Formulation 24 | AAV 2i8 | 55 | 0.358 | 31 | 99.3 |
| Formulation 24 | AAV 9 | 39 | 0.173 | 31 | 99.4 |
| Formulation 24 | AAV rh10 | 39 | 0.182 | 30 | 99.7 |
| Formulation 25 | AAV 2 | 309 | 0.196 | 387 | 88 |
| Formulation 25 | AAV 2i8 | 28 | 0.03 | | |
| Formulation 25 | AAV 9 | 30 | 0.0377 | 27 | 100 |
| Formulation 25 | AAV rh10 | 166 | 0.517 | 127 | 81.7 |
| Formulation 26 | AAV 2 | 397 | 0.600 | 28 | 79.4 |
| Formulation 26 | AAV 2i8 | 31 | 0.117 | | |
| Formulation 26 | AAV 9 | 507 | 0.615 | 27 | 99.7 |
| Formulation 26 | AAV rh10 | 32 | 0.123 | 27 | 99.7 |
| Formulation 27 | AAV 2 | 261 | 0.429 | 86 | 36.2 |
| Formulation 27 | AAV 2i8 | 56 | 0.37 | 34 | 99.9 |
| Formulation 27 | AAV 9 | 44 | 0.244 | 32 | 98.4 |
| Formulation 27 | AAV rh10 | 34 | 0.0645 | 30 | 100 |
| Formulation 28 | AAV 2 | 313 | 0.644 | 227 and | 34.3 each |
| | | | | 446 | |
| Formulation 23 | AAV 2i8 | 48 | 0.32 | 32 | 98.6 |
| Formulation 28 | AAV 9 | 39 | 0.154 | 31 | 100 |
| Formulation 28 | AAV rh10 | 43 | 0.274 | 30 | 98.7 |

The Formulations 23 and 26 comprising different AAV serotypes were further evaluated for genome titer (ITR qPCR) and packaging of % full capsids (SEC-HPLC). Results are summarized in Table 22.

TABLE 22
qPCR and SEC analysis of Formulations 23 and
26 comprising serotype AAV2, AAV9 or AAV rh10

| | | ITR qPCR | |
|---|---|---|---|
| Formulation | Serotype | titer (vg/mL) | SEC (vp/mL) |
| Control | AAV 2 | 6.1E12 | 1.3E13 |
| Control (buffer exchange) | AAV 2 | 2.2E12 | 3.6E12 |
| Control | AAV 9 | 6.4E13 | 9.9E13 |
| Control | AAV rh10 | 1.1E14 | 2.7E14 |
| Formulation 23 | AAV 2 | 9.0E11 | 7.5E11 |
| Formulation 23 | AAV 9 | 2.1E13 | 4.0E14 |
| Formulation 23 | AAV rh10 | 7.9E13 | 2.2E11 |
| Formulation 26 | AAV 2 | 1.7E12 | 1.9E12 |
| Formulation 26 | AAV 9 | 2.7E13 | 4.3E13 |
| Formulation 26 | AAV rh10 | 8.9E13 | 2.3E11 |

The Formulation 26 comprising AAV 2i8 serotype was further evaluated for genome titer (ITR qPCR) and packaging of % full capsids (SEC-HPLC). Results are summarized in Table 23.

TABLE 23
qPCR and SEC analysis of Formulation
26 comprising serotype AAV 2i8

| | | ITR qPCR | |
|---|---|---|---|
| Formulation | Serotype | titer (vg/mL) | SEC (vp/mL) |
| Control | AAV 2i8 | 3.7 ± 0.06E13 | 1.7 ± 0.068E14 |
| Control (buffer | AAV 2i8 | 3.7 ± 0.98E13 | 1.6 ± 0.45E14 |
| exchange) | | | |
| Formulation 26 | AAV 2i8 | 2.7 ± 0.11E13 | 1.2 ± 0.049E14 |

The Formulations 23 and 26 comprising different AAV serotypes were further evaluated for genome titer (ITR qPCR) and packaging of % full capsids (SEC-HPLC) after 4 freeze-thaw cycles. Results are summarized in Table 24.

TABLE 24
qPCR, SEC and DLS analysis of Formulations 23 and 26 comprising
serotype AAV2, AAV9 or AAV rh10 after 4 freeze-thaw cycles

| | | qPCR titer | SEC | DLS | |
|---|---|---|---|---|---|
| Formulation | Serotype | (vg/mL) | (vp/mL) | Size (nm) | PDI |
| Control (buffer | AAV 2 | 1.5E12 | 3.1E12 | 29 | 0.06 |
| exchange) | | | | | |
| Control (buffer | AAV 9 | 3.1E13 | 4.7E13 | 34 | 0.11 |
| exchange) | | | | | |
| Control (buffer | AAV rh10 | 1.1E14 | 1.9E14 | * | * |
| exchange) | | | | | |
| Formulation 23 | AAV 2 | 9.4E11 | 1.4E12 | 55 | 0.512 |
| Formulation 23 | AAV 9 | 3.2E13 | 5.2E13 | * | * |
| Formulation 23 | AAV rh10 | 9.1E13 | 1.6E14 | * | * |
| Formulation 26 | AAV 2 | 1.6E12 | 3.7E12 | 30 | 0.16 |
| Formulation 26 | AAV 9 | 2.3E13 | 4.3E13 | 31 | 0.09 |
| Formulation 26 | AAV rh10 | 8.9E13 | 1.9E14 | 30 | 0.04 |

*tube ruptured during centrifuge step

As seen from Tables 23 and 24, Formulation 26 is able to protect against any changes to stability of all 4 AAV serotypes that were tested, with and without freeze-thaw stress. This is important as one of the biggest challenge to AAV vector stability is freeze-thaw agitation.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A population of purified recombinantly expressed adeno associated virus (rAAV) particles, wherein the recombinantly expressed adeno associated virus particles are purified or isolated from a harvesting media by a process comprising:
a. purifying/isolating a plurality of recombinantly expressed virus particles from the harvesting media via affinity chromatography to produce an eluate (affinity chromatography eluate) comprising the plurality of recombinantly expressed virus particles, wherein an elution buffer for affinity chromatography (affinity elution buffer) comprises a predetermined amount of glycine, optionally, the affinity elution buffer is substantially free of weak acids or salts thereof; and optionally, the affinity elution buffer comprises an amino acid that is not glycine;
b. adjusting the affinity chromatography eluate for subsequent purification through anion exchange chromatography, wherein the adjusted affinity eluate comprises a predetermined amount of a weak acid or salt thereof or the adjusted affinity eluate comprises a predetermined amount of an amino acid that is not glycine in addition to the predetermined amount of glycine already present in affinity eluate;
c. purifying/isolating the plurality of recombinantly expressed virus particles from the adjusted eluate of affinity chromatography by anion exchange chromatography to produce a solution comprising a plurality of purified/isolated recombinantly expressed virus particles, wherein an equilibration buffer for anion exchange chromatography comprises a predetermined amount of a weak acid or a salt thereof; and, optionally,
wherein: (i) the population of purified rAAV comprises less than about 35% empty viral capsids; and/or (ii) the purified rAAV has a particle to infectivity ratio less than 2×10⁴ vg/TCID50; and/or (iii) a ratio of UV₂₆₀ to UV₂₈₀ in the anion exchange eluate is at least about 1.15× or higher than a ratio of UV₂₆₀ to UV₂₈₀ ratio in the adjusted affinity eluate.

2. The population of claim 1, wherein less than 5% of empty virus particles in the affinity eluate bind to anion exchange chromatography media.

3. The process of claim 2, wherein substantially no empty virus particles in the affinity eluate bind to anion exchange chromatography media.

4. The process of any one of claims 1-3, wherein the population of purified rAAV comprises less than about 10% empty viral capsids.

5. The process of claim 4, wherein the population of purified rAAV comprises less than about 5% empty viral capsids.

6. The process of claim 5, wherein the population of purified rAAV is substantially free of empty capsids.

7. The population of any one of claims 1-6, wherein the weak acid of 1 (b) is citric acid, acetic acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid or 3-methylbutanoic acid, optionally the weak acid is citric acid, citrate, acetic acid or succinic acid.

8. The population of any one claims 1-7, wherein the affinity elution buffer comprises histidine at a concentration of at least about 1 mM or more.

9. The population of any one claims 1-8, wherein the affinity elution buffer comprises histidine at a concentration from about 1 mM to about 50 mM.

10. The population of any one of claims 1-9, wherein the affinity elution buffer comprises glycine at a concentration of at least about 20 mM or more.

11. The process of any one of claims 1-10, wherein the affinity elution buffer comprises glycine at a concentration of from about 25 mM to about 100 mM.

12. The population of any one of claims 1-11, wherein the affinity elution buffer comprises a salt.

13. The population of any one of claims 1-12, wherein the affinity elution buffer comprises a salt at concentration of at least about 5 mM or more.

14. The population of any one of claims 1-13, wherein the affinity elution buffer comprises a salt concentration of from about 5 mM to about 15 mM.

15. The population of any one of claims 12-14, wherein the salt is a magnesium salt, a sodium salt, a potassium salt, an ammonium salt, a calcium salt, a copper salt, a cobalt salt, a manganese salt, a nickel salt or a zinc salt, optionally the salt is MgCl₂.

16. The population of any one of claims 1-15, wherein the affinity elution buffer comprises a polymer.

17. The population of any one of claims 1-16, wherein the affinity elution buffer comprises a polymer at a concentration of at least about 0.1% or more.

18. The population of any one of claims 1-17, wherein the affinity elution buffer comprises a polymer at a concentration of from about 0.1% to about 0.5%.

19. The population of any one of claims 16-18, the polymer is a non-ionic surfactant.

20. The population of any one of claims 1-19, wherein the affinity elution buffer has a low pH.

21. The population of any one of claims 1-20, wherein the affinity elution buffer has a pH lower than or equal to about 6.5.

22. The process of any one of claims 1-21, wherein the affinity elution buffer has a pH of from about 2.0 to about 3.0.

23. The population of any one of claims 1-22, wherein the affinity elution buffer comprises: about 75 mM glycine.

24. The population of any one of claim 1-7 or 10-23, wherein the affinity elution buffer comprises a weak acid or a salt thereof.

25. The population of claim 24, wherein the affinity elution buffer comprises the weak acid or a salt thereof at a concentration of at least about 50 mM or more.

26. The population of claim 24 or 25, wherein the affinity elution buffer comprises the weak acid or a salt thereof at a concentration of from about 50 mM to about 100 mM.

27. The population of any one of claims 24-26, wherein the weak acid is citric acid, acetic acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid or 3-methylbutanoic acid, or, optionally the weak acid is citric acid, citrate, acetic acid or succinic acid.

28. The population of any one of claims 24-27, wherein the weak acid is citric acid or a salt thereof.

29. The population of any one of claim 1-7, 10-22 or 24-28, wherein the affinity elution buffer comprises: about 50 mM glycine, about 75 mM citrate, about 10 mM MgCl₂, about 0.3% (w/v) P188 and has a pH of about 3.0.

30. The population of any one of claims 1-29, wherein the affinity elution buffer has conductivity in a range from about 5 mS/cm to about 8 mS/cm, optionally the affinity elution buffer has conductivity in a range from about 5.5 mS/cm to about 7 mS/cm.

31. The population of any one of claims 1-30, wherein the affinity elution buffer has an osmolarity in a range from about 100 mOms to about 225 mOms.

32. The population of any one of claims 1-31, wherein the equilibration buffer for anion exchange chromatography comprises the weak acid or a salt thereof in a concentration of at least about 0.5 mM.

33. The population of any one of claims 1-32, wherein the equilibration buffer comprises the weak acid or a salt thereof at a concentration from about 0.5 mM to about 15 mM.

34. The process of any one of claims 1-33, wherein the weak acid in the equilibration buffer is citric acid, acetic acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid or 3-methylbutanoic acid, or, optionally the weak acid is citric acid, citrate, acetic acid or succinic acid.

35. The population of any one of claims 1-34, wherein the weak acid or salt thereof in the equilibration buffer is citric acid or citrate.

36. The process of any one of claims claim 1-35, wherein adjusting the affinity chromatography eluate for subsequent purification through anion exchange chromatography comprises adding the weak acid or a salt thereof to the affinity eluate.

37. The population of any one of claims 1-36, wherein the weak acid or a salt thereof is added to the affinity eluate to a final a concentration of at least about 0.5 mM or higher.

38. The population of any one of claims 1-37, wherein the weak acid or a salt thereof is added to the affinity eluate to a final concentration from about 0.5 mM to about 15 mM.

39. The population of any one of claims 1-38, wherein the weak acid or a salt thereof added to the affinity eluate is citric acid or a salt thereof, optionally the salt is citrate.

40. The process of any one of claims claim 1-35, wherein adjusting the affinity chromatography eluate for subsequent purification through anion exchange chromatography comprises adding an amino acid to the affinity eluate.

41. The population of any one of claim 1-35 or 40, wherein the amino acid is added to the affinity eluate to a final a concentration of at least about 15 mM or higher.

42. The population of any one of claim 1-35 or 40-41, wherein the amino acid is added to the affinity eluate to a final concentration from about 15 mM to about 35 mM.

43. The population of any one of claim 1-35 or 40-42, wherein amino acid added to the affinity eluate is aspartate, glutamate, histidine, arginine, lysine, cysteine or tyrosine, optionally, the amino acid is histidine.

44. The population of any one of claims 1-43, wherein adjusting the affinity chromatography eluate for subsequent purification through anion exchange chromatography comprises diluting the affinity eluate.

45. The population of any one of claims 1-44, wherein adjusting the affinity eluate for anion exchange chromatography comprises diluting the eluate by at least 2× or more.

46. The population of any one of claims 1-45, wherein adjusting the affinity eluate for anion exchange chromatography comprises diluting the affinity eluate with a dilution buffer (anion exchange dilution buffer).

47. The population of claim 46, wherein the dilution buffer comprises a weak acid or a salt thereof.

48. The population of claim 47, wherein the dilution buffer comprises the weak acid or a salt thereof at a concentration of at least about 0.5 mM.

49. The population of any one of claim 47 or 48, wherein the dilution buffer comprises the weak acid or a salt thereof at a concentration from about 0.5 mM to about 15 mM.

50. The population of any one of claims 47-49, wherein the weak acid in the dilution buffer is citric acid, acetic acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid or 3-methylbutanoic acid, optionally the weak acid is citric acid, citrate, acetic acid or succinic acid.

51. The population of any one of claims 47-50, wherein the weak acid or a salt thereof in the dilution buffer is citric acid or a salt thereof, acetic acid or a salt thereof, or succinic acid or a salt thereof, optionally the weak acid is citric acid or a salt thereof.

52. The population of any one of claims 46-51, wherein the dilution buffer comprises a buffering agent.

53. The population of claim 52, wherein the buffering agent is acetate, histidine, phosphate, citrate, propionate, tricine, borate, or tris(hydroxymethyl)aminomethane (tris), optionally the buffering agent is bris-tris propane (BTP).

54. The population of any one of claims 52-53, wherein the dilution buffer comprises the buffering agent at a concentration of at least about 25 mM.

55. The population of any one of claims 52-54, wherein the dilution buffer comprises the buffering agent at a concentration of from about 25 mM to about 175 mM.

56. The population of any one of claims 46-55, wherein the dilution buffer comprises an amino acid.

57. The population of any one of claims 46-56, wherein the dilution buffer comprises an amino acid at a concentration of at least about 25 mM.

58. The population of any one of claims 46-57, wherein the dilution buffer comprises an amino acid at a concentration of from about 25 mM to about 175 mM.

59. The process of any one of claims 56-58, wherein the amino acid is aspartate, glutamate, histidine, arginine, lysine, cysteine or tyrosine, optionally, the amino acid is histidine.

60. The population of any one of claims 46-59, wherein the dilution buffer comprises a viscosity modifier.

61. The population of claim 60, wherein the viscosity modifier is a polyol, optionally selected from the group consisting of hydrocarbons, monosaccharides, disaccharides, trisaccharides and any combinations thereof.

62. The population of claim 61, wherein the polyol is sorbitol, mannitol, glycerol, propylene glycol, polyethylene glycol, dulcitol, sucrose, lactose, maltose, trehalose, dextran or any combinations thereof, optionally the glycerol, sorbitol, mannitol, dulcitol, sucrose, lactose, maltose, trehalose and any combinations thereof

63. The population of any one of claim 61-62, wherein the polyol is sorbitol, mannitol, glycerol, propylene glycol, polyethylene glycol, dulcitol, sucrose, lactose, maltose, trehalose, dextran or any combinations thereof, optionally the polyol is glycerol.

64. The population of any one of claims 60-63, wherein the dilution buffer comprises the viscosity modifier at a concentration of at least about 0.5% (v/v or w/v) or higher.

65. The population of any one of claims 60-64, wherein the dilution buffer comprises the viscosity modifier at a concentration of from about 0.5% to about 9.5%, (v/v or w/v).

66. The population of any one of claims 46-65, wherein the dilution buffer comprises a non-ionic surfactant.

67. The population of any one of claims 46-66, wherein the dilution buffer comprises a non-ionic surfactant at a concentration of at least about 0.05% (v/v or w/v) or higher.

68. The population of any one claims 46-67, wherein the dilution buffer comprises a non-ionic surfactant at a concentration of about 0.05% to about 0.95%, (v/v or w/v).

69. The population of any one of claims 66-68, wherein the non-ionic surfactant is selected from the group consisting of polyoxyethylene fatty alcohol ethers, polyoxyethylene alkylphenyl ethers, polyoxyethylene-polyoxypropylene block copolymers, alkylglucosides, alkylphenol ethoxylates, preferably polysorbates, polyoxyethylene alkyl phenyl ethers, and any combinations thereof.

70. The population of any one of claims 46-69, wherein the dilution buffer comprises a salt.

71. The population of any one of claims 46-70, wherein the dilution buffer comprises a salt at a concentration of at least about 0.1 mM.

72. The population of any one of claims 46-71, wherein the dilution buffer comprises a salt at a concentration of from about 0.1 mM to about 2 mM.

73. The population of any one of claims 70-72, wherein the salt is a magnesium salt, a sodium salt, a potassium salt, an ammonium salt, a calcium salt, a copper salt, a cobalt salt, a manganese salt, a nickel salt or a zinc salt, optionally the salt is MgCl₂.

74. The population of any one of claims 46-73, wherein the dilution buffer has a high pH.

75. The population of any one of claims 46-74, wherein the dilution buffer has a pH greater than or equal to about 8.

76. The population of any one of claim 46 or 52-75, wherein the dilution buffer comprises:
BTP, histidine, glycerol, PF68, MgCl₂ and has a high pH.

77. The population of any one of claims 46-76, wherein the dilution buffer comprises: BTP, histidine, PF68, MgCl₂, citric acid and has a high pH.

78. The population of any one of claims 1-77, wherein the dilution buffer has conductivity in a range from about 0.5 mS/cm to about 3 mS/cm.

79. The population of any one of claims 1-78, wherein the dilution buffer has an osmolarity of less than 900 mOsm.

80. The population of any one of claims 1-79, further comprising a step of removing or reducing amount of impurities (e.g., host cell DNA (hcDNA)) from the harvest media prior to affinity purification.

81. The population of claim 80, wherein said removing or reducing the amount of impurities comprises adding a cationic amine or nuclease to the harvest media.

82. The population of claim 80 or 81, wherein said removing or reducing the amount of impurities comprises adding a selective precipitation agent to harvest media.

83. The population of any one of claims 1-82, further comprising a step of lysing a host cell in the harvest media with a non-ionic surfactant prior purifying/isolating by affinity chromatography.

84. The population of claim 83, wherein the non-ionic surfactant is added to the harvest media to a final concentration of at least about 0.05% (v/v or w/v) or higher.

85. The population of claim 83 or 84, wherein the non-ionic surfactant is added to the harvest media to a final concentration of from about 0.05% to about 1% (v/v or w/v).

86. The population of any one of claims 83-85, wherein the non-ionic surfactant is mixed with the harvest media for a period of from about 15 minutes to about 2 hours.

87. The population of any one of claims 83-86, wherein the non-ionic surfactant is not Triton X-100.

88. The population of any one of claims 83-87, wherein the non-ionic surfactant is selected from the group consisting of polyoxyethylene fatty alcohol ethers, polyoxyethylene alkylphenyl ethers, polyoxyethylene-polyoxypropylene block copolymers, alkylglucosides, alkylphenol ethoxylates, preferably polysorbates, polyoxyethylene alkyl phenyl ethers, and any combinations thereof.

89. The population of any one of claims 1-88, wherein the recombinant adeno associated virus particle comprise rAAV virion.

90. The population of any one of claims 1-89, wherein the purified rAAV has a particle to infectivity ratio less than 2×10⁴ vg/TCID50.

91. The population of any one of claims 1-90, wherein the population of purified rAAV comprises less than about 10% empty viral capsids.

92. The population of any one of claims 1-91, wherein the wherein the rAAV is obtained by a method comprising transfecting a suspension mammalian cell line, and wherein the cells optionally are transfected in suspension.

93. The population any one of claim 92, wherein, the mammalian cell line is transfected in suspension with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements.

94. The population claim 92 or 93, wherein the mammalian cell line is derived from a human embryonic cell line.

95. The population of any one of claims 92-94, wherein the human embryonic cell line is suspension adapted, serum free cell line derived from a human embryonic kidney cell line.

96. The population of any one of claims 92-95, wherein the purified recombinant adeno-associated virus (rAAV) lacks prokaryotic sequences.

97. The population of any one of claims 1-96, wherein the population is comprised in a composition comprising the population and wherein: (i) wherein the composition exhibits substantially no aggregation of the rAAV particles after two or more freeze thaw cycles; and/or (ii) the purified rAAV particle retains its TCID₅₀/ml by at least about 80% after two or more freeze thaw cycles; and/or (iii) the purified rAAV has a particle to infectivity ratio less than 2×10⁴ vg/TCID₅₀; and/or (iv) the population of purified rAAV comprises less than about 35% empty viral capsids.

98. The population of claim 97, wherein the composition exhibits substantially no aggregation of the rAAV particles after two or more freeze thaw cycles.

99. The population of any one of claims 97-98, wherein the purified rAAV particle retains its TCID₅₀/ml by at least about 80% after two or more freeze thaw cycles.

100. The population of any one of claims 97-99, wherein the purified rAAV has a particle to infectivity ratio less than 2×10⁴ vg/TCID₅₀.

101. The population of any one of claims 97-100, wherein the population of purified rAAV comprises less than about 10% empty viral capsids.

102. The population of any one of claims 97-101, wherein the composition comprises the purified rAAV are at a concentration of from about 1e⁹ vg/ml to about 1e¹⁵ vg/ml.

103. The population of any one of claims 97-102, wherein the composition comprises the purified rAAV are at a concentration of from about 1e¹² vg/ml to about 1e¹⁵ vg/ml, optionally, the purified rAAV are at a concentration of from about 1e¹³ vg/ml to about 1e¹⁴ vg/ml.

104. The population of any one of claims 97-103, wherein the composition has a pH of about 6.5 to about 8, optionally, the composition has a pH of about 7 to about 8.

105. The population of any one of claims 97-104, wherein the composition comprises a buffer, a bulking agent and one or both of a non-ionic surfactant and a multivalent salt, and optionally: (i) the composition is substantially free of glycine; and/or (ii) the composition is substantially free of trehalose, trehalose dehydrate, Dextran T10 or Dextran T40; and/or (iii) the composition is substantially free of sodium salts, ammonium salts or potassium salts; and/or (iv) the composition is substantially free of polysorbate-80 (PS80).

106. The population of claim 105, wherein the buffer is PBS, Tris.HCl, phosphate, citric acid, histidine, tromethamine, succinic acid, malic acid, α-ketoglutaric acid, carbonate, protein buffers or any combinations thereof, optionally, the buffer is PBS, Tris or histidine buffer.

107. The population of claim 105 or 106, wherein the bulking agent is a polyol or providone (PVP K24).

108. The population of claim 107, wherein the polyol is sorbitol, mannitol, glycerol, propylene glycol, polyethylene glycol, dulcitol, sucrose, lactose or maltose, optionally the polyol is sorbitol or mannitol.

109. The population of any one of claims 105-108, wherein the non-ionic surfactant is selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof.

110. The population of any one of claims 105-109, wherein the non-ionic surfactant is selected from the group of Brij 010, BRIJ 020, Brij Cl 0, Brij S20, BrijS10, ECOSURF EH-14, ECOSURF EH-9, ECOSURF SA-15, IGEPAL CA-720, IGEPAL CO-630, IGEPAL CO-720, MERPOL HCS surfactant, MERPOL OJ surfactant, MERPOL SH surfactant, Pluronic 10R5, Pluronic 10R5, Pluronic F-68, Poloxamer P 188, Poloxamer P 338, Poloxamer P188, Poloxamer P407, Polyoxyethylene (12) tridecyl ether, TERGITOL 15-S-12, TERGITOL 15-S-7, TERGITOL 15-S-9, TERGITOL L-64, TERGITOL NP-10, TERGITOL NP-10, TERGITOL NP-11, TERGITOL NP-12, TERGITOL NP-8, TERGITOL NP-9, TERGITOL NP-9.5, TERGITOL NP-9.5, TERGITOL15-S-9, TERGITOLNP-13, TERGITOLNP-7, TWEEN 60 nonionic detergent, and any combinations thereof.

111. The population of any one of claims 105-110, wherein the multivalent salt is a calcium salt, citrate salt, sulfate salt or magnesium salt.

112. The population of any one of claims 105-111, wherein the composition has an osmolarity of less than 750 mOSm.

113. The population of any one of claims 105-112, wherein the composition has an osmolarity of from about 125 mOsm to about 500 mOsm.

114. The population any one of claims 105-113, wherein the composition is a pharmaceutical composition.

115. A process for purifying or isolating recombinantly expressed adeno associated virus particles from a harvesting media, the process comprising:
a. purifying/isolating a plurality of recombinantly expressed virus particles from the harvesting media via affinity chromatography to produce an eluate (affinity chromatography eluate) comprising the plurality of recombinantly expressed virus particles, wherein an elution buffer for affinity chromatography (affinity elution buffer) comprises a predetermined amount of glycine, optionally, the affinity elution buffer is substantially free of weak acids or salts thereof; and optionally, the affinity elution buffer comprises an amino acid that is not glycine;
b. adjusting the affinity chromatography eluate for subsequent purification through anion exchange chromatography, wherein the adjusted eluate comprises a predetermined amount of a weak acid or a salt thereof or the adjusted affinity eluate comprises a predetermined amount of an amino acid in addition to the predetermined amount of glycine already present in affinity eluate; and
c. purifying/isolating the plurality of recombinantly expressed virus particles from the adjusted eluate of affinity chromatography by anion exchange chromatography to produce a solution comprising a plurality of purified/isolated recombinantly expressed virus particles, wherein an equilibration buffer for anion exchange chromatography comprises a predetermined amount of a weak acid or a salt thereof, optionally the weak acid is citric acid, acetic acid or succinic acid.

116. The population of claim 115, wherein less than 5% of empty virus particles in the affinity eluate bind to anion exchange chromatography media.

117. The process of claim 116, wherein substantially no empty virus particles in the affinity eluate bind to anion exchange chromatography media.

118. The process of any one of claims 115-117, wherein less than 10% of the virus particles in the eluate from the anion exchange are empty viral particles.

119. The process of any one of claims 115-118, wherein in less than 5% of the virus particles in the eluate from the anion exchange are empty viral particles.

120. The process of any one of claim 1, wherein the eluate from the anion exchange is substantially free of empty virus particles.

121. The process of any one of claims 1-103, wherein the recombinantly expressed virus particles are recombinant adeno associated virus (rAAV) particle.

122. The process of any one of claims 115-121, wherein the weak acid of 115 (b) is citric acid, acetic acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid or 3-methylbutanoic acid, optionally the weak acid is citric acid, citrate, acetic acid or succinic acid.

123. The process of any one claims 115-122, wherein the affinity elution buffer comprises histidine at a concentration of at least about 1 mM or more.

124. The process of any one claims 115-123, wherein the affinity elution buffer comprises histidine at a concentration from about 1 mM to about 50 mM.

125. The process of any one of claims 115-124, wherein the affinity elution buffer comprises glycine at a concentration of at least about 20 mM or more.

126. The process of any one of claims 115-128, wherein the affinity elution buffer comprises glycine at a concentration of from about 25 mM to about 100 mM.

127. The process of any one of claims 115-126, wherein the affinity elution buffer comprises a salt.

128. The process of any one of claims 115-127, wherein the affinity elution buffer comprises a salt at concentration of at least about 5 mM or more.

129. The process of any one of claims 115-128, wherein the affinity elution buffer comprises a salt concentration of from about 5 mM to about 15 mM.

130. The process of any one of claims 127-129, wherein the salt is a magnesium salt, a sodium salt, a potassium salt, an ammonium salt, a calcium salt, a copper salt, a cobalt salt, a manganese salt, a nickel salt or a zinc salt, optionally the salt is MgCl₂.

131. The process of any one of claims 115-130, wherein the affinity elution buffer comprises a polymer.

132. The process of any one of claims 115-131, wherein the affinity elution buffer comprises a polymer at a concentration of at least about 0.1% or more.

133. The process of any one of claims 115-132, wherein the affinity elution buffer comprises a polymer at a concentration of from about 0.1% to about 0.5%.

134. The process of any one of claims 131-133, the polymer is a non-ionic surfactant.

135. The process of any one of claims 115-134, wherein the affinity elution buffer has a low pH.

136. The process of any one of claims 115-135, wherein the affinity elution buffer has a pH lower than or equal to about 6.5.

137. The process of any one of claims 115-136, wherein the affinity elution buffer has a pH of from about 2.0 to about 3.0.

138. The process of any one of claims 115-137, wherein the affinity elution buffer comprises: about 75 mM glycine.

139. The process of any one of claim 115-122 or 125-138, wherein the affinity elution buffer comprises a weak acid or a salt thereof.

140. The process of claim 139, wherein the affinity elution buffer comprises the weak acid or a salt thereof at a concentration of at least about 50 mM or more.

141. The process of claim 139 or 140, wherein the affinity elution buffer comprises the weak acid or a salt thereof at a concentration of from about 50 mM to about 100 mM.

142. The process of any one of claims 139-141, wherein the weak acid is citric acid, acetic acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid or 3-methylbutanoic acid, optionally the weak acid is citric acid, citrate, acetic acid or succinic acid.

143. The process of any one of claims 139-142, wherein the weak acid is citric acid or a salt thereof.

144. The process of any one of claim 115-122, 125-137 or 139-143, wherein the affinity elution buffer comprises: about 50 mM glycine, about 75 mM citrate, about 10 mM MgCl₂, about 0.3% (w/v) P188 and has a pH of about 3.0.

145. The process of any one of claims 115-144, wherein the affinity elution buffer has conductivity in a range from about 5 mS/cm to about 8 mS/cm.

146. The process of any one of claims 115-145, wherein the affinity elution buffer has an osmolarity in a range from about 100 mOms to about 225 mOms.

147. The process of any one of claims 115-146, wherein the equilibration buffer for anion exchange chromatography comprises the weak acid or a salt thereof in a concentration of at least about 0.5 mM.

148. The process of any one of claims 115-147, wherein the equilibration buffer comprises the weak acid or a salt thereof at a concentration from about 0.5 mM to about 15 mM.

149. The process of any one of claims 115-149, wherein the weak acid in the equilibration buffer is citric acid, acetic acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid or 3-methylbutanoic acid, optionally the weak acid is citric acid, citrate, acetic acid or succinic acid.

150. The process of any one of claims 115-149, wherein the weak acid or salt thereof in the equilibration buffer is citric acid or citrate.

151. The process of any one of claims claim 115-150, wherein adjusting the affinity chromatography eluate for subsequent purification through anion exchange chromatography comprises adding the weak acid or a salt thereof to the affinity eluate.

152. The process of any one of claims 115-151, wherein the weak acid or a salt thereof is added to the affinity eluate to a final a concentration of at least about 0.5 mM or higher.

153. The process of any one of claims 115-152, wherein the weak acid or a salt thereof is added to the affinity eluate to a final concentration from about 0.5 mM to about 15 mM.

154. The process of any one of claims 115-153, wherein the weak acid or a salt thereof added to the affinity eluate is citric acid or a salt thereof, optionally the salt is citrate.

155. The process of any one of claims claim 115-150, wherein adjusting the affinity chromatography eluate for subsequent purification through anion exchange chromatography comprises adding an amino acid to the affinity eluate.

156. The process of any one of claim 115-150 or 155, wherein the amino acid is added to the affinity eluate to a final a concentration of at least about 15 mM or higher.

157. The process of any one of claim 115-150 or 155-156, wherein the amino acid is added to the affinity eluate to a final concentration from about 15 mM to about 35 mM.

158. The process of any one of claim 115-150 or 155-157, wherein amino acid added to the affinity eluate is aspartate, glutamate, histidine, arginine, lysine, cysteine or tyrosine, optionally, the amino acid is histidine.

159. The process of any one of claims 115-158, wherein adjusting the affinity chromatography eluate for subsequent purification through anion exchange chromatography comprises diluting the affinity eluate.

160. The process of any one of claims 115-159, wherein adjusting the affinity eluate for anion exchange chromatography comprises diluting the eluate by at least 2× or more.

161. The process of any one of claims 115-160, wherein adjusting the affinity eluate for anion exchange chromatography comprises diluting the affinity eluate with a dilution buffer (anion exchange dilution buffer).

162. The process of claim 161, wherein the dilution buffer comprises a weak acid or a salt thereof.

163. The process of claim 162, wherein the dilution buffer comprises the weak acid or a salt thereof at a concentration of at least about 0.5 mM.

164. The process of any one of claim 162 or 163, wherein the dilution buffer comprises the weak acid or a salt thereof at a concentration from about 0.5 mM to about 15 mM.

165. The process of any one of claims 162-164, wherein the weak acid in the dilution buffer is citric acid, acetic acid, succinic acid, acetoacetic acid, adipic acid, alloxanic acid, ascorbic acid, aspartic acid, barbituric acid, boric acid, butanoic acid, butyric acid, carbonic acid, crotonic acid, diglycolic acid, dimethylmalonic acid, formic acid, fumaric acid, gluconic acid, glucuronic acid, glutamic acid, glutaric acid, glyceric acid, glycolic acid, hydroxyacetic acid, isocitric acid, itaconic acid, lactic acid, maleic acid, malic acid, malonic acid, mesaconic acid, mesotartaric acid, methylsuccinic acid, methymalonic acid, oxalic acid, oxaloacetic acid, pentanic acid, pentanoic acid, phosphoric acid, m-phthalic acid, o-phthalic acid, p-phthalic acid, propionic acid, pyruvic acid, salicylic acid, tartaric acid, tartronic acid, terephthalic acid, trans-crotonic acid, trichloroacetic acid, uric acid, α-tartaric acid, 2 oxo-butanoic acid, 2-methylbutanoic acid, 2-oxoglutaric acid, 3-butenoic acid or 3-methylbutanoic acid, optionally the weak acid is citric acid, citrate, acetic acid or succinic acid.

166. The process of any one of claims 162-165, wherein the weak acid or a salt thereof in the dilution buffer is citric acid or a salt thereof, acetic acid or a salt thereof, or succinic acid or a salt thereof, optionally the weak acid is citric acid or a salt thereof.

167. The process of any one of claims 162-166, wherein the dilution buffer comprises a buffering agent.

168. The process of claim 167, wherein the buffering agent is acetate, histidine, phosphate, citrate, propionate, tricine, borate, or tris(hydroxymethyl)aminomethane (tris), optionally the buffering agent is bris-tris propane (BTP).

169. The process of any one of claims 167-168, wherein the dilution buffer comprises the buffering agent at a concentration of at least about 25 mM.

170. The process of any one of claims 167-169, wherein the dilution buffer comprises the buffering agent at a concentration of from about 25 mM to about 175 mM.

171. The process of any one of claims 161-170, wherein the dilution buffer comprises an amino acid.

172. The process of any one of claims 161-171, wherein the dilution buffer comprises an amino acid at a concentration of at least about 25 mM.

173. The process of any one of claims 161-172, wherein the dilution buffer comprises an amino acid at a concentration of from about 25 mM to about 175 mM.

174. The process of any one of claims 161-173, wherein the amino acid is aspartate, glutamate, histidine, arginine, lysine, cysteine or tyrosine, optionally, the amino acid is histidine.

175. The process of any one of claims 161-174, wherein the dilution buffer comprises a viscosity modifier.

176. The process of claim 175, wherein the viscosity modifier is a polyol, optionally selected from the group consisting of hydrocarbons, monosaccharides, disaccharides, trisaccharides and any combinations thereof.

177. The process of claim 176, wherein the polyol is sorbitol, mannitol, glycerol, propylene glycol, polyethylene glycol, dulcitol, sucrose, lactose, maltose, trehalose, dextran or any combinations thereof, optionally the glycerol, sorbitol, mannitol, dulcitol, sucrose, lactose, maltose, trehalose and any combinations thereof

178. The process of any one of claims 176-177, wherein the polyol is sorbitol, mannitol, glycerol, propylene glycol, polyethylene glycol, dulcitol, sucrose, lactose, maltose, trehalose, dextran or any combinations thereof, optionally the polyol is glycerol.

179. The process of any one of claims 175-178, wherein the dilution buffer comprises the viscosity modifier at a concentration of at least about 0.5% (v/v or w/v) or higher.

180. The process of any one of claims 175-179, wherein the dilution buffer comprises the viscosity modifier at a concentration of from about 0.5% to about 9.5%, (v/v or w/v).

181. The process of any one of claims 161-180, wherein the dilution buffer comprises a non-ionic surfactant.

182. The process of any one of claims 161-181, wherein the dilution buffer comprises a non-ionic surfactant at a concentration of at least about 0.05% (v/v or w/v) or higher.

183. The process of any one claims 161-182, wherein the dilution buffer comprises a non-ionic surfactant at a concentration of about 0.05% to about 0.95%, (v/v or w/v).

184. The process of any one of claims 181-183, wherein the non-ionic surfactant is selected from the group consisting of polyoxyethylene fatty alcohol ethers, polyoxyethylene alkylphenyl ethers, polyoxyethylene-polyoxypropylene block copolymers, alkylglucosides, alkylphenol ethoxylates, preferably polysorbates, polyoxyethylene alkyl phenyl ethers, and any combinations thereof.

185. The process of any one of claims 161-184, wherein the dilution buffer comprises a salt.

186. The process of any one of claims 161-185, wherein the dilution buffer comprises a salt at a concentration of at least about 0.1 mM.

187. The process of any one of claims 161-186, wherein the dilution buffer comprises a salt at a concentration of from about 0.1 mM to about 2 mM.

188. The process of any one of claims 185-187, wherein the salt is a magnesium salt, a sodium salt, a potassium salt, an ammonium salt, a calcium salt, a copper salt, a cobalt salt, a manganese salt, a nickel salt or a zinc salt, optionally the salt is MgCl₂.

189. The process of any one of claims 161-188, wherein the dilution buffer has a high pH.

190. The process of any one of claims 161-189, wherein the dilution buffer has a pH greater than or equal to about 8.

191. The process of any one of claim 161 or 167-190, wherein the dilution buffer comprises: BTP, histidine, glycerol, PF68, MgCl₂ and has a high pH.

192. The process of any one of claims 161-191, wherein the dilution buffer comprises: BTP, histidine, PF68, MgCl₂, citric acid and has a high pH.

193. The process of any one of claims 161-192, wherein the dilution buffer has conductivity in a range from about 0.5 mS/cm to about 3 mS/cm.

194. The process of any one of claims 161-193, wherein the dilution buffer has an osmolarity of less than 900 mOsm.

195. The process of any one of claims 115-194, wherein less than 5% of empty virus particles in the affinity eluate bind to anion exchange chromatography media.

196. The process of any one of claims 115-195, wherein less than 10% of the virus particles in the eluate from the anion exchange are empty viral particles.

197. The process of any one of claims 115-196, wherein the eluate from the anion exchange is substantially free of empty virus particles.

198. The process of any one of claims 115-197, wherein a ratio of UV₂₆₀ to UV₂₈₀ in the anion exchange eluate is at least 1.15× higher than a ratio of UV₂₆₀ to UV₂₈₀ ratio in the adjusted affinity eluate.

199. The process of any one of claims 115-198, wherein the recombinant adeno associated virus particle comprise rAAV virion.

200. The process of any one of claims 115-199, wherein the purified rAAV has a particle to infectivity ratio less than 2×10⁴ vg/TCID50.

201. A population of recombinantly expressed virus particles purified or isolated by a method of any one of claims 115-200.

202. A composition comprising a population of recombinantly expressed virus particles purified or isolated by a method of any one of claims 115-200.

203. The composition of claim 202, wherein the composition is a pharmaceutical composition.

204. A population of purified recombinant adeno-associated virus (rAAV) lacking prokaryotic sequences, wherein, the purified rAAV has a particle to infectivity ratio less than 2×10⁴ vg/TCID50, wherein the population of purified rAAV comprises less than about 10% empty viral capsids, and wherein the purified rAAV optionally is obtained by a method comprising transfecting a suspension mammalian cell line, and wherein the cells optionally are transfected in suspension.

205. The population of purified recombinant adeno-associated virus (rAAV) of claim 204, wherein, the population comprises less than about 5% empty viral capsids.

206. The population of purified recombinant adeno-associated virus (rAAV) of any one of claims 204-205, wherein, the population is substantially devoid of empty viral capsids.

207. The population of purified recombinant adeno-associated virus (rAAV) any one of claims 204-206, wherein the mammalian cell line is derived from a human embryonic cell line.

208. The population of purified recombinant adeno-associated virus (rAAV) any one of claims 204-207, wherein the human embryonic cell line is suspension adapted, serum free cell line derived from a human embryonic kidney cell line.

209. A population of purified recombinant adeno-associated virus (rAAV) lacking prokaryotic sequences, wherein, the purified rAAV has a particle to infectivity ratio less than 2×10⁴ vg/TCID50, and wherein the purified rAAV is obtained by a method comprising transfecting a suspension mammalian cell line.

210. The population of purified recombinant adeno-associated virus (rAAV) of claim 209, wherein, the mammalian cell line is transfected in suspension with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements.

211. The population of purified recombinant adeno-associated virus (rAAV) of any one of claims 209-210, wherein the mammalian cell line is derived from a human embryonic cell line.

212. The population of purified recombinant adeno-associated virus (rAAV) of any one of claims 209-211, wherein the human embryonic cell line is suspension adapted, serum free cell line derived from a human embryonic kidney cell line.

213. A composition comprising a population of purified recombinant adeno-associated virus particles and a pH from about 6.5 to about 8.0, and wherein: (i) the purified rAAV has a particle to infectivity ratio less than 2×10⁴ vg/TCID50; and/or (ii) the population of purified rAAV comprises less than about 10% empty viral capsids.

214. The composition of claim 213, wherein the composition comprises the purified rAAV are at a concentration of from about 1e⁹ vg/ml to about 1e¹⁵ vg/ml.

215. The composition of any one of claims 213-2-14, wherein the composition comprises the purified rAAV are at a concentration of from about 1e¹² vg/ml to about 1e¹⁴ vg/ml, optionally, the purified rAAV are at a concentration of from about 1e¹³ vg/ml to about 1e¹⁴ vg/ml.

216. The composition of any one of claims 213-215, wherein the composition has a pH of from about 6.5 to about 8.0, optionally, the composition has a pH of from about 7 to about 8.0.

217. The composition of any one of claims 213-216, wherein the composition comprises a buffer.

218. The composition of claim 217, wherein the buffer is selected from the group consisting of PBS, Tris.HCl, phosphate, citric acid, histidine, tromethamine, succinic acid, malic acid, α-ketoglutaric acid, carbonate, protein buffers, and any combinations thereof.

219. The composition of claim 217 or 218, wherein the buffer has a salt concentration of from about 20 mM to about 750 mM.

220. The composition of any one of claims 214-219, wherein the composition has an ionic strength of at least 100 mM.

221. The composition of claim 220, wherein the composition has an ionic strength from about 125 mM to about 750 mM.

222. The composition of any one of claims 214-221, wherein the composition has an osmolarity of less than about 600 mOsm.

223. The composition of claim 222, wherein the composition has an osmolarity from about 125 mOsm to about 500 mOsm.

224. The composition of any one of claims 214-223, wherein the composition comprises one or more ions and/or salts thereof.

225. The composition of claim 224, wherein the ion is selected from the group consisting of sodium, potassium, chloride, ammonium, carbonate, nitrate, chlorate, chlorite, and calcium.

226. The composition of any one of claims 214-225, wherein the composition comprises a bulking agent.

227. The composition of claim 226, wherein the bulking agent is a polyol or providone (PVP K24).

228. The composition of any one of claims 226-227, wherein the bulking agent is selected from the group consisting of polyhydroxy hydrocarbons, monosaccharides, disaccharides, and trisaccharides.

229. The composition of any one of claims 226-228, wherein the bulking agent is selected from the group consisting of sorbitol, mannitol, glycerol, propylene glycol, polyethylene glycol, dulcitol, sucrose, lactose, maltose, trehalose, and dextran.

230. The composition of any one of claims 226-229, wherein the composition comprises the bulking agent at a concentration from about 0.5% (w/v) to about 10% (w/v).

231. The composition of any one of claims 214-230, wherein the composition comprises a non-ionic surfactant.

232. The composition of claim 231, wherein the non-ionic surfactant selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof.

233. The composition of claim 231 or 232, wherein the non-ionic surfactant is selected from the group consisting of TWEEN 60 nonionic detergent, PPG-PEG-PPG Pluronic 10R5, Pluronic F-68, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, Brij S10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof.

234. The composition of any one of claims 231-233, wherein the composition comprises the non-ionic surfactant at a concentration from about 0.005% (w/v) to about 0.015% (w/v).

235. The composition of any one of claims 214-234, wherein the composition comprises one or more multivalent ions or salts thereof.

236. The composition of claim 235, wherein the multivalent ions are selected from the group consisting of citrate, sulfate, magnesium and phosphate.

237. The composition of any one of claims 235-236, wherein the composition comprises the multivalent ions at a concentration from about 5 mM to about 150 mM.

238. The composition of claim 237, wherein the composition comprises the multivalent ions at a concentration from about 10 mM to about 50 mM.

239. The composition of any one of claims 214-238, wherein the composition comprises calcium α-d-heptagluconate at a concentration from about 0.0001% (w/v) to about 0.01% (w/v).

240. The composition of any one of claims 214-239, wherein the population of purified rAAV comprises less than about 5% empty viral capsids.

241. The composition of any one of claims 214-240, wherein the population of purified rAAV is substantially devoid of empty viral capsids.

242. The composition of any one of claims 214-241, wherein the purified rAAV has a particle to infectivity ratio less than 1.5×10⁴ vg/TCID50.

243. The composition of any one of claims 214-242, wherein the composition exhibits substantially no aggregation after two or more freeze thaw cycles.

244. The composition of any one of claims 214-243, wherein the claims wherein the purified rAAV particle retains its TCID₅₀/ml by at least about 80% after two or more freeze thaw cycles.

245. The composition of any one of claims 214-244, wherein the population of purified recombinant adeno-associated virus particles is the population of purified rAAV of any one of claims 115-200.
